(19) United States
(12) Patent Application Publication
(10) Pub. No.: US 2014/0363502 A1
(43) Pub. Date: Dec. 11, 2014

(54) Resveratrol-Containing Compositions and Methods of Use for Treatment of Macular Degeneration

(71) Applicant: Resveratrol Partners, LLC, Las Vegas, NV (US)

(72) Inventor: William F. Sardi, La Verne, CA (US)

(21) Appl. No.: 14/467,704

(22) Filed: Aug. 25, 2014

Related U.S. Application Data
(62) Division of application No. 12/212,494, filed on Sep. 17, 2008, now abandoned.

(51) Int. Cl. A61K 31/352; A61K 45/06; A61K 31/05

(52) U.S. Cl. CPC A61K 31/352; A61K 31/05; A61K 45/06
USPC 424/451; 514/733; 514/456; 514/167

(57) ABSTRACT

The present invention relates to a resveratrol-containing composition capable of modulating gene expression to an extent greater than that observed with resveratrol alone or with calorie restriction. The invention particularly pertains to such resveratrol-containing compositions that comprise resveratrol, a chelator, hyaluronic acid, and/or vitamin D and which upon administration to a recipient, increases the concentration or activity of a survival/longevity gene product and/or decreases the concentration or activity of a gene product that induces or causes cellular damage. Most preferably, the resveratrol-stabilizing composition will comprise the chelator phytic acid (inositol hexaphosphate; IP6), hyaluronic acid, and vitamin D. The invention further pertains to the use of such compositions in the treatment or prevention of cancer, cardiovascular disease, diseases associated with aging, and other conditions and illnesses.

### CROSS-REFERENCE TO RELATED APPLICATIONS

This application claims priority from U.S. patent application Ser. No. 10/971,017 (filed Oct. 25, 2004; pending), which application claims priority to 60/513,225 (filed on Oct. 23, 2003; lapsed), and to U.S. Patent Application Ser. Nos. 60/973,817 (filed on Sep. 20, 2007; pending), 61/023,227 (filed on Jan. 24, 2008; pending), 61/023,230 (filed on Jan. 24, 2008; pending), 61/023,234 (filed on Jan. 24, 2008; pending), 61/048,756 (filed on Apr. 29, 2008; pending) and), 61/048,769 (filed on Apr. 29, 2008; pending) all of which applications are herein incorporated by reference in their entirety.

### FIELD OF THE INVENTION

The present invention relates to a resveratrol-containing composition capable of modulating gene expression to an extent greater than that observed with resveratrol alone or with calorie restriction. The invention particularly pertains to such resveratrol-containing compositions that comprise resveratrol, a chelator, hyaluronic acid, and/or vitamin D and which, upon administration to a recipient, increases the concentration or activity of a survival/longevity gene product and/or decreases the concentration or activity of a gene product that induces or causes cellular damage. The invention particularly relates to the use of such compositions in the treatment or prevention of cancer, cardiovascular disease, diseases associated with aging, and other conditions and illnesses.

### BACKGROUND OF THE INVENTION

Despite a high level of risk factors such as cholesterol, diabetes, hypertension and a high intake of saturated fat, French males display the lowest mortality rate from ischaemic heart disease and cardiovascular diseases in Western industrialized nations (36% lower than the USA and 39% lower than the UK). The so-called ‘French Paradox’ (a low mortality rate specifically from cardiovascular diseases) may be due mainly to the regular consumption of wine (Renaud, S. et al. (1998) “The French Paradox And Wine Drinking,” Novartis Found. Symp. 216:208-222, 152-158).

Resveratrol (3,4′,5-trihydroxy-trans-stilbene) is a naturally occurring phenolic compound found, for example in grape skins, that has been demonstrated to have beneficial properties relating to health of humans (Das, S. et al. (2007) “Resveratrol: A Therapeutic Promise For Cardiovascular Diseases,” Recent Patents Cardiovasc. Drug Discov. 2(2):133-138; Mancuso, C. et al. (2007) “Natural antioxidants in Alzheimer's disease,” Expert Opin. Investig. Drugs. 16(12):1921-1931; Baumann L. (2007) “Botanical Ingredients In Cosmeceuticals,” J. Drugs Dermatol. 6(11):1084-1088; Meeran, S. M. et al. (2008) “Cell Cycle Control As A Basis For Cancer Chemoprevention Through Dietary Agents,” Front. Biosci. 13:2191-2202; de la Lastra, C. A. et al. (2007) “Resveratrol As An Antioxidant And Pro-Oxidant Agent: Mechanisms And Clinical Implications,” Biochem. Soc. Trans. 35(Pt 5):1156-1160; Das, S. et al. (2007) “Anti-Inflammatory Responses Of Resveratrol,” Inflamm. Allergy Drug Targets 6(3):168-173; Cucciolla, V. et al. (Epub 2007 Jul. 31) “Resveratrol: From Basic Science To The Clinic,” Cell Cycle 6(20):2495-2510; Opie, L. H. et al. (Epub 2007 Jun. 7) “The Red Wine Hypothesis: From Concepts To Protective Signaling Molecules,” Eur. Heart J. 28(14):1683-1693; Chen, Y. et al. (2007) “Review. Pro-And Anti-Angiogenesis Effects Of Resveratrol,” In Vivo 21(2):365-370 [Erratum in: In Vivo 21(6):1172 and 21(5):955]; Holme, A. L. et al. (2007) “Resveratrol In Cell Fate Decisions,” J. Bioenerg. Biomembr. 39(1):59-63; Athar, M. et al. (Epub 2007 Jan. 3) “Resveratrol: A Review Of Preclinical Studies For Human Cancer Prevention,” Toxicol. Appl. Pharmacol. 224(3):274-283). In particular, resveratrol is believed to be beneficial to the functioning of the heart and in extending the life of human cells. Resveratrol, when used in dietary supplements, is generally produced as an alcohol extract from plant sources.

Calorie restricted diets have been shown to enhance survival and longevity by up-regulating survival/longevity genes or down-regulating genes whose expression enhances cellular damage (Edwards, M. G. et al. (2007) “Gene Expression Profiling Of Aging Reveals Activation Of A P53-Mediated Transcriptional Program,” BMC Genomics 8:80; Anderson, R. M. et al. (2006) “Calorie Restriction: Progress During Mid-2005-Mid-2006,” Exp. Gerontol. 41(12):1247-1249; Weindruch, R. et al. (2001) “Microarray Profiling of Gene Expression in Aging and Its Alteration by Caloric Restriction in Mice,” J. Nutrition 131:918 S-923S; Lee, K.-C. et al. (2003) “Transcriptional Profiles Associated With Aging And Middle Age-Onset Caloric Restriction In Mouse Hearts,” Proc. Natl. Acad. Sci. (U.S.A.) 99(23): 14988-14993; Weindruch, R. et al. (2002) “Effects Of Caloric Restriction On Gene Expression,” Nestle Nutr. Workshop Ser. Clin. Perform. Programme 6:17-28; 28-32; Mulligan, J. D. et al. (Epub 2007 Nov. 1) “Downregulation Of Plasma Insulin Levels And Hepatic Ppar gamma Expression During The First Week Of Caloric Restriction In Mice,” Exp. Gerontol. 43(3):146-153; Rodgers, J. T. et al. (Epub 2007 Nov. 26) “Metabolic Adaptations Through The PGC-1 Alpha And SIRT1 Pathways,” FEBS Lett. 582(1):46-53; Swindell, W. R. (2007) “Gene Expression Profiling Of Long-Lived Dwarf Mice: Longevity-Associated Genes And Relationships With Diet, Gender And Aging,” BMC Genomics. 8:353; Zhu, M. et al. (Epub 2007 Jun. 6) “Adipogenic Signaling In Rat White Adipose Tissue: Modulation By Aging And Calorie Restriction,” Exp. Gerontol. 42(8):733-744; Chiarpotto, E. et al. (2006) “Molecular Mechanisms Of Calorie Restriction's Protection Against Age-Related Sclerosis,” IUBMB Life. 58(12):695-702; Lu, J. et al. (2007) “Different Gene Expression Of Skin Tissues Between Mice With Weight Controlled By Either Calorie Restriction Or Physical Exercise,” Exp. Biol. Med. (Maywood). 232(4):473-480; Masternak, M. M. et al. (2007) “pPARS In Calorie Restricted And Genetically Long-Lived Mice,” pPAR Res. 2007:28436; Fu, C. et al. (2006) “Tissue Specific And Non-Specific Changes In Gene Expression By Aging And By Early Stage CR,” Mech. Ageing Dev. 127(12):905-916). As indicated by the above-cited references, mice have been used extensively as a model for genetic expression comparisons with humans. Without limitation, the validity of murine models to human gene expression reflects the fact that 98% of human and murine gene are homologous, and that mice and humans have about the same number of genes (e.g., approximately 30,000).

Despite the established benefits of a calorie restricted diet, the severity of the required dietary regime has limited adoption of this approach to increasing longevity. It would therefore be desirable to provide an alternative route to obtaining the benefits of calorie restriction that would avoid the need for dietary regulation and that would be amenable to widespread adoption. The present invention is directed to this and other needs.

### SUMMARY OF THE INVENTION

The present invention relates to a resveratrol-containing composition capable of modulating gene expression to an extent greater than that observed with resveratrol alone or with calorie restriction. The invention particularly pertains to such resveratrol-containing compositions that comprise resveratrol, a chelator, hyaluronic acid, and/or vitamin D and which, upon administration to a recipient, increases the concentration or activity of a survival/longevity gene product and/or decreases the concentration or activity of a gene product that induces or causes cellular damage. Most preferably, the resveratrol-stabilizing composition will comprise the chelator phytic acid (inositol hexaphosphate; IP6), hyaluronic acid, and vitamin D. The invention further pertains to the use of such compositions to up-regulate a survival/longevity gene or down-regulate a gene whose expression enhances cellular damage upon administration to a recipient. The invention particularly relates to the use of such compositions in the treatment or prevention of cancer, cardiovascular disease, diseases associated with aging, and other conditions and illnesses.

In detail, the invention provides a resveratrol-containing composition that, upon administration to a recipient, modulates the concentration or activity, relative to resveratrol alone or calorie restriction, of the product of a survival/longevity gene or the product of a gene whose expression enhances cellular damage. Administration is preferably by oral ingestion.

The invention further provides the embodiments of such compositions wherein the modulation alters:
(A) oxidative phosphorylation;
(B) actin filament length or polymerization;
(C) intracellular transport;
(D) organelle biogenesis;
(E) insulin signaling;
(F) glycolysis;
(G) gluconeogenesis; or
(H) fatty acid metabolism.

The invention further provides the embodiments of such compositions wherein the gene product is a survival/longevity gene product, and especially wherein the gene product is sSirtuin 1, or the forkhead Foxo1 transcription factor.

The invention further provides the embodiments of such compositions wherein the gene product is a gene product that enhances cellular damage, and especially wherein the gene product is encoded by the uncoupling protein 3, Pgc-1, or pyruvate dehydrogenase kinase 4 genes.

The invention further provides the embodiments of such compositions wherein the composition comprises:

(a) trans-resveratrol; and

(b) a metal chelating agent;

wherein the trans-resveratrol is encapsulated to thereby substantially preserve the ability of the composition to modulate the concentration or activity of the product of the survival/longevity gene or the product of the gene whose expression enhances cellular damage, from loss due to exposure of the trans-resveratrol to light or oxygen.

The invention further provides a method of ameliorating a symptom associated with an existing disease of an individual or for preventing the onset of the symptom in an individual prior to the occurrence of the disease in the individual, which comprises administering to the individual, a resveratrol-containing composition that modulates the concentration or activity, relative to resveratrol alone or caloric restriction, of the product of a survival/longevity gene or the product of a gene whose expression enhances cellular damage, wherein the resveratrol is provided in an amount effective to cause a modulation of the concentration or activity of the gene that ameliorates the symptom of the disease, and wherein the disease is selected from the group consisting of: cardiovascular disease, cancer, macular degeneration, a disease associated with aging, and inflammation.

The invention further provides the embodiments of such method wherein the modulation alters:
(A) oxidative phosphorylation;
(B) actin filament length or polymerization;
(C) intracellular transport;
(D) organelle biogenesis;
(E) insulin signaling;
(F) glycolysis;
(G) gluconeogenesis; or
(H) fatty acid metabolism.

The invention further provides the embodiments of such method wherein the survival/longevity gene product is Sirtuin 1 or the forkhead Foxo1 transcription factor. The invention further provides the embodiments of such method wherein the gene whose expression enhances cellular damage encodes uncoupling protein 3 or pyruvate dehydrogenase kinase 4.

The invention further provides the embodiments of such method wherein said composition comprises:

(a) trans-resveratrol; and

(b) a metal chelating agent;

wherein the trans-resveratrol is encapsulated to thereby substantially preserve the ability of the composition to modulate the concentration or activity of the product of the survival/longevity gene or the product of the gene whose expression enhances cellular damage, from loss due to exposure of the trans-resveratrol to light or oxygen.

The invention further provides the embodiments of such method wherein the disease is cancer, or a disease associated with aging (especially a neurodegenerative disease).

The invention further provides the embodiments of such method wherein the composition additionally comprises quercetin, hyaluronic acid and/or vitamin D.

The invention further provides the embodiments of such method wherein the modulation is relative to resveratrol alone or wherein the modulation is relative to calorie restriction.

The invention further provides the embodiments of such methods wherein the gene product is a survival/longevity gene product, and especially wherein the gene product is Sirtuin 1, or the forkhead Foxo1 transcription factor.

The invention further provides the embodiments of such methods wherein the gene product is a gene product that enhances cellular damage, and especially wherein the gene product is encoded by the uncoupling protein 3, Pgc-1, or pyruvate dehydrogenase kinase 4 gene.

### BRIEF DESCRIPTION OF THE FIGURES

FIG. 1 shows the change in body weight of mice administered resveratrol or a composition of the present invention (Longevinex®) relative to control animals and animals maintained on a calorie restricted diet.

FIG. 2 shows the serum insulin level of mice administered resveratrol or a composition of the present invention (Longevinex®) relative to control animals and animals maintained on a calorie restricted diet.

FIG. 3 shows the serum glucose level of mice administered resveratrol (P=0.97) or a composition of the present invention (Longevinex®) (P=0.07) relative to control animals and animals maintained on a calorie restricted diet (P=0.10).

FIG. 4 shows a schematic of a mechanism of action that is consistent with the observed biological activities of the compositions of the present invention.

### DESCRIPTION OF THE PREFERRED EMBODIMENTS

The present invention relates to a resveratrol-containing composition (and especially a resveratrol-containing dietary composition (i.e., a composition amenable for oral ingestion by a recipient)) capable of modulating gene expression to an extent greater than that observed with resveratrol alone or with calorie restriction. The invention particularly pertains to such resveratrol-containing compositions that comprise resveratrol, a chelator, hyaluronic acid, and/or vitamin D and which up-regulate a survival/longevity gene or down-regulate a gene whose expression enhances cellular damage upon administration to a recipient. Most preferably, the resveratrol-stabilizing composition will comprise the chelator phytic acid (inositol hexaphosphate; IP6), hyaluronic acid, and vitamin D. The invention further pertains to the use of such compositions to up-regulate a survival/longevity gene or down-regulate a gene whose expression enhances cellular damage upon administration to a recipient. The mineral chelators of the present invention provide anti-aging effects, as evidence in differentiation of the genome.

A. Resveratrol

As used herein, the term “resveratrol” refers to the phytoalexin: 3,4′,5-trihydroxy-trans-stilbene having the structure:

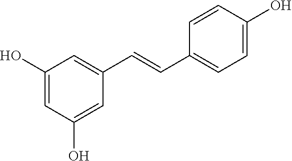 [SMILES: Oc1ccc(/C=C/c2cc(O)cc(O)c2)cc1]

Resveratrol has been ascribed multiple beneficial biological effects (see, U.S. Pat. No. 7,345,178, which listing of disclosed effects is herein incorporated by reference), including preventing or treating cardiovascular disease (see, e.g., Das, S. et al. (2007) “Resveratrol: A Therapeutic Promise For Cardiovascular Diseases,” Recent Patents Cardiovasc. Drug Discov. 2(2):133-138), Opie, L. H. et al. (Epub 2007 Jun. 7) “The Red Wine Hypothesis: From Concepts To Protective Signaling Molecules,” Eur. Heart J. 28(14):1683-1693; Bertelli, A. A. (Epub 2007 May 24) “Wine, Research And Cardiovascular Disease: Instructions For Use,” Atherosclerosis 195 (2):242-247; Providência, R. (2006) “Cardiovascular Protection From Alcoholic Drinks: Scientific Basis Of The French Paradox,” Rev. Port. Cardiol. 25(11):1043-1058; Maulik, N. (2006) “Reactive Oxygen Species Drives Myocardial Angiogenesis?” Antioxid. Redox Signal. 8(11-12):2161-2168; Olas, B. et al. (2005) “Resveratrol, A Phenolic Antioxidant With Effects On Blood Platelet Functions,” Platelets 16(5):251-260; Bradamante, S. et al. (2004) “Cardiovascular Protective Effects Of Resveratrol,” Cardiovasc Drug Rev. 22(3):169-188; Hao, H. D. et al. (2004) “Mechanisms Of Cardiovascular Protection By Resveratrol,” J. Med. Food 7(3):290-298), preventing or treating cancer (Jang, M. et al. (1997) “Cancer Chemopreventive Activity Of Resveratrol, A Natural Product Derived From Grapes,” Science 275:218-220; Das, S. et al. (2007) “Anti-Inflammatory Responses Of Resveratrol,” Inflamm. Allergy Drug Targets 6(3):168-173; de la Lastra, C. A. et al. (2007) “Resveratrol As An Antioxidant And Pro-Oxidant Agent: Mechanisms And Clinical Implications,” Biochem. Soc. Trans. 35(Pt 5):1156-1160; Athar, M. et al. (Epub 2007 Jan. 3) “Resveratrol: A Review Of Preclinical Studies For Human Cancer Prevention,” Toxicol. Appl. Pharmacol. 224(3):274-283), Mccran, S. M. et al. (2008) “Cell Cycle Control As A Basis For Cancer Chemoprevention Through Dietary Agents,” Front. Biosci. 13:2191-2202; Shankar, S. et al. (2007) “Chemoprevention By Resveratrol: Molecular Mechanisms And Therapeutic Potential,” Front. Biosci. 12:4839-4854; Delmas, D. et al. (2006) “Resveratrol As A Chemopreventive Agent: A Promising Molecule For Fighting Cancer,” Curr. Drug Targets 7(4):423-442; Signorelli, P. et al. (2005) “Resveratrol As An Anticancer Nutrient: Molecular Basis, Open Questions And Promises,” J. Nutr. Biochem. 16(8):449-466; preventing or treating macular degeneration (U.S. Patent Application Ser. No. 61/023,234; King, R. E. et al. (2005) “Resveratrol reduces oxidation and proliferation of human retinal pigment epithelial cells via extracellular signal-regulated kinase inhibition,” Chem. Biol. Interact. 151(2):143-149; Sparrow, J. R. et al. (2003) “A2E-epoxides damage DNA in retinal pigment epithelial cells. Vitamin E and other antioxidants inhibit A2E-epoxide formation,” J. Biol. Chem. 278(20):18207-18213), attenuating or preventing diseases associated with aging, and other conditions and illnesses, including the incidence or severity of neurodegenerative diseases such as Alzheimer's Disease and Parkinson's Disease (Baxter, R. A. (2008) “Anti-Aging Properties Of Resveratrol: Review And Report Of A Potent New Antioxidant Skin Care Formulation,” J. Cosmet. Dermatol. 7(1):2-7; Engel, N. et al. (2008) “Aging And Anti-Aging: Unexpected Side Effects Of Everyday Medication Through Sirtuin1 Modulation,” Int. J. Mol. Med. 21(2):223-232; Bickenbach, K. A. et al. (Epub 2007 Dec. 21) “Resveratrol Is An Effective Inducer Of Carg-Driven Tnf-Alpha Gene Therapy,” Cancer Gene Ther. 15(3):133-139; Putics, A. et al. (2008) “Resveratrol Induces The Heat-Shock Response And Protects Human Cells From Severe Heat Stress,” Antioxid. Redox Signal. 10(1):65-75; Bass, T. M. et al. (Epub 2007 Aug. 14) “Effects Of Resveratrol On Lifespan In Drosophila melanogaster And Caenorhabditis elegans,” Mech. Ageing Dev. 128(10):546-552; Stefani, M. et al. (Epub 2007 Sep. 5) “The Effect Of Resveratrol On A Cell Model Of Human Aging,” Ann. N.Y. Acad. Sci. 1114:407-418; Heiss, E. H. et al. (Epub 2007 Jul. 11) “Chronic Treatment With Resveratrol Induces Redox Stress-And Ataxia Telangiectasia-Mutated (Atm)-Dependent Senescence In P53-Positive Cancer Cells,” J. Biol. Chem. 282(37):26759-26766; Mayo Clinic (2007) “A Compound In Red Wine Makes Mice Live Longer, Healthier,” Mayo Clin. Health Lett. 25(5):4; Kim, D. et al. (Epub 2007 Jun. 21) “Sirt1 Deacetylase Protects Against Neurodegeneration In Models For Alzheimer's Disease And Amyotrophic Lateral Sclerosis,” EMBO J. 26(13):3169-3179), and anti-inflammatory activity (Das, S. et al. (2007) “Anti-Inflammatory Responses Of Resveratrol,” Inflamm. Allergy Drug Targets 6(3):168-173).

Reseveratrol may be synthesized chemically (Farina, A. et al. (2006) “An Improved Synthesis Of Resveratrol,” Nat. Prod. Res. 20(3):247-252), or, more preferably, may be extracted from plant sources. Resveratrol is found in at least 72 species of plants distributed among 31 genera and 12 families (see, Counet, C. et al. (2006) “Chocolate And Cocoa: New Sources Of Trans-Resveratrol And Trans-Piceid,” Food Chem. 98:649-657; Sang, M. et al. (1997) “Cancer Chemopreventive Activity Of Resveratrol, A Natural Product Derived From Grapes,” Science 275:218-220; Wang, Y. et al. (2002) “An LC-MS Method For Analyzing Total Resveratrol In Grape Juice, Cranberry Juice, And In Wine,” J. Agricult. Food Chem. 50(3):431-435). All of the families found to contain resveratrol belong to the spermatophytes division: Vitaceae, Myrtaceae, Dipterocarpaceae, Cyperaceae, Gnetaceae, Leguminosae, Pinaceae, Moraceae, Fagaceae, Liliaceae (Langcake, P. et al. (1976) “The Production Of Resveratrol By Vitis Vinifera And Other Members Of The Vitaceae As A Response To Infection Or Injury,” Physiol. Plant Pathol. 9:77-86; Yoshiaki, T. et al. (2002) “Biogenic Reactions On Stilbenetetramers From Vitaceaeous Plants,” Tetrahedron 58:9265-9271). Resveratrol has most often been reported in nonedible plants: vine, eucalyptus, spruce, and the tropical deciduous tree Bauhinia racemosa, Pterolobium Hexapetallum (Cassady, A. et al. (2000) “Isoflavones, Lignans, And Stilbenes-Origins, Metabolism And Potential Importance To Human Health,” J. Science Food Agric. 80:1044-1062; Soleas, G. J. et al. (1997) “Resveratrol: A Molecule Whose Time Has Come? And Gone?,” Clin. Biochem. 30:91-113). Reseveratrol is particularly found in grape skins and Giant Knotweed (see, Burns, J. et al. (2002) “Plant Foods and Herbal Sources of Resveratrol,” J. Agric. Food Chem. 50(11):3337-3340), cocoa and chocolate (Counet, C. et al. (2006) “Chocolate And Cocoa: New Sources Of Trans-Resveratrol And Trans-Piceid,” Food Chem. 98:649-657). Peanut sprouts are also a rich source of resveratrol.

B. Modulation of Gene Product Concentration or Activity

The invention pertains to compositions that, upon administration to a recipient, increase the concentration or activity of a survival/longevity gene product and/or decrease the concentration or activity of a gene product that induces or causes cellular damage. As used herein, such increase (or decrease) in concentration or activity may be accomplished by any mechanism. For example, such increase (or decrease) may reflect a modulation of gene expression resulting in either increased (or decreased) expression of the gene encoding the survival/longevity gene product, or a gene that regulates (e.g., induces or represses) or whose product regulates such expression or activity. Alternatively, or conjunctively, such increase (or decrease) in concentration or activity may reflect a modulation of the recipient's ability to degrade or stabilize any such gene products. Alternatively, or conjunctively, such increase (or decrease) in concentration or activity may reflect a modulation of the recipient's ability to enhance, accelerate, repress or decelerate the activity of any such gene products.

The modulation of concentration or activity discussed above may be a modulation of intracellular, intercellular and/or tissue concentration or activity of such survival/longevity gene products or such gene products that induce or cause cellular damage. Such modulation may be identified by assays of DNA expression, assays of gene product activity, assays of the level of gene product, assays of the rate of gene product turnover, etc. conducted in one or more types of cells, tissues, etc.

An increase in the concentration of a survival/longevity gene product may result from, for example, increased transcription of the gene that encodes the survival/longevity gene product, increased transcription of a gene that induces the expression of the gene that encodes the survival/longevity gene product, decreased transcription of a gene that represses the expression of the gene that encodes the survival/longevity gene product, decreased degradation or enhanced stabilization of expressed molecules of the survival/longevity gene product (leading to the enhanced accumulation of the survival/longevity gene product). Similarly, a decrease in the concentration of a survival/longevity gene product may result from, for example, decreased transcription of the gene that encodes the survival/longevity gene product, decreased transcription of a gene that induces the expression of the gene that encodes the survival/longevity gene product, increased transcription of a gene that represses the expression of the gene that encodes the survival/longevity gene product, increased degradation or decreased stabilization of expressed molecules of the survival/longevity gene product (leading to the enhanced dissipation of the survival/longevity gene product).

One aspect of the present invention thus relates to the use of resveratrol and resveratrol-containing compositions to modulate gene expression, and in particular, to modulate the expression of “survival/longevity” genes and/or “damage inducing” genes. As used herein, a compound is said to “modulate” gene expression if its administration results in a change in expression (relative to a control) of such genes of at least 10%. Modulation may involve an increase in expression (“up-regulation”) or it may involve a decrease in expression (“down-regulation”). The term up-regulate thus denotes an increase of expression of at least 10%, at least 20%, at least 50%, at least 2-fold, at least 5-fold, or most preferably at least 10-fold (relative to a control). The term down-regulate conversely denotes a decrease of expression of at least 10%, at least 20%, at least 50%, at least 2-fold, at least 5-fold, or most preferably at least 10-fold (relative to a control).

A second aspect of the present invention thus relates to the use of resveratrol and resveratrol-containing compositions to modulate the concentration or activity of expressed products of “survival/longevity” genes and/or “damage inducing” genes. As used herein, a compound is said to “modulate” the concentration or activity of such expressed products if its administration results in a change in an intracellular, intercellular or tissue concentration or activity (relative to a control) of such gene products of at least 10%. Modulation may, for example, involve an “enhanced accumulation” or an “enhanced activity” or, for example, it may involve a “diminished accumulation” or a “diminished activity.” The term “enhanced accumulation” (or “enhanced activity”) denotes an increase in concentration (or activity) of at least 10%, at least 20%, at least 50%, at least 2-fold, at least 5-fold, or most preferably at least 10-fold (relative to a control). The term “diminished accumulation” or “diminished activity.” conversely denotes a decrease in concentration (or activity) of at least 10%, at least 20%, at least 50%, at least 2-fold, at least 5-fold, or most preferably at least 10-fold (relative to a control).

As used herein, a “survival/longevity” gene is a gene whose expression contributes to an increase in the survival or longevity of a subject (e.g., a mammal, and particularly a human) expressing such gene. Conversely, a “damage inducing” gene is a gene whose expression contributes to DNA, cellular, or tissue damage in such subject. Such genes are responders to biological stressors, they initiate action in response to stressors such as radiation (e.g., sunlight, gamma rays, UV light, etc.), radiomimetic agents (e.g., vitamin D), heat, near starvation (calorie restriction, or its mimetic, resveratrol) by modulating their expression.

Examples of survival/longevity genes are provided in Table 1. Examples of genes whose expression enhances cellular damage are provided in Table 2. These Tables provide the gene's NCB1 “ENTREZGENE” accession number. Most preferably, such genes are human genes. The Sirtuin 1 gene is known to control the rate of aging of living organisms by virtue of its ability to produce DNA repair enzymes and mimics the beneficial effects of calorie restriction. The trans form of resveratrol (but not cis-resveratrol) activates the Sirtuin 1 gene (Alcendor, R. R. (2007) “Sirt1 Regulates Aging And Resistance To Oxidative Stress In The Heart,” Circulation Research 100(10):1512-1521; Howitz, K. T. et al. (2003) “Small Molecule Activators Of Sirtuins Extend Saccharomyces Cerevisiae Lifespan,” Nature 425:191-196). The invention particularly pertains to compositions that increase the concentration of the Sirtuin 1 survival/longevity gene product. The invention further particularly pertains to compositions that increase the concentration of the forkhead Foxo1 (daf-16, dFoxO) transcription factor survival/longevity gene product.

TABLE 1
Exemplary Survival/Longevity Genes

| | | | | |
|---|---|---|---|---|
| 39329 | A430102J17Rik | Ppp2ca | Shb | Tnfrsf11a |
| 39340 | A430110N23Rik | Ppp2r2d | Shmt2 | Tnfrsf18 |
| 0610007C21Rik | A530082C11Rik | Ppp2r3c | Shroom3 | Tnfrsf1a |
| 0610007L01Rik | A730008L03Rik | Ppp2r5c | Sirt1 | Tnfsf5ip1 |
| 0610010F05Rik | A830018L16Rik | Ppp4r1l | Skil | Tnip1 |
| 0610037L13Rik | A930001N09Rik | Ppp5c | Skiv2l2 | Tnks1bp1 |
| 0610037P05Rik | A930006D11Rik | Ppp6c | Slain2 | Tnni3 |
| 0610040B10Rik | A930018M24Rik | Ppt2 | Slc10a1 | Tnni3k |
| 0610042E11Rik | A930026I22Rik | Pqlc1 | Slc12a4 | Tnnt2 |
| 1110001A07Rik | Ahcyl1 | Prdm4 | Slc12a5 | Tnpo1 |
| 1110002B05Rik | Amd1 | Prdm5 | Slc16a1 | Tnpo2 |
| 1110003O08Rik | Ank | Prdx2 | Slc16a4 | Tnrc6a |
| 1110005A03Rik | Arhgap18 | Prdx3 | Slc1a5 | Tns4 |
| 1110007L15Rik | Arhgap20 | Preb | Slc1a6 | Tnxb |
| 1110007M04Rik | Arhgap24 | Prei4 | Slc20a1 | Toe1 |
| 1110008F13Rik | Arhgap29 | Prkab2 | Slc22a17 | Tollip |
| 1110008J03Rik | Arhgap4 | Prkaca | Slc22a5 | Tomm22 |
| 1110008P14Rik | Arhgap5 | Prkcbp1 | Slc25a11 | Tomm34 |
| 1110014K08Rik | Arhgap9 | Prkcdbp | Slc25a12 | Tomm40 |
| 1110018J18Rik | Arhgdia | Prkch | Slc25a17 | Tomm70a |
| 1110019J04Rik | Arhgef1 | Prkcn | Slc25a22 | Top1 |
| 1110020G09Rik | Arhgef12 | Prkcsh | Slc25a28 | Top2b |
| 1110028A07Rik | Arhgef17 | Prkcz | Slc25a3 | Topors |
| 1110028C15Rik | Arhgef2 | Prkrir | Slc25a32 | Tor1aip2 |
| 1110032E23Rik | B230117O15Rik | Prlr | Slc25a33 | Tpcn1 |
| 1110033M05Rik | B230118H07Rik | Prmt7 | Slc25a34 | Tpm1 |
| 1110036O03Rik | B230219D22Rik | Prodh | Slc25a36 | Tpm3 |
| 1110038B12Rik | B230312A22Rik | Prosc | Slc25a4 | Tpm4 |
| 1110038D17Rik | B230337E12Rik | Prpf6 | Slc25a42 | Tpp1 |
| 1110054O05Rik | B230380D07Rik | Prpsap1 | Slc25a46 | Tpp2 |
| 1110058L19Rik | B3galnt2 | Prr12 | Slc26a11 | Tppp3 |
| 1110059E24Rik | B630005N14Rik | Prrc1 | Slc27a1 | Tpr |
| 1110059G10Rik | B830007D08Rik | Prss12 | Slc29a1 | Tprkb |
| 1110067D22Rik | B830028B13Rik | Prune | Slc31a1 | Tpst1 |
| 1190017O12Rik | B930093H17Rik | Psap | Slc35a2 | Traf3ip2 |
| 1300010M03Rik | C030002C11Rik | Pscd1 | Slc35a3 | Traip |
| 1300012G16Rik | C030007I01Rik | Psd3 | Slc35b1 | Trak2 |
| 1500002I01Rik | C030044B11Rik | Psenen | Slc35b2 | Trappc2 |
| 1500002O20Rik | C030046I01Rik | Pskh1 | Slc36a2 | Trappc2l |
| 1500005K14Rik | C130057M05Rik | Psma2 | Slc39a1 | Trem3 |
| 1500011B03Rik | C130065N10Rik | Psma5 | Slc39a10 | Trex1 |
| 1500011K16Rik | C230091D08Rik | Psma6 | Slc39a8 | Trim11 |
| 1500031L02Rik | C430003N24Rik | Psma8 | Slc40a1 | Trim 12 |
| 1500034J01Rik | C730025P13Rik | Psmb7 | Slc44a1 | Trim23 |
| 1600012F09Rik | Cdc73 | Psmd11 | Slc47a1 | Trim26 |
| 1600015H20Rik | Col10a1 | Psmd12 | Slc4a2 | Trim29 |
| 1600027N09Rik | Col19a1 | Psmd4 | Slc4a4 | Trim3 |
| 1700001O22Rik | Col1a1 | Psmd6 | Slc4a7 | Triobp |
| 1700011B04Rik | Col1a2 | Psmd8 | Slc6a19 | Trip4 |
| 1700017H01Rik | Col23a1 | Pstk | Slc6a6 | Trmt11 |
| 1700020C11Rik | Col27a1 | Ptdss2 | Slc6a9 | Tro |
| 1700021C14Rik | Col4a3bp | Ptgfrn | Slc7a1 | Troap |
| 1700021F05Rik | Col5a1 | Ptms | Slc7a4 | Trpc1 |
| 1700023D09Rik | Col6a2 | Ptp4a3 | Slc7a7 | Trpc4ap |
| 1700029F09Rik | D030011O10Rik | Ptpla | Slc9a1 | Trpm4 |
| 1700029M20Rik | D030051N19Rik | Ptpn1 | Slco1a4 | Tsc22d1 |
| 1700030K09Rik | D230019N24Rik | Ptpn11 | Slco3a1 | Tsc22d4 |
| 1700040L02Rik | D330001F17Rik | Ptpn12 | Slco5a1 | Tsfm |
| 1700051A21Rik | D330017J20Rik | Ptpn20 | Slit3 | Tsga10 |
| 1700113I22Rik | D430015B01Rik | Ptpn3 | Slmap | Tsnax |
| 1700127D06Rik | D430018E03Rik | Ptpra | Slmo2 | Tspan13 |
| 1810007M14Rik | D530037H12Rik | Ptprg | Smarca2 | Tspan18 |
| 1810011O10Rik | D630023B12Rik | Ptprs | Smarcc2 | Tspan4 |
| 1810012P15Rik | D830046C22Rik | Pttg1 | Smarcd3 | Tspan7 |
| 1810013L24Rik | D930017J03Rik | Puf60 | Smchd1 | Tssc4 |
| 1810015C04Rik | D930020B18Rik | Pum1 | Smcr7 | Tsta3 |
| 1810020D17Rik | E030018N11Rik | Pus1 | Smn1 | Ttc1 |
| 1810021J13Rik | E130014J05Rik | Pxmp3 | Smndc1 | Ttc28 |
| 1810022K09Rik | E130303B06Rik | Pxn | Smoc2 | Ttc32 |
| 1810026B05Rik | E330021D16Rik | Qk | Smpd1 | Ttc33 |
| 1810029B16Rik | E430010N07Rik | Rab1 | Smpdl3a | Ttc35 |
| 1810030N24Rik | E430018J23Rik | Rab11a | Smtn | Ttc9c |
| 1810034K20Rik | EG226654 | Rab11b | Smtnl2 | Tub |
| 1810035L17Rik | EG622645 | Rab2 | Smu1 | Tuba4a |
| 1810044A24Rik | EG633640 | Rab20 | Smurf1 | Tuba8 |
| 1810049H13Rik | ENSMUSG00000050599 | Rab21 | Smyd1 | Tubb2c |
| 1810058I24Rik | ENSMUSG00000071543 | Rab24 | Smyd4 | Tubb5 |
| 1810059G22Rik | ENSMUSG00000074466 | Rab30 | Snapap | Tufm |
| 1810063B05Rik | ENSMUSG00000074670 | Rab33b | Snapc1 | Tug1 |
| 1810073N04Rik | ENSMUSG00000075401 | Rab35 | Snf1lk2 | Tulp4 |
| 2010106G01Rik | Exdl1 | Rab3a | Snora65 | Twsg1 |
| 2010109N14Rik | G3bp1 | Rab3gap1 | Snrk | Txlna |
| 2010111I01Rik | Galnt3 | Rab3gap2 | Snrp70 | Txlnb |
| 2010200O16Rik | Galnt4 | Rab3il1 | Snrpb2 | Txndc1 |
| 2010305A19Rik | Galntl4 | Rab43 | Snrpd3 | Txndc10 |
| 2010309E21Rik | Gart | Rab6 | Snx12 | Txndc12 |
| 2010315B03Rik | Kcna5 | Rab8b | Snx13 | Txndc14 |
| 2010320M18Rik | Kcna7 | Rabep1 | Snx16 | Txndc4 |
| 2010321M09Rik | Kcng2 | Rabgap1l | Socs3 | Txnip |
| 2210010L05Rik | Kcnj3 | Rac1 | Socs4 | Txnl1 |
| 2210020M01Rik | Kcnj5 | Rad17 | Sorbs1 | Txnl4 |
| 2210408I21Rik | Kcnk3 | Rad23b | Sorcs2 | Txnl4b |
| 2310001A20Rik | Kcnv2 | Rad54l2 | Sort1 | Tyk2 |
| 2310002L09Rik | Kctd10 | Rag1ap1 | Sost | Uaca |
| 2310007O11Rik | Kctd2 | Ralgps2 | Sox17 | Ubac1 |
| 2310011J03Rik | Kctd7 | Ramp2 | Sox4 | Ubap1 |
| 2310014D11Rik | LOC100044376 | Ranbp10 | Sox9 | Ubash3a |
| 2310014F07Rik | LOC100044968 | Ranbp2 | Sp3 | Ubd |
| 2310016C16Rik | LOC100045002 | Rap1a | Spag9 | Ube1c |
| 2310026E23Rik | LOC100045020 | Rap1gap | Spcs1 | Ube1l2 |
| 2310030G06Rik | LOC100045522 | Rap2a | Speer7-ps1 | Ube1x |
| 2310033F14Rik | LOC100045629 | Rap2b | Spg3a | Ube2b |
| 2310036O22Rik | LOC100046086 | Raph1 | Spg7 | Ube2d2 |
| 2310038H17Rik | LOC100046343 | Rara | Spin1 | Ube2d3 |
| 2310042E22Rik | LOC100046855 | Rarb | Spink10 | Ube2e1 |
| 2310043N10Rik | LOC100047028 | Rarg | Spna2 | Ube2f |
| 2310044H10Rik | LOC100047385 | Rasa1 | Spnb1 | Ube2h |
| 2310046A06Rik | LOC100047539 | Rasa3 | Spnb2 | Ube2n |
| 2310047A01Rik | LOC100047601 | Rasl2-9 | Spop | Ube2o |
| 2310047H23Rik | LOC100047794 | Rassf7 | Spry2 | Ube2q2 |
| 2310047M10Rik | LOC100047915 | Rb1cc1 | Sqstm1 | Ube2v1 |
| 2310061J03Rik | LOC100048376 | Rbbp6 | Srd5a2l2 | Ube2v2 |
| 2310067B10Rik | LOC100048397 | Rbj | Srebf1 | Ube2w |
| 2310076G13Rik | LOC100048439 | Rbm12 | Srebf2 | Ube3a |
| 2410001C21Rik | LOC100048863 | Rbm20 | Sri | Ubl4 |
| 2410002O22Rik | LOC640441 | Rbm24 | Srl | Ubl7 |
| 2410003K15Rik | LOC668206 | Rbm27 | Srp19 | Ublcp1 |
| 2410004B18Rik | LOC675709 | Rbm28 | Srpr | Ubtf |
| 2410005O16Rik | LOC677447 | Rbm38 | Ssbp3 | Ubxd7 |
| 2410012H22Rik | Mtm1 | Rbm39 | Ssbp4 | Uchl5 |
| 2410017P07Rik | OTTMUSG00000001305 | Rbms1 | Ssh1 | Ucp2 |
| 2410017P09Rik | OTTMUSG00000016644 | Rbms2 | Ssr3 | Ucp3 |
| 2410018C17Rik | P2ry5 | Rbmxrt | Sstr5 | Ufm1 |
| 2410018C20Rik | P2ry6 | Rbpms | Ssu72 | Ugcgl2 |
| 2410019A14Rik | Pabpc3 | Rcan2 | St13 | Ugp2 |
| 2410022L05Rik | Pah | Rcl1 | St3gal6 | Umps |
| 2410042D21Rik | Paics | Rdh13 | St6galnac6 | Ung |
| 2510003E04Rik | Paip1 | Reep5 | St7 | Unk |
| 2510042H12Rik | Paip2 | Rem2 | St8sia2 | Uqcc |
| 2610001J05Rik | Palm | Retsat | St8sia4 | Uqcrc1 |
| 2610008E11Rik | Papd1 | Rev1 | Stab1 | Uqcrfs1 |
| 2610019F03Rik | Papd5 | Rfc2 | Stard10 | Usp11 |
| 2610024B07Rik | Papola | Rfc4 | stat6 | Usp19 |
| 2610028D06Rik | Papolg | Rfesd | Stbd1 | Usp2 |
| 2610029I01Rik | Paqr7 | Rfng | Stch | Usp21 |
| 2610030H06Rik | Paqr9 | Rfwd3 | Stip1 | Usp22 |
| 2610101N10Rik | Pard3 | Rfx1 | Stk11 | Usp34 |
| 2610200G18Rik | Pard6g | Rgl1 | Stk19 | Usp36 |
| 2610209M04Rik | Parp12 | Rgma | Stk38 | Usp45 |
| 2610301F02Rik | Pbef1 | Rgs12 | Stk39 | Usp47 |
| 2610507B11Rik | Pbld | Rgs5 | Stom | Usp52 |
| 2610528E23Rik | Pbrm1 | Rhbdd2 | Strn3 | Usp54 |
| 2700029M09Rik | Pcbp2 | Rhbdd3 | Stx6 | Usp9y |
| 2700038N03Rik | Pcca | Rhbdf1 | Stxbp2 | Utp6 |
| 2700097O09Rik | Pcdh7 | Rhd | Styx | Utrn |
| 2810004N23Rik | Pcdh9 | Rhobtb1 | Suhw3 | Uvrag |
| 2810008M24Rik | Pcgf3 | Rhobtb2 | Sulf2 | Uxt |
| 2810410M20Rik | Pcgf6 | Rhoq | Supt5h | V1ra5 |
| 2810422O20Rik | Pcm1 | Ric8 | Supv3l1 | Vamp4 |
| 2810423A18Rik | Pcmt1 | Ring1 | Surf4 | Vasn |
| 2810430I11Rik | Pcnt | Rlbp1 | Svep1 | Vbp1 |
| 2810455D13Rik | Pcnx | Rmnd1 | Sybl1 | Vdac1 |
| 2900002H16Rik | Pcp4 | Rmnd5b | Syk | Vdac2 |
| 2900006B11Rik | Pcp4l1 | Rnaset2a | Syn2 | Vdac3 |
| 2900008C10Rik | Pcsk7 | Rnd1 | Syngr2 | Vdp |
| 2900011G08Rik | Pctk1 | Rnf11 | Synj2bp | Vegfa |
| 2900024O10Rik | Pctk2 | Rnf13 | Synpo | Vegfb |
| 3010003L21Rik | Pctk3 | Rnf139 | Sypl | Vegfc |
| 3010027C24Rik | Pdcd10 | Rnf14 | Taf2 | Vezf1 |
| 3110003A17Rik | Pdcd4 | Rnf149 | Taf6 | Vkorc1l1 |
| 3110031B13Rik | Pdcd6 | Rnf167 | Tanc1 | Vldlr |
| 3110043O21Rik | Pdcl | Rnf168 | Taok2 | Vps16 |
| 3110073H01Rik | Pdcl3 | Rnf187 | Taok3 | Vps18 |
| 3110080E11Rik | Pde1a | Rnf2 | Tap1 | Vps29 |
| 3110082I17Rik | Pde2a | Rnf31 | Tap2 | Vps35 |
| 3222402P14Rik | Pde4dip | Rnf34 | Tapt1 | Vps36 |
| 3321401G04Rik | Pde6a | Rnf5 | Tardbp | Vps37b |
| 4432414F05Rik | Pde7a | Rnf6 | Tatdn3 | Vps4b |
| 4631424J17Rik | Pdgfa | Rock1 | Tbc1d10b | Vps54 |
| 4632404M16Rik | Pdha1 | Rorc | Tbc1d15 | Vwf |
| 4632411B12Rik | Pdia3 | RP23-136K12.4 | Tbc1d19 | Wac |
| 4732416N19Rik | Pdia6 | rp9 | Tbc1d20 | Wapal |
| 4732418C07Rik | Pdk4 | Rpap2 | Tbc1d2b | Was |
| 4832420A03Rik | Pdlim4 | Rpe | Tbc1d5 | Wbp4 |
| 4833408C14Rik | Pdlim5 | Rpl15 | Tbc1d7 | Wdfy1 |
| 4833439L19Rik | Pds5b | Rpl27a | Tbcb | Wdr13 |
| 4921506J03Rik | Pdss1 | Rpl37 | Tbcc | Wdr21 |
| 4921509O07Rik | Pdxk | Rpl39 | Tbce | Wdr22 |
| 4921513H07Rik | Pdzd11 | Rpl3l | Tbcel | Wdr23 |
| 4921517N04Rik | Pecam1 | Rpl7l1 | Tbkbp1 | Wdr3 |
| 4930402E16Rik | Pef1 | Rpl8 | Tbpl1 | Wdr47 |
| 4930426L09Rik | Per1 | Rplp2 | Tbx19 | Wdr5b |
| 4930429B21Rik | Per3 | Rpo1-3 | Tbx20 | Wdr92 |
| 4930432L08Rik | Perp | Rpo1-4 | Tcap | Wdsof1 |
| 4930432O21Rik | Pex11c | Rpp30 | Tcea1 | Wfdc3 |
| 4930448F12Rik | Pex12 | Rprml | Tcea3 | Wipi2 |
| 4930453O09Rik | Pex19 | Rps11 | Tcf15 | Wnk1 |
| 4930455C21Rik | Pex5 | Rps26 | Tcf20 | Wtap |
| 4930466F19Rik | Pex6 | Rps6 | Tcf25 | Wwp2 |
| 4930486A15Rik | Pex7 | Rps6ka1 | Tcfe2a | Xbp1 |
| 4930505O20Rik | Pfdn1 | Rps6ka4 | Tcof1 | Xdh |
| 4930513N20Rik | Pfdn4 | Rrad | Tcp1 | Xlr5a |
| 4930523C07Rik | Pfdn5 | Rragc | Tcp11l2 | Xpnpep1 |
| 4930524O07Rik | Pfkp | Rragd | Tcta | Xpr1 |
| 4930544L04Rik | Pfn2 | Rras2 | Tead4 | Xrcc1 |
| 4930551A22Rik | Pgam2 | Rrbp1 | Tef | Xrcc6 |
| 4930554H23Rik | Pgbd5 | Rrp1 | Tesk1 | Yap1 |
| 4930557J02Rik | Pggt1b | Rrp9 | Tex2 | Yeats2 |
| 4930570C03Rik | Pgr | Rsad1 | Tex261 | Yif1a |
| 4930570E01Rik | Phc2 | Rspry1 | Tfam | Yipf3 |
| 4930573O21Rik | Phc3 | Rtp3 | Tfb2m | Yipf4 |
| 4930579G24Rik | Phf14 | Rufy1 | Tfpi | Yipf7 |
| 4932442K08Rik | Phf17 | Rufy3 | Tfrc | Ypel2 |
| 4933402C05Rik | Phf20l1 | Rusc1 | Tgds | Ypel3 |
| 4933403F05Rik | Phf3 | Rwdd1 | Tgfbr1 | Ywhaq |
| 4933404K13Rik | Phf6 | Rxrb | Thbs4 | Zadh1 |
| 4933407I18Rik | Phka2 | Rxrg | Thnsl2 | Zbed3 |
| 4933411K20Rik | Phkb | Ryk | Thoc1 | Zbtb43 |
| 4933413C19Rik | Phkg1 | Ryr2 | Thoc4 | Zbtb5 |
| 4933421A08Rik | Phlda1 | S3-12 | Thrb | Zc3h11a |
| 4933426M11Rik | Phldb1 | Sae2 | Tie1 | Zc3h12c |
| 4933428L01Rik | Phpt1 | Safb | Tigd2 | Zc3h15 |
| 4933429D07Rik | Phyh | Samd5 | Timm22 | Zc3h6 |
| 4933433P14Rik | Pias4 | Samd8 | Timm50 | Zc3h8 |
| 4933434E20Rik | Picalm | Samd9l | Timp3 | Zcchc6 |
| 4933440H19Rik | Pigz | Saps3 | Timp4 | Zdhhc13 |
| 5033414K04Rik | Pik3ca | Sar1a | Tinagl | Zdhhc3 |
| 5033421C21Rik | Pik3ip1 | Sars | Tjap1 | Zeb1 |
| 5033423K11Rik | Pip5k1c | Sat1 | Tk2 | Zfand5 |
| 5033430J17Rik | Pir | Satb2 | Tle6 | Zfml |
| 5330423I11Rik | Pitpna | Sbds | Tlk2 | Zfp106 |
| 5330439A09Rik | Pitpnb | Sbf2 | Tln1 | Zfp110 |
| 5430402E10Rik | Pitpnc1 | Sbk1 | Tloc1 | Zfp187 |
| 5430402P08Rik | Pitpnm1 | Sc4mol | Tm2d1 | Zfp191 |
| 5430407P10Rik | Pkd1 | Scamp3 | Tm2d2 | Zfp213 |
| 5730470L24Rik | Pkia | Scap | Tm2d3 | Zfp236 |
| 5730507A11Rik | Pkm2 | Scara5 | Tm4sf1 | Zfp238 |
| 5730536A07Rik | Pkp2 | Scarb1 | Tm6sf2 | Zfp26 |
| 5730601F06Rik | Pla2g10 | Scarb2 | Tm9sf2 | Zfp260 |
| 5830404H04Rik | Pla2g2d | Sccpdh | Tm9sf3 | Zfp277 |
| 5830415L20Rik | Pla2g5 | Scfd1 | Tmc1 | Zfp289 |
| 5830428H23Rik | Plcd1 | Schip1 | Tmcc1 | Zfp30 |
| 5830432E09Rik | Plce1 | Scmh1 | Tmcc3 | Zfp313 |
| 5830436I19Rik | Pld3 | Scn4b | Tmco1 | Zfp319 |
| 5830457O10Rik | Pldn | Scoc | Tmed7 | Zfp322a |
| 5830469G19Rik | Plec1 | Scube2 | Tmem103 | Zfp335 |
| 5830487K18Rik | Plekhh3 | Scyl1 | Tmem109 | Zfp341 |
| 5930434B04Rik | Plekhj1 | Scyl3 | Tmem110 | Zfp35 |
| 6230429P13Rik | Plekhm2 | Sdcbp | Tmem112b | Zfp383 |
| 6330403M23Rik | Plekhn1 | Sdccag10 | Tmem115 | Zfp384 |
| 6330407G11Rik | Plod3 | Sdha | Tmem119 | Zfp414 |
| 6330409N04Rik | Plp2 | Sdhd | Tmem123 | Zfp422 |
| 6330415G19Rik | Pls3 | Sds | Tmem126b | Zfp422-rs1 |
| 6330417G04Rik | Pltp | Sec11a | Tmem132a | Zfp512 |
| 6330503C03Rik | Plvap | Sec14l1 | Tmem142a | Zfp516 |
| 6330564D18Rik | Plxnb2 | Sec22b | Tmem142c | Zfp560 |
| 6330569M22Rik | Pmm1 | Sec23a | Tmem147 | Zfp568 |
| 6430548M08Rik | Pno1 | Sec31a | Tmem14c | Zfp579 |
| 6530404N21Rik | Pnpla1 | Sec61a1 | Tmem157 | Zfp597 |
| 6530413G14Rik | Pnpla2 | Sec61a2 | Tmem159 | Zfp608 |
| 6620401M08Rik | Pnpla6 | Sele | Tmem167 | Zfp628 |
| 6720462K09Rik | Pnrc2 | Sema3b | Tmem168 | Zfp629 |
| 6720475J19Rik | Podn | Sephs2 | Tmem16f | Zfp639 |
| 6820401H01Rik | Poldip3 | Sepp1 | Tmem176a | Zfp644 |
| 7030402D04Rik | Polg2 | Serbp1 | Tmem176b | Zfp650 |
| 7030407E18Rik | Polr2d | Serinc1 | Tmem182 | Zfp651 |
| 7420416P09Rik | Polr2f | Serpinb6a | Tmem188 | Zfp667 |
| 8030463A06Rik | Polr2h | Serpinb9 | Tmem19 | Zfp672 |
| 8030475D13Rik | Polr2i | Sertad2 | Tmem30a | Zfp68 |
| 8430436O14Rik | Polr2k | Set | Tmem37 | Zfp703 |
| 9030411M15Rik | PolrSgl | Setd7 | Tmem38a | Zfp715 |
| 9030418K01Rik | Polr3k | Setd8 | Tmem38b | Zfp719 |
| 9030425P06Rik | Pot1a | Setx | Tmem41a | Zfp740 |
| 9130011J15Rik | Pou6f1 | Sf3a1 | Tmem41b | Zfp758 |
| 9230110F11Rik | Ppap2b | Sf3b1 | Tmem46 | Zfp817 |
| 9230114K14Rik | Ppard | Sf3b2 | Tmem50a | Zfp82 |
| 9330109K16Rik | Pparg | Sfrp5 | Tmem55b | Zfyve21 |
| 9330120H11Rik | Ppargc1a | Sfrs1 | Tmem57 | Zhx2 |
| 9430010O03Rik | Pphln1 | Sfrs10 | Tmem64 | Zhx3 |
| 9430013L17Rik | Ppic | Sfrs2ip | Tmem69 | Zic2 |
| 9530018H14Rik | Ppif | Sfrs7 | Tmem70 | Zkscan17 |
| 9530018I07Rik | Ppig | Sfrs9 | Tmem77 | Zmat2 |
| 9530097N15Rik | Ppil2 | Sfxn3 | Tmem85 | Zmat5 |
| 9930024M15Rik | Ppl | Sgca | Tmem86a | Zmym4 |
| A030007L17Rik | Ppm1f | Sgcg | Tmem93 | Zmynd10 |
| A230046K03Rik | Ppme1 | Sgk2 | Tmem9b | Znrf1 |
| A230051G13Rik | Ppp1ca | Sgta | Tmlhe | Zrsr1 |
| A230062G08Rik | Ppp1r11 | Sh2d3c | Tmod1 | Zscan12 |
| A230067G21Rik | Ppp1r12a | Sh2d4a | Tmod4 | Zswim6 |
| A230091C14Rik | Ppp1r12c | ShSbgrl | Tmub1 | Zyg11b |
| A330043J11Rik | Ppp1r13l | Sh3bp5l | Tmub2 | Zyx |
| A330076H08Rik | Ppp1r2 | Sh3d19 | Tnfaip1 | Zzef1 |
| A430005L14Rik | Ppp1r3c | Sh3kbp1 | Tnfaip8l1 | |

TABLE 2
Exemplary Genes Whose Expression Enhances Cellular Damage

| | | | | |
|---|---|---|---|---|
| AA407175 | Blvra | Ddah2 | Gltscr2 | Llgl1 |
| AA415038 | Bmi1 | Ddb1 | Glud1 | Lmbrd1 |
| AA987161 | Bmp6 | Ddb2 | Glul | Lmln |
| Aadacl1 | Bmpr1a | Ddit3 | Gm104 | Lmna |
| Aars | Bnip3 | Ddr1 | Gm561 | Lmo4 |
| Aasdhppt | Brd3 | Ddr2 | Gmeb1 | Lmtk2 |
| AB182283 | Btaf1 | Ddx1 | Gmfb | LOC100040515 |
| Abca4 | Btbd14b | Ddx17 | Gmppa | LOC100043489 |
| Abca7 | Btbd2 | Ddx39 | Gna-rs1 | LOC100046468 |
| Abcb4 | Btbd3 | Ddx51 | Gnb2 | LOC100046982 |
| Abcb7 | Btbd6 | Ddx54 | Gnb4 | LOC552902 |
| Abcd1 | Btf3l4 | Ddx58 | Gne | Lonp1 |
| Abce1 | Btnl9 | Ddx6 | Gng10 | Lor |
| Abhd1 | Bxdc2 | Deb1 | Gnl3 | Lpgat1 |
| Abhd12 | C130094E24 | Dedd | Gnpda1 | Lphn1 |
| Abhd4 | C2 | Defb1 | Golga2 | Lrch1 |
| Abi1 | C77058 | Defb5 | Golga7 | Lrch4 |
| Abi2 | C78441 | Defcr15 | Golgb1 | Lrp10 |
| Ablim2 | C78651 | Depdc7 | Got1 | Lrp2bp |
| Abra | C79741 | Derl2 | Got2 | Lrp6 |
| Abtb1 | C86942 | Des | Gpaa1 | Lrpap1 |
| Acaa2 | C87259 | Dfna5h | Gpam | Lrrc1 |
| Acad11 | Cab39 | Dgat2 | Gpatch1 | Lrrc20 |
| Acad9 | Cabin1 | Dgcr2 | Gpbp1 | Lrrc39 |
| Acadl | Cacna1g | Dgka | Gpbp1l1 | Lrrc3b |
| Acads | Cacna1h | Dgke | Gpc1 | Lrrc40 |
| Acadvl | Cacybp | Dguok | Gpc6 | Lrrc44 |
| Acbd3 | Cadm4 | Dhodh | Gpd1l | Lsm14a |
| Acbd5 | Calm1 | Dhrs1 | Gper | Lsm14b |
| Acbd6 | Calr | Dhrs7 | Gpkow | Lsm3 |
| Ace | Calr3 | Dhx30 | Gpr115 | Ltb4dh |
| Aco2 | Caml | Dhx32 | Gpr137 | Ltbp3 |
| Acot5 | Camsap1 | Dhx34 | Gpr175 | Ltbp4 |
| Acox3 | Cand2 | Dhx8 | Gpr22 | Ly6a |
| Acsl1 | Canx | Dhx9 | Gpr4 | Lypla1 |
| Acss2 | Capg | Diablo | Gpr98 | Lypla2 |
| Acta1 | Capn1 | Diap1 | Gpsn2 | Lyrm4 |
| Actb | Capns1 | Diras1 | Gpt2 | Lyrm5 |
| Actn1 | Caprin1 | Dirc2 | Gpx3 | Lysmd2 |
| Actn2 | Card 10 | Dkk3 | Gramd1a | Lysmd3 |
| Actn4 | Caskin2 | Dld | Grb14 | Lztfl1 |
| Actr1b | Casp8ap2 | Dll4 | Grina | Lztr1 |
| Actr2 | Casp9 | Dlst | Grk1 | M6prbp1 |
| Acvr1b | Casq1 | Dmd | Grk5 | Macf1 |
| Acvr2a | Cav1 | Dmpk | Grlf1 | Macrod1 |
| Acvrl1 | Cav2 | Dmtf1 | Grm8 | Maf1 |
| Acyp1 | Cbfb | Dmwd | Grn | Magea5 |
| Acyp2 | Cblb | Dmxl2 | Grpel2 | Magee1 |
| Adal | Cbr1 | Dnahc9 | Gsdmdc1 | Magi3 |
| Adam10 | Cbx3 | Dnaja3 | Gsn | Mall |
| Adam15 | Ccar1 | Dnajb1 | Gsta4 | Man2b1 |
| Adam21 | Ccdc12 | Dnajb4 | Gstcd | Maob |
| Adamts10 | Ccdc122 | Dnajb9 | Gstm1 | Map1lc3a |
| Adamts2 | Ccdc125 | Dnajc12 | Gstm2 | Map1lc3b |
| Adamts7 | Ccdc127 | Dnajc3a | Gstm5 | Map2k1ip1 |
| Adamts9 | Ccdc3 | Dnajc7 | Gstm7 | Map2k2 |
| Adar | Ccdc34 | Dnm2 | Gstp1 | Map3k1 |
| Adcy1 | Ccdc47 | Dock11 | Gstt1 | Map3k12 |
| Adcy2 | Ccdc58 | Dock6 | Gtf2a1 | Map3k2 |
| Adcy3 | Ccdc69 | Dom3z | Gtf2a2 | Map3k7 |
| Adcy6 | Ccdc7 | Dopey1 | Gtf2e1 | Map3k7ip1 |
| Add1 | Ccdc72 | Dot1l | Gtf2e2 | Map4k5 |
| Adh5 | Ccdc85b | Dpagt1 | Gtf2h3 | Mapbpip |
| Adra1b | Ccdc88a | Dph3 | Gtf2h4 | Mapk14 |
| Adrbk1 | Ccdc90a | Dpp8 | Gtf3c1 | Mapk6 |
| Aebp1 | Ccdc90b | Dpp9 | Gtf3c4 | Mapkapk2 |
| Aes | Ccl9 | Dpysl2 | Gtpbp1 | Mapre1 |
| Afap1l1 | Ccm2 | Dpysl3 | Gtpbp2 | Marcks |
| Aff4 | Ccnd3 | Dr1 | Gulo | Mat2a |
| Afg3l1 | Ccng1 | Drg1 | Gyg | Mat2b |
| Aga | Ccnh | Dstn | Gyk | Matn4 |
| Agbl5 | Ccni | Dtnbp1 | Gys1 | Maz |
| Agpat1 | Ccnl2 | Dus3l | H2afv | Mbc2 |
| Agpat5 | Ccnt2 | Dusp1 | H2afy | Mbd2 |
| Agrn | Ccr1 | Dusp6 | H2-Bl | Mbd3 |
| Agtr1a | Ccr1l1 | Dusp8 | H2-Oa | Mbd5 |
| Agxt2l2 | Ccr5 | Dvl2 | H2-T24 | Mboat5 |
| Ahdc1 | Ccs | Dync1h1 | H6pd | Mbtps1 |
| Ahr | Cct5 | Dync1li2 | Hadh | Mbtps2 |
| Ahsa1 | Cct7 | Dyrk1a | Hadha | Mcf2l |
| Al118078 | Cd151 | E2f6 | Hadhb | Mctp2 |
| Al225934 | Cd163 | Eaf1 | Hand2 | Mdfic |
| Al413194 | Cd200 | Eapp | Hars | Mdh2 |
| Al428479 | Cd207 | Ears2 | Hars2 | Med13 |
| Al429363 | Cd36 | Ebag9 | Hat1 | Med16 |
| Al462493 | Cd38 | Ece1 | Hax1 | Med19 |
| Al480535 | Cd74 | Ecm1 | Hccs | Med25 |
| Al506816 | Cd83 | Ecm2 | Hcfc1r1 | Med30 |
| Al597468 | Cd93 | Edaradd | Hcfc2 | Med7 |
| Al662270 | Cd97 | Edg3 | Hdac2 | Mef2b |
| Al662476 | Cdadc1 | Eea1 | Hdac4 | Mef2c |
| Al747699 | Cdc27 | Eef1a1 | Hdac7a | Mef2d |
| Al790298 | Cdc2l5 | Eef1b2 | Hdhd2 | Megf11 |
| Al837181 | Cdc2l6 | Eef1e1 | Hdlbp | Megf8 |
| Al848100 | Cdc37 | Eef2 | Heatr5b | Mel13 |
| Al852064 | Cdc42ep3 | Efcab2 | Heatr6 | Mertk |
| Al987944 | Cdgap | Efemp2 | Hectd1 | Mesdc2 |
| Ak7 | Cdh13 | Efnb3 | Heph | Metrnl |
| Akap13 | Cdipt | Egf | Herpud1 | Mett10d |
| Akap2 | Cdk5rap3 | Egfl7 | Heyl | Mex3c |
| Akp2 | Cdk7 | Egflam | Hfe2 | Mfap4 |
| Akr1a4 | Cdv3 | Egfr | Hgs | Mfge8 |
| Akr1b8 | Cebpz | Egln1 | Hhatl | Mfn1 |
| Akr7a5 | Cenpa | Egln3 | Hiat1 | Mfsd8 |
| Akt1s1 | Cenpq | Egr1 | Hiatl1 | Mgam |
| Alas1 | Cental | Ehbp1l1 | Hibadh | Mgat1 |
| Aldh1a3 | Centa2 | Ehd4 | Hif1a | Mgat4b |
| Aldh2 | Centb2 | Ei24 | Higd1b | Mgp |
| Aldh4a1 | Centd1 | Eif1ay | Hint3 | Mgrn1 |
| Aldh7a1 | Centd2 | Eif2ak1 | Hirip3 | Mif4gd |
| Aldh9a1 | Centg2 | Eif2s2 | Hivep2 | Mkl1 |
| Alg12 | Cetn3 | Eif3e | Hk3 | Mknk1 |
| Alg13 | Cfl1 | Eif4a1 | Hlf | Mlf1 |
| Alg5 | Cflar | Eif4a2 | Hmcn1 | Mlkl |
| Alkbh6 | Cgnl1 | Eif4b | Hmg20b | Mll2 |
| Alkbh8 | Cqrrf1 | Eif4e2 | Hmgb1 | Mllt1 |
| Als2cr2 | Chac1 | Eif4ebp1 | Hmgb3 | Mllt6 |
| Anapc10 | Chac2 | Eif4g3 | Hmgcl | Mlx |
| Anapc2 | Chchd4 | Eif5 | Hmgcs1 | Mlxip |
| Angptl2 | Chd1 | Eif5b | Hnrpab | Mlycd |
| Ank1 | Chd2 | Elac2 | Hnrph3 | Mme |
| Ankhd1 | Chd4 | Elf2 | Hnrpk | Mmp15 |
| Ankrd1 | Chmp1b | Elk3 | Hnrpl | Mmp1b |
| Ankrd10 | Chmp2b | Ell | Hnrpll | Mmp2 |
| Ankrd13a | Chordc1 | Ell2 | Hnrpr | Mmrn2 |
| Ankrd13c | Chrac1 | Elovl5 | Hnrpul1 | Mobkl3 |
| Ankrd13d | Chrd | Elp3 | Hoxd11 | Mocos |
| Ankrd25 | Chrng | Elp4 | Hrasls | Mocs2 |
| Ankrd28 | Chst14 | Eltd1 | Hrc | Morc2a |
| Ankrd32 | Chuk | Emb | Hs2st1 | Mosc2 |
| Ankrd37 | Churc1 | Emd | Hsd17b11 | Mospd1 |
| Ankrd38 | Ciao1 | Eme2 | Hsd17b13 | Mpa2l |
| Ankrd9 | Cib1 | Emg1 | Hsd17b4 | Mpp6 |
| Anp32b | Cic | Emilin1 | Hsdl2 | Mpv17 |
| Anxa3 | Cilp2 | Eml2 | Hsp110 | Mrfap1 |
| Anxa6 | Cisd2 | Enc1 | Hspa1b | Mrgprf |
| Aoc3 | Cish | Eng | Hspa5 | Mrpl1 |
| Ap1s2 | Ckm | Eno3 | Hspb2 | Mrpl15 |
| Ap2a2 | Ckmt2 | Enpep | Hspb3 | Mrpl17 |
| Ap2b1 | Clcn5 | Enpp5 | Hspb6 | Mrpl19 |
| Ap2m1 | Cldnd1 | Entpd5 | Hspb7 | Mrpl28 |
| Ap4m1 | Clec2d | Entpd6 | Hspe1 | Mrpl30 |
| Ap4s1 | Clic1 | Ep300 | Htra1 | Mrpl32 |
| Apbb1 | Clic4 | Epb4.1l3 | Htra3 | Mrpl36 |
| Aplp2 | Clint1 | Epha4 | Hus1 | Mrpl38 |
| Apobec2 | Clk3 | Ephb1 | Hyal4 | Mrpl4 |
| Apod | Cln5 | Ephb4 | Iah1 | Mrpl41 |
| Apoe | Clock | Epm2aip1 | Ibrdc2 | Mrps17 |
| Apool | Clptm1 | Epn1 | Id1 | Mrps18c |
| Appl1 | Clstn1 | Eps15l1 | Ide | Mrps22 |
| Appl2 | Cltc | Erc1 | Idh3a | Mrps5 |
| Arf1 | Cmpk | Ergic3 | Idh3b | Mrs2l |
| Arf3 | Cmya5 | Erlin1 | Ifi30 | Msl31 |
| Arg2 | Cndp2 | Ero1lb | Ifit3 | Msra |
| Arid4b | Cnot6l | Errfi1 | Ifnar1 | Msrb2 |
| Arl1 | Cnot7 | Esco1 | Ifnar2 | Msrb3 |
| Arl2bp | Cntfr | Esd | Ifngr1 | Msx1 |
| Arl3 | Cntn4 | Esf1 | Ifngr2 | Mtap4 |
| Arl4a | Commd1 | Esrrg | Ift122 | Mtap7d1 |
| Arl5b | Commd3 | Etfa | Ift57 | Mtbp |
| Arpc1a | Commd4 | Etnk1 | Igfbp4 | Mtch2 |
| Arpc1b | Commd5 | Ets2 | Igfbp6 | Mterf |
| Arpc2 | Comp | Ewsr1 | Igsf11 | Mterfd1 |
| Arpc4 | Cope | Exoc5 | Igsf3 | Mterfd2 |
| Arrb1 | Copg | Exosc1 | Igsf8 | Mterfd3 |
| Art5 | Cops2 | Exosc10 | Ihpk1 | Mtif3 |
| Asb1 | Cops7a | Exosc7 | Ikbkap | Mtmr1 |
| Asb14 | Coq10b | Exosc9 | Il10rb | Mtmr3 |
| Asb5 | Coq9 | Ext1 | Il13ra1 | Mtrr |
| Ascc3l1 | Coro1b | Eya3 | Il18bp | Mustn1 |
| Asnsd1 | Cox 11 | F11r | Il6st | Mxd4 |
| Asph | Cox4i2 | F13b | Ilk | Mxi1 |
| Atad2b | Cox5a | F5 | Ilvbl | Mxra8 |
| Atf3 | Cox8a | Fads3 | Immp1l | Mybbp1a |
| Atg10 | Cp | Fahd2a | Immp2l | Mybpc3 |
| Atg3 | Cpeb4 | Fam18b | Immt | Myc |
| Atg4d | Cpm | Fancg | Imp3 | Mycbp |
| Atg5 | Cpsf1 | Fap | Impa2 | Myct1 |
| Atp11b | Cpsf3 | Fas | Impad1 | Myd116 |
| Atp13a1 | Cpt1a | Fastkd1 | Ints8 | Myd88 |
| Atp1a2 | Cpt1b | Fastkd2 | Ipmk | Myef2 |
| Atp5h | Cramp1l | Fbln1 | Ipo13 | Myh14 |
| Atp5s | Crat | Fbln2 | Ipo7 | Myh6 |
| Atp6ap2 | Crbn | Fbp2 | Ipo8 | Myl4 |
| Atp6v0a2 | Creb1 | Fbxl2 | Iqsec1 | Myl7 |
| Atp6v0d1 | Creb3l1 | Fbxl6 | Iqwd1 | Mylip |
| Atp6v1b2 | Crebbp | Fbxo3 | Irf4 | Myo10 |
| Atp6v1f | Crebzf | Fbxo30 | Irs1 | Myo1c |
| Atp9a | Creg1 | Fbxw4 | Isca2 | Myo9b |
| Atp9b | Crip1 | Fbxw5 | Isg20 | Myocd |
| Atpaf1 | Crip2 | Fcer2a | Isyna1 | Myom1 |
| Atpbd1c | Cript | Fcgr4 | Itfq3 | Mypn |
| Atpif1 | Crnkl1 | Fdx1 | Itgb1bp1 | N6amt1 |
| Atr | Crot | Fem1c | Itgb1bp2 | N6amt2 |
| Atxn2 | Cry1 | Fert2 | Itgb1bp3 | Naca |
| Atxn7l1 | Cryab | Fgfr1op | Itgb2 | Nagpa |
| AU020772 | Crybb1 | Filip1 | Itgb5 | Nars |
| AU041133 | Cryz | Fkbp10 | Itih3 | Nat5 |
| Aup1 | Csdc2 | Fkbp5 | Itk | Nbeal1 |
| AV009015 | Cse1l | Fkbp8 | Itm2b | Nckap1l |
| AV024533 | Csf2ra | Flcn | Itm2c | Ndst4 |
| AV025504 | Csl | Flii | Itpr3 | Ndufa5 |
| Avpr1a | Csnk1a1 | Flot1 | Ivns1abp | Ndufab1 |
| AW046287 | Csnk1d | Flot2 | Jam2 | Ndufaf1 |
| AW112010 | Csnk2a1 | Flywch1 | Jmjd1c | Ndufb3 |
| AW209491 | Cst3 | Fmn1 | Jmjd2a | Ndufb7 |
| AW555464 | Cst8 | Fmo2 | Jmjd6 | Ndufb8 |
| AW556556 | Cstf2 | Fmr1 | Josd2 | Ndufc1 |
| AW742931 | Ctage5 | Fnbp1l | Jtv1 | Ndufc2 |
| Azi2 | Ctcf | Fnip1 | Jun | Ndufs1 |
| Azin1 | Ctgf | Foxa3 | Kbtbd10 | Ndufs2 |
| Bach1 | Ctps | Foxj2 | Kbtbd5 | Ndufs3 |
| Bag4 | Ctsb | Foxk1 | Kcnip2 | Ndufs5 |
| Bambi | Ctsf | Foxk2 | Khk | Ndufs7 |
| Banp | Ctss | Foxo1 | Kif1b | Ndufs8 |
| Bat1a | Ctsz | Foxp1 | Kif1c | Ndufv1 |
| Bat2 | Cttnbp2nl | Frag1 | Kif21a | Ndufv2 |
| Baz1a | Cul1 | Frap1 | Kif2a | Nedd4 |
| Baz1b | Cul3 | Frmd4b | Kif3a | Neil1 |
| BB217526 | Cxcl12 | Frmd5 | Kif5b | Nek3 |
| Bbc3 | Cxcl14 | Fscn1 | Klc3 | Nf2 |
| Bbs10 | Cxxc1 | Fth1 | Klf11 | Nfu1 |
| BC003331 | Cxxc5 | Ftl1 | Klf13 | Ngly1 |
| BC003885 | Cyb561 | Fuca2 | Klf15 | Ngrn |
| BC003965 | Cyb5b | Fundc2 | Klf16 | Nid1 |
| BC010304 | Cyb5r3 | Furin | Klf4 | Nif3l1 |
| BC010981 | Cyb5r4 | Fus | Klf7 | Ninj1 |
| BC011248 | Cybasc3 | Fxc1 | Klhdc1 | Nipbl |
| BC013529 | Cyc1 | Fxyd1 | Klhdc3 | Nkiras1 |
| BC016495 | Cyfip1 | Fxyd5 | Klhl13 | Nkiras2 |
| BC019943 | Cyp1b1 | Fzd10 | Klhl22 | Nlgn2 |
| BC020077 | Cyp27a1 | Fzd2 | Klhl23 | Nmnat1 |
| BC021395 | Cyp2f2 | Fzd9 | Klhl24 | Npc1 |
| BC023882 | Cys1 | G0s2 | Klhl4 | Nppb |
| BC024659 | D030063E12 | G6pc2 | Klhl9 | Nras |
| BC024814 | D0H4S114 | Gaa | Klk1b24 | Nrd1 |
| BC025076 | D10Ertd641e | Gab1 | Kpna1 | Nrp1 |
| BC028440 | D13Ertd787e | Gabpa | Kpna4 | Nrp2 |
| BC028528 | D14Ertd16e | Gadd45b | Krr1 | Nrtn |
| BC030183 | D14Ertd581e | Gadd45g | Kti12 | Nsmaf |
| BC030308 | D15Ertd50e | Gale | Ktn1 | Nt5c2 |
| BC030336 | D16H22S680E | Galk1 | L1cam | Nt5c3 |
| BC031353 | D19Ertd721e | Gapdh | l7Rn6 | Nub1 |
| BC031781 | D19Ertd737e | Gapvd1 | Lace1 | Nwd1 |
| BC032203 | D19Wsu162e | Garnl1 | Lactb | Oasl2 |
| BC034069 | D1Bwg1363e | Gas6 | Lama2 | Ogg1 |
| BC037034 | D2Ertd391e | Gata4 | Lamb2 | Oplah |
| BC037112 | D3Ertd254e | Gba2 | Laptm4a | Pfkfb2 |
| BC038479 | D3Wsu106e | Gbas | Larp1 | Pfkfb4 |
| BC039210 | D4Ertd429e | Gbe1 | Larp2 | Pgc1 |
| BC043098 | D4Ertd571e | Gbf1 | Larp4 | Pgls |
| BC043476 | D6Wsu176e | Gcdh | Larp5 | Pygb |
| BC048679 | D8Ertd457e | Gdi1 | Lcmt1 | Rars |
| BC049349 | D8Ertd54e | Gdi2 | Lcmt2 | Rars2 |
| BC057893 | D8Ertd620e | Gdpd1 | Ldb1 | Rere |
| Bcam | D8Ertd82e | Gdpd5 | Ldb3 | Rnpepl1 |
| Bcat2 | D9Ertd402e | Gemin5 | Ldhb | RP23-233B9.8 |
| Bckdha | Daam1 | Ggta1 | Ldhd | Rsrc2 |
| Bckdk | Dad1 | Ghitm | Leo1 | Smad1 |
| Bcl2l13 | Dap | Gimap4 | Lgals3bp | Smad3 |
| Bcl6b | Dapk2 | Gimap8 | Lgals7 | Smad6 |
| Bclaf1 | Daxx | Git1 | Lgmn | Ucp3 |
| Bdp1 | Dbh | Gja3 | Lgr4 | X83328 |
| Bet1 | Dcn | Gle1l | Lgr6 | Xk |
| Bgn | Dctn1 | Glg1 | Lias | Xpo4 |
| Bhlhb2 | Dctn2 | GN1 | Limd1 | Xpo6 |
| Bhlhb3 | Dcun1d1 | Glo1 | Lims2 | Xpot |
| Bicd2 | Dcun1d2 | Glod4 | Lipe | Xrn1 |
| Birc4 | Dcun1d5 | Gls | Lix1l | |

C. Preferred Compositions of the Present Invention

The invention particularly pertains to resveratrol-containing compositions in which the specific activity of the resveratrol has been stabilized or enhanced. As used herein, the term “specific activity” refers to the ratio of the extent of gene modulation (relative to control) per amount (mass) of administered resveratrol.

Preferably, such compositions will comprise a chelator, hyaluronic acid, and/or vitamin D. The invention particularly pertains to such compositions that comprise resveratrol (preferably, the compositions of the present invention will provide a composition dosage of from about 10 mg to about 2 g, more preferably from about 100 mg to about 500 mg), and at least one compound selected from the group consisting of an antioxidant (chelator), hyaluronic acid, and vitamin D. Preferably, the compositions of the present invention will contain resveratrol, an antioxidant, hyaluronic acid, and vitamin D.

As used herein the term “chelator” refers to an organic compound that bonds with and removes free metal ions from solution. Examples of suitable chelators include ethylenediaminetetraacetic acid (EDTA), histidine, antibiotic drugs of the tetracycline family, pyridoxal 2-chlorobenzoyl hydrazone, dcsferrioxamine, dexrazoxane, deferasirox, pyoverdine, pseudan, citrate, NDGA (nordihydroguaiaretic acid: 1,4-bis[3,4-dihydroxyphenyl]2,3-dimethylbutane), ferulic acid and phytic acid. Preferably, the compositions of the present invention will provide a composition dosage of chelator of from about 1 g to about 15 g, more preferably from about 2 g to about 12 g.

Phytic acid is a particularly preferred chelator for the purposes of the present invention. As used herein, the term “phytic acid” refers to inositol hexaphosphate ((2,3,4,5,6-pentaphosphonooxycyclohexyl) dihydrogen phosphate; also known as “IP6”;) (see, Thorne Research, Inc. (2002) “Inositol Hexaphosphate. Monograph” Altern. Med. Rev. 7(3):244-248; Vucenik, I. et al. (2006) “Protection Against Cancer By Dietary IP6 And Inositol,” Nutr. Cancer. 55(2):109-125; López, M. A. et al. (2004) “Iron Availability: An Updated Review,” Int. J. Food Sci. Nutr. 55(8):597-606; Singh, R. P. et al. (2005) “Prostate Cancer And Inositol Hexaphosphate: Efficacy And Mechanisms,” Anticancer Res. 25(4):2891-2903; Vucenik, I. et al. (2006) “Cancer Inhibition By Inositol Hexaphosphate (IP6) And Inositol: From Laboratory To Clinic,” J. Nutr. 133(11 Suppl 1):3778S-3784S; Raboy, V. (2003) “Myo-Inositol-1,2,3,4,5,6-Hexakisphosphate,” Phytochemistry. 64(6):1033-19043; Vohra, A. et al. (2003) “Phytases: Microbial Sources, Production, Purification, And Potential Biotechnological Applications,” Crit. Rev. Biotechnol. 23(1):29-60; Fox, C. H. et al. (2002) “Phytic Acid (IP6), Novel Broad Spectrum Anti-Neoplastic Agent: A Systematic Review,” Complement Ther. Med. 10(4):229-234; Grascs, F. et al. (1999) “Phytate (IP6) Is A Powerful Agent For Preventing Calcifications In Biological Fluids: Usefulness In Renal Lithiasis Treatment,” Anticancer Res. 19(5a):3717-3722; Jariwalla, R. J. (1999) “Inositol Hexaphosphate (IP6) As An Anti-Neoplastic And Lipid-Lowering Agent,” Anticancer Res. 19(5a):3699-3702; Katayama T. (1999) “Hypolipidemic Action Of Phytic Acid (IP6): Prevention Of Fatty Liver,” Anticancer Res. 19(5a):3695-3698). The structure of phytic acid is provided below:

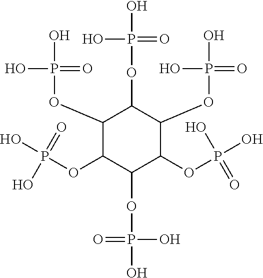 [SMILES: O=P(O)(O)OC1C(OP(=O)(O)O)C(OP(=O)(O)O)C(OP(=O)(O)O)C(OP(=O)(O)O)C1OP(=O)(O)O]

Phytic acid is found in substantial amounts in whole grains, cereals, legumes, nuts, and seeds, and is the primary energy source for the germinating plant (Graf, E. (1983) “Applications of Phytic Acid,” J. Am. Oil. Chem. Soc 60:1861-1867). Phytic acid and its lower phosphorylated forms (such as IP3) are also found in most mammalian cells, where they assist in regulating a variety of important cellular functions (Szwergold, B. S. et al. (1987) “Observation Of Inositol Pentakis-And Hexakisphosphates In Mammalian Tissues By ³¹P NMR,” Biochem. Biophys. Res. Commun. 264:874-881). Phytic Acid is preferably provided in the form of rice bran (Srinivasan, M. (2007) “Ferulic Acid: Therapeutic Potential Through Its Antioxidant Property,” J. Clin. Biochem. Nutr. (2007) 40(2):92-100; Kim, M. J. et al. (2007) “Ferulic Acid Supplementation Prevents Trimethyltin-Induced Cognitive Deficits in Mice,” Biosci. Biotechnol. Biochem. (2007) 71(4):1063-1068). Phytic acid is reported to function as an antioxidant by chelating divalent cations such as copper and iron, thereby preventing the generation of reactive oxygen species responsible for cell injury and carcinogenesis (Harland, B. F. et al. (1987) “Phytate In Foods,” World Rev. Nutr. Diet 52:235-259). The preferred composition dosage of phytic acid (for example, as rice bran) is in the range of 2000-12,000 mg.

As used herein, the term “hyaluronic acid” (also known as hyaluronan) refers to linear polymer composed of repeating disaccharides of D-glucuronic acid and D-N-acetylglucosamine, linked together via alternating β-1,4 and β-1,3 glycosidic bonds ([−β (1,4)-GlcUA-β (1,3)-GlcNAc−]ₙ). Hyaluronic acid can be 25,000 disaccharide repeats (n) in length:

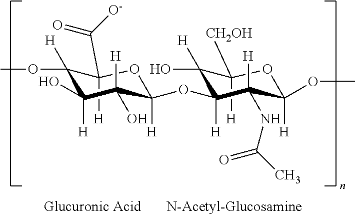

Hyaluronic acid is a water-retaining molecule that is generated naturally in the human body but in decreasing amounts as the body ages. Hyaluronic acid is a multifunctional glycosaminoglycan that forms the basis of the pericellular matrix of cells. Hyaluronic acid is synthesized by 3 different but related enzymes (hyaluronan synthases: HAS1, HAS2 and HAS3 (Weigel, P. H. et al. (1997) “Hyaluronan Synthases,” J. Biol. Chem. 272:13997-14000; Tammi, M. I. et al. (2002) “Hyaluronan And Homeostasis: A Balancing Act,” J. Biol. Chem. 277:4581-4584; Kakehi, K. et al. (2003) “Hyaluronic Acid: Separation And Biological Implications,” J. Chromatogr. B Analyt. Technol. Biomed. Life Sci. 797(1-2):347-355; Radaeva, I. F. et al. (1997) “Hyaluronic Acid: Biological Role, Structure, Synthesis, Isolation, Purification, And Application (Review)” Prikl. Biokhim. Mikrobiol. 33 (2):133-137; Stoolmiller, A. C. et al. (1970) “The Biosynthesis Of Hyaluronic Acid By Group A Streptococcus,” Expos. Annu. Biochim. Med. 30:65-78). U.S. Patent Application Publication 2004/0234497 discloses the use of hyaluronic acid for cancer drug delivery. The entire disclosure of that publication is incorporated herein by reference.

Hyaluronic acid has been traditionally extracted from rooster combs, from bovine or fish vitreous humor, from microbial production or from other sources (Rangaswamy, V. et al. (Epub 2007 Oct. 24) “An Efficient Process For Production And Purification Of Hyaluronic Acid From Streptococcus Equi Subsp. Zooepidemicus,” Biotechnol. Lett. 30(3):493-496; Gao, F. et al. (2006) “Preparation And Characterization Of Hyaluronan Oligosaccharides For Angiogenesis Study,” J. Biomed. Mater. Res. B Appl. Biomater. 78(2):385-392; Blank, L. M. et al. (2005) “Stable Production Of Hyaluronic Acid In Streptococcus Zooepidemicus Chemostats Operated At High Dilution Rate,” Biotechnol. Bioeng. 90(6):685-693; Kakehi, K. et al. (2003) “Hyaluronic Acid: Separation And Biological Implications,” J. Chromatogr. B Analyt. Technol. Biomed Life Sci. 797(1-2):347-355; Volpi, N. et al. (2003) “Purification And Characterization Of Hyaluronic Acid From The Mollusc Bivalve Mytilus Galloprovincialis,” Biochimie 85(6):619-625; Tawada, A. et al. (2002) “Large-Scale Preparation, Purification, And Characterization Of Hyaluronan Oligosaccharides From 4-Mers To 52-Mers,” Glycobiology. 2002 July; 12(7):421-426; Mahoney, D. J. et al. (2001) “Novel Methods For The Preparation And Characterization Of Hyaluronan Oligosaccharides Of Defined Length,” Glycobiology. 11(12):1025-1033; Mcdonald, J. et al. (2002) “Hyaluronan Minireview Series,” J. Biol. Chem. 277(7):4575-4579; Radaeva, I. F. et al. (1997) “Hyaluronic Acid: Biological Role, Structure, Synthesis, Isolation, Purification, And Application (Review)” Prikl. Biokhim. Mikrobiol. 33(2):133-137). Most preferably, the hyaluronic acid of the present invention is obtained from rooster combs. Hyaluronic acid is widely available commercially, and such preparations are suitable for the purposes of the present invention. Preferably, the compositions of the present invention will provide a composition dosage of hyaluronic acid of from about 1 mg to about 400 mg, more preferably from about 50 mg to about 200 mg.

As used herein, the term “Vitamin D” refers to a fat-soluble prohormone. Two major forms of vitamin D are vitamin D₂ (ergocalciferol) and vitamin D₃ (cholecalciferol) (DeLuca, H. F. et al. (1998) “Mechanisms And Functions Of Vitamin D,” Nutr. Rev. 56:S4-S10):

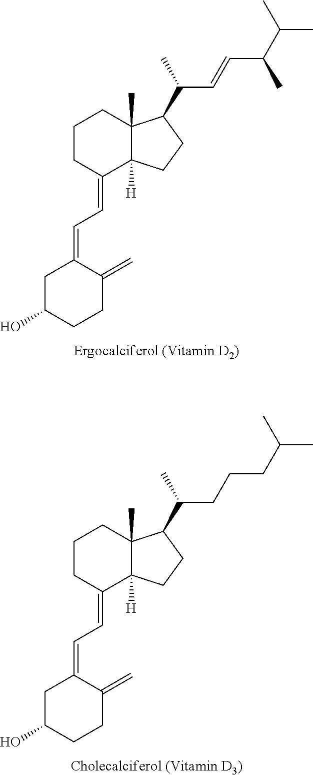 [SMILES: C=C1CC[C@H](O)C/C1=C/C=C1\CCC[C@]2(C)[C@@H]([C@H](C)/C=C/[C@H](C)C(C)C)CC[C@@H]12 ; C=C1CC[C@H](O)C/C1=C/C=C1\CCC[C@]2(C)[C@@H]([C@H](C)CCCC(C)C)CC[C@@H]12]

Vitamin D exhibits many biological actions. While vitamin D is widely known for its ability to stave off bone disease (rickets in growing children, osteoporosis in senior adults), it is becoming a central player in the battle against cancer. Regarding the role of vitamin D in immunity and cancer, vitamin D improves the chemotactic (affinity for) neutrophils to mobilize and migrate. Patients with rickets due to vitamin D deficiency are observed to have sluggish neutrophils that cannot migrate properly. Vitamin D stimulates the maturation of monocytes to macrophages. This results in an enlarged army of immune fighting cells to mount against tumors. Vitamin D is widely available commercially, and such preparations are suitable for the purposes of the present invention.

Vitamin D is essential for optimal muscle, bone, brain, immune and cardiovascular health and is undergoing re-discovery by aging researchers worldwide. Vitamin D supplementation up to 2000 IU has been shown to significantly reduce mortality rates, thus adding vitamin D to the lineup of molecules now considered to be true longevity factors (Autier, P. et al. (2007) “Vitamin D Supplementation And Total Mortality: A Meta-Analysis Of Randomized Controlled Trials,” Arch Intern Med. 167(16):1730-1737). Its anti-calcifying properties (Zittermann, A. et al. (2007) “Vitamin D And Vascular Calcification,” Curr. Opin. Lipidology 18(1):41-46) qualify vitamin D as another powerful agent that inhibits progressive overmineralization in the human body with advancing age and parallels the action of other mineral chelators in the compositions of the present invention. While the 1200 IU dose is three times more than the Recommended Daily Allowance, it is well within the Safe Upper Limit established by the National Academy of Sciences (2000 IU) and corresponds with a supplemental dosage recently found to be beneficial in a human clinical trial (Lappe, J. M. et al. (2007) “Vitamin D and calcium supplementation reduces cancer risk: results of a randomized trial,” Amer. J. Clin. Nutr. 85(6):1586-1591). A 2,000 IU dosage is roughly equivalent the natural vitamin D3 produced by 15-30 minutes of total-body summer sun exposure at noontime at a southern latitude, for which no side effects have been reported. Preferably, the compositions of the present invention will provide a composition dosage of vitamin D of from about 100 IU to about 100,000 IU, more preferably from about 1,000 IU to about 50,000 IU.

The compositions of the present invention may contain additional components, including additional active components that act to enhance resveratrol biological activity and inactive compounds (e.g., flavorants, sweeteners, dyes, vitamins, amino acids (e.g., lysine, proline, etc.), minerals, nutrients, etc.).

In particular, quercetin (3,3′,4′,5,7-pentahydroxy-2-phenylchromen-4-one)

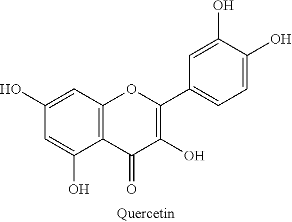 [SMILES: O=c1c(O)c(-c2ccc(O)c(O)c2)oc2cc(O)cc(O)c12]

butein, fisetin, myricetin, kaempferol, cis-resveratrol or piceatannol may be added to the compositions.

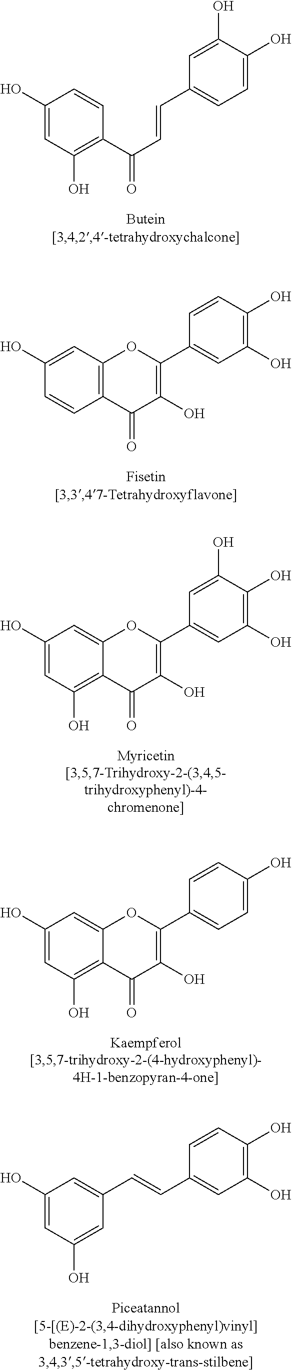 [SMILES: O=C(/C=C/c1ccc(O)c(O)c1)c1ccc(O)cc1O ; O=c1c(O)c(-c2ccc(O)c(O)c2)oc2cc(O)ccc12 ; O=c1c(O)c(-c2cc(O)c(O)c(O)c2)oc2cc(O)cc(O)c12 ; O=c1c(O)c(-c2ccc(O)cc2)oc2cc(O)cc(O)c12 ; Oc1cc(O)cc(/C=C/c2ccc(O)c(O)c2)c1]

Such compounds are described, for example by Stecher, G. et al. (2001) (“Determination Of Flavonoids And Stilbenes In Red Wine And Related Biological Products By HPLC And HPLC-ESI-MS-MS,” Fresenius J. Anal Chem. 2001 September; 371(1):73-80).

Resveratrol is glucuronated in the human liver, which may reduce its bioavailability. Flavonoids, such as quercetin, inhibit resveratrol glucuronidation and thus may act to improve resveratrol bioavailability (see, de Santi, C. et al. (2000) “Glucuronidation Of Resveratrol, A Natural Product Present In Grape And Wine, In The Human Liver,” Xenobiotica 30(11):1047-1054); De Santi, C. et al. (2000) “Sulphation Of Resveratrol, A Natural Compound Present In Wine, And Its Inhibition By Natural Flavonoids,” Xenobiotica 30(9):857-866; De Santi, C. et al. (2000) “Sulphation Of Resveratrol, A Natural Product Present In Grapes And Wine, In The Human Liver And Duodenum,” Xenobiotica 30(6):609-617). Quercetin may also act synergistically with resveratrol or independently of resveratrol to provide beneficial function (Kampkötter, A. et al. (Epub 2007 Oct. 16) “Increase Of Stress Resistance And Lifespan Of Caenorhabditis Elegans By Quercetin,” Comp. Biochem. Physiol. B Biochem. Mol. Biol. 149(2):314-323). (See also, Kaindl, U. et al. (2008) “The Dietary Antioxidants Resveratrol And Quercetin Protect Cells From Exogenous Pro-Oxidative Damage,” Food Chem. Toxicol. 46(4):1320-1326; Melzig, M. F et al. (2002) “Induction Of Neutral Endopeptidase And Angiotensin-Converting Enzyme Activity Of Sk-N-Sh Cells In Vitro By Quercetin And Resveratrol,” Pharmazie. 57(8):556-558; Hsu, C. L. et al. (2006) “Induction Of Cell Apoptosis In 3T3-L1 Pre-Adipocytes By Flavonoids Is Associated With Their Antioxidant Activity,” Molec. Nutr. Food Res. 50(11):1072-1079; Chan, M. M. et al. (2000) “Synergy Between Ethanol And Grape Polyphenols, Quercetin, And Resveratrol, In The Inhibition Of The Inducible Nitric Oxide Synthase Pathway,” Biochem. Pharmacol. 60(10):1539-1548; Nicholson S. K. et al. (2008) “Effects Of Dietary Polyphenols On Gene Expression In Human Vascular Endothelial Cells,” Proc. Nutr. Soc. 67(1):42-47; Lemos, C. et al. (2007) “Modulation Of Folate Uptake In Cultured Human Colon Adenocarcinoma Caco-2 Cells By Dietary Compounds,” Eur. J. Nutr 46(6):329-336).

Emulsifiers, fillers, binding agents, and the like may also be included in the compositions of the present invention.

In one embodiment, the compositions of the present invention comprise a novel combination of small molecules such as quercetin and resveratrol with widespread preventive and therapeutic health properties; and/or collagen-building nutrients (such as vitamin C-ascorbate, lysine, proline, etc.); and a glycosaminoglycan such as a shortened (low molecular weight) chain of hyaluronic acid (HA) or its singular components (glucosamine, glucuronate) or chondroitin sulfate, which are linear disaccharides (sugar-like molecules) that serve as structural components of cartilage, but in this combination serve as synergistic co-healing agents in non-cellular (connective) tissue that surrounds living cells. The combination of glycosaminoglycans (hyaluronan, glucuronate or chondroitin) and/or ascorbate, lysine or proline, to encourage the generation of collagen, and small molecules, that operate on intra-cellular basis, comprise therapeutic or preventive action that encompasses the total tissue matrix. The combination of the present invention is intended for human or animal oral intake as a dietary supplement. In a sub-embodiment thereof, such compositions may comprise a combination of resveratrol and hyaluronan in a dietary supplement that serves to heal a variety of illnesses including some cancers. Resveratrol is known to be an anti-cancer molecule and to have other healing and longevity enhancing properties. Hyaluronan (hyaluronic acid, HA) is taken as an oral supplement or can be given intravenously to target cancer cells. When combined with or attached to other molecules, hyaluronan will deliver other anti-cancer and healing agents such as resveratrol to tumor sites. The combination may or may not include a chelating agent, an antioxidant and/or an emulsifier as described in my above-referenced co-pending patent application. When encapsulated or otherwise applied together, with or without those additives, resveratrol and HA have powerful healing properties for animals and humans.

Most preferably, the compositions of the present invention stabilize resveratrol specific activity such that the resveratrol of the compositions has a specific activity that is greater than that of resveratrol maintained in the presence of oxygen gas, or maintained in the absence of a chelator, hyaluronic acid, or vitamin D. Preferably, the amounts of the non-resveratrol constituents of the compositions will stabilize the composition's resveratrol so that it exhibits at least 10% more activity, at least 20% more activity, at least 50% more activity, at least 2-times the activity, at least 5-times the activity, or at least 10-times the activity of resveratrol maintained in the presence of oxygen gas, or maintained in the absence of a chelator, hyaluronic acid, or vitamin D and so that it remains capable of exhibiting such specific activity over extended periods (for example, 1, 2, 4, 6, 10, 12, 18, 24, or 36 months or longer) at ambient conditions of temperature and humidity (i.e., without need for special precautions as to temperature or humidity).

D. Packaging of the Preferred Compositions of the Present Invention

Resveratrol is typically unstable to light and oxidation (Shaanxi University of Science & Technology, Xianyang China (2007) “Study On The Stability Of Resveratrol In Rhizoma Polygoni cuspidate,” Zhong Yao Cai. 30(7):805-80). The resveratrol of the present invention is preferably prepared, packaged and/or stored in a manner that maximizes its specific activity. It is preferred to prepare, package and/or store resveratrol in low light (or in the dark) and/or in low oxygen, so as to minimize light-induced degradation (e.g., photo-isomerization) or oxygen-induced degradation. The preferred compositions of the present invention are formulated as dietary supplements for oral ingestion in the form of a pill, lozenge, capsule, elixir, syrup, etc. Other modalities of administration may alternatively be employed (e.g., intranasal, parenteral, intravenous, intraarterial, topical, etc.).

In a first example of such preferred packaging, the compositions of the present invention are formulated as air-tight capsules in which encapsulation is conducted so as to prevent or minimize exposure to oxygen. In one embodiment, such encapsulation is conducted in an oxygen-free environment. For example, the components of the compositions of the present invention may be inserted into a capsule in an inert gas (e.g., nitrogen, argon, etc.) environment. Preferably, a nitrogen bubble (e.g., 5-20% of the capsule volume) may be introduced into the capsule to further stabilize and protect the components against oxidation (see, PCT Publication No. WO 01/08631, herein incorporated by reference). That international application has a corresponding U.S. patent application. Suitable capsules useful in the encapsulation of resveratrol and other oxidation prone ingredients of dietary supplements include Licaps® (Capsugel), an air-tight gelatin capsule. The presence of phytic acid, which has the ability to protect the components from metal-induced oxidation, augments such anti-oxidation precautions. A particularly preferred example of such a resveratrol-containing composition is Longevinex® (Resveratrol Partners, LLC, San Dimas, Calif.), which comprises resveratrol and phytic acid. Longevinex® contains as active ingredients (per capsule): 5 mg Vitamin E (as mixed tocopherols), 215 mg total resveratrol (obtained from French red wine and giant knotwood (Polygonum cuspidatum), and providing 100 mg of trans-resveratrol), 25 mg quercetin dihydrate, 75 mg phytic acid (rice bran extract), 380 mg rice bran oil, 55 mg sunflower lecithin.

Once a composition has been sealed into an air-tight capsule, it is important to maintain a low or no-oxygen environment in the packaging surrounding the capsules in order to protect the composition from oxidation should a break or leak occur in the sealed capsule. Therefore, an oxygen absorbing packette is preferably employed to reduce the presence of free oxygen. Vacuum or nitrogen-flushed packaging (bottles, pill cases, etc.) in air-tight materials is desirable.

In an alternative embodiment, the components and compositions of the present invention may be prepared as a microencapsulated process (see, generally, Rubiana, M. et al. (2004) “Drug Delivery Systems: Past, Present, and Future,” Current Drug Targets, 5(5):449-455). Micro-encapsulation is a process by which tiny particles or droplets (ranging in size from a few nanometers to one micron) are coated with a protective layer to create small capsules with controlled properties. Suitable micron-sized, encapsulated, preparations can be obtained using the microencapsulation processes of Maxx Performance Inc. (Chester, N.Y.), Blue California (Rancho Santa Margarita, Calif.), Southwest Research Institute (San Antonio, Tex.), Coating Place, Inc. (Verona, Wis.), Microtek Laboratories (Dayton, Ohio), Particle Sciences, Inc. (Bethlehem, Pa.), etc. 3^{rd}-generation Longevinex® (“Longevinex-3®”) (Resveratrol Partners, LLC), which contains Vitamin D3, Vitamin E, Resveratrol, Quercetin, and Phytic Acid is a particularly preferred microencapsulated form of the compositions of the present invention.

The present invention further comprises a practical method of stabilizing quercetin and other easily oxidized dietary supplement ingredients which may come in contact with oxidizing metals.

E. Utility of the Compositions of the Present invention

The compositions of the present invention enhance resveratrol's specific activity. The compositions of the present invention therefore find utility in the treatment of diseases (or in the amelioration of the symptoms of diseases) such as cardiovascular disease, cancer, macular degeneration, aging, neurodegenerative diseases (e.g., Alzheimer's Disease, Parkinson's Disease, etc.) and inflammation in which the modulation of expression of “survival/longevity” genes and/or “damage inducing” genes is desired. Over time, as minerals such as calcium and iron accumulate in the human body, genes respond in deleterious ways. Liu, Y. et al. (2005) “Global Genomic Approaches To The Iron-Regulated Proteome,” Ann. Clin. Lab. Sci. 35(3):230-239; Templeton, D. M. et al. (2003) “Genetic Regulation Of Cell Function In Response To Iron Overload Or Chelation,” Biochim. Biophys. Acta. 1619(2):113-124; Ikeda, H. et al. (1992) “Evidence That An Iron Chelator Regulates Collagen Synthesis By Decreasing The Stability Of Procollagen mRNA,” Hepatology 15(2):282-287. The present invention has particular utility in the treatment of macular degeneration, cancer and the conditions of aging.

1. Macular Degeneration

The prolongation of the human lifespan over the past few decades in the US has spawned the proliferation of macular degeneration, an age-related eye disease. While not resulting in total vision loss, the disease robs older adults of their central vision used for reading as well as color vision. Macular degeneration affects the visual center of the eye, called the macula. The macula is part of the retina where color-vision cells (cones) are located.

Macular degeneration is a progressive, age-related disease that can be broken down into four stages:
1. Beginning in about the third decade of life, the inability of the “garbage cleaning” cells, called the retinal pigment epithelia (RPE), to engulf and remove cellular debris from the back of the eyes, results in the formation of small microscopic deposits called lipofuscin (ly-poh-fus-kin). Lipofuscin is from by iron and copper-induced oxidation of cellular debris and its accumulation correlates with premature aging and shortened lifespan of organisms. The prevalence of macular degeneration is greater in Caucasians than persons with darkly-pigmented skin and Caucasians have more lipofuscin deposits in their retinas. Some of this cellular debris in the retina is comprised of used-up vitamin A that is shed from night-vision (rod) cells each morning in the human eye. The failure of the RPE cells to function results from accumulation of iron and calcium within the RPE.
2. Later, in about the fifth decade of life, there is progressive calcification of an underlying cellophane-thin retinal layer called Bruch's membrane, which resides between the RPE and the blood supply layer (choroid). While drusen that forms within the retina is partially composed of cholesterol, this lipid does not originate from the blood circulation or the liver where most cholesterol is produced. Calcifications within Bruch's membrane further impairs the exit of lipids (fats), protein, and cellular debris, from the photoreceptor layer, which results in the formation of yellow spots called drusen on the retina. Drusen can be observed during an eye examination using an ophthalmoscope. There is currently no method of removing drusen.
3. The death of the RPE cells is the third stage of this progressive disease. This is sometimes called RPE dropout. As the RPE cells are either impaired or have died, and Bruch's membrane is clogged with calcium, the photoreceptors then cannot be nourished and also begin to die off. There is currently no treatment for stages 1-3 of macular degeneration. Stage 1-3 is called the “dry” form of macular degeneration because it has not resulted in hemorrhage or edema or new blood vessel formation. About 85% of macular degeneration patients have the “dry” form of this disease.
4. As breaks in Bruch's membrane occur, or Bruch's membrane becomes totally calcified, the photoreceptor layer is deprived of oxygen and new blood vessels form (called neovascularization) which can invade the photoreceptor layer in the macula and impair vision; or there may be leakage of blood serum or frank release of red blood cells, which results in edema or hemorrhage. This is the more advanced and sight-threatening form of macular degeneration, often called “wet” macular degeneration because of the presence of the leakage of blood serum or red blood cells into the photoreceptor layer. This stage of the disease, if caught early, can be treated with laser beams, which can seal up leaky blood vessels. However, this treatment is only effective in delaying the progression of the disease, not curing it.

The cell cleansing process facilitated by the lysosomes cannot keep up with the accumulation of metabolic waste over a lifetime. The parafoveal ring, where rod cell density is highest, and therefore more discs of used-up vitamin A are shed, is where macular degeneration begins, and where the highest concentration of lipofuscin is observed in the retina. Eventually, the RPE cells die off with advancing age, which increases the burden on the remaining RPE cells to maintain a healthy retina.

In the past, lipofuscin has been considered a harmless wear-and-tear byproduct of cellular metabolism. One aspect of the present invention relates to the recognition that lipofuscin, which forms from iron and copper-induced oxidation, and hardens within lysosomal bodies within retinal pigment epithelial cells, sensitizes the retina to damage by mild amounts of radiation and oxidation. The retina becomes increasingly sensitive to blue-light damage with advancing age. Drusen formation within the retina is associated with RPE cell inability to produce superoxide dismutase, an endogenous antioxidant enzyme. Mice deficient in superoxide dismutase develop features that are typical of age-related macular degeneration in humans. Superoxide dismutase protects retinal cells against unbound (free) iron. High iron diets and cellular environments have been shown to reduce superoxide dismutase activity.

Retinal photoreceptors and retinal pigment epithelial cells are believed to be especially vulnerable to damage by low-molecular weight complexes of iron. Since antioxidants in the blood circulation may not always be able to cross the blood-retinal barrier, the retina produces its own protective antioxidants that bind iron. Iron chelators inhibit the adverse effects of unbound (free) iron (not bound to proteins). Heme oxygenase also serves in a similar manner to iron chelators to prevent retinal damage induced by loose iron.

Numerous agents have been used experimentally to clear up lipofuscin and drusen. Statin drugs, commonly used to reduce blood serum levels of cholesterol, have also been tested to prevent lipofuscin deposits in animals. Statin drugs reduced lipofuscin formation but were toxic to the liver and brought about the early death of these animals. Piracetam, a derivative of the neurotransmitter GABA, now available as a dietary supplement, has been used successfully to reduce lipofuscin formation in brain tissues. Sorbinil is an enzyme inhibiting drug (aklose reductase inhibitor) that underwent unsuccessful human trials in the 1990s to prevent retinal problems associated with diabetes. Sorbinil has been shown to partially reduce lipofuscin deposits in the retinal pigment epithelium cells of rodents. Hydergine is a drug used to treat senile dementia. In a rodent study, hydergine was reported to have reduced brain lipofuscin levels, but also led to the early demise of the animals. The East Indian spice turmeric contains an antioxidant molecule called curcumin. Curcumin has been used in an experimental mouse study to reduce lipofuscin in the brain. Purslane is a flowering plant rich in magnesium, beta carotene and omega-3 oil. The provision of purslane to mice has been shown to reduce lipofuscin deposition in the brain of mice.

In a lab dish study, sulforaphane, an antioxidant molecule found in Brussels sprouts and broccoli in 1992, has been used successfully to reduce lipofuscin deposits in RPE cells exposed to blue light.

Intraperitoneal administration of lipoic acid to aged rats leads to a reduction and elevation in lipofuscin and enzyme activity, respectively, in the cortex, cerebellum, striatum, hippocampus, and hypothalamus of the brain. These results suggest that lipoic acid, a natural metabolic antioxidant, should be useful as a therapeutic tool in preventing neuronal dysfunction in aged individuals. Lipoic acid, a natural antioxidant produced within living tissues, and also available as a dietary supplement, has been shown to protect RPE cells from oxidative damage in lab dish studies.

Lipofuscin formation dramatically increases in brain tissues following alcohol consumption. Supplementation with high-dose grape seed flavonols prevents increase lipofuscin formation. Lipofuscin is an end-product of lipid peroxidation which dramatically increases following ethanol consumption.

Oolong and green tea drinks reverse the cognitive impairment and lipofuscin formation in mice. Epigallocatechin-3-gallate (EGCG), the major constituent of green tea, upregulates the activity of heme oxygenase in lab dish studies. Heme oxygenase is a protective enzyme against iron-induced oxidation, which occurs in the retina.

It has been shown that the provision of supplemental estrogen decreases lipofuscin deposition in brain tissues. In a lab dish study, the provision of lutein and zeaxanthin to RPE cells reduced lipofuscin formation. In rodents given supplemental acetyl-L-carnitine, a decline in lipofuscin deposits has been measured in brain cells.

U.S. Pat. No. 5,747,536 describes the combined therapeutic use of L-carnitine, lower alkanoyl L-carnitines or the pharmacologically acceptable salts thereof, with resveratrol, resveratrol derivatives or resveratrol-containing natural products, for producing a medicament for the prophylaxis and treatment of cardiovascular disorders, peripheral vascular diseases and peripheral diabetic neuropathy.

Melanin is an iron-binding antioxidant in the retina. As melanin levels decline in the retina with advancing age, there is a greater accumulation of lipofuscin.

In one embodiment, the present invention relates to a composition comprising a combination of:
(a) A chelator such as inositol hexaphosphate (IP6), trans resveratrol, quercetin, or any polyphenol or bioflavonoid for metal(s) such as iron, copper, heavy metals;
(b) A calcium chelator, such as inositol hexaphosphate (IP6);
(c) A heme oxygenase activator, such as trans resveratrol, piceatannol, or any of resveratrol's natural analogs, or similar small molecules such as fisetin, myricetin, quercetin or other bioflavonoids;
(d) An agent that lowers the affinity of oxygen for red blood cells, such as inositol hexaphosphate (IP6); and, optionally
(e) Other antioxidants such as vitamin E, lutein/zeaxanthin, alpha lipoic acid.

The formulation functions to:
(1) Limit oxidation in retinal tissues (photoreceptors, retinal pigment epithelial cells (RPE), choroid, specifically mitochondria and lysosomes in RPE cells);
(2) Inhibit accumulation of lipofuscin deposits;
(3) Inhibit formation of drusen; and
(4) Limit calcifications to retinal tissues, especially Bruch's membrane.

2. Cancer

A major challenge in cancer therapy is to selectively target cytotoxic agents to tumor cells (Luo, Y. et al. (2000) “A Hyaluronic Acid-Taxol Antitumor Bioconjugate Targeted To Cancer Cells,” Biomacromolecules 1(2):208-218). To decrease undesirable side effects of small molecule anticancer agents, many targeting approaches have been examined. One of the most promising methods involves the combination or covalent attachment of the cytotoxin with a macromolecular carrier, and in particular with hyaluronic acid (Luo, Y. et al. (1999) “Synthesis And Selective Cytotoxicity Of A Hyaluronic Acid-Antitumor Bioconjugate,” Bioconjug. Chem. 10(5):755-763; Luo, Y. et al. (2000) “A Hyaluronic Acid-Taxol Antitumor Bioconjugate Targeted To Cancer Cells,” Biomacromolecules 1(2):208-218; Luo, Y. et al. (1999) “Hyaluronic Acid-N-Hydroxysuccinimide: A Useful Intermediate For Bioconjugation,” Bioconjug. Chem. 12(6):1085-1088; Luo, Y. et al. (2002) “Targeted Delivery Of Doxorubicin By HPMA Copolymer-Hyaluronan Bioconjugates,” Pharm. Res. 19(4):396-402).

In one embodiment, the present invention relates to a resveratrol- and hyaluronic acid-containing composition for the treatment of cancer comprising: resveratrol, hyaluronan, and optionally vitamin D and/or IP6. It is believed that these components act synergistically with one another to mediate an effect in curing and/or in preventing cancer in humans and/or in improving immunity (e.g., immune system response) in patients threatened by tumors. This aspect of the present invention is based in part upon the recognition that natural molecules can boost cancer immunity, possibly in a manner similar to that observed in cancer-proof mice.

Upon provision with such composition, the sentinels of the innate immune system, dendritic cells, can be alerted and neutrophils, macrophages and natural killer cell activity can be significantly enhanced. The enhancement of vitamin D receptors via resveratrol is yet another major advantage of a combination approach to treat or prevent cancer. This approach appears to be more appropriate for senior adults, the highest risk group for cancer, who are often immune-compromised due to poor nutrition or lack of nutrient absorption. The fact that this therapy can now be immediately measured for effectiveness by non-invasive cancer cell counting technology means that expensive and equivocal tests on animals may not be required to prove efficacy.

Vitamin D exhibits many biological actions. While vitamin D is widely known for its ability to stave off bone disease (rickets in growing children, osteoporosis in senior adults), it is becoming a central player in the battle against cancer. Only recently is it also gaining attention as an antibiotic. Vitamin D-deficient mice exhibit a defective response from phagocyte cells in the face of infection or inflammation. Vitamin D deficiency is frequently associated with recurrent infections. Only about half of the macrophage cells accumulate at the site of inflammation in vitamin D-deficient animals compared to animals whose vitamin D levels are adequate.

To delve deeper into the role of vitamin D in immunity and cancer, vitamin D improves the chemotactic (affinity for) neutrophils to mobilize and migrate. Patients with rickets due to vitamin D deficiency are observed to have sluggish neutrophils that cannot migrate properly. Vitamin D stimulates the maturation of monocytes to macrophages. This results in an enlarged army of immune fighting cells to mount against tumors. Greater attention is now being given to vitamin D as an anti-cancer weapon because of studies which show supplemental vitamin D drastically reduces the risk for all types of cancer. A study that employed 1100 IU of vitamin D3 produced a 60-77% reduction in cancer risk among women in Nebraska in just a 4-year period.

Even though cancer risk is lowest in sunnier and Equatorial areas geographically, where vitamin D levels are higher in sun-exposed populations, the protective effect of vitamin D against cancer has been repeatedly dismissed or discounted. The consumption of vitamin D orally eliminates the concern of skin cancer emanating from overexposure to unfiltered sun rays. One of the latest analyses shows that the risk of colon cancer can be halved by taking 2000 IU of vitamin D per day and that the risk for breast cancer can be halved by taking 3500 IU of vitamin D per day. The median dietary intake of vitamin D is only about 230 IU per day, so the prospect of food fortification or supplementation to prevent or treat cancer now becomes real.

In order for tissues to utilize and benefit from vitamin D they must have proteins in their outer coat (cell membrane) that are designed to receive and bind to vitamin D. For example, about 80% of human breast tumors produce vitamin D cell receptors, though gene expression (production) of vitamin D receptor is at low levels.

Vitamin D's ability to inhibit cancer may be heightened when it is aided by weak estrogen-like molecules in the diet. Resveratrol, an estrogen-like molecule commonly found in red wine, upregulates the vitamin D receptor in breast cancer cells without increasing cancer growth. Resveratrol, in effect, can sensitize breast cancer cells to the anti-cancer properties of vitamin D.

Laboratory experiments show that low-dose vitamin D3 does not reduce breast tumor cell growth but when combined with resveratrol, tumor cell numbers declines by 40%. At higher concentrations vitamin D3 reduces the number of breast cancer cells in a lab dish by about 25%, and this decline improves to 50% when combined with resveratrol. Whereas estrogen increases vitamin D receptor gene expression, it also stimulates breast tumor growth. Resveratrol does not have this drawback. Resveratrol potentiates or “weaponizes” the cancer-inhibiting effect of vitamin D.

Furthermore, resveratrol by itself has been shown to calm the response of phagocytes to foreign invaders like germs and tumor cells. Resveratrol dampens production of reactive oxygen species (free radicals) and normalizes particle ingestion in macrophage cells. Therefore, resveratrol prevents the over-response of immune cells that can produce autoimmunity.

Resveratrol blocks cancer in so many ways that it is difficult to find a pathway for cancer that is not obstructed by resveratrol. Resveratrol induces the cell energy compartments in tumor cells, called mitochondria, to release an enzyme called cytochrome C oxidase that usually leads to a cascade of other enzymes that induce programmed cell death, called apoptosis. But a recent experiment also shows that resveratrol releases cytochrome C from ovarian tumor cells that leads to rapid cell death via a process called autophagy, a process where enzymes produced inside the tumor cell actually digest its innards (kind of a form of intracellular cannibalism). This is a form of cell suicide that resveratrol activates in tumor cells, but not healthy cells.

The contribution of innate immunity in surveillance of tumors is comparatively neglected in cancer biology. Phagocytosis, or “cell eating” is the cornerstone of the innate immune response. Focus has been directed to dendritic cells which are believed to be sentinels of the innate immune response. A limited number of immune-boosting agents have been investigated.

Skepticism surrounds interest in innate immune approaches to cancer treatment. For example, patients taking immune-suppressing don't necessarily develop cancer with more frequency. However, this may be misunderstood. An over-responsive immune system may lead to more tissue and organ damage that can be mortal to cancer patients. Most of the drugs used for breast cancer therapy induce immune suppression.

Nature's most potent iron chelator is inositol hexaphosphate (IP6), which is found in seeds and the bran fraction of whole grains. A low dosage of IP6 has been found to suppress the growth of rhabdomyosarcoma cells by 50%. Removal of IP6 allows these tumor cells to recover and grow once again. IP6-treated mice with injected tumors exhibit tumors that are 50 times smaller than non-treated mice. IP6 has also been shown to reduce the growth of injected fibrosarcoma cells in mice and prolong their survival.

In examining the immune enhancing properties of IP6 it has been shown that it boosts production of free radicals (superoxide) and the cell digesting action of neutrophils in the presence of bacteria. IP6 increases the release of interleukin-8.

The action of natural killer cells, which are involved in tumor cell destruction, is enhanced by IP6.

In one embodiment, the hyaluronic acid of such composition is conjugated to a chemotherapeutic agent. The invention particularly pertains to such compositions in which the chemotherapeutic agent is taxol. The invention particularly pertains to such compositions that additionally and preferably comprise a chelator, and/or vitamin D. Most malignant solid tumors contain elevated levels of Hyaluronic Acid (Rooney, P. et al. (1995) “The Role Of Hyaluronan In Tumour Neovascularization (Review),” Int. J. Cancer 60(5):632-636) and these high levels of HA production provide a matrix that facilitates invasion (Hua, Q. et al. (1993) “Internalization Of Hyaluronan By Chondrocytes Occurs Via Receptor-Mediated Endocytosis,” J. Cell. Sci. 106(Pt 1):365-375; Luo, Y. et al. (2000) “A Hyaluronic Acid-Taxol Antitumor Bioconjugate Targeted To Cancer Cells,” Biomacromolecules 1(2):208-218). Thus chemotherapeutic agents that are conjugated to Hyaluronic Acid target tumor cells, and can provide an effective anti-tumor dosage at lower overall concentration.

In brief, a preferred method of conjugation entails forming an NHS (N-hydroxy-succimimide derivative of the chemotherapeutic agent. Such a derivative can be made by adding a molar excess of dry pyridine to a stirred solution of Taxol and succinic anhydride in CH₂Cl₂ at room temperature. The reaction mixture is then stirred for several days at room temperature and then concentrated in vacuo. The residue is dissolved in 5 ml of CH₂Cl₂ and the produced Taxol-2′-hemisuccinate can be purified on silica gel (washed with hexane; eluted with ethyl acetate) to give the desired product (Luo, Y. et al. (1999) “Synthesis And Selective Cytotoxicity Of A Hyaluronic Acid-Antitumor Bioconjugate,” Bioconjug. Chem. 10(5):755-763).

The N-hydroxy-succimimide derivative of the chemotherapeutic agent is then conjugated to adipic dihydrazido-functionalized hyaluronic acid. Adipic dihydrazido-functionalized hyaluronic acid is preferably prepared as described by Pouyani, T. et al. (1994) (“Functionalized Derivatives Of Hyaluronic Acid Oligosaccharides—Drug Carriers And Novel Biomaterials,” Bioconjugate Chem. 5:339-347); Pouyani, T. et al. (1994) (“Novel Hydrogels Of Hyaluronic Acid: Synthesis, Surface Morphology, And Solid-State NMR,” J. Am. Chem. Soc. 116:7515-7522); Vercruysse, K. P. et al. (1997) (“Synthesis And In Vitro Degradation Of New Polyvalent Hydrazide Cross-Linked Hydrogels Of Hyaluronic Acid,” Bioconjugate Chem. 8:686-694). Thus, hyaluronic acid is preferably dissolved in water and an excess of adipic dihydrazide (ADH). The pH of the reaction mixture is adjusted to 4.75 by addition acid. Next, 1 equivalent of 1-Ethyl-3-[3-(dimethylamino)-propyl]carbodiimide (EDCI) is added in solid form. The pH of the reaction mixture is maintained at 4.75 by addition of acid. The reaction is quenched by addition of 0.1 N NaOH to adjust the pH of reaction mixture to 7.0. The reaction mixture is then transferred to pretreated dialysis tubing (Mw cutoff 3,500) and dialyzed exhaustively against 100 mM NaCl, then 25% EtOH/H2O and finally water. The solution is then filtered through 0.2 m cellulose acetate membrane, flash frozen, and lyophilized (Luo, Y. et al. (1999) “Synthesis And Selective Cytotoxicity Of A Hyaluronic Acid-Antitumor Bioconjugate,” Bioconjug. Chem. 10(5):755-763).

3. Aging

Calcification and rusting are major accelerators of aging. The human body is composed of cells that must continually be replaced or renewed from within, and a gooey substance that fills space between cells called collagen or connective tissue which also must be continually regenerated. As the human body ages at the cellular level, there is a slow accumulation of cellular debris called lipofuscin. The formation of lipofuscin is facilitated by the progressive accumulation of iron and calcium within cell bodies called lysosomes and mitochondria. A cell cleansing and renewal process called autophagy prevents the accumulation of lipofuscin. Progressive inability to remove cellular debris results in declining cell function and then premature death of the cell. A young cell efficiently removes debris from within. An old cell cannot efficiently remove debris and accumulates lipofuscin.

Calcification and rusting of cells impairs the cleansing of cellular debris (lipofuscin) from cells by enzymes produced by lysosomes, and results in impairment of cellular energy (ATP) produced by the mitochondria within cells. The compositions of the present invention inhibit and/or reverse cellular aging and/or connective tissue aging, and in particular, inhibit and/or reverse cellular aging and/or connective tissue aging caused by an accumulation of major minerals (e.g., iron, calcium, etc.). As a consequence, recipients of the compositions of the present invention exhibit enhanced longevity and enhanced cellular and connective tissue health and structure.

The human body ages at the cellular level by the slow accumulation of cellular debris called lipofuscin, which is facilitated by the progressive accumulation of iron and calcium within cell bodies called lysosomes and mitochondria. A cell cleansing and renewal process called autophagy prevents the accumulation of lipofuscin during the years of youthful growth, but this lysosomal mechanism declines once full growth is achieved due to accumulation of intracellular iron and calcium. Progressive inability to remove cellular debris results declining cell function and then premature death of the cell. A young cell efficiently removes debris from within. An old cell cannot efficiently remove debris and accumulates lipofuscin. The mitochondria, which provides cellular energy for lysosomal bodies to perform their cell cleansing activity, also becomes progressively calcified and ironized once childhood growth ceases. Only about 5% of mitochondria are functioning by age 80. Iron and calcium chelators are proposed to remedy mitochondrial aging which impacts cellular functions such as lysosomal enzymatic activity

The human body ages within connective tissue by failure of cells called fibroblasts to regenerate collagen and hyaluronic acid, the latter being a space-filling, water-holding molecule. Collagen formation is facilitated by vitamins and amino acids in the diet (vitamin C, lysine, proline). Fibroblasts can be stimulated to produce hyaluronic acid by estrogen, made naturally in the body, and by estrogen-like molecules found in plants, called phytoestrogens, provided in the diet of by hyaluronic acid itself. Young females, by virtue of the ability to produce estrogen, exhibit thicker hair, smoother skin and more flexible joints, due to the abundance of hyaluronic acid. All of these being attributes of youthfulness.

The inability to regenerate hyaluronic acid results in tissues losing their physical integrity by virtue of loss of the space-filling properties of hyaluronic acid. Without adequate hyaluronic acid, a dehydrated state results and tissues shrink and shrivel up. For example, skin that is lacking hyaluronic acid will appear wrinkled and dry. Joint spaces will lack the cushioning and space-filling needed to prevent bone from rubbing on bone. The eyes will begin to shrink in size. Hair will thin due to the lack of hydration. These are the most prominent visible or cosmetic signs of aging.

In one embodiment, the present invention invention addresses both cellular and extracellular (connective tissue) aging, thus (a) preserving youthful function of living cells by removal of excess minerals, largely calcium and iron, from cells, this facilitating autophagy (cleanup of cellular debris, such as lipofuscin, via lysosomal enzymes) and (b) invigorating and preserving production of hyaluronan by stimulation of fibroblasts by HA, phytoestrogens (resveratrol, quercetin, genistein, are a few), to inhibition of degradation of HA by provision of metal chelators, such as phytic acid, ferulate, quercetin, resveratrol, etc.

In one embodiment, the present invention is a dietary supplement that addresses both cellular and non-cellular aging by its ability to:
(a) stimulate renewal of living cells from within via enzymatic degradation of cellular debris by intracellular lysosomal bodies. This is facilitated by the inclusion of metal (iron, copper, heavy metal) and calcium chelating molecules within the formula. Lysosomes lose their ability to enzymatically digest cellular debris with the progressive accumulation of iron, copper and other metals, and the crystallization of calcium.
(b) stimulate fibroblasts to produce hyaluronic acid at youthful levels again. This is accomplished by provision of orally-consumed molecules that stimulate fibroblasts to produce hyaluronic acid.
(c) metal chelating molecules that help maintain youthful lysosomal function are identified as antioxidants, like vitamin E or vitamin C, lipoic acid, metal chelators like IP6 phytate, quercetin, bioflavonoids or polyphenols, resveratrol. Resveratrol works by its ability to stimulate production of heme oxygenase, an enzyme that helps to control iron.
(d) molecules that inhibit crystallization of calcium are magnesium and IP6 phytate.
(e) orally consumed molecules that stimulate fibroblasts to produce hyaluronic acid are hyaluronic acid, glucosamine, chondroitin, or estrogen-like molecules such as genistein, lignans, hydroxytyrosol, or other molecules configured like estrogen. Orally consumed HA stimulates greater HA and chondroitin synthesis. Similarly, glucosamine stimulate fibroblasts to produce HA. Alternatively, or additionally, glucosamine stimulates synovial production of hyaluronic acid, which is primarily responsible for the lubricating and shock-absorbing properties of synovial fluid” (McCarty, M. F. (1998) “Enhanced Synovial Production Of Hyaluronic Acid May Explain Rapid Clinical Response To High-Dose Glucosamine In Osteoarthritis,” Medical Hypotheses 50:507-510, 1998).
(f) orally consumed molecules that stimulate production of collagen are vitamin C, proline and lysine.

In such embodiment, the present invention relates to a resveratrol and hyaluronic acid-containing dietary supplement that restores youthful function and appearance to human cells and tissue. The invention particularly pertains to such compositions that additionally comprise a chelator, and/or vitamin D. Most preferably, the composition will comprise the chelator phytic acid (inositol hexaphosphate; IP6). The compositions of the present invention synergistically enhance the specific activity of the resveratrol and/or hyaluronic acid, and thus the compositions of the present invention provide an enhancement of activity above and beyond that obtained with the components administered individually. In such embodiment, the invention relates to a method for restoring youthful function and appearance to human cells and tissues comprising the following steps:
(a) stimulating renewal of living cells from within via enzymatic degradation of cellular debris by intracellular lysosomal bodies (preferably by providing a metal chelating molecule that helps maintain youthful lysosomal function, such molecules comprising antioxidants, such as vitamin E or vitamin C, lipoic acid, metal chelators like IP6 phytate, quercetin, bioflavonoids or polyphenols, and/or resveratrol); and
(b) stimulating fibroblasts to produce hyaluronic acid (comprises providing orally consumed molecules that stimulate fibroblasts to produce hyaluronic acid, such orally consumed molecules comprising, for example, hyaluronic acid, glucosamine, chondroitin, and/or estrogen-like molecules such as genistein, lignans, hydroxytyrosol, or other molecules configured like estrogen).
   Preferably, such stimulation is achieved by the dietary administration of a composition comprising the stated compounds, more preferably in combination with an orally consumable molecule that stimulates production of collagen, such molecules comprising, for example, vitamin C, proline and/or lysine.

Without intending to be bound by any mechanism, when administered together, the four preferred components are believed to act in the following manner:

(a) Vitamin D:
Vitamin D3 works as an agent that mimics the response to a biological stressor, solar radiation. In particular, vitamin D3 upregulates protective genes involved in activation of the immune system, particularly neutrophil count and motility, and aids in overcoming the decline in endogenous vitamin D3 production with advancing age due to thickening of the skin, which reduces sun/skin production of vitamin D. Furthermore, vitamin D3 works synergistically to breakdown IP6 to IP3, thought to be a major active molecule. Resveratrol also works synergistically to sensitize cells to vitamin D3 (sensitizes the vitamin D receptor on the cell surface). Vitamin D serves to break down IP6 to IP3, which is its primary active form. Vitamin D is also believed to act as an immune system enhancing agent, boosting innate immunity in humans. In this capacity, vitamin D has been shown experimentally to have important cancer-preventive and cancer-curing properties.

(b) Resveratrol:
Resveratrol increases the sensitivity of the vitamin D receptor on the surface of cells, and thus is believed to act as an enhancing agent for vitamin D and as an anti-cancer agent. Resveratrol up-regulates the vitamin D receptor on the surface of cancer cells, and sensitizes cancer cells to vitamin D (Wietzke, J. A. et al. (2003) “Phytoestrogen Regulation Of A Vitamin D3 Receptor Promoter And 1,25-Dihydroxyvitamin D3 Actions In Human Breast Cancer Cells,” J. Steroid Biochem. Molec. Biol. 84(2-3):149-157; Wietzke, J. A. et al. (2005) “Regulation Of The Human Vitamin D3 Receptor Promoter In Breast Cancer Cells Is Mediated Through Sp1 Sites,” Molec. Cell. Endocrinol. 230(1-2): 59-68). Resveratrol is also believed to be a monoamine oxidase inhibitor (MAO Inhibitor).

(c) Hyaluronic Acid:
Hyaluronic acid is the water gelling molecule of the human body which serves as its scaffolding and hydrating agent. As aging progresses, less Hyaluronic acid is produced, resulting in wrinkled skin, thinning hair, unlubricated joints. The chelators of the present composition also help to preserve hyaluronic acid in the body. The hyaluronic acid component and the mineral chelating components (e.g., resveratrol, quercetin, phytic acid IP6, ferulate) work as a total anti-aging strategy to maintain youthful function within cells and connective tissues. Hyaluronic acid is believed to have an affinity to cancer cells. It is believed to serve as a delivery and targeting (drug delivery agent) molecule in blood circulation and to address aging of the connective tissue. The collapse and loss of integrity of connective tissue between cells provides the signs of aging (e.g., skin wrinkling, hair thinning, joint stiffness, loss of stature, etc.). The addition of hyaluronic acid to the compositions of the present invention is believed to activate fibroblast cells in the human body to produce additional hyaluronic acid, thus serving to preserve connective tissue (collagen) in a youthful state (Yadav, A. K. et al. (2008) “An Insight On Hyaluronic Acid In Drug Targeting And Drug Delivery,” J. Drug Target. 16(2):91-107; Liao, Y. H. et al. (2005) “Hyaluronan: Pharmaceutical Characterization And Drug Delivery,” Drug Deliv. 12(6):327-342; Joddar, B. et al. (2006) “Elastogenic Effects Of Exogenous Hyaluronan Oligosaccharides On Vascular Smooth Muscle Cells,” Biomaterials 27(33):5698-5707; Girish, K. S. et al. (2007) “The Magic Glue Hyaluronan And Its Eraser Hyaluronidase: A Biological Overview,” Life Sci. 80(21): 1921-1943).

(d) Phytic Acid:
Phytic Acid, preferably in the form of rice bran, is believed to act as an iron and copper chelator and as an inhibitor of calcium crystallization. Phytic Acid also is believed to reduce the availability of metallic minerals that serve as growth factors in tumor cells. It is also believed to serve as a neutrophil priming and motility agent. Additionally, phytic acid has been found to be neuroprotective, and thus to attenuate the severity of conditions associated with neurodegenerative diseases (especially Parkinson's Disease, camptocormia, and Alzheimer's Disease) (Xu, Q. et al. (Epub 2007 Dec. 27) “Neuroprotective Effect Of The Natural Iron Chelator, Phytic Acid In A Cell Culture Model Of Parkinson's Disease,” Toxicology 245 (1-2): 101-108). The components of the compositions of the present invention are believed to enhance such neuroprotection.

The iron chelator, quercetin, if present, is believed to serve to increase immediate bioavailability of resveratrol by permitting more passes through the liver before it is metabolized.

The individual components of the composition are believed to act synergistically to enhance the effect of, for example, resveratrol. Without intending to be limited thereby, it is proposed that the body's control or chelation of iron and calcium regulates the rate of aging after full growth has been achieved. During childhood growth all the iron and calcium are directed towards production of new bone and new red blood cells (hemoglobin). The cessation of childhood growth results in excess iron, copper and calcium, which then progressively (a) calcifies and (b) rusts tissues. The lysosomes begin to accumulate iron and calcium, which results in their dysfunction. The mitochondria begin to malfunction as they also progressively rust and calcify. The compositions of the present invention are believed to be capable of limiting or slowing the progressive rusting and calcification of cells and cellular organelles to thereby facilitate a slowing or reversal of the aging process. The chelation is what controls the genes. Genes are then favorably upregulated or downregulated. Resveratrol and a copper chelator are believed to act: (1) as controllers of calcium concentration via upregulation of osteocalcin, the hormone that helps retain calcium in bones and (2) as controllers of iron concentration via heme oxygenase, an antioxidant enzyme.

MAO inhibitors and iron chelators have been proposed as treatments for Parkinson's disease (Youdim, M. B. et al. (2004) “Novel Bifunctional Drugs Targeting Monoamine Oxidase Inhibition And Iron Chelation As An Approach To Neuroprotection In Parkinson's Disease And Other Neurodegenerative Diseases,” J. Neural. Transm. 111(10-11): 1455-1471; Yáñez, M. et al. (2006) “(−)-Trans-Epsilon-Viniferin, A Polyphenol Present In Wines, Is An Inhibitor Of Noradrenaline And 5-Hydroxytryptamine Uptake And Of Monoamine Oxidase Activity,” Eur. J. Pharmacol. 542(1-3):54-60; Bureau, G. et al. (2008) “Resveratrol And Quercetin, Two Natural Polyphenols, Reduce Apoptotic Neuronal Cell Death Induced By Neuroinflammation,” J. Neurosci. Res. 86(2):403-410; Singh, A. et al. (2003) “Quercetin Potentiates L-Dopa Reversal Of Drug-Induced Catalepsy In Rats: Possible COMT/MAO Inhibition,” Pharmacol. 68(2):81-88; Gao, X. et al. (2007) “Prospective Study Of Dietary Pattern And Risk Of Parkinson Disease,” Am. J. Clin. Nutr. 86(5):1486-1494; Johnson, S. (2001) “Is Parkinson's Disease The Heterozygote Form Of Wilson's Disease: PD=½ WD?,” Med. Hypotheses 56(2):171-173). The compositions of the present invention which contain the MAO inhibitor and copper chelator, resveratrol, the iron chelator and MAO inhibitor, quercetin, and the broad metal chelator, phytic acid are particularly preferred for the treatment of neurodegenerative diseases (especially Parkinson's Disease, camptocormia, and Alzheimer's Disease) or in the amelioration of the symptoms of such diseases.

Having now generally described the invention, the same will be more readily under-stood through reference to the following examples, which are provided by way of illustration and are not intended to be limiting of the present invention unless specified.

### Example 1

### Comparative Effects of Resveratrol and the Compositions of the Present Invention

In order to determine if the compositions of the present invention were more effective than resveratrol alone in mediating a resveratrol biological activity, an analysis of gene expression was conducted, comparing the modulation of gene expression achieved by calorie restriction to the modulation of gene expression achieved by the compositions of the present invention.

Accordingly, the ability of resveratrol alone and the resveratrol-containing compositions of the present invention to up-regulate survival/longevity genes or down-regulate genes whose expression enhances cellular damage was compared using the expression profile of a calorie restricted (“CR”) animal as a positive control and the expression profile of a normally fed animal as a negative control. Male B6CHF1 mice (2 months of age) were thus either placed on a 40% calorie restricted diet, provided commercially obtained trans-resveratrol (Sigma Chemical; 1.25 mg/kg per day), provided a resveratrol-containing composition of the present invention (Longevinex®; Resveratrol Associates, LLC; 100 mg trans-resveratrol containing capsule per 80 kg human per day (i.e., 2.5 mg/kg per day of resveratrol (1.25 mg/kg per day trans-resveratrol) 0.31 mg/kg per day quercetin dihydrate, 0.94 mg/kg per day rice bran extract, 4.75 mg/kg per day rice bran oil and 0.70 mg/kg per day sunflower lecithin)). The mice were monitored until they had reached five months of age.

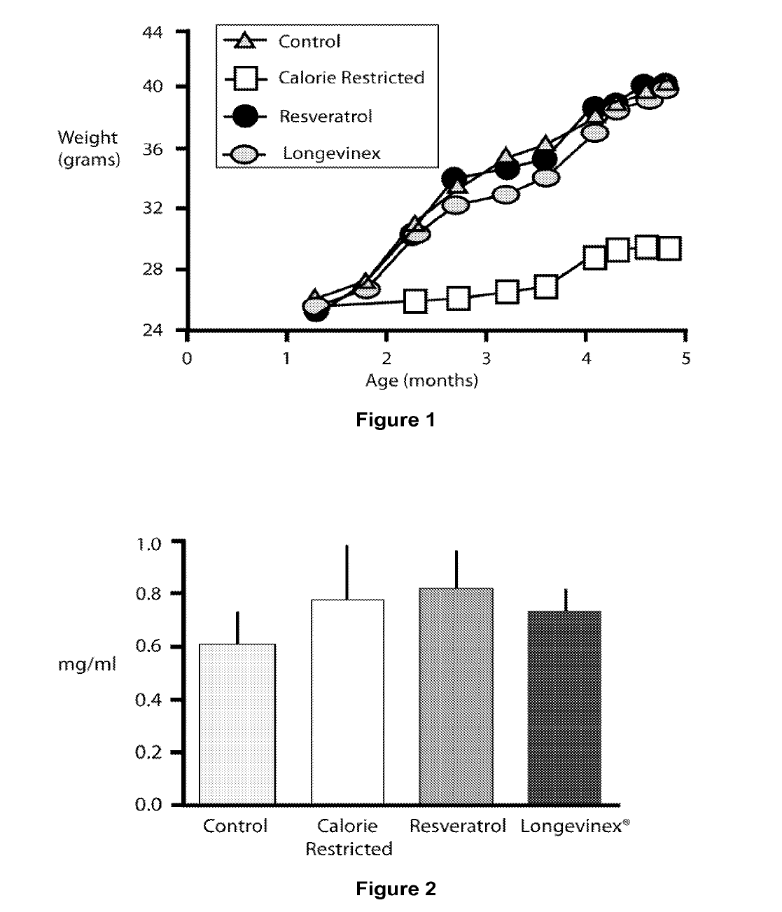

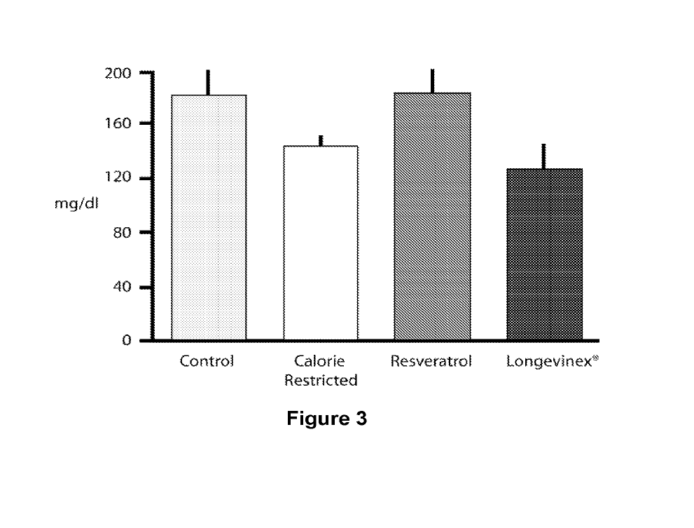

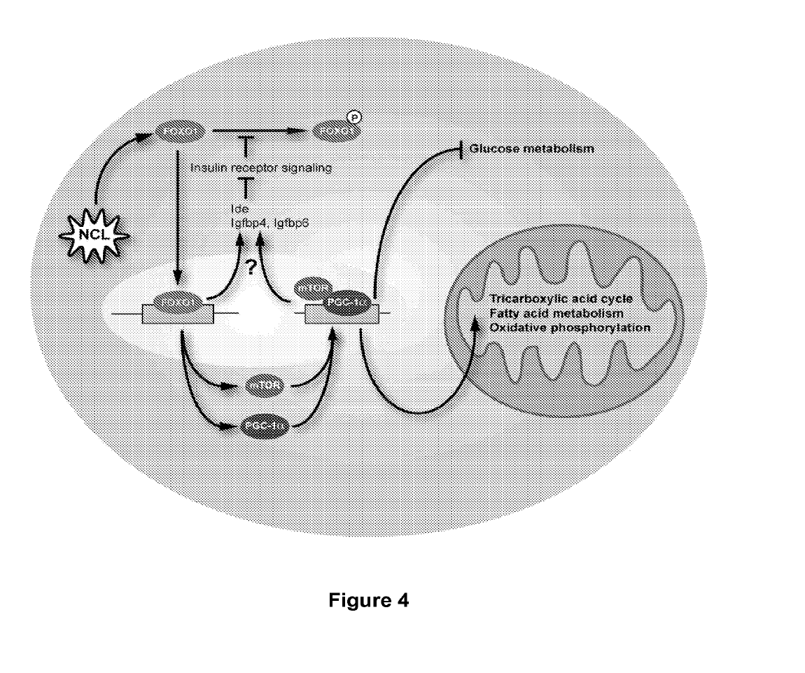

Body weight, serum glucose levels, serum insulin levels and lipid peroxidation in brain and muscle tissue were measured. The results showed that Longevinex® did not result in an increase in weight that was distinguishable from control animals (FIG. 1). Serum insulin levels were found to be approximately the same as that observed in the calorie restricted animals (FIG. 2). Serum glucose levels were found to be lower than that observed in the calorie restricted animals (FIG. 3).

### Example 2

### Comparative Effects of Resveratrol and the Compositions of the Present Invention on Gene Expression in Cardiac Tissue

The profile of expressed genes in the cardiac tissue of mice receiving resveratrol or a composition of the present invention (Longevinex®) was compared to that of mice placed on a calorie restricted diet and control mice. Gene expression was monitored using an Affymetrix MG430 2.0 Array, containing 45,101 probe sets per array. In cases in which the array represented the same gene with multiple probes, the probe set with the highest signal intensity was employed. Unknown genes (including uncharacterized ESTs and cDNA sequences were not analyzed. Thus, the array provided a means for analyzing 20,341 genes having a single Entrez Gene ID. Analysis was conducted substantially as described by Lee, C.-K. et al. (2002) “Transcriptional Profiles Associated With Aging And Middle Age-Onset Caloric Restriction In Mouse Hearts,” Proc. Natl. Acad. Sci. (U.S.A.) 99:14988-14993, herein incorporated by reference. The mean of all arrays in a group were calculated. The means of treated groups were compared to the mean of the control group, and the statistical significance of any differences were determined using two-tailed t-tests (P<0.01). The results of the analysis are presented in Table 3 (CO, control; CR, calorie restricted; RES, resveratrol; LGX, Longevinex®; FC, fold change. FC is calculated as the mean of the treated group divided by the mean of the control group, and this value is then log-transformed (base 2) for statistical purposes. As an example, a gene that is expressed at 100 in the control and 200 in a treated group would be have an Fc of 2 (i.e., a twofold increase in expression); a gene that is expressed at 100 in the control and 50 in the treated group, would have an Fc of −2 (i.e., a twofold decrease in expression).

Treatment of human umbilical vein epithelial cells with ferulic acid, quercetin or resveratrol has been reported to result in changes to gene expression of greater than 2-fold down-regulation of 363 genes, and greater than 2-fold up-regulation of 233 genes of 10,000 genes probed (Nicholson, S. K. et al. (2008) “Effects Of Dietary Polyphenols On Gene Expression In Human Vascular Endothelial Cells,” Proc. Nutr. Soc. 67(1):42-47). In contrast, Table 3 shows that 2,829 genes were found to exhibit a statistically significant change in expression in treated vs. control mice. Of these genes, 7% were found to exhibit altered expression in mice that had been subjected to only calorie restriction; 8% were found to exhibit altered expression in mice subjected only to resveratrol. Combining calorie restriction with resveratrol administration failed to alter the expression of any additional genes. In contrast, administration of Longevinex® was found to alter the expression of 61% of the 2,829 genes. Administration of Longevinex® to calorie restricted mice was found to alter the expression of an additional 2% of the genes. Administration of Longevinex® to mice receiving resveratrol was found to alter the expression of an additional 21% of the genes. Thus, Longevinex® alone or in combination with other regimens was found to affect 85% (2,406) of the total genes showing altered expression.

TABLE 3

| | | CO | CR | RES | LGX | FC | FC | FC | |
|---|---|---|---|---|---|---|---|---|---|
| Probe Set ID | Treatment | mean | mean | mean | mean | CR | Res | Lgx | Entrez Info |
| 1415670_at | Lgx only | 987 | 1139 | 1187 | 1378 | 1.15 | 1.20 | 1.40 | Copg |
| 1415671_at | Res & Lgx | 2454 | 2539 | 2030 | 2071 | 1.03 | −1.21 | −1.19 | Atp6v0d1 |
| 1415672_at | Res & Lgx | 3213 | 2819 | 2637 | 2262 | −1.14 | −1.22 | −1.42 | Golga7 |
| 1415677_at | Res & Lgx | 706 | 747 | 933 | 934 | 1.06 | 1.32 | 1.32 | Dhrs1 |
| 1415679_at | Lgx only | 4825 | 4129 | 3776 | 3154 | −1.17 | −1.28 | −1.53 | Psenen |
| 1415684_at | Lgx only | 638 | 573 | 490 | 381 | −1.11 | −1.30 | −1.67 | Atg5 |
| 1415696_at | Res only | 6629 | 7063 | 5491 | 5988 | 1.07 | −1.21 | −1.11 | Sar1a |
| 1415700_a_at | Res & Lgx | 5461 | 5179 | 3791 | 2603 | −1.05 | −1.44 | −2.10 | Ssr3 |
| 1415704_a_at | Lgx only | 5010 | 4472 | 3850 | 3285 | −1.12 | −1.30 | −1.52 | Cdv3 |
| 1415707_at | Lgx only | 1111 | 1317 | 1468 | 1535 | 1.19 | 1.32 | 1.38 | Anapc2 |
| 1415714_a_at | Res & Lgx | 4941 | 4632 | 2720 | 2379 | −1.07 | −1.82 | −2.08 | 2610209M04Rik |
| 1415723_at | Res & Lgx | 4272 | 3466 | 3385 | 3062 | −1.23 | −1.26 | −1.40 | Eif5 |
| 1415733_a_at | Lgx only | 13615 | 13963 | 9719 | 9991 | 1.03 | −1.40 | −1.36 | 1110019J04Rik |
| 1415735_at | Lgx only | 2025 | 2617 | 3355 | 3642 | 1.29 | 1.66 | 1.80 | Ddb1 |
| 1415736_at | Res & Lgx | 4957 | 3953 | 2536 | 1805 | −1.25 | −1.95 | −2.75 | Pfdn5 |
| 1415738_at | Res & Lgx | 1914 | 1941 | 1575 | 1395 | 1.01 | −1.22 | −1.37 | Txndc12 |
| 1415742_at | Lgx only | 749 | 837 | 856 | 987 | 1.12 | 1.14 | 1.32 | Aup1 |
| 1415746_at | Lgx only | 726 | 949 | 1075 | 1501 | 1.31 | 1.48 | 2.07 | Cic |
| 1415749_a_at | Lgx only | 2368 | 2397 | 2111 | 1912 | 1.01 | −1.12 | −1.24 | Rragc |
| 1415754_at | Lgx only | 3512 | 3195 | 3099 | 2411 | −1.10 | −1.13 | −1.46 | Polr2f |
| 1415755_a_at | Lgx only | 2983 | 3602 | 3890 | 4068 | 1.21 | 1.30 | 1.36 | Ube2v1 |
| 1415756_a_at | Res & Lgx | 3581 | 3290 | 2710 | 2706 | −1.09 | −1.32 | −1.32 | Snapap |
| 1415757_at | Lgx only | 901 | 1073 | 1189 | 1337 | 1.19 | 1.32 | 1.48 | Gbf1 |
| 1415764_at | Lgx only | 3381 | 3218 | 2545 | 2419 | −1.05 | −1.33 | −1.40 | Zc3h11a |
| 1415783_at | Lgx only | 4869 | 4728 | 4237 | 3960 | −1.03 | −1.15 | −1.23 | Vps35 |
| 1415788_at | Lgx only | 1490 | 1572 | 1169 | 1046 | 1.05 | −1.27 | −1.42 | Ublcp1 |
| 1415791_at | Res & Lgx | 2983 | 2870 | 2364 | 1805 | −1.04 | −1.26 | −1.65 | Rnf34 |
| 1415797_at | Lgx only | 319 | 488 | 522 | 805 | 1.53 | 1.64 | 2.53 | Ddr1 |
| 1415802_at | Res & Lgx | 8566 | 8379 | 4763 | 4548 | −1.02 | −1.80 | −1.88 | Slc16a1 |
| 1415812_at | Lgx only | 31926 | 36029 | 41778 | 58920 | 1.13 | 1.31 | 1.85 | Gsn |
| 1415814_at | CR only | 1760 | 2239 | 1514 | 1433 | 1.27 | −1.16 | −1.23 | Atp6v1b2 |
| 1415816_at | Res only | 5617 | 5279 | 4428 | 5553 | −1.06 | −1.27 | −1.01 | Cct7 |
| 1415818_at | Lgx only | 2554 | 2785 | 2838 | 3335 | 1.09 | 1.11 | 1.31 | Anxa6 |
| 1415830_at | Lgx only | 586 | 494 | 424 | 318 | −1.19 | −1.38 | −1.84 | Orc5l |
| 1415834_at | Res only | 579 | 703 | 419 | 430 | 1.21 | −1.38 | −1.35 | Dusp6 |
| 1415840_at | Res & Lgx | 1636 | 1461 | 974 | 856 | −1.12 | −1.68 | −1.91 | Elovl5 |
| 1415850_at | Res & Lgx | 824 | 880 | 1129 | 1093 | 1.07 | 1.37 | 1.33 | Rasa3 |
| 1415856_at | Lgx only | 148 | 168 | 172 | 242 | 1.14 | 1.16 | 1.63 | Emb |
| 1415875_at | Res only | 83 | 38 | 34 | 51 | −2.19 | −2.43 | −1.63 | 3010003L21Rik |
| 1415876_a_at | Lgx only | 13888 | 14344 | 16149 | 18965 | 1.03 | 1.16 | 1.37 | Rps26 |
| 1415879_a_at | Lgx only | 33385 | 37008 | 41034 | 49898 | 1.11 | 1.23 | 1.49 | Rplp2 |
| 1415882_at | Res only | 20588 | 18774 | 17207 | 17741 | −1.10 | −1.20 | −1.16 | Ghitm |
| 1415886_at | All | 637 | 966 | 1017 | 1132 | 1.51 | 1.60 | 1.78 | Sh2d3c |
| 1415901_at | Lgx only | 1435 | 1699 | 1682 | 1903 | 1.18 | 1.17 | 1.33 | Plod3 |
| 1415907_at | Lgx only | 1656 | 1817 | 2329 | 2446 | 1.10 | 1.41 | 1.48 | Ccnd3 |
| 1415909_at | Res & Lgx | 1788 | 1949 | 1414 | 1451 | 1.09 | −1.26 | −1.23 | Stip1 |
| 1415915_at | Res & Lgx | 2843 | 3059 | 3559 | 3668 | 1.08 | 1.25 | 1.29 | Ddx1 |
| 1415930_a_at | Lgx only | 14546 | 13685 | 14019 | 11677 | −1.06 | −1.04 | −1.25 | Map1lc3b |
| 1415935_at | Res & Lgx | 1687 | 1988 | 2011 | 2446 | 1.18 | 1.19 | 1.45 | Smoc2 |
| 1415947_at | CR only | 7499 | 6219 | 8373 | 7163 | −1.21 | 1.12 | −1.05 | Creg1 |
| 1415951_at | Lgx only | 496 | 588 | 587 | 717 | 1.19 | 1.18 | 1.45 | Fkbp10 |
| 1415961_at | Lgx only | 2152 | 2177 | 2503 | 3192 | 1.01 | 1.16 | 1.48 | Itm2c |
| 1415966_a_at | Lgx only | 29789 | 33932 | 35207 | 35979 | 1.14 | 1.18 | 1.21 | Ndufv1 |
| 1415971_at | CR only | 1825 | 1273 | 1649 | 1440 | −1.43 | −1.11 | −1.27 | Marcks |
| 1415974_at | Lgx only | 1694 | 2075 | 2211 | 2392 | 1.22 | 1.31 | 1.41 | Map2k2 |
| 1415977_at | Lgx only | 444 | 603 | 658 | 715 | 1.36 | 1.48 | 1.61 | Isyna1 |
| 1415987_at | Lgx only | 8838 | 9568 | 10218 | 11109 | 1.08 | 1.16 | 1.26 | Hdlbp |
| 1415990_at | CR & Lgx | 27024 | 32941 | 29714 | 35847 | 1.22 | 1.10 | 1.33 | Vdac2 |
| 1415991_a_at | Res & Lgx | 3051 | 3384 | 4293 | 4892 | 1.11 | 1.41 | 1.60 | Klhdc3 |
| 1415996_at | All | 10650 | 22628 | 23188 | 27989 | 2.12 | 2.18 | 2.63 | Txnip |
| 1415998_at | Lgx only | 33371 | 40497 | 35239 | 46095 | 1.21 | 1.06 | 1.38 | Vdac1 |
| 1416013_at | Res & Lgx | 926 | 1171 | 1314 | 1772 | 1.26 | 1.42 | 1.91 | Pld3 |
| 1416014_at | All | 1518 | 981 | 801 | 662 | −1.55 | −1.90 | −2.29 | Abce1 |
| 1416016_at | Res & Lgx | 232 | 236 | 387 | 358 | 1.02 | 1.67 | 1.55 | Tap1 |
| 1416019_at | Res only | 336 | 291 | 275 | 269 | −1.15 | −1.22 | −1.25 | Dr1 |
| 1416027_at | Res & Lgx | 2414 | 2298 | 1834 | 1569 | −1.05 | −1.32 | −1.54 | Pdcd6 |
| 1416032_at | Lgx only | 1053 | 1425 | 1776 | 1638 | 1.35 | 1.69 | 1.56 | Tmem109 |
| 1416046_a_at | Lgx only | 4982 | 5500 | 3916 | 3426 | 1.10 | −1.27 | −1.45 | Fuca2 |
| 1416048_at | Res & Lgx | 1089 | 1174 | 1589 | 1635 | 1.08 | 1.46 | 1.50 | Phc2 |
| 1416050_a_at | Lgx only | 903 | 1023 | 1209 | 1283 | 1.13 | 1.34 | 1.42 | Scarb1 |
| 1416051_at | Lgx only | 128 | 165 | 178 | 252 | 1.28 | 1.39 | 1.97 | C2 |
| 1416061_at | Lgx only | 1318 | 1255 | 1052 | 964 | −1.05 | −1.25 | −1.37 | Tbc1d15 |
| 1416064_a_at | Res only | 12341 | 12004 | 9273 | 10433 | −1.03 | −1.33 | −1.18 | Hspa5 |
| 1416069_at | CR only | 1055 | 1396 | 1197 | 1313 | 1.32 | 1.13 | 1.24 | Pfkp |
| 1416079_a_at | Lgx only | 1323 | 1494 | 1456 | 1611 | 1.13 | 1.10 | 1.22 | Arpc1a |
| 1416082_at | Res & Lgx | 13983 | 13415 | 11321 | 10270 | −1.04 | −1.24 | −1.36 | Rab1 |
| 1416091_at | Lgx only | 1996 | 2372 | 2622 | 3315 | 1.19 | 1.31 | 1.66 | Mtap4 |
| 1416106_at | Lgx only | 1108 | 939 | 1035 | 775 | −1.18 | −1.07 | −1.43 | Kti12 |
| 1416111_at | CR & Lgx | 368 | 212 | 288 | 138 | −1.74 | −1.28 | −2.68 | Cd83 |
| 1416112_at | Lgx only | 7587 | 9334 | 8583 | 12194 | 1.23 | 1.13 | 1.61 | Cox8a |
| 1416113_at | Lgx only | 1810 | 1965 | 2224 | 2559 | 1.09 | 1.23 | 1.41 | Fkbp8 |
| 1416125_at | Lgx only | 550 | 764 | 772 | 809 | 1.39 | 1.40 | 1.47 | Fkbp5 |
| 1416129_at | Lgx only | 1270 | 1118 | 1078 | 734 | −1.14 | −1.18 | −1.73 | Errfi1 |
| 1416140_a_at | Lgx only | 1672 | 1904 | 1977 | 1952 | 1.14 | 1.18 | 1.17 | Dhx30 |
| 1416142_at | Lgx only | 614 | 690 | 511 | 333 | 1.12 | −1.20 | −1.85 | Rps6 |
| 1416155_at | Res & Lgx | 3723 | 3890 | 2824 | 2551 | 1.04 | −1.32 | −1.46 | Hmgb3 |
| 1416175_a_at | Res & Lgx | 21409 | 19416 | 15845 | 13003 | −1.10 | −1.35 | −1.65 | Vdac3 |
| 1416176_at | Lgx only | 2064 | 1801 | 1869 | 1358 | −1.15 | −1.10 | −1.52 | Hmgb1 |
| 1416177_at | Res & Lgx | 884 | 734 | 669 | 666 | −1.20 | −1.32 | −1.33 | Rbmxrt |
| 1416181_at | Res only | 1982 | 1958 | 1504 | 1705 | −1.01 | −1.32 | −1.16 | Mesdc2 |
| 1416183_a_at | Lgx only | 40384 | 48786 | 54318 | 64528 | 1.21 | 1.35 | 1.60 | Ldhb |
| 1416185_a_at | Lgx only | 4447 | 4224 | 3331 | 2724 | −1.05 | −1.33 | −1.63 | Adh5 |
| 1416186_at | Lgx only | 404 | 340 | 342 | 213 | −1.19 | −1.18 | −1.90 | Pnrc2 |
| 1416195_at | Lgx only | 1575 | 1576 | 1933 | 2022 | 1.00 | 1.23 | 1.28 | RP23-136K12.4 |
| 1416209_at | CR & Lgx | 3673 | 4359 | 4108 | 4135 | 1.19 | 1.12 | 1.13 | Glud1 |
| 1416210_at | Lgx only | 2605 | 2387 | 2225 | 2041 | −1.09 | −1.17 | −1.28 | Imp3 |
| 1416223_at | Lgx only | 629 | 633 | 838 | 882 | 1.01 | 1.33 | 1.40 | Sh3bp5l |
| 1416226_at | Lgx only | 1514 | 1558 | 2186 | 2127 | 1.03 | 1.44 | 1.40 | Arpc1b |
| 1416238_at | Lgx only | 982 | 1045 | 1322 | 1446 | 1.06 | 1.35 | 1.47 | Tie1 |
| 1416240_at | Lgx only | 22627 | 18896 | 18958 | 17768 | −1.20 | −1.19 | −1.27 | Psmb7 |
| 1416252_at | Lgx only | 1031 | 1322 | 1235 | 1464 | 1.28 | 1.20 | 1.42 | Stk38 |
| 1416254_a_at | Lgx only | 432 | 478 | 497 | 673 | 1.11 | 1.15 | 1.56 | Vps16 |
| 1416256_a_at | CR only | 4550 | 5806 | 4704 | 5339 | 1.28 | 1.03 | 1.17 | Tubb5 |
| 1416259_at | CR & Lgx | 573 | 393 | 417 | 336 | −1.46 | −1.37 | −1.71 | Pex12 |
| 1416261_at | Lgx only | 328 | 394 | 350 | 470 | 1.20 | 1.07 | 1.43 | Tmem19 |
| 1416268_at | Lgx only | 4666 | 4083 | 4613 | 3784 | −1.14 | −1.01 | −1.23 | Ets2 |
| 1416271_at | CR & Lgx | 3787 | 4661 | 4246 | 5035 | 1.23 | 1.12 | 1.33 | Perp |
| 1416272_at | Lgx only | 4874 | 4932 | 3921 | 3693 | 1.01 | −1.24 | −1.32 | Map2k1ip1 |
| 1416280_at | Lgx only | 1640 | 1595 | 1340 | 1270 | −1.03 | −1.22 | −1.29 | Sae2 |
| 1416283_at | Res & Lgx | 1026 | 1225 | 1415 | 1355 | 1.19 | 1.38 | 1.32 | Gart |
| 1416284_at | Lgx only | 5006 | 5498 | 6000 | 6739 | 1.10 | 1.20 | 1.35 | Mrpl28 |
| 1416292_at | Res & Lgx | 13464 | 12624 | 11145 | 11453 | −1.07 | −1.21 | −1.18 | Prdx3 |
| 1416294_at | Lgx only | 1274 | 1556 | 1654 | 2003 | 1.22 | 1.30 | 1.57 | Scamp3 |
| 1416300_a_at | Lgx only | 81172 | 93900 | 86598 | 106206 | 1.16 | 1.07 | 1.31 | Slc25a3 |
| 1416312_at | Res only | 1570 | 1597 | 1351 | 1503 | 1.02 | −1.16 | −1.04 | Rars |
| 1416315_at | Lgx only | 1812 | 2063 | 2221 | 2496 | 1.14 | 1.23 | 1.38 | Abhd4 |
| 1416326_at | Lgx only | 8540 | 9522 | 10268 | 12292 | 1.11 | 1.20 | 1.44 | Crip1 |
| 1416329_at | CR only | 1231 | 933 | 1064 | 976 | −1.32 | −1.16 | −1.26 | Cyfip1 |
| 1416331_a_at | Lgx only | 4998 | 5244 | 6828 | 7398 | 1.05 | 1.37 | 1.48 | Nfe2l1 |
| 1416339_a_at | Lgx only | 1730 | 1851 | 1929 | 2312 | 1.07 | 1.12 | 1.34 | Prkcsh |
| 1416340_a_at | Lgx only | 1678 | 1836 | 2208 | 2386 | 1.09 | 1.32 | 1.42 | Man2b1 |
| 1416350_at | Lgx only | 209 | 134 | 177 | 85 | −1.56 | −1.18 | −2.46 | Klf16 |
| 1416366_at | Lgx only | 27546 | 25654 | 21110 | 19836 | −1.07 | −1.30 | −1.39 | Ndufc2 |
| 1416368_at | Lgx only | 3559 | 3865 | 3913 | 4425 | 1.09 | 1.10 | 1.24 | Gsta4 |
| 1416369_at | Lgx only | 728 | 542 | 558 | 430 | −1.34 | −1.30 | −1.69 | Hiatl1 |
| 1416371_at | Lgx only | 934 | 773 | 973 | 1314 | −1.21 | 1.04 | 1.41 | Apod |
| 1416384_a_at | Lgx only | 3771 | 3990 | 4230 | 4569 | 1.06 | 1.12 | 1.21 | Cope |
| 1416393_at | Lgx only | 5020 | 5227 | 4128 | 2942 | 1.04 | −1.22 | −1.71 | Emg1 |
| 1416405_at | Lgx only | 5254 | 5912 | 7260 | 10205 | 1.13 | 1.38 | 1.94 | Bgn |
| 1416411_at | Lgx only | 3752 | 3573 | 3504 | 3347 | −1.05 | −1.07 | −1.12 | Gstm2 |
| 1416412_at | Res & Lgx | 716 | 471 | 369 | 274 | −1.52 | −1.94 | −2.61 | Nsmaf |
| 1416414_at | Lgx only | 461 | 561 | 703 | 739 | 1.22 | 1.52 | 1.60 | Emilin1 |
| 1416417_a_at | Lgx only | 29794 | 33340 | 35044 | 38096 | 1.12 | 1.18 | 1.28 | Ndufb7 |
| 1416424_at | Lgx only | 3969 | 4305 | 4914 | 5377 | 1.08 | 1.24 | 1.35 | M6prbp1 |
| 1416425_at | Lgx only | 3908 | 4090 | 4560 | 4505 | 1.05 | 1.17 | 1.15 | Pex19 |
| 1416427_at | Lgx only | 14038 | 14608 | 14480 | 17881 | 1.04 | 1.03 | 1.27 | Ccni |
| 1416436_a_at | Res & Lgx | 3463 | 3513 | 2761 | 2608 | 1.01 | −1.25 | −1.33 | Uqcc |
| 1416438_at | Lgx only | 1704 | 1946 | 2041 | 2300 | 1.14 | 1.20 | 1.35 | Puf60 |
| 1416452_at | Lgx only | 5918 | 6059 | 6368 | 7090 | 1.02 | 1.08 | 1.20 | Oat |
| 1416455_a_at | Lgx only | 49845 | 53641 | 55445 | 64019 | 1.08 | 1.11 | 1.28 | Cryab |
| 1416457_at | Lgx only | 1208 | 1356 | 1592 | 1930 | 1.12 | 1.32 | 1.60 | Ddah2 |
| 1416462_at | Res & Lgx | 3796 | 3321 | 2539 | 2621 | −1.14 | −1.49 | −1.45 | Caprin1 |
| 1416478_a_at | Lgx only | 49370 | 54196 | 49980 | 56965 | 1.10 | 1.01 | 1.15 | Mdh2 |
| 1416479_a_at | Lgx only | 3314 | 4024 | 3824 | 5065 | 1.21 | 1.15 | 1.53 | Tmem14c |
| 1416494_at | Lgx only | 24034 | 24945 | 28712 | 32059 | 1.04 | 1.19 | 1.33 | Ndufs5 |
| 1416496_at | CR & Lgx | 9192 | 10747 | 9867 | 10827 | 1.17 | 1.07 | 1.18 | Mrfap1 |
| 1416498_at | CR & Lgx | 1234 | 1020 | 1151 | 968 | −1.21 | −1.07 | −1.28 | Ppic |
| 1416502_a_at | Res & Lgx | 1327 | 1624 | 1659 | 1918 | 1.22 | 1.25 | 1.45 | Preb |
| 1416506_at | Lgx only | 5718 | 5208 | 4876 | 4228 | −1.10 | −1.17 | −1.35 | Psma6 |
| 1416510_at | Lgx only | 5555 | 6339 | 5912 | 6679 | 1.14 | 1.06 | 1.20 | Mrpl4 |
| 1416513_at | Lgx only | 1582 | 2144 | 2762 | 2762 | 1.35 | 1.75 | 1.75 | Lamb2 |
| 1416514_a_at | Lgx only | 888 | 805 | 648 | 439 | −1.10 | −1.37 | −2.02 | Fscn1 |
| 1416517_at | Lgx only | 411 | 441 | 486 | 580 | 1.07 | 1.18 | 1.41 | Pnpla6 |
| 1416524_at | Lgx only | 4034 | 4167 | 4527 | 5256 | 1.03 | 1.12 | 1.30 | Spop |
| 1416540_at | Lgx only | 1017 | 1340 | 1386 | 1769 | 1.32 | 1.36 | 1.74 | Hgs |
| 1416547_at | Lgx only | 12925 | 12431 | 11457 | 10620 | −1.04 | −1.13 | −1.22 | Ndufb3 |
| 1416555_at | Res & Lgx | 2837 | 2868 | 2221 | 2008 | 1.01 | −1.28 | −1.41 | Ei24 |
| 1416563_at | Lgx only | 1374 | 1532 | 1043 | 788 | 1.11 | −1.32 | −1.74 | Ctps |
| 1416576_at | Lgx only | 230 | 236 | 432 | 441 | 1.02 | 1.88 | 1.92 | Socs3 |
| 1416587_a_at | Res & Lgx | 372 | 605 | 623 | 822 | 1.63 | 1.68 | 2.21 | Xrcc1 |
| 1416595_at | Lgx only | 1722 | 1750 | 1272 | 1269 | 1.02 | −1.35 | −1.36 | Mrps22 |
| 1416604_at | Lgx only | 55121 | 62820 | 59589 | 71198 | 1.14 | 1.08 | 1.29 | Cyc1 |
| 1416612_at | Lgx only | 424 | 520 | 635 | 826 | 1.23 | 1.50 | 1.95 | Cyp1b1 |
| 1416621_at | Res & Lgx | 869 | 1007 | 1063 | 1287 | 1.16 | 1.22 | 1.48 | Llgl1 |
| 1416629_at | Lgx only | 412 | 524 | 548 | 620 | 1.27 | 1.33 | 1.50 | Slc1a5 |
| 1416634_at | Res & Lgx | 3167 | 2719 | 1899 | 1420 | −1.16 | −1.67 | −2.23 | 5730536A07Rik |
| 1416635_at | Res & Lgx | 4444 | 3643 | 3384 | 2684 | −1.22 | −1.31 | −1.66 | Smpdl3a |
| 1416637_at | Lgx only | 525 | 603 | 661 | 780 | 1.15 | 1.26 | 1.49 | Slc4a2 |
| 1416647_at | Lgx only | 5775 | 6096 | 7099 | 7557 | 1.06 | 1.23 | 1.31 | Bckdha |
| 1416648_at | Lgx only | 2599 | 3174 | 3890 | 4330 | 1.22 | 1.50 | 1.67 | Dync1h1 |
| 1416656_at | Lgx only | 1412 | 1659 | 1596 | 1864 | 1.18 | 1.13 | 1.32 | Clic1 |
| 1416668_at | Lgx only | 10958 | 10417 | 8267 | 7237 | −1.05 | −1.33 | −1.51 | Ttc35 |
| 1416680_at | Res & Lgx | 2599 | 1810 | 1455 | 813 | −1.44 | −1.79 | −3.20 | Ube3a |
| 1416683_at | Lgx only | 1687 | 2209 | 2493 | 2585 | 1.31 | 1.48 | 1.53 | Plxnb2 |
| 1416690_at | Res & Lgx | 1379 | 1680 | 1929 | 1834 | 1.22 | 1.40 | 1.33 | Gtpbp2 |
| 1416699_at | Res only | 6446 | 6175 | 4968 | 5208 | −1.04 | −1.30 | −1.24 | 1110008F13Rik |
| 1416703_at | Lgx only | 2541 | 2311 | 2251 | 1680 | −1.10 | −1.13 | −1.51 | Mapk14 |
| 1416706_at | Res only | 408 | 412 | 258 | 254 | 1.01 | −1.58 | −1.61 | Rpe |
| 1416708_a_at | Lgx only | 807 | 815 | 975 | 1044 | 1.01 | 1.21 | 1.29 | Gramd1a |
| 1416709_a_at | Res & Lgx | 1287 | 1148 | 920 | 920 | −1.12 | −1.40 | −1.40 | Ngrn |
| 1416713_at | Lgx only | 920 | 1162 | 1424 | 2002 | 1.26 | 1.55 | 2.18 | Tppp3 |
| 1416730_at | Lgx only | 436 | 492 | 540 | 650 | 1.13 | 1.24 | 1.49 | Rcl1 |
| 1416731_at | Lgx only | 1678 | 1349 | 1389 | 1118 | −1.24 | −1.21 | −1.50 | Top2b |
| 1416737_at | Lgx only | 1600 | 2387 | 2293 | 2936 | 1.49 | 1.43 | 1.84 | Gys1 |
| 1416740_at | Lgx only | 1068 | 1202 | 1516 | 1606 | 1.13 | 1.42 | 1.50 | Col5a1 |
| 1416749_at | CR only | 4558 | 5528 | 3766 | 3810 | 1.21 | −1.21 | −1.20 | Htra1 |
| 1416752_at | Lgx only | 18842 | 22289 | 23077 | 26541 | 1.18 | 1.22 | 1.41 | Ldb3 |
| 1416755_at | CR & Lgx | 1571 | 1157 | 1295 | 897 | −1.36 | −1.21 | −1.75 | Dnajb1 |
| 1416766_at | Lgx only | 2214 | 2261 | 2504 | 2834 | 1.02 | 1.13 | 1.28 | Mosc2 |
| 1416791_a_at | Lgx only | 1176 | 1162 | 1174 | 1630 | −1.01 | −1.00 | 1.39 | Nxf1 |
| 1416805_at | Lgx only | 1361 | 1327 | 1132 | 959 | −1.03 | −1.20 | −1.42 | 1110032E23Rik |
| 1416808_at | Res & Lgx | 2839 | 2486 | 1876 | 1518 | −1.14 | −1.51 | −1.87 | Nid1 |
| 1416819_at | CR only | 7318 | 8511 | 7739 | 8109 | 1.16 | 1.06 | 1.11 | Cdc37 |
| 1416824_at | Res & Lgx | 6820 | 5799 | 4916 | 4447 | −1.18 | −1.39 | −1.53 | B230118H07Rik |
| 1416832_at | Lgx only | 179 | 250 | 238 | 315 | 1.40 | 1.33 | 1.76 | Slc39a8 |
| 1416836_at | Lgx only | 2043 | 2339 | 2345 | 2519 | 1.15 | 1.15 | 1.23 | Lrp10 |
| 1416841_at | Lgx only | 761 | 651 | 582 | 494 | −1.17 | −1.31 | −1.54 | 1110059E24Rik |
| 1416842_at | Lgx only | 5799 | 5337 | 5053 | 4625 | −1.09 | −1.15 | −1.25 | Gstm5 |
| 1416845_at | Res & Lgx | 253 | 365 | 392 | 412 | 1.44 | 1.55 | 1.63 | Tmem132a |
| 1416867_at | Res only | 539 | 510 | 644 | 503 | −1.06 | 1.19 | −1.07 | Bet1 |
| 1416883_at | Res & Lgx | 3457 | 4227 | 5144 | 5330 | 1.22 | 1.49 | 1.54 | Clptm1 |
| 1416884_at | Lgx only | 1755 | 1451 | 1259 | 1064 | −1.21 | −1.39 | −1.65 | Cbx3 |
| 1416896_at | Res only | 354 | 336 | 521 | 404 | −1.05 | 1.47 | 1.14 | Rps6ka1 |
| 1416903_at | Lgx only | 2268 | 2513 | 2807 | 2977 | 1.11 | 1.24 | 1.31 | Nucb1 |
| 1416912_at | Lgx only | 2816 | 2469 | 2287 | 1840 | −1.14 | −1.23 | −1.53 | 6330407G11Rik |
| 1416928_at | Res & Lgx | 295 | 289 | 197 | 136 | −1.02 | −1.50 | −2.16 | Rbm12 |
| 1416931_at | Lgx only | 695 | 726 | 597 | 533 | 1.05 | −1.16 | −1.30 | Nif3l1 |
| 1416933_at | Res & Lgx | 1482 | 1837 | 2077 | 2466 | 1.24 | 1.40 | 1.66 | Por |
| 1416940_at | Lgx only | 9984 | 10810 | 10055 | 11650 | 1.08 | 1.01 | 1.17 | Ppif |
| 1416943_at | Lgx only | 1721 | 1477 | 1227 | 872 | −1.16 | −1.40 | −1.97 | Ube2e1 |
| 1416953_at | CR only | 2136 | 3722 | 2891 | 2928 | 1.74 | 1.35 | 1.37 | Ctgf |
| 1416963_at | Res only | 4471 | 4553 | 3878 | 4212 | 1.02 | −1.15 | −1.06 | Ubac1 |
| 1416981_at | Lgx only | 926 | 912 | 1189 | 1623 | −1.02 | 1.28 | 1.75 | Foxo1 |
| 1416990_at | Lgx only | 926 | 985 | 1107 | 1231 | 1.06 | 1.19 | 1.33 | Rxrb |
| 1417000_at | Lgx only | 448 | 513 | 582 | 661 | 1.14 | 1.30 | 1.48 | Abtb1 |
| 1417006_at | Lgx only | 917 | 923 | 1024 | 1208 | 1.01 | 1.12 | 1.32 | Commd4 |
| 1417007_a_at | Lgx only | 2381 | 1901 | 1585 | 1243 | −1.25 | −1.50 | −1.92 | Vps4b |
| 1417008_at | Lgx only | 4995 | 5792 | 7236 | 8553 | 1.16 | 1.45 | 1.71 | Crat |
| 1417010_at | Lgx only | 1566 | 1338 | 1138 | 897 | −1.17 | −1.38 | −1.75 | Zfp238 |
| 1417018_at | Lgx only | 442 | 453 | 487 | 682 | 1.03 | 1.10 | 1.54 | Efemp2 |
| 1417026_at | Lgx only | 1683 | 1749 | 1357 | 1217 | 1.04 | −1.24 | −1.38 | Pfdn1 |
| 1417044_at | Lgx only | 959 | 1047 | 1076 | 1385 | 1.09 | 1.12 | 1.44 | Lcmt1 |
| 1417049_at | CR only | 693 | 515 | 650 | 556 | −1.34 | −1.07 | −1.25 | Rhd |
| 1417061_at | All | 1418 | 1003 | 1945 | 1761 | −1.41 | 1.37 | 1.24 | Slc40a1 |
| 1417065_at | CR only | 667 | 1043 | 914 | 758 | 1.56 | 1.37 | 1.14 | Egr1 |
| 1417068_a_at | Res & Lgx | 1030 | 1108 | 1271 | 1589 | 1.08 | 1.23 | 1.54 | Ptpn1 |
| 1417073_a_at | Lgx only | 2797 | 2634 | 1839 | 1346 | −1.06 | −1.52 | −2.08 | Qk |
| 1417075_at | Lgx only | 2020 | 1802 | 1853 | 1594 | −1.12 | −1.09 | −1.27 | 2010309E21Rik |
| 1417081_a_at | Lgx only | 3594 | 4012 | 4252 | 4459 | 1.12 | 1.18 | 1.24 | Syngr2 |
| 1417082_at | Lgx only | 1946 | 1607 | 1455 | 1101 | −1.21 | −1.34 | −1.77 | Anp32b |
| 1417091_at | Lgx only | 520 | 510 | 358 | 207 | −1.02 | −1.45 | −2.51 | Chuk |
| 1417105_at | Lgx only | 5064 | 4514 | 4396 | 4198 | −1.12 | −1.15 | −1.21 | Trappc2l |
| 1417109_at | Res & Lgx | 1215 | 1681 | 1773 | 1883 | 1.38 | 1.46 | 1.55 | Tinagl |
| 1417112_at | Res & Lgx | 3493 | 4015 | 4629 | 4787 | 1.15 | 1.33 | 1.37 | Arl2bp |
| 1417124_at | Lgx only | 7386 | 6930 | 5048 | 4243 | −1.07 | −1.46 | −1.74 | Dstn |
| 1417127_at | Lgx only | 53 | 106 | 136 | 186 | 2.01 | 2.58 | 3.52 | Msx1 |
| 1417142_at | Res & Lgx | 852 | 935 | 1104 | 1181 | 1.10 | 1.30 | 1.39 | 4932442K08Rik |
| 1417146_at | Res & Lgx | 605 | 807 | 893 | 1039 | 1.33 | 1.48 | 1.72 | 2410018C20Rik |
| 1417165_at | Lgx only | 2346 | 2465 | 2903 | 2874 | 1.05 | 1.24 | 1.23 | Mbd2 |
| 1417168_a_at | Lgx only | 2358 | 2585 | 3131 | 3801 | 1.10 | 1.33 | 1.61 | Usp2 |
| 1417170_at | Lgx only | 1459 | 1405 | 1174 | 1121 | −1.04 | −1.24 | −1.30 | Lztfl1 |
| 1417174_at | Res & Lgx | 1039 | 958 | 738 | 798 | −1.08 | −1.41 | −1.30 | 1810021J13Rik |
| 1417177_at | Lgx only | 462 | 447 | 437 | 628 | −1.03 | −1.06 | 1.36 | Galk1 |
| 1417180_at | Res & Lgx | 754 | 837 | 1024 | 1133 | 1.11 | 1.36 | 1.50 | Pcsk7 |
| 1417185_at | Lgx only | 9497 | 9130 | 8221 | 7632 | −1.04 | −1.16 | −1.24 | Ly6a |
| 1417190_at | Lgx only | 4733 | 4553 | 5431 | 6212 | −1.04 | 1.15 | 1.31 | Pbef1 |
| 1417191_at | Lgx only | 3776 | 3337 | 3338 | 2706 | −1.13 | −1.13 | −1.40 | Dnajb9 |
| 1417207_at | Lgx only | 80 | 144 | 149 | 248 | 1.79 | 1.86 | 3.09 | Dvl2 |
| 1417209_at | Lgx only | 1828 | 1950 | 2173 | 2377 | 1.07 | 1.19 | 1.30 | Sertad2 |
| 1417226_at | Lgx only | 812 | 921 | 967 | 1224 | 1.13 | 1.19 | 1.51 | Fbxw4 |
| 1417228_at | Lgx only | 535 | 649 | 639 | 835 | 1.21 | 1.19 | 1.56 | Capn1 |
| 1417233_at | Res & Lgx | 2926 | 2760 | 2274 | 1898 | −1.06 | −1.29 | −1.54 | Chchd4 |
| 1417238_at | Lgx only | 767 | 878 | 883 | 1108 | 1.15 | 1.15 | 1.44 | Ewsr1 |
| 1417239_at | Lgx only | 1936 | 1557 | 1139 | 998 | −1.24 | −1.70 | −1.94 | Cetn3 |
| 1417240_at | Res & Lgx | 1783 | 2528 | 2699 | 3377 | 1.42 | 1.51 | 1.89 | Zyx |
| 1417241_at | Res & Lgx | 3699 | 3394 | 2641 | 1970 | −1.09 | −1.40 | −1.88 | X83328 |
| 1417258_at | Res & Lgx | 4947 | 4979 | 3931 | 3307 | 1.01 | −1.26 | −1.50 | Cct5 |
| 1417271_a_at | Lgx only | 4442 | 4716 | 5165 | 6247 | 1.06 | 1.16 | 1.41 | Eng |
| 1417273_at | Res & Lgx | 8604 | 12748 | 23951 | 27986 | 1.48 | 2.78 | 3.25 | Pdk4 |
| 1417285_a_at | Lgx only | 32713 | 36339 | 37425 | 43067 | 1.11 | 1.14 | 1.32 | Ndufa5 |
| 1417291_at | Res & Lgx | 1425 | 1410 | 2175 | 2102 | −1.01 | 1.53 | 1.47 | Tnfrsf1a |
| 1417294_at | Lgx only | 1787 | 1927 | 2326 | 2186 | 1.08 | 1.30 | 1.22 | Akr7a5 |
| 1417297_at | All | 145 | 259 | 295 | 450 | 1.79 | 2.03 | 3.10 | Itpr3 |
| 1417304_at | Lgx only | 198 | 308 | 367 | 470 | 1.56 | 1.85 | 2.37 | Chrd |
| 1417306_at | Lgx only | 427 | 464 | 581 | 668 | 1.09 | 1.36 | 1.56 | Tyk2 |
| 1417307_at | Lgx only | 1236 | 1290 | 1344 | 1544 | 1.04 | 1.09 | 1.25 | Dmd |
| 1417308_at | Lgx only | 15382 | 20160 | 20634 | 26200 | 1.31 | 1.34 | 1.70 | Pkm2 |
| 1417311_at | Lgx only | 47051 | 55061 | 51583 | 64453 | 1.17 | 1.10 | 1.37 | Crip2 |
| 1417312_at | Lgx only | 1316 | 1798 | 2877 | 4398 | 1.37 | 2.19 | 3.34 | Dkk3 |
| 1417327_at | Lgx only | 3779 | 3050 | 2884 | 2338 | −1.24 | −1.31 | −1.62 | Cav2 |
| 1417334_at | Lgx only | 308 | 321 | 400 | 466 | 1.04 | 1.30 | 1.51 | Stk19 |
| 1417349_at | Lgx only | 3676 | 3837 | 3192 | 2742 | 1.04 | −1.15 | −1.34 | Pldn |
| 1417357_at | Lgx only | 1014 | 1076 | 1189 | 1373 | 1.06 | 1.17 | 1.35 | Emd |
| 1417367_at | Lgx only | 11894 | 11153 | 11043 | 9572 | −1.07 | −1.08 | −1.24 | Ppp2ca |
| 1417369_at | Lgx only | 2848 | 3279 | 3554 | 4089 | 1.15 | 1.25 | 1.44 | Hsd17b4 |
| 1417373_a_at | CR & Res | 57174 | 74618 | 40192 | 59746 | 1.31 | −1.42 | 1.04 | Tuba4a |
| 1417382_at | CR & Res | 5433 | 4021 | 4266 | 4657 | −1.35 | −1.27 | −1.17 | Entpd5 |
| 1417389_at | Lgx only | 4932 | 5697 | 6811 | 9117 | 1.16 | 1.38 | 1.85 | Gpc1 |
| 1417392_a_at | Lgx only | 386 | 416 | 569 | 884 | 1.08 | 1.47 | 2.29 | Slc7a7 |
| 1417394_at | Lgx only | 1516 | 1224 | 998 | 705 | −1.24 | −1.52 | −2.15 | Klf4 |
| 1417397_at | Lgx only | 364 | 447 | 480 | 576 | 1.23 | 1.32 | 1.58 | Slc9a1 |
| 1417398_at | CR & Lgx | 2047 | 1670 | 1999 | 1532 | −1.23 | −1.02 | −1.34 | Rras2 |
| 1417399_at | Lgx only | 4367 | 4901 | 4815 | 5943 | 1.12 | 1.10 | 1.36 | Gas6 |
| 1417402_at | Lgx only | 3863 | 4030 | 3439 | 3158 | 1.04 | −1.12 | −1.22 | 1190017O12Rik |
| 1417409_at | Lgx only | 1660 | 1563 | 1378 | 1058 | −1.06 | −1.20 | −1.57 | Jun |
| 1417423_at | All | 1931 | 2829 | 3104 | 4003 | 1.47 | 1.61 | 2.07 | Grina |
| 1417433_at | Res only | 1695 | 1694 | 1291 | 1499 | −1.00 | −1.31 | −1.13 | Lypla2 |
| 1417437_at | CR only | 643 | 802 | 739 | 810 | 1.25 | 1.15 | 1.26 | Xrcc6 |
| 1417441_at | Lgx only | 970 | 856 | 665 | 557 | −1.13 | −1.46 | −1.74 | Dnajc12 |
| 1417446_at | Res & Lgx | 817 | 1066 | 1009 | 1405 | 1.31 | 1.24 | 1.72 | Slc12a4 |
| 1417466_at | Lgx only | 22355 | 20101 | 19656 | 17738 | −1.11 | −1.14 | −1.26 | Rgs5 |
| 1417475_at | Lgx only | 541 | 682 | 670 | 846 | 1.26 | 1.24 | 1.57 | Atp13a1 |
| 1417476_at | Lgx only | 1158 | 1444 | 1386 | 1454 | 1.25 | 1.20 | 1.26 | Fbxw5 |
| 1417478_a_at | Res & Lgx | 2285 | 1692 | 1323 | 979 | −1.35 | −1.73 | −2.34 | Ppp2r3c |
| 1417490_at | Lgx only | 17449 | 20356 | 20005 | 21458 | 1.17 | 1.15 | 1.23 | Ctsb |
| 1417493_at | Lgx only | 1896 | 1748 | 1628 | 1370 | −1.09 | −1.17 | −1.38 | Bmi1 |
| 1417494_a_at | Lgx only | 3168 | 2535 | 2703 | 2081 | −1.25 | −1.17 | −1.52 | Cp |
| 1417503_at | Lgx only | 813 | 899 | 1044 | 1230 | 1.11 | 1.29 | 1.51 | Rfc2 |
| 1417507_at | Res only | 303 | 313 | 206 | 250 | 1.04 | −1.47 | −1.21 | Cyb561 |
| 1417516_at | Res & Lgx | 1417 | 1442 | 975 | 1093 | 1.02 | −1.45 | −1.30 | Ddit3 |
| 1417533_a_at | Res & Lgx | 3526 | 3994 | 5408 | 5733 | 1.13 | 1.53 | 1.63 | Itgb5 |
| 1417536_at | Lgx only | 1523 | 1489 | 1327 | 1156 | −1.02 | −1.15 | −1.32 | Zmat2 |
| 1417544_a_at | Lgx only | 1725 | 2092 | 2186 | 2613 | 1.21 | 1.27 | 1.52 | Flot2 |
| 1417552_at | Lgx only | 368 | 330 | 315 | 219 | −1.12 | −1.17 | −1.68 | Fap |
| 1417562_at | Res & Lgx | 5225 | 5748 | 7049 | 8132 | 1.10 | 1.35 | 1.56 | Eif4ebp1 |
| 1417564_at | Lgx only | 1045 | 942 | 849 | 682 | −1.11 | −1.23 | −1.53 | Med7 |
| 1417574_at | Lgx only | 4249 | 4227 | 4829 | 5635 | −1.01 | 1.14 | 1.33 | Cxcl12 |
| 1417578_a_at | Lgx only | 1318 | 1466 | 1614 | 1933 | 1.11 | 1.23 | 1.47 | Gmppa |
| 1417581_at | Lgx only | 327 | 311 | 206 | 118 | −1.05 | −1.59 | −2.77 | Dhodh |
| 1417588_at | CR only | 70 | 24 | 64 | 43 | −2.88 | −1.10 | −1.62 | Galnt3 |
| 1417590_at | Res & Lgx | 1193 | 1414 | 1657 | 1976 | 1.19 | 1.39 | 1.66 | Cyp27a1 |
| 1417592_at | Lgx only | 1276 | 1544 | 1813 | 1967 | 1.21 | 1.42 | 1.54 | Frap1 |
| 1417606_a_at | Lgx only | 8113 | 8165 | 6538 | 5977 | 1.01 | −1.24 | −1.36 | Calr |
| 1417611_at | Lgx only | 410 | 483 | 562 | 660 | 1.18 | 1.37 | 1.61 | Tmem37 |
| 1417614_at | Lgx only | 52366 | 62118 | 63982 | 71743 | 1.19 | 1.22 | 1.37 | Ckm |
| 1417626_at | Lgx only | 20898 | 23134 | 29205 | 31629 | 1.11 | 1.40 | 1.51 | Pde4dip |
| 1417629_at | CR only | 550 | 726 | 507 | 529 | 1.32 | −1.08 | −1.04 | Prodh |
| 1417631_at | Res only | 576 | 686 | 816 | 800 | 1.19 | 1.42 | 1.39 | Mknk1 |
| 1417636_at | Lgx only | 357 | 496 | 550 | 864 | 1.39 | 1.54 | 2.42 | Slc6a9 |
| 1417637_a_at | Lgx only | 740 | 721 | 597 | 561 | −1.03 | −1.24 | −1.32 | Hmg20b |
| 1417659_at | Res & Lgx | 2296 | 1809 | 1478 | 837 | −1.27 | −1.55 | −2.74 | Vps29 |
| 1417664_a_at | Lgx only | 413 | 416 | 589 | 585 | 1.01 | 1.43 | 1.42 | Ndrg3 |
| 1417665_a_at | Lgx only | 1641 | 1906 | 2136 | 2541 | 1.16 | 1.30 | 1.55 | Cpsf1 |
| 1417669_at | Lgx only | 1917 | 2203 | 2177 | 2365 | 1.15 | 1.14 | 1.23 | Abhd12 |
| 1417673_at | Res & Lgx | 9046 | 10264 | 7598 | 7548 | 1.13 | −1.19 | −1.20 | Grb14 |
| 1417680_at | Lgx only | 1039 | 914 | 800 | 514 | −1.14 | −1.30 | −2.02 | Kcna5 |
| 1417681_at | Lgx only | 2481 | 2417 | 2057 | 1755 | −1.03 | −1.21 | −1.41 | Nudt21 |
| 1417683_at | Lgx only | 3002 | 3170 | 2902 | 2615 | 1.06 | −1.03 | −1.15 | Diablo |
| 1417693_a_at | Lgx only | 2110 | 2468 | 2597 | 3325 | 1.17 | 1.23 | 1.58 | Gab1 |
| 1417712_at | Lgx only | 9771 | 8265 | 8035 | 6481 | −1.18 | −1.22 | −1.51 | Eif2s2 |
| 1417715_a_at | Lgx only | 37732 | 44662 | 43599 | 56266 | 1.18 | 1.16 | 1.49 | Got2 |
| 1417722_at | Lgx only | 2172 | 2787 | 2703 | 3093 | 1.28 | 1.24 | 1.42 | Pgls |
| 1417724_at | Lgx only | 963 | 827 | 712 | 620 | −1.17 | −1.35 | −1.55 | Thoc4 |
| 1417727_at | Lgx only | 595 | 659 | 764 | 845 | 1.11 | 1.28 | 1.42 | Sfrs9 |
| 1417728_at | Lgx only | 1083 | 1178 | 1375 | 1363 | 1.09 | 1.27 | 1.26 | Mbd3 |
| 1417730_at | Res & Lgx | 759 | 785 | 1047 | 1112 | 1.03 | 1.38 | 1.47 | Ext1 |
| 1417762_a_at | Lgx only | 15317 | 17412 | 16383 | 18728 | 1.14 | 1.07 | 1.22 | Rpl8 |
| 1417775_at | Lgx only | 385 | 504 | 537 | 605 | 1.31 | 1.40 | 1.57 | Rpo1-4 |
| 1417777_at | Lgx only | 300 | 380 | 359 | 417 | 1.27 | 1.20 | 1.39 | Ltb4dh |
| 1417778_at | Lgx only | 592 | 523 | 478 | 322 | −1.13 | −1.24 | −1.84 | Zfp35 |
| 1417791_a_at | Lgx only | 752 | 638 | 524 | 553 | −1.18 | −1.43 | −1.36 | Zfml |
| 1417807_at | Lgx only | 404 | 370 | 322 | 268 | −1.09 | −1.25 | −1.51 | 2700038N03Rik |
| 1417814_at | CR only | 1089 | 821 | 1021 | 931 | −1.33 | −1.07 | −1.17 | Pla2g5 |
| 1417825_at | Res & Lgx | 10020 | 8920 | 8393 | 8135 | −1.12 | −1.19 | −1.23 | Esd |
| 1417827_at | Lgx only | 1987 | 1887 | 1725 | 1334 | −1.05 | −1.15 | −1.49 | Ngly1 |
| 1417840_at | Lgx only | 655 | 635 | 537 | 493 | −1.03 | −1.22 | −1.33 | 1500031L02Rik |
| 1417842_at | CR only | 735 | 604 | 602 | 618 | −1.22 | −1.22 | −1.19 | Caml |
| 1417865_at | Res only | 1877 | 1747 | 1566 | 1714 | −1.07 | −1.20 | −1.09 | Tnfaip1 |
| 1417868_a_at | Lgx only | 1651 | 1315 | 950 | 698 | −1.26 | −1.74 | −2.36 | Ctsz |
| 1417889_at | Lgx only | 11804 | 10711 | 10232 | 8376 | −1.10 | −1.15 | −1.41 | Apobec2 |
| 1417893_at | Lgx only | 350 | 308 | 313 | 275 | −1.14 | −1.12 | −1.27 | Sfxn3 |
| 1417912_at | Lgx only | 4436 | 4290 | 4051 | 3653 | −1.03 | −1.10 | −1.21 | Tmem93 |
| 1417916_a_at | Lgx only | 2557 | 2471 | 2075 | 1814 | −1.03 | −1.23 | −1.41 | Fxc1 |
| 1417928_at | Lgx only | 930 | 1019 | 1145 | 1635 | 1.10 | 1.23 | 1.76 | Pdlim4 |
| 1417933_at | Lgx only | 1003 | 1029 | 1202 | 1834 | 1.03 | 1.20 | 1.83 | Igfbp6 |
| 1417936_at | Res & Lgx | 627 | 556 | 388 | 317 | −1.13 | −1.62 | −1.98 | Ccl9 |
| 1417951_at | Res & Lgx | 39914 | 46728 | 50392 | 54619 | 1.17 | 1.26 | 1.37 | Eno3 |
| 1417953_at | Lgx only | 721 | 903 | 916 | 1106 | 1.25 | 1.27 | 1.53 | D6Wsu176e |
| 1417963_at | CR only | 1666 | 1274 | 1799 | 1621 | −1.31 | 1.08 | −1.03 | Pltp |
| 1417970_at | Lgx only | 2618 | 2363 | 2180 | 1814 | −1.11 | −1.20 | −1.44 | Atp5s |
| 1417974_at | Lgx only | 1516 | 1151 | 1001 | 799 | −1.32 | −1.51 | −1.90 | Kpna4 |
| 1417983_a_at | Lgx only | 654 | 514 | 464 | 439 | −1.27 | −1.41 | −1.49 | Ube2v2 |
| 1417985_at | All | 980 | 658 | 657 | 521 | −1.49 | −1.49 | −1.88 | Nrarp |
| 1418000_a_at | Lgx only | 44199 | 45002 | 51725 | 57791 | 1.02 | 1.17 | 1.31 | Itm2b |
| 1418004_a_at | Lgx only | 1596 | 1844 | 2175 | 2741 | 1.16 | 1.36 | 1.72 | Tmem176b |
| 1418007_at | Lgx only | 559 | 548 | 518 | 370 | −1.02 | −1.08 | −1.51 | 1810007M14Rik |
| 1418025_at | Res & Lgx | 1814 | 1542 | 1141 | 1035 | −1.18 | −1.59 | −1.75 | Bhlhb2 |
| 1418031_at | Res & Lgx | 433 | 559 | 602 | 614 | 1.29 | 1.39 | 1.42 | Myo9b |
| 1418048_at | Lgx only | 2291 | 1894 | 1822 | 1403 | −1.21 | −1.26 | −1.63 | 1110059G10Rik |
| 1418049_at | Lgx only | 564 | 707 | 732 | 893 | 1.25 | 1.30 | 1.58 | Ltbp3 |
| 1418058_at | Lgx only | 1896 | 1822 | 1556 | 1532 | −1.04 | −1.22 | −1.24 | Eltd1 |
| 1418085_at | Lgx only | 119 | 169 | 209 | 189 | 1.42 | 1.76 | 1.59 | Prkcz |
| 1418090_at | Lgx only | 393 | 473 | 619 | 747 | 1.20 | 1.58 | 1.90 | Plvap |
| 1418093_a_at | Lgx only | 180 | 168 | 242 | 294 | −1.07 | 1.34 | 1.63 | Egf |
| 1418124_at | Res & Lgx | 3982 | 4177 | 4816 | 5241 | 1.05 | 1.21 | 1.32 | Tmem85 |
| 1418128_at | Lgx only | 2612 | 3481 | 3429 | 3842 | 1.33 | 1.31 | 1.47 | Adcy6 |
| 1418148_at | Res & Lgx | 1168 | 915 | 1700 | 1493 | −1.28 | 1.46 | 1.28 | Abhd1 |
| 1418181_at | Lgx only | 6615 | 7164 | 10513 | 10742 | 1.08 | 1.59 | 1.62 | Ptp4a3 |
| 1418183_a_at | Lgx only | 2041 | 1857 | 1603 | 1381 | −1.10 | −1.27 | −1.48 | Pscd1 |
| 1418186_at | Lgx only | 1322 | 1528 | 1723 | 2106 | 1.16 | 1.30 | 1.59 | Gstt1 |
| 1418187_at | Lgx only | 2289 | 2761 | 3034 | 3198 | 1.21 | 1.33 | 1.40 | Ramp2 |
| 1418209_a_at | Res & Lgx | 3685 | 3274 | 2816 | 2402 | −1.13 | −1.31 | −1.53 | Pfn2 |
| 1418223_at | Res & Lgx | 2693 | 2594 | 2189 | 2015 | −1.04 | −1.23 | −1.34 | Sec11a |
| 1418228_at | Lgx only | 3494 | 3467 | 2926 | 2282 | −1.01 | −1.19 | −1.53 | Nfu1 |
| 1418244_at | Res & Lgx | 3970 | 4253 | 2852 | 2563 | 1.07 | −1.39 | −1.55 | Nat5 |
| 1418261_at | All | 412 | 712 | 724 | 860 | 1.73 | 1.76 | 2.09 | Syk |
| 1418275_a_at | Lgx only | 977 | 937 | 845 | 806 | −1.04 | −1.16 | −1.21 | Elf2 |
| 1418277_at | Lgx only | 5601 | 5071 | 4822 | 3833 | −1.10 | −1.16 | −1.46 | rp9 |
| 1418296_at | Lgx only | 743 | 895 | 936 | 1162 | 1.20 | 1.26 | 1.56 | Fxyd5 |
| 1418302_at | Res & Lgx | 1279 | 1585 | 1792 | 2129 | 1.24 | 1.40 | 1.66 | Ppt2 |
| 1418306_at | CR only | 212 | 99 | 186 | 157 | −2.15 | −1.14 | −1.35 | Crybb1 |
| 1418308_at | Lgx only | 312 | 316 | 235 | 144 | 1.02 | −1.33 | −2.17 | Hus1 |
| 1418310_a_at | CR only | 27 | 74 | 31 | 71 | 2.71 | 1.13 | 2.59 | Rlbp1 |
| 1418325_at | Res only | 530 | 450 | 302 | 356 | −1.18 | −1.75 | −1.49 | Sephs2 |
| 1418327_at | Lgx only | 6638 | 6594 | 5327 | 4629 | −1.01 | −1.25 | −1.43 | 1110058L19Rik |
| 1418328_at | Lgx only | 9663 | 10359 | 12241 | 14054 | 1.07 | 1.27 | 1.45 | Cpt1b |
| 1418364_a_at | Res & Lgx | 6214 | 6280 | 8701 | 10097 | 1.01 | 1.40 | 1.62 | Ftl1 |
| 1418373_at | Res & Lgx | 20321 | 25840 | 28141 | 36744 | 1.27 | 1.38 | 1.81 | Pgam2 |
| 1418384_at | Lgx only | 2697 | 2580 | 2446 | 2128 | −1.05 | −1.10 | −1.27 | Apool |
| 1418394_a_at | All | 1313 | 1651 | 1851 | 2002 | 1.26 | 1.41 | 1.52 | Cd97 |
| 1418395_at | Res only | 2083 | 2050 | 2622 | 2487 | −1.02 | 1.26 | 1.19 | Slc47a1 |
| 1418421_at | Lgx only | 409 | 447 | 356 | 219 | 1.09 | −1.15 | −1.87 | Bcl6b |
| 1418427_at | Lgx only | 4160 | 3689 | 3518 | 2931 | −1.13 | −1.18 | −1.42 | Kif5b |
| 1418433_at | Lgx only | 2097 | 2223 | 2417 | 2813 | 1.06 | 1.15 | 1.34 | Cab39 |
| 1418456_a_at | Lgx only | 1322 | 1134 | 961 | 777 | −1.17 | −1.37 | −1.70 | Cxcl14 |
| 1418461_at | Lgx only | 137 | 207 | 161 | 226 | 1.52 | 1.18 | 1.65 | Sh3d19 |
| 1418462_at | Lgx only | 938 | 943 | 764 | 665 | 1.01 | −1.23 | −1.41 | Exosc9 |
| 1418464_at | Lgx only | 54 | 55 | 98 | 186 | 1.02 | 1.80 | 3.42 | Matn4 |
| 1418467_at | CR & Lgx | 1961 | 2626 | 2477 | 2773 | 1.34 | 1.26 | 1.41 | Smarcd3 |
| 1418479_at | CR & Lgx | 971 | 752 | 799 | 628 | −1.29 | −1.22 | −1.55 | Vps54 |
| 1418483_a_at | Lgx only | 801 | 694 | 755 | 588 | −1.15 | −1.06 | −1.36 | Ggta1 |
| 1418495_at | Res only | 310 | 349 | 161 | 194 | 1.13 | −1.93 | −1.60 | Zc3h8 |
| 1418506_a_at | Res only | 20857 | 23088 | 24680 | 25457 | 1.11 | 1.18 | 1.22 | Prdx2 |
| 1418518_at | Lgx only | 701 | 821 | 854 | 1036 | 1.17 | 1.22 | 1.48 | Furin |
| 1418528_a_at | Res & Lgx | 4483 | 4424 | 3749 | 3167 | −1.01 | −1.20 | −1.42 | Dad1 |
| 1418530_at | Lgx only | 282 | 250 | 196 | 154 | −1.13 | −1.44 | −1.84 | Nup160 |
| 1418532_at | Lgx only | 180 | 202 | 332 | 354 | 1.12 | 1.85 | 1.97 | Fzd2 |
| 1418551_at | Lgx only | 34184 | 47198 | 44002 | 56324 | 1.38 | 1.29 | 1.65 | Mybpc3 |
| 1418560_at | Lgx only | 42487 | 47719 | 46215 | 55194 | 1.12 | 1.09 | 1.30 | Pdha1 |
| 1418563_at | Res & Lgx | 1576 | 1497 | 1007 | 795 | −1.05 | −1.57 | −1.98 | Serbp1 |
| 1418578_at | Lgx only | 718 | 782 | 1230 | 1501 | 1.09 | 1.71 | 2.09 | Dgka |
| 1418583_at | Lgx only | 4503 | 4409 | 3800 | 2964 | −1.02 | −1.19 | −1.52 | Hint3 |
| 1418584_at | Lgx only | 2929 | 2748 | 2413 | 2257 | −1.07 | −1.21 | −1.30 | Ccnh |
| 1418589_a_at | Res & Lgx | 5966 | 5443 | 3817 | 3292 | −1.10 | −1.56 | −1.81 | Mlf1 |
| 1418593_at | Lgx only | 967 | 1039 | 1128 | 1548 | 1.08 | 1.17 | 1.60 | Taf6 |
| 1418595_at | Lgx only | 3838 | 4270 | 5239 | 5576 | 1.11 | 1.37 | 1.45 | S3-12 |
| 1418604_at | CR only | 118 | 179 | 95 | 148 | 1.51 | −1.24 | 1.25 | Avpr1a |
| 1418621_at | Lgx only | 6696 | 7127 | 7710 | 8254 | 1.06 | 1.15 | 1.23 | Rab2 |
| 1418640_at | Lgx only | 445 | 396 | 366 | 260 | −1.12 | −1.22 | −1.71 | Sirt1 |
| 1418644_a_at | Res & Lgx | 2290 | 2528 | 3079 | 3754 | 1.10 | 1.34 | 1.64 | Stk11 |
| 1418646_at | Lgx only | 576 | 557 | 801 | 953 | −1.03 | 1.39 | 1.65 | Gna-rs1 |
| 1418649_at | Res & Lgx | 1817 | 1689 | 1010 | 1064 | −1.08 | −1.80 | −1.71 | Egln3 |
| 1418658_at | Lgx only | 3201 | 2867 | 2597 | 2170 | −1.12 | −1.23 | −1.48 | 2410005O16Rik |
| 1418659_at | Lgx only | 655 | 451 | 573 | 399 | −1.45 | −1.14 | −1.64 | Clock |
| 1418665_at | Res only | 432 | 341 | 817 | 509 | −1.27 | 1.89 | 1.18 | Impa2 |
| 1418681_at | Lgx only | 167 | 97 | 90 | 44 | −1.73 | −1.85 | −3.80 | Alg13 |
| 1418700_at | Res only | 2166 | 2223 | 2693 | 2292 | 1.03 | 1.24 | 1.06 | Lias |
| 1418703_at | Res & Lgx | 1788 | 1937 | 2728 | 2973 | 1.08 | 1.53 | 1.66 | Rbms1 |
| 1418714_at | Lgx only | 176 | 149 | 306 | 402 | −1.19 | 1.74 | 2.28 | Dusp8 |
| 1418726_a_at | Lgx only | 96588 | 107808 | 105192 | 136735 | 1.12 | 1.09 | 1.42 | Tnnt2 |
| 1418739_at | CR only | 115 | 61 | 145 | 180 | −1.88 | 1.26 | 1.56 | Sgk2 |
| 1418749_at | Lgx only | 829 | 963 | 993 | 1502 | 1.16 | 1.20 | 1.81 | Psd3 |
| 1418759_at | CR only | 65 | 25 | 85 | 74 | −2.61 | 1.30 | 1.14 | Ptpn20 |
| 1418763_at | Res only | 1354 | 1349 | 1037 | 1054 | −1.00 | −1.31 | −1.28 | Nit2 |
| 1418773_at | Lgx only | 997 | 1127 | 1373 | 1342 | 1.13 | 1.38 | 1.35 | Fads3 |
| 1418775_at | Res & Lgx | 1330 | 1611 | 1775 | 2047 | 1.21 | 1.33 | 1.54 | Al837181 |
| 1418782_at | Lgx only | 1857 | 2604 | 2789 | 2699 | 1.40 | 1.50 | 1.45 | Rxrg |
| 1418817_at | Lgx only | 868 | 707 | 713 | 565 | −1.23 | −1.22 | −1.54 | Chmp1b |
| 1418835_at | Lgx only | 1187 | 1202 | 996 | 645 | 1.01 | −1.19 | −1.84 | Phlda1 |
| 1418838_at | Lgx only | 1243 | 1517 | 1333 | 1771 | 1.22 | 1.07 | 1.43 | Abcd1 |
| 1418840_at | Lgx only | 667 | 575 | 556 | 501 | −1.16 | −1.20 | −1.33 | Pdcd4 |
| 1418846_at | CR only | 103 | 199 | 99 | 162 | 1.93 | −1.04 | 1.57 | Ap4m1 |
| 1418847_at | Lgx only | 28 | 28 | 56 | 92 | −1.04 | 1.95 | 3.24 | Arg2 |
| 1418861_at | Res only | 887 | 950 | 1409 | 1371 | 1.07 | 1.59 | 1.55 | Pias4 |
| 1418863_at | Lgx only | 1860 | 2280 | 2226 | 2561 | 1.23 | 1.20 | 1.38 | Gata4 |
| 1418869_a_at | Lgx only | 678 | 738 | 805 | 954 | 1.09 | 1.19 | 1.41 | Pus1 |
| 1418874_a_at | Lgx only | 5030 | 5073 | 6091 | 6369 | 1.01 | 1.21 | 1.27 | Psmd4 |
| 1418885_a_at | Res & Lgx | 906 | 1107 | 1173 | 1863 | 1.22 | 1.30 | 2.06 | Idh3b |
| 1418899_at | Lgx only | 1067 | 834 | 819 | 715 | −1.28 | −1.30 | −1.49 | Ufm1 |
| 1418924_at | Lgx only | 89 | 173 | 226 | 307 | 1.95 | 2.54 | 3.45 | Rassf7 |
| 1418926_at | Res & Lgx | 3012 | 2659 | 2326 | 2255 | −1.13 | −1.29 | −1.34 | Zeb1 |
| 1418928_a_at | Lgx only | 958 | 1057 | 1130 | 1663 | 1.10 | 1.18 | 1.74 | 2310038H17Rik |
| 1418929_at | Lgx only | 410 | 472 | 537 | 581 | 1.15 | 1.31 | 1.42 | Ift57 |
| 1418933_at | Res only | 8 | 28 | 16 | 13 | 3.76 | 2.08 | 1.68 | Slc1a6 |
| 1418947_at | CR only | 247 | 343 | 289 | 319 | 1.39 | 1.17 | 1.29 | Nek3 |
| 1418952_at | Res & Lgx | 9461 | 9278 | 12035 | 12244 | −1.02 | 1.27 | 1.29 | Txlnb |
| 1418967_a_at | Lgx only | 302 | 261 | 219 | 209 | −1.16 | −1.38 | −1.45 | St7 |
| 1418968_at | Lgx only | 2166 | 1884 | 1630 | 1473 | −1.15 | −1.33 | −1.47 | Rb1cc1 |
| 1418986_a_at | Lgx only | 1097 | 1006 | 854 | 642 | −1.09 | −1.28 | −1.71 | Uxt |
| 1418987_at | Res only | 161 | 117 | 260 | 269 | −1.38 | 1.61 | 1.67 | Pla2g2d |
| 1418988_at | Res & Lgx | 2768 | 2378 | 2028 | 1916 | −1.16 | −1.36 | −1.44 | Pex7 |
| 1418996_a_at | Lgx only | 3436 | 3503 | 3043 | 2693 | 1.02 | −1.13 | −1.28 | Lyrm5 |
| 1419013_at | Res only | 438 | 501 | 605 | 499 | 1.14 | 1.38 | 1.14 | Gpatch1 |
| 1419026_at | Lgx only | 910 | 978 | 1211 | 1131 | 1.08 | 1.33 | 1.24 | Daxx |
| 1419037_at | Lgx only | 1248 | 1228 | 1010 | 1005 | −1.02 | −1.24 | −1.24 | Csnk2a1 |
| 1419062_at | Lgx only | 646 | 672 | 717 | 873 | 1.04 | 1.11 | 1.35 | Epb4.1l3 |
| 1419070_at | CR only | 274 | 536 | 312 | 423 | 1.96 | 1.14 | 1.54 | Cys1 |
| 1419072_at | Lgx only | 1253 | 1214 | 1154 | 1035 | −1.03 | −1.09 | −1.21 | Gstm7 |
| 1419074_at | Lgx only | 669 | 635 | 434 | 385 | −1.05 | −1.54 | −1.74 | Chac2 |
| 1419081_at | Lgx only | 620 | 617 | 514 | 392 | −1.00 | −1.21 | −1.58 | Atg10 |
| 1419109_at | Res & Lgx | 19560 | 22347 | 25933 | 31691 | 1.14 | 1.33 | 1.62 | Hrc |
| 1419131_at | CR only | 10 | 65 | 30 | 43 | 6.52 | 2.99 | 4.25 | F13b |
| 1419144_at | Lgx only | 328 | 358 | 484 | 596 | 1.09 | 1.48 | 1.81 | Cd163 |
| 1419158_a_at | CR only | 760 | 935 | 726 | 788 | 1.23 | −1.05 | 1.04 | Hars2 |
| 1419164_at | Res & Lgx | 1598 | 1612 | 1126 | 698 | 1.01 | −1.42 | −2.29 | Zfp260 |
| 1419169_at | Lgx only | 1118 | 1026 | 941 | 817 | −1.09 | −1.19 | −1.37 | Mapk6 |
| 1419170_at | Lgx only | 2926 | 2624 | 2049 | 1889 | −1.12 | −1.43 | −1.55 | Tmem157 |
| 1419174_at | Lgx only | 1433 | 1234 | 1044 | 1065 | −1.16 | −1.37 | −1.34 | 2410004B18Rik |
| 1419182_at | Lgx only | 525 | 545 | 632 | 749 | 1.04 | 1.20 | 1.43 | Svep1 |
| 1419186_a_at | Res & Lgx | 779 | 684 | 511 | 470 | −1.14 | −1.52 | −1.66 | St8sia4 |
| 1419214_at | Res only | 146 | 199 | 310 | 284 | 1.36 | 2.12 | 1.94 | Tnfrsf11a |
| 1419238_at | Lgx only | 154 | 255 | 316 | 301 | 1.66 | 2.06 | 1.96 | Abca7 |
| 1419258_at | Lgx only | 1711 | 1423 | 1294 | 1111 | −1.20 | −1.32 | −1.54 | Tcea1 |
| 1419272_at | Res only | 428 | 435 | 585 | 498 | 1.02 | 1.37 | 1.16 | Myd88 |
| 1419292_at | Lgx only | 3158 | 3740 | 4270 | 4656 | 1.18 | 1.35 | 1.47 | Htra3 |
| 1419295_at | Res only | 209 | 211 | 411 | 276 | 1.01 | 1.96 | 1.32 | Creb3l1 |
| 1419297_at | CR only | 149 | 66 | 122 | 147 | −2.25 | −1.22 | −1.01 | H2-Oa |
| 1419302_at | Res & Lgx | 794 | 919 | 1108 | 1247 | 1.16 | 1.40 | 1.57 | Heyl |
| 1419333_at | Lgx only | 392 | 461 | 414 | 572 | 1.18 | 1.06 | 1.46 | 1110008J03Rik |
| 1419352_at | Lgx only | 1989 | 1829 | 1498 | 1340 | −1.09 | −1.33 | −1.48 | I7Rn6 |
| 1419354_at | Lgx only | 1196 | 1159 | 1578 | 1790 | −1.03 | 1.32 | 1.50 | Klf7 |
| 1419358_at | Lgx only | 207 | 323 | 185 | 348 | 1.56 | −1.11 | 1.68 | Sorcs2 |
| 1419366_at | Res & Lgx | 642 | 733 | 782 | 1009 | 1.14 | 1.22 | 1.57 | Zmat5 |
| 1419375_at | Res & Lgx | 1257 | 1211 | 921 | 872 | −1.04 | −1.36 | −1.44 | Wbp4 |
| 1419398_a_at | Lgx only | 10797 | 11078 | 10041 | 8759 | 1.03 | −1.08 | −1.23 | Reep5 |
| 1419415_a_at | CR only | 1168 | 905 | 1009 | 964 | −1.29 | −1.16 | −1.21 | Rarg |
| 1419428_a_at | Lgx only | 1374 | 1692 | 1993 | 2038 | 1.23 | 1.45 | 1.48 | Gaa |
| 1419429_at | Res & Lgx | 79 | 137 | 161 | 223 | 1.74 | 2.04 | 2.83 | Cntfr |
| 1419452_at | Lgx only | 1034 | 914 | 835 | 680 | −1.13 | −1.24 | −1.52 | Uchl5 |
| 1419455_at | Lgx only | 6361 | 6701 | 5697 | 5310 | 1.05 | −1.12 | −1.20 | Il10rb |
| 1419470_at | Res & Lgx | 865 | 849 | 644 | 649 | −1.02 | −1.34 | −1.33 | Gnb4 |
| 1419477_at | Res only | 35 | 98 | 190 | 144 | 2.81 | 5.44 | 4.12 | Clec2d |
| 1419484_a_at | Lgx only | 20734 | 22346 | 11461 | 5733 | 1.08 | −1.81 | −3.62 | Gbas |
| 1419491_at | Res only | 13 | 40 | 52 | 67 | 3.14 | 4.08 | 5.30 | Defb1 |
| 1419495_at | Lgx only | 2055 | 1752 | 1771 | 1374 | −1.17 | −1.16 | −1.50 | Immp2l |
| 1419499_at | Lgx only | 7940 | 7261 | 10653 | 11012 | −1.09 | 1.34 | 1.39 | Gpam |
| 1419518_at | All | 7504 | 12289 | 3758 | 4518 | 1.64 | −2.00 | −1.66 | Tuba8 |
| 1419527_at | Lgx only | 403 | 437 | 548 | 829 | 1.08 | 1.36 | 2.06 | Comp |
| 1419550_a_at | Lgx only | 2491 | 2352 | 1855 | 1537 | −1.06 | −1.34 | −1.62 | Stk39 |
| 1419569_a_at | Res only | 681 | 700 | 1171 | 1519 | 1.03 | 1.72 | 2.23 | Isg20 |
| 1419584_at | Lgx only | 521 | 505 | 846 | 843 | −1.03 | 1.62 | 1.62 | Ttc28 |
| 1419609_at | CR only | 53 | 133 | 65 | 112 | 2.53 | 1.24 | 2.13 | Ccr1 |
| 1419630_a_at | CR & Lgx | 549 | 668 | 630 | 755 | 1.22 | 1.15 | 1.37 | Trim11 |
| 1419631_at | Res & Lgx | 119 | 146 | 218 | 228 | 1.23 | 1.84 | 1.92 | Was |
| 1419645_at | Lgx only | 463 | 390 | 335 | 288 | −1.19 | −1.38 | −1.61 | Cstf2 |
| 1419657_a_at | CR & Lgx | 1163 | 874 | 858 | 623 | −1.33 | −1.36 | −1.87 | Slc25a36 |
| 1419660_at | Lgx only | 4264 | 3723 | 4383 | 3253 | −1.15 | 1.03 | −1.31 | 1600012F09Rik |
| 1419687_at | Lgx only | 8909 | 9784 | 12046 | 13075 | 1.10 | 1.35 | 1.47 | Macrod1 |
| 1419736_a_at | Res & Lgx | 3408 | 3084 | 2455 | 1934 | −1.10 | −1.39 | −1.76 | Eif1ay |
| 1419762_at | Res & Lgx | 38 | 84 | 155 | 154 | 2.18 | 4.03 | 4.01 | Ubd |
| 1419787_a_at | Lgx only | 312 | 385 | 461 | 581 | 1.24 | 1.48 | 1.86 | Zfp628 |
| 1419824_a_at | Res & Lgx | 455 | 428 | 270 | 228 | −1.06 | −1.69 | −2.00 | A230062G08Rik |
| 1419952_at | Lgx only | 27 | 55 | 105 | 168 | 2.03 | 3.90 | 6.26 | 1700023D09Rik |
| 1420099_at | CR only | 203 | 411 | 349 | 286 | 2.02 | 1.72 | 1.40 | D13Ertd787e |
| 1420123_at | Res & Lgx | 3511 | 3401 | 5048 | 5686 | −1.03 | 1.44 | 1.62 | Tcta |
| 1420183_at | Lgx only | 234 | 252 | 360 | 451 | 1.08 | 1.54 | 1.93 | Lor |
| 1420325_at | Res only | 57 | 130 | 144 | 132 | 2.26 | 2.52 | 2.30 | Cramp1l |
| 1420329_at | Lgx only | 567 | 517 | 392 | 323 | −1.10 | −1.45 | −1.76 | 4930455C21Rik |
| 1420339_at | Res only | 309 | 242 | 217 | 225 | −1.28 | −1.43 | −1.37 | LOC100047915 |
| 1420374_at | Res & Lgx | 758 | 989 | 1238 | 1240 | 1.31 | 1.63 | 1.64 | Foxj2 |
| 1420375_at | Res & Lgx | 401 | 402 | 615 | 593 | 1.00 | 1.53 | 1.48 | Kif3a |
| 1420377_at | Lgx only | 89 | 105 | 134 | 173 | 1.18 | 1.50 | 1.94 | St8sia2 |
| 1420387_at | Res & Lgx | 2021 | 2029 | 1347 | 1414 | 1.00 | −1.50 | −1.43 | Mpv17 |
| 1420388_at | Lgx only | 160 | 221 | 219 | 350 | 1.38 | 1.37 | 2.19 | Prss12 |
| 1420405_at | Lgx only | 181 | 177 | 327 | 423 | −1.02 | 1.81 | 2.34 | Slco1a4 |
| 1420427_a_at | CR only | 1499 | 1159 | 1676 | 1564 | −1.29 | 1.12 | 1.04 | Dhx32 |
| 1420497_a_at | Res only | 446 | 471 | 707 | 611 | 1.06 | 1.58 | 1.37 | Cebpz |
| 1420502_at | Lgx only | 1203 | 971 | 967 | 740 | −1.24 | −1.24 | −1.63 | Sat1 |
| 1420507_a_at | Res & Lgx | 787 | 742 | 523 | 391 | −1.06 | −1.50 | −2.01 | 3110031B13Rik |
| 1420513_at | Res & Lgx | 4347 | 3651 | 3096 | 2640 | −1.19 | −1.40 | −1.65 | Efcab2 |
| 1420580_at | Res only | 108 | 171 | 205 | 173 | 1.59 | 1.90 | 1.60 | 4930429B21Rik |
| 1420617_at | CR & Lgx | 1660 | 2030 | 1377 | 1316 | 1.22 | −1.21 | −1.26 | Cpeb4 |
| 1420619_a_at | Lgx only | 9782 | 11497 | 12736 | 14220 | 1.18 | 1.30 | 1.45 | Aes |
| 1420654_a_at | Lgx only | 836 | 1010 | 530 | 414 | 1.21 | −1.58 | −2.02 | Gbe1 |
| 1420657_at | CR & Lgx | 978 | 1762 | 1973 | 2727 | 1.80 | 2.02 | 2.79 | Ucp3 |
| 1420684_at | Lgx only | 184 | 272 | 311 | 338 | 1.48 | 1.70 | 1.84 | Acox3 |
| 1420693_at | Lgx only | 12144 | 13502 | 14705 | 16745 | 1.11 | 1.21 | 1.38 | Myom1 |
| 1420703_at | Lgx only | 230 | 242 | 309 | 412 | 1.05 | 1.34 | 1.79 | Csf2ra |
| 1420707_a_at | Res only | 15 | 45 | 86 | 56 | 3.03 | 5.79 | 3.77 | Traip |
| 1420711_a_at | Res & Lgx | 1740 | 1664 | 1104 | 1160 | −1.05 | −1.58 | −1.50 | Pxmp3 |
| 1420715_a_at | Res only | 264 | 353 | 394 | 398 | 1.34 | 1.49 | 1.51 | Pparg |
| 1420727_a_at | Lgx only | 834 | 685 | 683 | 642 | −1.22 | −1.22 | −1.30 | Tmlhe |
| 1420770_at | Lgx only | 119 | 204 | 196 | 229 | 1.71 | 1.64 | 1.92 | Klk1b24 |
| 1420812_at | Res only | 636 | 684 | 880 | 887 | 1.08 | 1.38 | 1.39 | Hdac7a |
| 1420815_at | Res & Lgx | 6531 | 5930 | 4529 | 3754 | −1.10 | −1.44 | −1.74 | Gdi2 |
| 1420829_a_at | Res & Lgx | 1575 | 1422 | 1004 | 915 | −1.11 | −1.57 | −1.72 | Ywhaq |
| 1420850_at | Lgx only | 811 | 697 | 641 | 504 | −1.16 | −1.27 | −1.61 | Crnkl1 |
| 1420851_at | Lgx only | 742 | 595 | 613 | 560 | −1.25 | −1.21 | −1.32 | Pard6g |
| 1420858_at | Lgx only | 4637 | 3856 | 3968 | 3017 | −1.20 | −1.17 | −1.54 | Pkia |
| 1420886_a_at | Res & Lgx | 3739 | 3833 | 4963 | 5400 | 1.03 | 1.33 | 1.44 | Xbp1 |
| 1420890_at | Res & Lgx | 5214 | 4425 | 2406 | 1933 | −1.18 | −2.17 | −2.70 | Hccs |
| 1420895_at | Lgx only | 917 | 770 | 710 | 650 | −1.19 | −1.29 | −1.41 | Tgfbr1 |
| 1420909_at | Lgx only | 2678 | 2641 | 1885 | 1364 | −1.01 | −1.42 | −1.96 | Vegfa |
| 1420911_a_at | Lgx only | 3466 | 3820 | 3678 | 4193 | 1.10 | 1.06 | 1.21 | Mfge8 |
| 1420925_at | Lgx only | 72 | 124 | 151 | 175 | 1.71 | 2.08 | 2.42 | Tub |
| 1420960_at | Res only | 96 | 110 | 192 | 231 | 1.15 | 1.99 | 2.40 | Fancg |
| 1420965_a_at | Lgx only | 874 | 1154 | 1487 | 1587 | 1.32 | 1.70 | 1.82 | Enc1 |
| 1420969_at | Lgx only | 466 | 483 | 535 | 830 | 1.04 | 1.15 | 1.78 | Btbd14b |
| 1420981_a_at | Lgx only | 827 | 972 | 964 | 1187 | 1.18 | 1.17 | 1.44 | Lmo4 |
| 1420990_at | Res only | 312 | 320 | 517 | 236 | 1.02 | 1.65 | −1.32 | Chd1 |
| 1420991_at | Lgx only | 53384 | 52666 | 61602 | 72604 | −1.01 | 1.15 | 1.36 | Ankrd1 |
| 1421019_at | Lgx only | 4065 | 3950 | 3600 | 3047 | −1.03 | −1.13 | −1.33 | 1700021F05Rik |
| 1421025_at | Lgx only | 849 | 1055 | 1040 | 1273 | 1.24 | 1.22 | 1.50 | Agpat1 |
| 1421027_a_at | Res & Lgx | 1926 | 1835 | 1240 | 1194 | −1.05 | −1.55 | −1.61 | Mef2c |
| 1421042_at | Res only | 445 | 458 | 615 | 583 | 1.03 | 1.38 | 1.31 | Arhgef2 |
| 1421054_at | Lgx only | 720 | 688 | 568 | 429 | −1.05 | −1.27 | −1.68 | Xpo4 |
| 1421087_at | CR & Lgx | 659 | 948 | 669 | 1049 | 1.44 | 1.02 | 1.59 | Per3 |
| 1421096_at | Res & Lgx | 343 | 231 | 143 | 153 | −1.48 | −2.39 | −2.24 | Trpc1 |
| 1421099_at | Lgx only | 201 | 206 | 279 | 348 | 1.03 | 1.39 | 1.74 | Bhlhb3 |
| 1421140_a_at | Lgx only | 966 | 1184 | 1048 | 1522 | 1.23 | 1.09 | 1.57 | Foxp1 |
| 1421164_a_at | Lgx only | 790 | 1029 | 1101 | 1483 | 1.30 | 1.39 | 1.88 | Arhgef1 |
| 1421174_at | CR only | 170 | 74 | 105 | 131 | −2.29 | −1.62 | −1.30 | Irf4 |
| 1421254_a_at | Res & Lgx | 8190 | 8322 | 11932 | 11052 | 1.02 | 1.46 | 1.35 | Sgcg |
| 1421265_a_at | Lgx only | 2787 | 3702 | 3156 | 4067 | 1.33 | 1.13 | 1.46 | Rbm38 |
| 1421287_a_at | Res & Lgx | 1918 | 2165 | 2346 | 2394 | 1.13 | 1.22 | 1.25 | Pecam1 |
| 1421292_a_at | Lgx only | 1159 | 1309 | 1372 | 1641 | 1.13 | 1.18 | 1.42 | A730008L03Rik |
| 1421301_at | Res & Lgx | 25 | 50 | 65 | 79 | 2.01 | 2.64 | 3.20 | Zic2 |
| 1421361_at | Res only | 7 | 12 | 14 | 17 | 1.71 | 1.97 | 2.36 | Grk1 |
| 1421373_at | Lgx only | 150 | 180 | 225 | 291 | 1.20 | 1.50 | 1.94 | Cox4i2 |
| 1421374_a_at | Res & Lgx | 22933 | 26328 | 30566 | 37711 | 1.15 | 1.33 | 1.64 | Fxyd1 |
| 1421425_a_at | Lgx only | 16449 | 16822 | 17946 | 19197 | 1.02 | 1.09 | 1.17 | Rcan2 |
| 1421444_at | Res only | 74 | 60 | 18 | 28 | −1.23 | −4.13 | −2.61 | Pgr |
| 1421468_at | Lgx only | 901 | 957 | 947 | 1325 | 1.06 | 1.05 | 1.47 | Kcnj3 |
| 1421530_a_at | Lgx only | 27 | 71 | 53 | 97 | 2.66 | 1.98 | 3.62 | Grm8 |
| 1421534_at | Lgx only | 811 | 795 | 808 | 606 | −1.02 | −1.00 | −1.34 | Dfna5h |
| 1421541_a_at | Lgx only | 21 | 44 | 48 | 100 | 2.09 | 2.27 | 4.69 | Mef2b |
| 1421654_a_at | Lgx only | 1679 | 1993 | 2038 | 2187 | 1.19 | 1.21 | 1.30 | Lmna |
| 1421657_a_at | Lgx only | 586 | 697 | 816 | 882 | 1.19 | 1.39 | 1.51 | Sox17 |
| 1421712_at | Res only | 44 | 104 | 121 | 85 | 2.35 | 2.74 | 1.93 | Sele |
| 1421729_a_at | Res & Lgx | 681 | 584 | 500 | 370 | −1.17 | −1.36 | −1.84 | Fert2 |
| 1421733_a_at | Res & Lgx | 1129 | 1104 | 1432 | 1650 | −1.02 | 1.27 | 1.46 | Tpst1 |
| 1421743_a_at | Res & Lgx | 9703 | 10330 | 11559 | 11327 | 1.06 | 1.19 | 1.17 | Pcbp2 |
| 1421750_a_at | Lgx only | 2246 | 2231 | 1829 | 1446 | −1.01 | −1.23 | −1.55 | Vbp1 |
| 1421797_a_at | Lgx only | 557 | 651 | 646 | 772 | 1.17 | 1.16 | 1.39 | Snx12 |
| 1421808_at | Res only | 72 | 37 | 25 | 26 | −1.92 | −2.88 | −2.71 | Defb5 |
| 1421810_at | Lgx only | 868 | 921 | 932 | 1143 | 1.06 | 1.07 | 1.32 | Dgcr2 |
| 1421813_a_at | Lgx only | 18627 | 21854 | 22085 | 24924 | 1.17 | 1.19 | 1.34 | Psap |
| 1421820_a_at | Lgx only | 2152 | 2587 | 2644 | 3110 | 1.20 | 1.23 | 1.45 | Nf2 |
| 1421826_at | Lgx only | 702 | 623 | 580 | 445 | −1.13 | −1.21 | −1.58 | Dll4 |
| 1421861_at | Lgx only | 1285 | 1602 | 1652 | 1882 | 1.25 | 1.28 | 1.46 | Clstn1 |
| 1421871_at | Lgx only | 1104 | 837 | 695 | 549 | −1.32 | −1.59 | −2.01 | Sh3bgrl |
| 1421872_at | Lgx only | 911 | 910 | 719 | 556 | −1.00 | −1.27 | −1.64 | Rab24 |
| 1421880_at | Res only | 970 | 1061 | 1162 | 1121 | 1.09 | 1.20 | 1.16 | Mtmr1 |
| 1421887_a_at | Lgx only | 10860 | 11944 | 14384 | 16154 | 1.10 | 1.32 | 1.49 | Aplp2 |
| 1421894_a_at | Lgx only | 1285 | 1111 | 1077 | 876 | −1.16 | −1.19 | −1.47 | Tpp2 |
| 1421900_at | Lgx only | 704 | 851 | 937 | 1086 | 1.21 | 1.33 | 1.54 | Eif2ak1 |
| 1421910_at | CR & Lgx | 616 | 466 | 496 | 388 | −1.32 | −1.24 | −1.58 | Tcf20 |
| 1421929_at | Res & Lgx | 584 | 771 | 1070 | 1011 | 1.32 | 1.83 | 1.73 | Epha4 |
| 1421960_at | Lgx only | 251 | 313 | 373 | 451 | 1.25 | 1.48 | 1.79 | Adcy3 |
| 1421985_a_at | Lgx only | 1206 | 1190 | 1259 | 970 | −1.01 | 1.04 | −1.24 | Eif4e2 |
| 1422063_a_at | Res & Lgx | 303 | 432 | 514 | 657 | 1.43 | 1.70 | 2.17 | Pex5 |
| 1422085_at | Res only | 89 | 141 | 178 | 209 | 1.58 | 2.00 | 2.34 | Tbx19 |
| 1422122_at | Lgx only | 45 | 52 | 102 | 145 | 1.15 | 2.26 | 3.23 | Fcer2a |
| 1422157_a_at | Res & Lgx | 1891 | 1621 | 1425 | 1408 | −1.17 | −1.33 | −1.34 | Itgb1bp1 |
| 1422160_at | Res & Lgx | 194 | 137 | 98 | 86 | −1.42 | −1.99 | −2.24 | H2-T24 |
| 1422183_a_at | Res & Lgx | 456 | 433 | 718 | 774 | −1.05 | 1.57 | 1.70 | Adra1b |
| 1422185_a_at | Lgx only | 2280 | 2364 | 3023 | 3264 | 1.04 | 1.33 | 1.43 | Cyb5r3 |
| 1422202_at | All | 187 | 261 | 266 | 299 | 1.39 | 1.42 | 1.60 | Thrb |
| 1422250_at | Res only | 74 | 95 | 175 | 109 | 1.29 | 2.36 | 1.47 | Map3k2 |
| 1422253_at | Res only | 72 | 82 | 22 | 25 | 1.14 | −3.30 | −2.84 | Col10a1 |
| 1422273_at | CR only | 17 | 64 | 65 | 79 | 3.66 | 3.75 | 4.54 | Mmp1b |
| 1422303_a_at | Lgx only | 115 | 142 | 161 | 192 | 1.24 | 1.40 | 1.67 | Tnfrsf18 |
| 1422325_at | CR only | 99 | 54 | 92 | 79 | −1.85 | −1.08 | −1.25 | Magea5 |
| 1422349_at | Res only | 108 | 91 | 204 | 164 | −1.19 | 1.88 | 1.51 | Ccr1l1 |
| 1422368_at | CR only | 63 | 20 | 70 | 73 | −3.19 | 1.12 | 1.17 | V1ra5 |
| 1422429_at | Res & Lgx | 3906 | 3945 | 3274 | 3064 | 1.01 | −1.19 | −1.27 | Rnf14 |
| 1422431_at | Res & Lgx | 174 | 193 | 294 | 266 | 1.11 | 1.69 | 1.53 | Magee1 |
| 1422442_at | Res & Lgx | 2592 | 2482 | 1984 | 1860 | −1.04 | −1.31 | −1.39 | Smu1 |
| 1422443_at | CR only | 2188 | 2545 | 2408 | 2383 | 1.16 | 1.10 | 1.09 | Xpnpep1 |
| 1422470_at | CR & Lgx | 10633 | 8053 | 8886 | 6881 | −1.32 | −1.20 | −1.55 | Bnip3 |
| 1422476_at | Lgx only | 632 | 824 | 773 | 895 | 1.30 | 1.22 | 1.42 | Ifi30 |
| 1422479_at | CR only | 1246 | 1673 | 1146 | 1250 | 1.34 | −1.09 | 1.00 | Acss2 |
| 1422505_at | Res & Lgx | 2509 | 2529 | 2006 | 2084 | 1.01 | −1.25 | −1.20 | Chrac1 |
| 1422514_at | Lgx only | 811 | 912 | 1085 | 1253 | 1.12 | 1.34 | 1.54 | Aebp1 |
| 1422521_at | Lgx only | 1147 | 1461 | 1635 | 2058 | 1.27 | 1.43 | 1.79 | Dctn1 |
| 1422536_at | Lgx only | 92593 | 100541 | 105298 | 145101 | 1.09 | 1.14 | 1.57 | Tnni3 |
| 1422559_at | Res & Lgx | 2320 | 2134 | 1617 | 1469 | −1.09 | −1.44 | −1.58 | Ube2n |
| 1422562_at | Lgx only | 4072 | 5149 | 5749 | 7406 | 1.26 | 1.41 | 1.82 | Rrad |
| 1422568_at | Lgx only | 4621 | 4163 | 3766 | 3704 | −1.11 | −1.23 | −1.25 | Ndel1 |
| 1422579_at | Lgx only | 2795 | 2515 | 2100 | 1950 | −1.11 | −1.33 | −1.43 | Hspe1 |
| 1422580_at | Lgx only | 21473 | 23970 | 33904 | 66655 | 1.12 | 1.58 | 3.10 | Myl4 |
| 1422589_at | Lgx only | 1557 | 2101 | 1799 | 2359 | 1.35 | 1.16 | 1.51 | Rab3a |
| 1422594_at | Res only | 1164 | 1044 | 782 | 887 | −1.12 | −1.49 | −1.31 | 5730470L24Rik |
| 1422597_at | Lgx only | 1172 | 1880 | 1299 | 1581 | 1.60 | 1.11 | 1.35 | Mmp15 |
| 1422598_at | Lgx only | 376 | 482 | 558 | 667 | 1.28 | 1.48 | 1.77 | Casq1 |
| 1422601_at | Lgx only | 678 | 602 | 614 | 451 | −1.13 | −1.11 | −1.50 | Serpinb9 |
| 1422622_at | Lgx only | 1987 | 2361 | 2556 | 2886 | 1.19 | 1.29 | 1.45 | Nos3 |
| 1422624_at | Res & Lgx | 585 | 523 | 324 | 366 | −1.12 | −1.80 | −1.60 | Rev1 |
| 1422631_at | CR only | 459 | 290 | 354 | 311 | −1.58 | −1.30 | −1.47 | Ahr |
| 1422636_at | Lgx only | 484 | 476 | 356 | 311 | −1.02 | −1.36 | −1.55 | Dmtf1 |
| 1422647_at | Lgx only | 551 | 486 | 766 | 945 | −1.13 | 1.39 | 1.72 | Ring1 |
| 1422654_at | Res & Lgx | 6849 | 8679 | 9035 | 10593 | 1.27 | 1.32 | 1.55 | Sgca |
| 1422656_at | Lgx only | 348 | 337 | 280 | 194 | −1.03 | −1.24 | −1.79 | Rasl2-9 |
| 1422669_at | Res & Lgx | 1326 | 1103 | 925 | 768 | −1.20 | −1.43 | −1.72 | Ebag9 |
| 1422678_at | Lgx only | 8205 | 10614 | 8228 | 11571 | 1.29 | 1.00 | 1.41 | Dgat2 |
| 1422687_at | Res only | 342 | 422 | 540 | 502 | 1.23 | 1.58 | 1.47 | Nras |
| 1422704_at | Lgx only | 912 | 889 | 790 | 692 | −1.03 | −1.16 | −1.32 | Gyk |
| 1422710_a_at | Lgx only | 204 | 256 | 533 | 712 | 1.26 | 2.62 | 3.50 | Cacna1h |
| 1422731_at | Lgx only | 1938 | 1979 | 1826 | 1508 | 1.02 | −1.06 | −1.29 | Limd1 |
| 1422750_a_at | Lgx only | 12 | 15 | 16 | 26 | 1.27 | 1.32 | 2.13 | Zmynd10 |
| 1422754_at | Lgx only | 5302 | 6134 | 5414 | 7120 | 1.16 | 1.02 | 1.34 | Tmod1 |
| 1422759_a_at | Lgx only | 716 | 910 | 1014 | 972 | 1.27 | 1.42 | 1.36 | Xpo6 |
| 1422771_at | Lgx only | 662 | 616 | 845 | 1033 | −1.07 | 1.28 | 1.56 | Smad6 |
| 1422794_at | Lgx only | 2668 | 2498 | 2540 | 2197 | −1.07 | −1.05 | −1.21 | Cul3 |
| 1422797_at | Lgx only | 2497 | 2556 | 2816 | 3053 | 1.02 | 1.13 | 1.22 | Mapbpip |
| 1422799_at | Lgx only | 1417 | 2232 | 2331 | 3007 | 1.58 | 1.64 | 2.12 | Bat2 |
| 1422801_at | Lgx only | 1868 | 2054 | 2333 | 2401 | 1.10 | 1.25 | 1.29 | G3bp1 |
| 1422811_at | Lgx only | 1842 | 1949 | 3745 | 4459 | 1.06 | 2.03 | 2.42 | Slc27a1 |
| 1422819_at | Lgx only | 1868 | 1541 | 1669 | 1324 | −1.21 | −1.12 | −1.41 | Mrpl36 |
| 1422820_at | Res & Lgx | 1160 | 1556 | 2287 | 3124 | 1.34 | 1.97 | 2.69 | Lipe |
| 1422845_at | Res & Lgx | 2601 | 2532 | 1767 | 1768 | −1.03 | −1.47 | −1.47 | Canx |
| 1422855_at | Lgx only | 1072 | 1244 | 1287 | 1505 | 1.16 | 1.20 | 1.40 | Cpsf3 |
| 1422858_at | Res & Lgx | 647 | 600 | 471 | 385 | −1.08 | −1.37 | −1.68 | Trip4 |
| 1422869_at | CR only | 322 | 486 | 356 | 429 | 1.51 | 1.11 | 1.33 | Mertk |
| 1422880_at | Res only | 3821 | 4166 | 5178 | 4697 | 1.09 | 1.36 | 1.23 | Sypl |
| 1422884_at | Res & Lgx | 2609 | 2458 | 1699 | 1360 | −1.06 | −1.54 | −1.92 | Snrpd3 |
| 1422888_at | Res & Lgx | 2451 | 2326 | 1824 | 1740 | −1.05 | −1.34 | −1.41 | Rnf5 |
| 1422895_at | Lgx only | 1484 | 1345 | 1128 | 1085 | −1.10 | −1.32 | −1.37 | Vamp4 |
| 1422904_at | Lgx only | 1644 | 2057 | 2434 | 3071 | 1.25 | 1.48 | 1.87 | Fmo2 |
| 1422919_at | Lgx only | 1447 | 1561 | 1174 | 1081 | 1.08 | −1.23 | −1.34 | Hrasls |
| 1422927_at | Lgx only | 9559 | 8873 | 9036 | 7917 | −1.08 | −1.06 | −1.21 | Yipf7 |
| 1422975_at | Lgx only | 270 | 215 | 239 | 143 | −1.25 | −1.13 | −1.88 | Mme |
| 1423025_a_at | Res & Lgx | 4676 | 4201 | 3674 | 3128 | −1.11 | −1.27 | −1.50 | Schip1 |
| 1423038_at | CR only | 739 | 613 | 619 | 676 | −1.21 | −1.19 | −1.09 | Stx6 |
| 1423044_at | Lgx only | 3560 | 3888 | 3058 | 2626 | 1.09 | −1.16 | −1.36 | Prosc |
| 1423047_at | Lgx only | 1895 | 2061 | 2077 | 2332 | 1.09 | 1.10 | 1.23 | Tollip |
| 1423049_a_at | Lgx only | 87808 | 100652 | 95761 | 125022 | 1.15 | 1.09 | 1.42 | Tpm1 |
| 1423067_at | Lgx only | 1021 | 1177 | 1166 | 1322 | 1.15 | 1.14 | 1.29 | Cdk5rap3 |
| 1423072_at | Res & Lgx | 9143 | 7426 | 14144 | 14099 | −1.23 | 1.55 | 1.54 | 6720475J19Rik |
| 1423073_at | Res & Lgx | 2730 | 2611 | 1976 | 1859 | −1.05 | −1.38 | −1.47 | Cmpk |
| 1423078_a_at | Res only | 281 | 268 | 138 | 193 | −1.05 | −2.03 | −1.45 | Sc4mol |
| 1423083_at | Lgx only | 829 | 634 | 640 | 576 | −1.31 | −1.30 | −1.44 | Rab33b |
| 1423085_at | Lgx only | 2081 | 2337 | 2335 | 2878 | 1.12 | 1.12 | 1.38 | Efnb3 |
| 1423086_at | Lgx only | 581 | 574 | 944 | 865 | −1.01 | 1.62 | 1.49 | Npc1 |
| 1423104_at | Lgx only | 1861 | 2159 | 1551 | 1285 | 1.16 | −1.20 | −1.45 | Irs1 |
| 1423107_at | Res & Lgx | 13435 | 12780 | 18773 | 18511 | −1.05 | 1.40 | 1.38 | Ube2b |
| 1423115_at | Lgx only | 1874 | 2293 | 2021 | 2497 | 1.22 | 1.08 | 1.33 | St6galnac6 |
| 1423116_at | Lgx only | 367 | 369 | 418 | 514 | 1.01 | 1.14 | 1.40 | Dom3z |
| 1423117_at | Lgx only | 1641 | 1974 | 2060 | 2325 | 1.20 | 1.26 | 1.42 | Pum1 |
| 1423120_at | Lgx only | 1872 | 1820 | 2582 | 2866 | −1.03 | 1.38 | 1.53 | Ide |
| 1423145_a_at | Lgx only | 67091 | 70639 | 67884 | 94069 | 1.05 | 1.01 | 1.40 | Tcap |
| 1423159_at | Lgx only | 8820 | 7770 | 8104 | 7185 | −1.14 | −1.09 | −1.23 | Dld |
| 1423167_at | Lgx only | 2048 | 1950 | 1592 | 1289 | −1.05 | −1.29 | −1.59 | Mobkl3 |
| 1423185_a_at | Lgx only | 1046 | 1030 | 1125 | 1433 | −1.01 | 1.08 | 1.37 | Ubap1 |
| 1423195_at | Lgx only | 770 | 772 | 644 | 545 | 1.00 | −1.20 | −1.41 | Hiat1 |
| 1423210_a_at | Lgx only | 15838 | 13912 | 13292 | 11593 | −1.14 | −1.19 | −1.37 | Nola3 |
| 1423238_at | CR only | 10141 | 11875 | 10734 | 10997 | 1.17 | 1.06 | 1.08 | Itgb1bp2 |
| 1423245_at | Lgx only | 2212 | 2559 | 2650 | 2839 | 1.16 | 1.20 | 1.28 | Cops7a |
| 1423247_at | Res & Lgx | 1654 | 1535 | 1377 | 1412 | −1.08 | −1.20 | −1.17 | Txndc4 |
| 1423283_at | Res only | 2245 | 2516 | 2704 | 2323 | 1.12 | 1.20 | 1.03 | Pitpna |
| 1423289_a_at | Lgx only | 590 | 520 | 490 | 411 | −1.14 | −1.21 | −1.44 | 1810029B16Rik |
| 1423296_at | Res & Lgx | 4260 | 3917 | 3455 | 3149 | −1.09 | −1.23 | −1.35 | Psmd8 |
| 1423315_at | Res only | 239 | 239 | 393 | 245 | −1.00 | 1.64 | 1.03 | Bbc3 |
| 1423332_at | Lgx only | 7044 | 6555 | 6145 | 5521 | −1.07 | −1.15 | −1.28 | Sdcbp |
| 1423347_at | Lgx only | 1717 | 1446 | 1257 | 946 | −1.19 | −1.37 | −1.81 | Sec23a |
| 1423362_at | Lgx only | 1116 | 1401 | 1546 | 2127 | 1.26 | 1.38 | 1.91 | Sort1 |
| 1423365_at | Lgx only | 220 | 341 | 321 | 487 | 1.55 | 1.46 | 2.22 | Cacna1g |
| 1423368_at | Res only | 26862 | 24889 | 23668 | 24004 | −1.08 | −1.13 | −1.12 | Laptm4a |
| 1423369_at | Lgx only | 512 | 450 | 400 | 277 | −1.14 | −1.28 | −1.84 | Fmr1 |
| 1423373_at | Lgx only | 1662 | 1543 | 1231 | 1041 | −1.08 | −1.35 | −1.60 | Rpp30 |
| 1423383_a_at | Lgx only | 3010 | 3451 | 3134 | 3991 | 1.15 | 1.04 | 1.33 | Osbpl9 |
| 1423393_at | Res & Lgx | 9550 | 11356 | 12552 | 12982 | 1.19 | 1.31 | 1.36 | Clic4 |
| 1423407_a_at | Res & Lgx | 1285 | 1527 | 1976 | 2493 | 1.19 | 1.54 | 1.94 | Fbln2 |
| 1423423_at | Res & Lgx | 3996 | 3941 | 3047 | 3212 | −1.01 | −1.31 | −1.24 | Pdia3 |
| 1423425_at | Lgx only | 639 | 706 | 761 | 947 | 1.10 | 1.19 | 1.48 | 1300012G16Rik |
| 1423431_a_at | Res & Lgx | 1593 | 1952 | 2320 | 2336 | 1.23 | 1.46 | 1.47 | Mybbp1a |
| 1423440_at | Lgx only | 631 | 546 | 529 | 467 | −1.15 | −1.19 | −1.35 | 1110001A07Rik |
| 1423441_at | Lgx only | 4060 | 3903 | 3850 | 3452 | −1.04 | −1.05 | −1.18 | Tfb2m |
| 1423448_at | Res & Lgx | 4469 | 4669 | 6840 | 6850 | 1.04 | 1.53 | 1.53 | Rab11b |
| 1423449_a_at | Lgx only | 1823 | 2337 | 2121 | 2533 | 1.28 | 1.16 | 1.39 | Actn4 |
| 1423459_at | Lgx only | 3896 | 3424 | 3126 | 2505 | −1.14 | −1.25 | −1.56 | Cops2 |
| 1423474_at | Res & Lgx | 2213 | 2036 | 1686 | 1390 | −1.09 | −1.31 | −1.59 | Top1 |
| 1423485_at | Lgx only | 523 | 580 | 631 | 930 | 1.11 | 1.21 | 1.78 | Rad54l2 |
| 1423486_at | Res & Lgx | 4116 | 3309 | 2762 | 2491 | −1.24 | −1.49 | −1.65 | Cript |
| 1423490_at | Lgx only | 3791 | 3965 | 3044 | 2767 | 1.05 | −1.25 | −1.37 | Fbxo3 |
| 1423494_at | CR only | 38 | 105 | 93 | 45 | 2.79 | 2.46 | 1.20 | 2310042E22Rik |
| 1423512_at | Lgx only | 664 | 554 | 571 | 494 | −1.20 | −1.16 | −1.35 | AW209491 |
| 1423529_at | Lgx only | 76 | 62 | 55 | 20 | −1.22 | −1.38 | −3.78 | G6pc2 |
| 1423535_at | Lgx only | 4603 | 3572 | 3482 | 2393 | −1.29 | −1.32 | −1.92 | LOC100047794 |
| 1423557_at | Res & Lgx | 4123 | 4659 | 2949 | 2995 | 1.13 | −1.40 | −1.38 | Ifngr2 |
| 1423565_at | Lgx only | 6741 | 7261 | 5935 | 5619 | 1.08 | −1.14 | −1.20 | Paics |
| 1423577_at | CR only | 866 | 691 | 713 | 759 | −1.25 | −1.22 | −1.14 | Ankrd32 |
| 1423588_at | Res & Lgx | 1204 | 1118 | 984 | 929 | −1.08 | −1.22 | −1.30 | Arpc4 |
| 1423599_a_at | Lgx only | 741 | 579 | 555 | 467 | −1.28 | −1.33 | −1.59 | Pdcl |
| 1423609_a_at | Lgx only | 2102 | 2341 | 2391 | 3062 | 1.11 | 1.14 | 1.46 | Mgat1 |
| 1423611_at | CR only | 736 | 949 | 973 | 1010 | 1.29 | 1.32 | 1.37 | Akp2 |
| 1423620_at | Lgx only | 428 | 413 | 295 | 207 | −1.04 | −1.45 | −2.07 | Cenpq |
| 1423629_at | Lgx only | 842 | 1015 | 1126 | 1457 | 1.21 | 1.34 | 1.73 | Dnm2 |
| 1423642_at | CR & Res | 11579 | 15048 | 8041 | 9198 | 1.30 | −1.44 | −1.26 | Tubb2c |
| 1423643_at | Lgx only | 1071 | 1196 | 843 | 821 | 1.12 | −1.27 | −1.30 | Ddx39 |
| 1423647_a_at | Res only | 344 | 396 | 605 | 457 | 1.15 | 1.76 | 1.33 | Zdhhc3 |
| 1423648_at | CR only | 1771 | 2210 | 1681 | 1872 | 1.25 | −1.05 | 1.06 | Pdia6 |
| 1423657_at | Res & Lgx | 1515 | 1485 | 1236 | 1166 | −1.02 | −1.23 | −1.30 | Cdipt |
| 1423662_at | Res & Lgx | 1443 | 1423 | 991 | 902 | −1.01 | −1.46 | −1.60 | Atp6ap2 |
| 1423663_at | CR only | 1057 | 1384 | 1080 | 1178 | 1.31 | 1.02 | 1.12 | Flcn |
| 1423667_at | All | 8373 | 7112 | 6577 | 5613 | −1.18 | −1.27 | −1.49 | Mat2a |
| 1423669_at | Lgx only | 1075 | 1350 | 1555 | 2209 | 1.26 | 1.45 | 2.05 | Col1a1 |
| 1423670_a_at | CR & Lgx | 3169 | 3700 | 3314 | 3615 | 1.17 | 1.05 | 1.14 | Srpr |
| 1423676_at | Lgx only | 58661 | 62536 | 61203 | 80373 | 1.07 | 1.04 | 1.37 | Atp5h |
| 1423685_at | Lgx only | 1108 | 1452 | 1375 | 1574 | 1.31 | 1.24 | 1.42 | Aars |
| 1423694_at | CR only | 1790 | 2077 | 2089 | 1818 | 1.16 | 1.17 | 1.02 | Kctd10 |
| 1423697_at | Lgx only | 6981 | 6288 | 6277 | 5248 | −1.11 | −1.11 | −1.33 | Psmd6 |
| 1423710_at | Lgx only | 9222 | 11179 | 10001 | 11429 | 1.21 | 1.08 | 1.24 | Dlst |
| 1423711_at | Res & Lgx | 2316 | 2307 | 1924 | 1627 | −1.00 | −1.20 | −1.42 | Ndufaf1 |
| 1423725_at | Lgx only | 985 | 705 | 674 | 762 | −1.40 | −1.46 | −1.29 | Pls3 |
| 1423734_at | Lgx only | 5006 | 5612 | 5707 | 6496 | 1.12 | 1.14 | 1.30 | Rac1 |
| 1423737_at | Lgx only | 24235 | 26450 | 27218 | 29730 | 1.09 | 1.12 | 1.23 | Ndufs3 |
| 1423753_at | CR only | 1118 | 734 | 898 | 856 | −1.52 | −1.24 | −1.31 | Bambi |
| 1423759_a_at | Res & Lgx | 4031 | 3513 | 2876 | 2736 | −1.15 | −1.40 | −1.47 | Tmco1 |
| 1423766_at | Res & Lgx | 1610 | 1380 | 1025 | 987 | −1.17 | −1.57 | −1.63 | Pak1ip1 |
| 1423767_at | Lgx only | 2146 | 2025 | 1774 | 1497 | −1.06 | −1.21 | −1.43 | 2810410M20Rik |
| 1423771_at | Lgx only | 815 | 750 | 811 | 1094 | −1.09 | −1.00 | 1.34 | Prkcdbp |
| 1423773_at | Lgx only | 4627 | 3985 | 3629 | 2816 | −1.16 | −1.28 | −1.64 | Gpbp1 |
| 1423780_at | Lgx only | 10898 | 11906 | 12709 | 12518 | 1.09 | 1.17 | 1.15 | Hibadh |
| 1423785_at | Lgx only | 11606 | 13426 | 11821 | 15052 | 1.16 | 1.02 | 1.30 | Egln1 |
| 1423790_at | Lgx only | 1487 | 1751 | 2032 | 2310 | 1.18 | 1.37 | 1.55 | Dap |
| 1423793_at | Res & Lgx | 936 | 1073 | 1319 | 1580 | 1.15 | 1.41 | 1.69 | D2Ertd391e |
| 1423810_at | CR only | 1783 | 2180 | 1746 | 1816 | 1.22 | −1.02 | 1.02 | Ppme1 |
| 1423822_a_at | Lgx only | 730 | 653 | 569 | 558 | −1.12 | −1.28 | −1.31 | Tmem168 |
| 1423845_at | Lgx only | 460 | 477 | 805 | 739 | 1.04 | 1.75 | 1.61 | Csdc2 |
| 1423847_at | Res & Lgx | 231 | 270 | 401 | 391 | 1.17 | 1.73 | 1.69 | Ncapd2 |
| 1423849_a_at | Lgx only | 616 | 713 | 916 | 1030 | 1.16 | 1.49 | 1.67 | Clk3 |
| 1423852_at | Lgx only | 50 | 52 | 68 | 154 | 1.03 | 1.36 | 3.07 | Tmem46 |
| 1423857_at | Res & Lgx | 13121 | 11472 | 8712 | 6533 | −1.14 | −1.51 | −2.01 | Mrpl30 |
| 1423881_at | Lgx only | 1929 | 2007 | 2122 | 2477 | 1.04 | 1.10 | 1.28 | Saps3 |
| 1423882_at | Lgx only | 933 | 840 | 739 | 618 | −1.11 | −1.26 | −1.51 | Rfwd3 |
| 1423883_at | Lgx only | 21573 | 22608 | 26023 | 29695 | 1.05 | 1.21 | 1.38 | Acsl1 |
| 1423892_at | CR only | 3733 | 2926 | 4320 | 4066 | −1.28 | 1.16 | 1.09 | Apbb1 |
| 1423896_a_at | Lgx only | 7953 | 9503 | 10177 | 13002 | 1.19 | 1.28 | 1.63 | Rnf187 |
| 1423907_a_at | Lgx only | 21309 | 20866 | 21974 | 17327 | −1.02 | 1.03 | −1.23 | Ndufs8 |
| 1423909_at | Lgx only | 670 | 657 | 729 | 1002 | −1.02 | 1.09 | 1.50 | Tmem176a |
| 1423919_at | Lgx only | 445 | 348 | 353 | 351 | −1.28 | −1.26 | −1.27 | BC023882 |
| 1423927_at | Res & Lgx | 510 | 626 | 737 | 770 | 1.23 | 1.45 | 1.51 | Slc35b2 |
| 1423939_a_at | Lgx only | 6662 | 5968 | 5430 | 4027 | −1.12 | −1.23 | −1.65 | Yif1a |
| 1423947_at | Res & Lgx | 1479 | 1457 | 2188 | 2164 | −1.01 | 1.48 | 1.46 | 1110008P14Rik |
| 1423951_at | Lgx only | 936 | 768 | 718 | 654 | −1.22 | −1.30 | −1.43 | Tm2d3 |
| 1423958_a_at | Lgx only | 1601 | 1398 | 1213 | 998 | −1.15 | −1.32 | −1.60 | Ttc33 |
| 1423960_at | Res & Lgx | 1368 | 1371 | 1946 | 2010 | 1.00 | 1.42 | 1.47 | Mboat5 |
| 1423961_at | Res & Lgx | 1414 | 1142 | 839 | 602 | −1.24 | −1.69 | −2.35 | LOC100045629 |
| 1423967_at | Lgx only | 903 | 1044 | 1073 | 1359 | 1.16 | 1.19 | 1.51 | Palm |
| 1423969_at | All | 723 | 538 | 512 | 442 | −1.34 | −1.41 | −1.64 | Nup37 |
| 1423972_at | Lgx only | 31981 | 34252 | 35126 | 43175 | 1.07 | 1.10 | 1.35 | Etfa |
| 1423973_a_at | Lgx only | 644 | 716 | 819 | 1000 | 1.11 | 1.27 | 1.55 | Arf3 |
| 1423978_at | Lgx only | 920 | 1504 | 1209 | 1467 | 1.64 | 1.31 | 1.59 | Sbk1 |
| 1423991_at | Lgx only | 392 | 425 | 448 | 564 | 1.08 | 1.14 | 1.44 | Nol14 |
| 1423993_at | Lgx only | 4392 | 3844 | 3630 | 3056 | −1.14 | −1.21 | −1.44 | Atp6v1f |
| 1424000_a_at | Lgx only | 20066 | 22495 | 23422 | 27778 | 1.12 | 1.17 | 1.38 | Rps11 |
| 1424002_at | Res & Lgx | 2495 | 2175 | 1827 | 1785 | −1.15 | −1.37 | −1.40 | Pdcl3 |
| 1424005_at | Lgx only | 3828 | 3557 | 3275 | 3040 | −1.08 | −1.17 | −1.26 | B230219D22Rik |
| 1424010_at | Lgx only | 168 | 233 | 382 | 732 | 1.38 | 2.27 | 4.36 | Mfap4 |
| 1424025_at | Res & Lgx | 5858 | 5688 | 4312 | 4383 | −1.03 | −1.36 | −1.34 | BC013529 |
| 1424027_at | Res only | 619 | 902 | 924 | 853 | 1.46 | 1.49 | 1.38 | Pxn |
| 1424028_at | Lgx only | 874 | 861 | 614 | 481 | −1.02 | −1.42 | −1.82 | 5830457O10Rik |
| 1424033_at | Res & Lgx | 935 | 735 | 479 | 332 | −1.27 | −1.95 | −2.82 | Sfrs7 |
| 1424036_at | Lgx only | 1422 | 1354 | 1874 | 1752 | −1.05 | 1.32 | 1.23 | Prpf6 |
| 1424038_a_at | Lgx only | 2801 | 3278 | 3181 | 3602 | 1.17 | 1.14 | 1.29 | 2310044H10Rik |
| 1424040_at | Lgx only | 5989 | 7521 | 7112 | 8135 | 1.26 | 1.19 | 1.36 | Mtap7d1 |
| 1424053_a_at | Res & Lgx | 5291 | 5313 | 6583 | 6840 | 1.00 | 1.24 | 1.29 | Tcf25 |
| 1424054_at | Res & Lgx | 1063 | 1467 | 1588 | 1882 | 1.38 | 1.49 | 1.77 | Btbd2 |
| 1424058_at | Lgx only | 640 | 771 | 767 | 944 | 1.21 | 1.20 | 1.48 | Prrc1 |
| 1424066_at | Lgx only | 990 | 1038 | 1084 | 1324 | 1.05 | 1.09 | 1.34 | Dus3l |
| 1424077_at | Res only | 654 | 544 | 493 | 479 | −1.20 | −1.33 | −1.37 | Gdpd1 |
| 1424081_at | Res & Lgx | 380 | 271 | 291 | 277 | −1.40 | −1.31 | −1.37 | Pcgf6 |
| 1424099_at | Lgx only | 2023 | 1663 | 1332 | 1265 | −1.22 | −1.52 | −1.60 | 2310016C16Rik |
| 1424101_at | Res & Lgx | 874 | 934 | 533 | 583 | 1.07 | −1.64 | −1.50 | Hnrpl |
| 1424105_a_at | Lgx only | 3516 | 2620 | 3158 | 2273 | −1.34 | −1.11 | −1.55 | Pttg1 |
| 1424109_a_at | Res & Lgx | 6904 | 7061 | 6174 | 6463 | 1.02 | −1.12 | −1.07 | Glo1 |
| 1424115_at | CR & Lgx | 1970 | 2313 | 2291 | 2496 | 1.17 | 1.16 | 1.27 | Ppp5c |
| 1424121_at | Lgx only | 986 | 890 | 677 | 523 | −1.11 | −1.46 | −1.88 | Commd1 |
| 1424126_at | CR & Res | 6092 | 9782 | 4437 | 5967 | 1.61 | −1.37 | −1.02 | Alas1 |
| 1424134_at | Lgx only | 646 | 529 | 499 | 436 | −1.22 | −1.29 | −1.48 | Rspry1 |
| 1424138_at | Lgx only | 1703 | 1927 | 1979 | 2218 | 1.13 | 1.16 | 1.30 | Rhbdf1 |
| 1424139_at | Lgx only | 5935 | 4960 | 4416 | 3478 | −1.20 | −1.34 | −1.71 | Rap1a |
| 1424140_at | Res only | 146 | 192 | 243 | 196 | 1.32 | 1.67 | 1.35 | Gale |
| 1424141_at | Lgx only | 2745 | 3324 | 3767 | 4195 | 1.21 | 1.37 | 1.53 | Hectd1 |
| 1424147_at | Res & Lgx | 3868 | 4234 | 2946 | 2570 | 1.09 | −1.31 | −1.50 | Ahsa1 |
| 1424149_at | CR only | 435 | 531 | 523 | 573 | 1.22 | 1.20 | 1.32 | Nsmce2 |
| 1424150_at | Lgx only | 438 | 540 | 561 | 688 | 1.23 | 1.28 | 1.57 | Gdpd5 |
| 1424151_at | Res & Lgx | 2327 | 2380 | 1795 | 1679 | 1.02 | −1.30 | −1.39 | Jtv1 |
| 1424154_a_at | Lgx only | 4155 | 3735 | 3761 | 3368 | −1.11 | −1.10 | −1.23 | Isca2 |
| 1424159_at | Res & Lgx | 618 | 578 | 459 | 454 | −1.07 | −1.34 | −1.36 | 1300010M03Rik |
| 1424160_at | Lgx only | 563 | 486 | 499 | 420 | −1.16 | −1.13 | −1.34 | Alg5 |
| 1424162_at | CR only | 115 | 37 | 144 | 125 | −3.15 | 1.25 | 1.08 | Trim29 |
| 1424163_at | Lgx only | 615 | 642 | 714 | 803 | 1.04 | 1.16 | 1.31 | Rmnd5b |
| 1424167_a_at | Lgx only | 367 | 456 | 568 | 734 | 1.24 | 1.55 | 2.00 | Pmm1 |
| 1424175_at | CR & Lgx | 4138 | 5751 | 4597 | 7356 | 1.39 | 1.11 | 1.78 | Tef |
| 1424178_at | Lgx only | 8368 | 8313 | 7872 | 6995 | −1.01 | −1.06 | −1.20 | Tmem38a |
| 1424179_at | Lgx only | 642 | 594 | 541 | 431 | −1.08 | −1.19 | −1.49 | Plekhj1 |
| 1424184_at | Lgx only | 33037 | 35163 | 37503 | 45711 | 1.06 | 1.14 | 1.38 | Acadvl |
| 1424191_a_at | Lgx only | 613 | 688 | 706 | 837 | 1.12 | 1.15 | 1.36 | Tmem41a |
| 1424209_at | Lgx only | 1349 | 1298 | 1166 | 988 | −1.04 | −1.16 | −1.37 | Rars2 |
| 1424210_at | Res & Lgx | 892 | 764 | 657 | 670 | −1.17 | −1.36 | −1.33 | Erlin1 |
| 1424211_at | CR & Lgx | 1496 | 2374 | 1415 | 1975 | 1.59 | −1.06 | 1.32 | Slc25a33 |
| 1424216_a_at | Lgx only | 1633 | 1442 | 1293 | 1181 | −1.13 | −1.26 | −1.38 | Papola |
| 1424223_at | Lgx only | 7099 | 8501 | 6040 | 6025 | 1.20 | −1.18 | −1.18 | 1700020C11Rik |
| 1424236_at | Lgx only | 741 | 789 | 838 | 950 | 1.06 | 1.13 | 1.28 | Tbc1d10b |
| 1424237_at | Res only | 1047 | 1010 | 1262 | 1184 | −1.04 | 1.21 | 1.13 | Zfp639 |
| 1424247_at | Lgx only | 543 | 591 | 600 | 802 | 1.09 | 1.10 | 1.48 | Erc1 |
| 1424249_a_at | All | 434 | 271 | 226 | 252 | −1.60 | −1.92 | −1.72 | Arhgap9 |
| 1424255_at | Res & Lgx | 2458 | 2688 | 3230 | 3339 | 1.09 | 1.31 | 1.36 | Supt5h |
| 1424258_at | Lgx only | 955 | 696 | 690 | 604 | −1.37 | −1.38 | −1.58 | Polr2d |
| 1424261_at | Res & Lgx | 1144 | 871 | 714 | 580 | −1.31 | −1.60 | −1.97 | Zfp672 |
| 1424274_at | Res & Lgx | 1795 | 1574 | 1426 | 1084 | −1.14 | −1.26 | −1.66 | Vdp |
| 1424276_at | Lgx only | 410 | 338 | 308 | 258 | −1.22 | −1.33 | −1.59 | Snx16 |
| 1424280_at | Res & Lgx | 2125 | 1767 | 1658 | 1182 | −1.20 | −1.28 | −1.80 | Mospd1 |
| 1424303_at | Lgx only | 90 | 76 | 59 | 26 | −1.19 | −1.53 | −3.47 | Depdc7 |
| 1424309_a_at | Res & Lgx | 2945 | 2961 | 2220 | 1975 | 1.01 | −1.33 | −1.49 | Mocs2 |
| 1424318_at | Lgx only | 2148 | 2040 | 1851 | 1802 | −1.05 | −1.16 | −1.19 | 1110067D22Rik |
| 1424321_at | Lgx only | 351 | 263 | 228 | 169 | −1.34 | −1.54 | −2.07 | Rfc4 |
| 1424324_at | Lgx only | 956 | 712 | 539 | 403 | −1.34 | −1.77 | −2.37 | Esco1 |
| 1424346_at | Lgx only | 2287 | 1920 | 1820 | 1658 | −1.19 | −1.26 | −1.38 | Ppp6c |
| 1424349_a_at | Res & Lgx | 2326 | 1877 | 1516 | 1263 | −1.24 | −1.53 | −1.84 | Lpgat1 |
| 1424356_a_at | Lgx only | 770 | 697 | 694 | 603 | −1.10 | −1.11 | −1.28 | Metrnl |
| 1424359_at | Lgx only | 1036 | 1229 | 2242 | 2519 | 1.19 | 2.16 | 2.43 | Oplah |
| 1424361_at | Lgx only | 294 | 408 | 530 | 653 | 1.39 | 1.80 | 2.22 | BC019943 |
| 1424372_at | Res & Lgx | 3282 | 2988 | 2215 | 2091 | −1.10 | −1.48 | −1.57 | Mrpl32 |
| 1424374_at | Lgx only | 1936 | 1845 | 1535 | 1363 | −1.05 | −1.26 | −1.42 | Gimap4 |
| 1424377_at | Lgx only | 1007 | 971 | 895 | 801 | −1.04 | −1.12 | −1.26 | BC003885 |
| 1424380_at | Lgx only | 434 | 432 | 512 | 559 | −1.01 | 1.18 | 1.29 | Vps37b |
| 1424384_a_at | Lgx only | 726 | 981 | 1007 | 1092 | 1.35 | 1.39 | 1.50 | Znrf1 |
| 1424390_at | Lgx only | 597 | 493 | 481 | 472 | −1.21 | −1.24 | −1.26 | Nupl1 |
| 1424391_at | CR & Lgx | 3544 | 3195 | 3213 | 2673 | −1.11 | −1.10 | −1.33 | Nrd1 |
| 1424403_a_at | Lgx only | 254 | 221 | 348 | 376 | −1.15 | 1.37 | 1.48 | Rufy3 |
| 1424406_at | Lgx only | 1180 | 1319 | 1489 | 1520 | 1.12 | 1.26 | 1.29 | Bcl2l13 |
| 1424408_at | Lgx only | 3655 | 3925 | 4168 | 4751 | 1.07 | 1.14 | 1.30 | Lims2 |
| 1424416_at | Lgx only | 2199 | 2535 | 2429 | 2712 | 1.15 | 1.10 | 1.23 | Nkiras2 |
| 1424424_at | Lgx only | 1819 | 1642 | 1309 | 892 | −1.11 | −1.39 | −2.04 | Slc39a1 |
| 1424430_at | Res only | 647 | 598 | 475 | 466 | −1.08 | −1.36 | −1.39 | Mterfd2 |
| 1424433_at | Res & Lgx | 3043 | 3150 | 2599 | 2241 | 1.04 | −1.17 | −1.36 | Msrb2 |
| 1424434_at | Res & Lgx | 2817 | 2667 | 2219 | 2042 | −1.06 | −1.27 | −1.38 | BC024814 |
| 1424447_at | Lgx only | 404 | 418 | 462 | 566 | 1.03 | 1.14 | 1.40 | 1700030K09Rik |
| 1424461_at | Res & Lgx | 4865 | 4886 | 3994 | 3716 | 1.00 | −1.22 | −1.31 | Dctn2 |
| 1424463_at | Res & Lgx | 1299 | 1043 | 945 | 877 | −1.24 | −1.37 | −1.48 | 2210010L05Rik |
| 1424465_at | Lgx only | 957 | 881 | 710 | 583 | −1.09 | −1.35 | −1.64 | Ccdc58 |
| 1424467_at | Lgx only | 1725 | 2308 | 2228 | 2625 | 1.34 | 1.29 | 1.52 | Phldb1 |
| 1424473_at | Lgx only | 911 | 823 | 725 | 502 | −1.11 | −1.26 | −1.81 | Polr2h |
| 1424479_at | Lgx only | 39 | 46 | 64 | 119 | 1.18 | 1.64 | 3.02 | Cst8 |
| 1424500_at | Lgx only | 1020 | 947 | 744 | 637 | −1.08 | −1.37 | −1.60 | Utp6 |
| 1424505_at | Lgx only | 1309 | 1213 | 1172 | 895 | −1.08 | −1.12 | −1.46 | Rmnd1 |
| 1424510_at | Lgx only | 783 | 733 | 627 | 574 | −1.07 | −1.25 | −1.36 | Nudt6 |
| 1424517_at | CR only | 403 | 529 | 450 | 495 | 1.31 | 1.12 | 1.23 | Ccdc12 |
| 1424520_at | Res only | 687 | 597 | 550 | 610 | −1.15 | −1.25 | −1.13 | 2010305A19Rik |
| 1424526_a_at | Lgx only | 630 | 568 | 533 | 439 | −1.11 | −1.18 | −1.43 | Tgds |
| 1424527_at | Lgx only | 1547 | 1612 | 1940 | 2049 | 1.04 | 1.25 | 1.32 | Ppp2r2d |
| 1424531_a_at | Lgx only | 1693 | 2053 | 2103 | 2408 | 1.21 | 1.24 | 1.42 | Tcea3 |
| 1424539_at | Res & Lgx | 4074 | 4359 | 3294 | 2993 | 1.07 | −1.24 | −1.36 | Ubl4 |
| 1424541_at | Res & Lgx | 5299 | 4888 | 3970 | 2986 | −1.08 | −1.33 | −1.77 | Tmem70 |
| 1424545_at | Res & Lgx | 4029 | 3665 | 2845 | 2585 | −1.10 | −1.42 | −1.56 | BC003965 |
| 1424553_at | Lgx only | 3262 | 3958 | 4239 | 4357 | 1.21 | 1.30 | 1.34 | Hhatl |
| 1424559_at | Lgx only | 301 | 268 | 241 | 157 | −1.13 | −1.25 | −1.92 | Rpap2 |
| 1424562_a_at | Lgx only | ##### | 112473 | 113888 | 167622 | 1.08 | 1.10 | 1.61 | Slc25a4 |
| 1424564_at | Lgx only | 1274 | 1360 | 1557 | 1708 | 1.07 | 1.22 | 1.34 | 2410001C21Rik |
| 1424572_a_at | Lgx only | 1146 | 1213 | 1654 | 1775 | 1.06 | 1.44 | 1.55 | H2afy |
| 1424585_at | Lgx only | 895 | 932 | 1128 | 1160 | 1.04 | 1.26 | 1.30 | Ranbp10 |
| 1424594_at | Lgx only | 3378 | 3499 | 3621 | 4257 | 1.04 | 1.07 | 1.26 | Lgals7 |
| 1424595_at | Lgx only | 1220 | 1320 | 1490 | 2039 | 1.08 | 1.22 | 1.67 | F11r |
| 1424598_at | Lgx only | 2389 | 1604 | 1838 | 1438 | −1.49 | −1.30 | −1.66 | Ddx6 |
| 1424614_at | Lgx only | 2262 | 2581 | 3033 | 2935 | 1.14 | 1.34 | 1.30 | Frag1 |
| 1424635_at | Lgx only | 40976 | 41854 | 46791 | 54473 | 1.02 | 1.14 | 1.33 | Eef1a1 |
| 1424639_a_at | Lgx only | 1837 | 2131 | 2120 | 2408 | 1.16 | 1.15 | 1.31 | Hmgcl |
| 1424642_at | Res & Lgx | 839 | 793 | 585 | 514 | −1.06 | −1.43 | −1.63 | Thoc1 |
| 1424643_at | Lgx only | 361 | 461 | 466 | 524 | 1.28 | 1.29 | 1.45 | Tcof1 |
| 1424644_at | Lgx only | 751 | 910 | 920 | 970 | 1.21 | 1.22 | 1.29 | Tbcc |
| 1424669_at | Res & Lgx | 1167 | 1216 | 852 | 883 | 1.04 | −1.37 | −1.32 | Zfyve21 |
| 1424682_at | Lgx only | 844 | 922 | 1107 | 1316 | 1.09 | 1.31 | 1.56 | Atpbd1c |
| 1424683_at | Lgx only | 15130 | 13625 | 13449 | 10806 | −1.11 | −1.12 | −1.40 | 1810015C04Rik |
| 1424686_at | Lgx only | 397 | 485 | 579 | 584 | 1.22 | 1.46 | 1.47 | Heatr6 |
| 1424700_at | Lgx only | 1348 | 1564 | 1695 | 1953 | 1.16 | 1.26 | 1.45 | Tmem38b |
| 1424715_at | All | 1674 | 1255 | 2440 | 2317 | −1.33 | 1.46 | 1.38 | Retsat |
| 1424720_at | Lgx only | 1275 | 1563 | 1533 | 1790 | 1.23 | 1.20 | 1.40 | Mgat4b |
| 1424727_at | Lgx only | 73 | 105 | 145 | 185 | 1.43 | 1.99 | 2.54 | Ccr5 |
| 1424728_at | CR & Lgx | 991 | 816 | 861 | 760 | −1.21 | −1.15 | −1.30 | BC011248 |
| 1424736_at | Res & Lgx | 18836 | 21343 | 24570 | 28063 | 1.13 | 1.30 | 1.49 | Eef2 |
| 1424744_at | Lgx only | 318 | 193 | 275 | 639 | −1.65 | −1.16 | 2.01 | Sds |
| 1424745_at | Lgx only | 533 | 595 | 714 | 779 | 1.11 | 1.34 | 1.46 | Agxt2l2 |
| 1424746_at | Res & Lgx | 5066 | 5452 | 6046 | 7004 | 1.08 | 1.19 | 1.38 | Kif1c |
| 1424749_at | Res & Lgx | 491 | 453 | 785 | 669 | −1.08 | 1.60 | 1.36 | Wdfy1 |
| 1424776_a_at | Lgx only | 1323 | 1302 | 1746 | 1740 | −1.02 | 1.32 | 1.32 | Slc25a28 |
| 1424777_at | Lgx only | 572 | 536 | 703 | 792 | −1.07 | 1.23 | 1.38 | Wdr21 |
| 1424790_at | Lgx only | 1894 | 2211 | 3370 | 4182 | 1.17 | 1.78 | 2.21 | Slc25a42 |
| 1424791_a_at | Lgx only | 2967 | 3682 | 3547 | 3765 | 1.24 | 1.20 | 1.27 | Bcam |
| 1424795_a_at | Res only | 62 | 70 | 170 | 154 | 1.13 | 2.74 | 2.48 | 1700001O22Rik |
| 1424819_a_at | Lgx only | 557 | 580 | 605 | 719 | 1.04 | 1.09 | 1.29 | Ric8 |
| 1424827_a_at | Lgx only | 5109 | 5113 | 4471 | 3838 | 1.00 | −1.14 | −1.33 | Csnk1a1 |
| 1424842_a_at | Lgx only | 554 | 516 | 684 | 759 | −1.07 | 1.24 | 1.37 | Arhgap24 |
| 1424850_at | Lgx only | 392 | 341 | 315 | 258 | −1.15 | −1.24 | −1.52 | Map3k1 |
| 1424873_at | Res & Lgx | 906 | 801 | 637 | 641 | −1.13 | −1.42 | −1.41 | Rnf2 |
| 1424878_at | CR & Lgx | 255 | 508 | 463 | 632 | 1.99 | 1.82 | 2.48 | Lrch4 |
| 1424898_at | CR only | 144 | 51 | 82 | 114 | −2.84 | −1.76 | −1.27 | Slc10a1 |
| 1424912_at | Lgx only | 417 | 508 | 500 | 569 | 1.22 | 1.20 | 1.36 | Slc25a17 |
| 1424918_at | Res & Lgx | 893 | 857 | 631 | 559 | −1.04 | −1.42 | −1.60 | Tbc1d19 |
| 1424929_a_at | Lgx only | 239 | 284 | 385 | 449 | 1.19 | 1.61 | 1.88 | Trim26 |
| 1424942_a_at | CR only | 55 | 147 | 154 | 55 | 2.69 | 2.81 | 1.00 | Myc |
| 1424954_a_at | Lgx only | 657 | 753 | 904 | 1030 | 1.15 | 1.38 | 1.57 | Pip5k1c |
| 1424956_at | Lgx only | 246 | 311 | 380 | 513 | 1.26 | 1.55 | 2.09 | Ahdc1 |
| 1424978_at | Res only | 23 | 79 | 87 | 88 | 3.44 | 3.76 | 3.82 | Odf4 |
| 1424988_at | Lgx only | 1652 | 1684 | 1806 | 2065 | 1.02 | 1.09 | 1.25 | Mylip |
| 1424990_at | Lgx only | 890 | 960 | 1037 | 1205 | 1.08 | 1.17 | 1.35 | Tmem142a |
| 1424996_at | Lgx only | 650 | 688 | 746 | 875 | 1.06 | 1.15 | 1.35 | Cflar |
| 1425024_at | CR only | 104 | 28 | 94 | 50 | −3.79 | −1.11 | −2.09 | E430018J23Rik |
| 1425057_at | Res only | 88 | 164 | 237 | 176 | 1.85 | 2.68 | 1.98 | Pbld |
| 1425079_at | CR only | 40 | 118 | 72 | 60 | 2.96 | 1.79 | 1.51 | Tm6sf2 |
| 1425114_at | Res & Lgx | 795 | 765 | 561 | 544 | −1.04 | −1.42 | −1.46 | Rbbp6 |
| 1425143_a_at | Lgx only | 25337 | 27260 | 28070 | 34606 | 1.08 | 1.11 | 1.37 | Ndufs1 |
| 1425158_at | Res & Lgx | 1320 | 1302 | 1751 | 1907 | −1.01 | 1.33 | 1.44 | Tbx20 |
| 1425164_a_at | Lgx only | 265 | 328 | 383 | 483 | 1.24 | 1.44 | 1.82 | Phkg1 |
| 1425189_a_at | Res & Lgx | 3583 | 3540 | 2889 | 2383 | −1.01 | −1.24 | −1.50 | Mrpl15 |
| 1425214_at | Lgx only | 324 | 237 | 207 | 133 | −1.37 | −1.57 | −2.44 | P2ry6 |
| 1425225_at | Lgx only | 94 | 142 | 190 | 249 | 1.51 | 2.02 | 2.64 | Fcgr4 |
| 1425228_a_at | Lgx only | 1536 | 1652 | 1255 | 1078 | 1.08 | −1.22 | −1.43 | Dguok |
| 1425243_at | Lgx only | 115 | 120 | 196 | 325 | 1.04 | 1.70 | 2.82 | Cd207 |
| 1425257_at | Lgx only | 7 | 19 | 41 | 16 | 2.89 | 6.34 | 2.39 | Acot5 |
| 1425270_at | Res & Lgx | 3017 | 2790 | 2111 | 1975 | −1.08 | −1.43 | −1.53 | Kif1b |
| 1425274_at | Res & Lgx | 4607 | 4976 | 3462 | 3212 | 1.08 | −1.33 | −1.43 | Asph |
| 1425314_at | Res only | 27 | 57 | 96 | 118 | 2.10 | 3.57 | 4.36 | Gpr98 |
| 1425332_at | Lgx only | 11194 | 12729 | 12529 | 16323 | 1.14 | 1.12 | 1.46 | Zfp106 |
| 1425333_at | Lgx only | 410 | 538 | 624 | 856 | 1.31 | 1.52 | 2.09 | Rab43 |
| 1425340_a_at | CR & Lgx | 1281 | 1481 | 1447 | 1573 | 1.16 | 1.13 | 1.23 | Ptpra |
| 1425341_at | Lgx only | 2691 | 3341 | 3786 | 4027 | 1.24 | 1.41 | 1.50 | Kcnk3 |
| 1425350_a_at | Res & Lgx | 106 | 147 | 186 | 212 | 1.38 | 1.75 | 2.00 | Myef2 |
| 1425455_a_at | Lgx only | 4248 | 4028 | 3479 | 3392 | −1.05 | −1.22 | −1.25 | Churc1 |
| 1425480_at | Res & Lgx | 2113 | 2091 | 1616 | 1298 | −1.01 | −1.31 | −1.63 | Cnot6l |
| 1425492_at | Res & Lgx | 2530 | 2180 | 1751 | 1792 | −1.16 | −1.44 | −1.41 | Bmpr1a |
| 1425519_a_at | Res & Lgx | 1769 | 1610 | 2963 | 2821 | −1.10 | 1.67 | 1.59 | Cd74 |
| 1425521_at | Lgx only | 337 | 279 | 205 | 188 | −1.21 | −1.64 | −1.79 | Paip1 |
| 1425558_at | Res only | 136 | 59 | 28 | 101 | −2.32 | −4.94 | −1.34 | Klc3 |
| 1425589_at | Lgx only | 148 | 111 | 123 | 38 | −1.34 | −1.21 | −3.88 | Hsd17b13 |
| 1425617_at | Lgx only | 243 | 296 | 355 | 486 | 1.22 | 1.46 | 1.99 | Dhx9 |
| 1425639_at | Lgx only | 209 | 211 | 342 | 342 | 1.01 | 1.63 | 1.64 | Centa2 |
| 1425646_at | Lgx only | 141 | 96 | 96 | 68 | −1.46 | −1.47 | −2.08 | BC016495 |
| 1425674_a_at | Lgx only | 2104 | 1965 | 1606 | 1495 | −1.07 | −1.31 | −1.41 | Ssu72 |
| 1425677_a_at | Lgx only | 11130 | 10594 | 9470 | 8378 | −1.05 | −1.18 | −1.33 | Ank1 |
| 1425682_a_at | Lgx only | 518 | 419 | 373 | 288 | −1.24 | −1.39 | −1.80 | Tprkb |
| 1425702_a_at | Lgx only | 574 | 571 | 734 | 946 | −1.01 | 1.28 | 1.65 | Enpp5 |
| 1425706_a_at | Lgx only | 371 | 337 | 391 | 239 | −1.10 | 1.05 | −1.55 | Ddb2 |
| 1425718_a_at | Lgx only | 33649 | 29835 | 25700 | 23101 | −1.13 | −1.31 | −1.46 | Ivns1abp |
| 1425742_a_at | Lgx only | 11487 | 11684 | 12973 | 13668 | 1.02 | 1.13 | 1.19 | Tsc22d1 |
| 1425753_a_at | Res only | 701 | 793 | 434 | 484 | 1.13 | −1.61 | −1.45 | Ung |
| 1425760_a_at | Lgx only | 378 | 450 | 540 | 627 | 1.19 | 1.43 | 1.66 | Pitpnm1 |
| 1425764_a_at | Lgx only | 2958 | 3344 | 3747 | 4049 | 1.13 | 1.27 | 1.37 | Bcat2 |
| 1425780_a_at | Lgx only | 1362 | 1035 | 1202 | 963 | −1.32 | −1.13 | −1.41 | Tmem167 |
| 1425792_a_at | Res & Lgx | 599 | 793 | 1003 | 995 | 1.32 | 1.67 | 1.66 | Rorc |
| 1425795_a_at | Res & Lgx | 2655 | 2581 | 1359 | 1207 | −1.03 | −1.95 | −2.20 | Map3k7 |
| 1425826_a_at | Lgx only | 6893 | 8508 | 10213 | 13158 | 1.23 | 1.48 | 1.91 | Sorbs1 |
| 1425894_at | CR only | 315 | 164 | 315 | 398 | −1.92 | 1.00 | 1.26 | Mrgprf |
| 1425895_a_at | Lgx only | 2855 | 3457 | 3880 | 4028 | 1.21 | 1.36 | 1.41 | Id1 |
| 1425904_at | Res only | 34 | 52 | 104 | 62 | 1.53 | 3.06 | 1.82 | Satb2 |
| 1425930_a_at | Lgx only | 698 | 638 | 680 | 563 | −1.09 | −1.03 | −1.24 | Mlx |
| 1425933_a_at | Res & Lgx | 1038 | 1091 | 859 | 854 | 1.05 | −1.21 | −1.21 | Nt5c2 |
| 1425940_a_at | Lgx only | 184 | 208 | 306 | 319 | 1.13 | 1.66 | 1.73 | Ssbp3 |
| 1425978_at | Res & Lgx | 1850 | 1652 | 1140 | 1026 | −1.12 | −1.62 | −1.80 | Myocd |
| 1425993_a_at | Res & Lgx | 1379 | 1079 | 897 | 848 | −1.28 | −1.54 | −1.63 | Hsp110 |
| 1426000_at | Lgx only | 164 | 196 | 167 | 317 | 1.19 | 1.02 | 1.93 | Oxtr |
| 1426016_a_at | Lgx only | 55 | 65 | 130 | 164 | 1.18 | 2.39 | 3.00 | Tro |
| 1426068_at | Lgx only | 935 | 868 | 767 | 692 | −1.08 | −1.22 | −1.35 | Slc7a4 |
| 1426089_a_at | Lgx only | 1823 | 1590 | 1565 | 1314 | −1.15 | −1.17 | −1.39 | BC003331 |
| 1426100_a_at | Lgx only | 218 | 225 | 310 | 338 | 1.03 | 1.42 | 1.55 | Tk2 |
| 1426114_at | Res & Lgx | 5491 | 5041 | 29756 | 25956 | −1.09 | 5.42 | 4.73 | Hnrpab |
| 1426118_a_at | Lgx only | 1454 | 1702 | 1714 | 1820 | 1.17 | 1.18 | 1.25 | Tomm40 |
| 1426179_a_at | Lgx only | 910 | 1026 | 1204 | 1351 | 1.13 | 1.32 | 1.48 | Twsg1 |
| 1426187_a_at | Lgx only | 1603 | 1568 | 1266 | 1056 | −1.02 | −1.27 | −1.52 | Hax1 |
| 1426195_a_at | Lgx only | 29737 | 32186 | 35405 | 39796 | 1.08 | 1.19 | 1.34 | Cst3 |
| 1426235_a_at | Res & Lgx | 2374 | 2866 | 5732 | 6147 | 1.21 | 2.41 | 2.59 | Glul |
| 1426241_a_at | Lgx only | 771 | 982 | 908 | 1062 | 1.27 | 1.18 | 1.38 | Scmh1 |
| 1426249_at | Res & Lgx | 1302 | 1459 | 1804 | 2108 | 1.12 | 1.39 | 1.62 | Adrbk1 |
| 1426254_at | Lgx only | 1276 | 1145 | 917 | 731 | −1.12 | −1.39 | −1.75 | Tm2d1 |
| 1426257_a_at | Res & Lgx | 1948 | 2192 | 2318 | 2595 | 1.13 | 1.19 | 1.33 | Sars |
| 1426263_at | Lgx only | 1203 | 1175 | 1108 | 911 | −1.02 | −1.09 | −1.32 | Cadm4 |
| 1426269_at | Res & Lgx | 684 | 560 | 450 | 371 | −1.22 | −1.52 | −1.85 | Sybl1 |
| 1426277_at | Lgx only | 386 | 336 | 445 | 519 | −1.15 | 1.15 | 1.34 | C730025P13Rik |
| 1426279_at | Lgx only | 770 | 726 | 591 | 516 | −1.06 | −1.30 | −1.49 | 5830415L20Rik |
| 1426285_at | Lgx only | 2453 | 2580 | 2755 | 3223 | 1.05 | 1.12 | 1.31 | Lama2 |
| 1426286_at | Lgx only | 335 | 275 | 211 | 119 | −1.22 | −1.59 | −2.81 | Noc3l |
| 1426297_at | Lgx only | 466 | 452 | 754 | 680 | −1.03 | 1.62 | 1.46 | Tcfe2a |
| 1426307_at | Res & Lgx | 1936 | 1817 | 1474 | 1311 | −1.07 | −1.31 | −1.48 | Cyb5r4 |
| 1426337_a_at | CR & Lgx | 32 | 84 | 49 | 110 | 2.64 | 1.55 | 3.45 | Tead4 |
| 1426344_at | Lgx only | 475 | 506 | 511 | 653 | 1.07 | 1.08 | 1.38 | Gle1l |
| 1426347_at | CR only | 783 | 635 | 743 | 686 | −1.23 | −1.05 | −1.14 | 2010321M09Rik |
| 1426353_at | Lgx only | 1157 | 1203 | 1210 | 1377 | 1.04 | 1.05 | 1.19 | Stat6 |
| 1426380_at | Lgx only | 4215 | 4149 | 4647 | 5122 | −1.02 | 1.10 | 1.22 | Eif4b |
| 1426386_at | Res & Lgx | 1464 | 1483 | 1190 | 993 | 1.01 | −1.23 | −1.47 | Rpl7l1 |
| 1426390_a_at | Lgx only | 22906 | 23329 | 19114 | 18319 | 1.02 | −1.20 | −1.25 | Arf1 |
| 1426398_at | Lgx only | 989 | 893 | 750 | 661 | −1.11 | −1.32 | −1.50 | Ube2w |
| 1426400_a_at | Res & Lgx | 9253 | 10079 | 10609 | 11708 | 1.09 | 1.15 | 1.27 | Capns1 |
| 1426406_at | Res & Lgx | 2553 | 2947 | 3706 | 4584 | 1.15 | 1.45 | 1.80 | Setd8 |
| 1426416_a_at | Res & Lgx | 1595 | 1370 | 1005 | 953 | −1.16 | −1.59 | −1.67 | Yipf4 |
| 1426423_at | Res & Lgx | 820 | 1004 | 1130 | 1251 | 1.22 | 1.38 | 1.53 | Shmt2 |
| 1426436_at | Lgx only | 760 | 764 | 675 | 568 | 1.00 | −1.13 | −1.34 | Tmem159 |
| 1426440_at | Lgx only | 2254 | 2820 | 2177 | 2947 | 1.25 | −1.04 | 1.31 | Dhrs7 |
| 1426444_at | Res only | 314 | 254 | 467 | 460 | −1.24 | 1.48 | 1.46 | Rhbdd2 |
| 1426445_at | Lgx only | 1978 | 2219 | 2502 | 2536 | 1.12 | 1.26 | 1.28 | Ctage5 |
| 1426446_at | Lgx only | 312 | 312 | 438 | 458 | 1.00 | 1.40 | 1.47 | 6430548M08Rik |
| 1426452_a_at | Lgx only | 202 | 256 | 129 | 77 | 1.26 | −1.57 | −2.64 | Rab30 |
| 1426455_at | Lgx only | 402 | 394 | 369 | 274 | −1.02 | −1.09 | −1.47 | Sdccag10 |
| 1426457_at | Lgx only | 9329 | 9212 | 7693 | 6876 | −1.01 | −1.21 | −1.36 | Slmap |
| 1426468_at | Res only | 1223 | 1032 | 910 | 976 | −1.18 | −1.34 | −1.25 | 0610037L13Rik |
| 1426477_at | Lgx only | 1012 | 893 | 905 | 676 | −1.13 | −1.12 | −1.50 | Rasa1 |
| 1426480_at | Lgx only | 2278 | 2147 | 2786 | 3030 | −1.06 | 1.22 | 1.33 | Sbds |
| 1426481_at | Res & Lgx | 1112 | 1028 | 1505 | 1624 | −1.08 | 1.35 | 1.46 | Klhl22 |
| 1426482_at | Lgx only | 3505 | 2977 | 2762 | 2410 | −1.18 | −1.27 | −1.45 | Prkrir |
| 1426495_at | Lgx only | 348 | 251 | 218 | 208 | −1.39 | −1.59 | −1.68 | 2410042D21Rik |
| 1426539_at | Lgx only | 295 | 365 | 390 | 465 | 1.24 | 1.32 | 1.58 | Usp11 |
| 1426567_a_at | Res only | 434 | 445 | 254 | 264 | 1.02 | −1.71 | −1.64 | Pqlc1 |
| 1426586_at | Lgx only | 17315 | 21329 | 19691 | 21472 | 1.23 | 1.14 | 1.24 | Slc25a11 |
| 1426596_a_at | All | 539 | 412 | 417 | 219 | −1.31 | −1.29 | −2.46 | Smn1 |
| 1426607_at | Lgx only | 304 | 267 | 198 | 175 | −1.14 | −1.54 | −1.73 | EG633640 |
| 1426613_a_at | Lgx only | 1480 | 1201 | 1246 | 838 | −1.23 | −1.19 | −1.77 | Snrpb2 |
| 1426614_at | Lgx only | 741 | 922 | 870 | 1015 | 1.25 | 1.18 | 1.37 | Prkcbp1 |
| 1426624_a_at | Res & Lgx | 2908 | 3005 | 4008 | 4108 | 1.03 | 1.38 | 1.41 | Ypel3 |
| 1426629_at | Lgx only | 450 | 528 | 534 | 613 | 1.17 | 1.19 | 1.36 | Dhx8 |
| 1426643_at | Lgx only | 959 | 1059 | 1176 | 1265 | 1.10 | 1.23 | 1.32 | Elp3 |
| 1426646_at | Lgx only | 3289 | 3191 | 2689 | 2507 | −1.03 | −1.22 | −1.31 | 9130011J15Rik |
| 1426648_at | Lgx only | 3467 | 4510 | 4856 | 5396 | 1.30 | 1.40 | 1.56 | Mapkapk2 |
| 1426670_at | Res & Lgx | 701 | 789 | 1130 | 1113 | 1.13 | 1.61 | 1.59 | Agrn |
| 1426671_a_at | Lgx only | 1307 | 1327 | 1062 | 766 | 1.02 | −1.23 | −1.71 | Rbm39 |
| 1426675_at | Lgx only | 1376 | 1180 | 884 | 626 | −1.17 | −1.56 | −2.20 | Tomm70a |
| 1426681_at | Lgx only | 284 | 309 | 369 | 440 | 1.09 | 1.30 | 1.55 | Unk |
| 1426682_at | Lgx only | 1328 | 1222 | 1113 | 852 | −1.09 | −1.19 | −1.56 | LOC100046343 |
| 1426688_at | Lgx only | 30012 | 35064 | 33818 | 42859 | 1.17 | 1.13 | 1.43 | Sdha |
| 1426690_a_at | Res & Lgx | 1211 | 1395 | 1784 | 2490 | 1.15 | 1.47 | 2.06 | Srebf1 |
| 1426691_at | Lgx only | 818 | 960 | 944 | 1189 | 1.17 | 1.15 | 1.45 | Tjap1 |
| 1426700_a_at | Lgx only | 310 | 327 | 417 | 406 | 1.06 | 1.35 | 1.31 | Usp52 |
| 1426717_at | Res & Lgx | 1834 | 1766 | 1219 | 1092 | −1.04 | −1.50 | −1.68 | Nipa2 |
| 1426718_at | Res & Lgx | 972 | 829 | 713 | 726 | −1.17 | −1.36 | −1.34 | Skiv2l2 |
| 1426731_at | Lgx only | 15714 | 18402 | 19327 | 23750 | 1.17 | 1.23 | 1.51 | Des |
| 1426741_a_at | Lgx only | 1254 | 1098 | 1044 | 860 | −1.14 | −1.20 | −1.46 | Fastkd2 |
| 1426743_at | Lgx only | 3106 | 3174 | 2664 | 2485 | 1.02 | −1.17 | −1.25 | Appl2 |
| 1426752_at | Res only | 1165 | 945 | 826 | 990 | −1.23 | −1.41 | −1.18 | Phf17 |
| 1426760_at | Res & Lgx | 1194 | 1065 | 823 | 781 | −1.12 | −1.45 | −1.53 | Ipo8 |
| 1426773_at | Lgx only | 6830 | 8123 | 9064 | 9354 | 1.19 | 1.33 | 1.37 | Mfn1 |
| 1426774_at | Res & Lgx | 607 | 595 | 485 | 347 | −1.02 | −1.25 | −1.75 | Parp12 |
| 1426794_at | Lgx only | 934 | 1249 | 1207 | 1600 | 1.34 | 1.29 | 1.71 | Ptprs |
| 1426799_at | Lgx only | 826 | 683 | 610 | 489 | −1.21 | −1.35 | −1.69 | Rab8b |
| 1426819_at | Lgx only | 9939 | 6798 | 6585 | 3448 | −1.46 | −1.51 | −2.88 | LOC100048439 |
| 1426820_at | Lgx only | 5084 | 5617 | 5406 | 6361 | 1.10 | 1.06 | 1.25 | 2610507B11Rik |
| 1426830_a_at | Lgx only | 3633 | 3395 | 3097 | 2585 | −1.07 | −1.17 | −1.41 | Ahcyl1 |
| 1426833_at | Lgx only | 846 | 870 | 1103 | 1089 | 1.03 | 1.30 | 1.29 | Eif4g3 |
| 1426854_a_at | Lgx only | 4159 | 4032 | 3462 | 3261 | −1.03 | −1.20 | −1.28 | Set |
| 1426857_a_at | Lgx only | 5854 | 4850 | 5610 | 4740 | −1.21 | −1.04 | −1.23 | Hsdl2 |
| 1426866_at | Lgx only | 571 | 766 | 674 | 802 | 1.34 | 1.18 | 1.41 | Chst14 |
| 1426886_at | Res & Lgx | 872 | 844 | 723 | 633 | −1.03 | −1.21 | −1.38 | Cln5 |
| 1426895_at | Res & Lgx | 897 | 707 | 604 | 450 | −1.27 | −1.48 | −1.99 | Zfp191 |
| 1426898_at | Res only | 391 | 401 | 533 | 528 | 1.03 | 1.36 | 1.35 | Map3k7ip1 |
| 1426900_at | Lgx only | 986 | 807 | 644 | 600 | −1.22 | −1.53 | −1.64 | Jmjd1c |
| 1426948_at | Res & Lgx | 3583 | 4241 | 4782 | 5859 | 1.18 | 1.33 | 1.64 | Tpr |
| 1426952_at | Lgx only | 524 | 503 | 478 | 259 | −1.04 | −1.10 | −2.02 | Arhgap18 |
| 1426964_at | Lgx only | 1856 | 1813 | 1314 | 1265 | −1.02 | −1.41 | −1.47 | 3110003A17Rik |
| 1426965_at | Lgx only | 402 | 405 | 499 | 568 | 1.01 | 1.24 | 1.41 | Rap2a |
| 1426969_at | Res & Lgx | 551 | 378 | 376 | 287 | −1.46 | −1.46 | −1.92 | Trim23 |
| 1426976_at | Lgx only | 3711 | 3721 | 4100 | 4747 | 1.00 | 1.10 | 1.28 | Usp47 |
| 1426979_at | Lgx only | 993 | 1192 | 1354 | 1332 | 1.20 | 1.36 | 1.34 | Mlxip |
| 1426982_at | Res & Lgx | 535 | 607 | 880 | 853 | 1.13 | 1.65 | 1.59 | Flywch1 |
| 1426984_at | Res & Lgx | 291 | 540 | 554 | 854 | 1.86 | 1.91 | 2.94 | 2310067B10Rik |
| 1426992_at | Res & Lgx | 1745 | 1757 | 1076 | 1052 | 1.01 | −1.62 | −1.66 | Xpr1 |
| 1427028_at | Lgx only | 556 | 635 | 615 | 891 | 1.14 | 1.11 | 1.60 | Lgr6 |
| 1427039_at | Lgx only | 1834 | 2329 | 2407 | 2983 | 1.27 | 1.31 | 1.63 | Epn1 |
| 1427040_at | Lgx only | 1252 | 987 | 952 | 855 | −1.27 | −1.31 | −1.46 | Mdfic |
| 1427045_at | Lgx only | 778 | 817 | 1248 | 1424 | 1.05 | 1.60 | 1.83 | Synpo |
| 1427051_at | Res & Lgx | 454 | 548 | 673 | 770 | 1.21 | 1.48 | 1.70 | Tnks1bp1 |
| 1427058_at | Res only | 8484 | 8530 | 6619 | 7155 | 1.01 | −1.28 | −1.19 | Eif4a1 |
| 1427073_at | Res only | 1956 | 2206 | 1536 | 1648 | 1.13 | −1.27 | −1.19 | Lace1 |
| 1427084_a_at | Lgx only | 686 | 606 | 508 | 463 | −1.13 | −1.35 | −1.48 | Map4k5 |
| 1427099_at | Res only | 320 | 486 | 646 | 461 | 1.52 | 2.02 | 1.44 | Maz |
| 1427117_at | Lgx only | 619 | 616 | 790 | 979 | −1.01 | 1.28 | 1.58 | Mtmr3 |
| 1427120_at | Lgx only | 835 | 688 | 573 | 468 | −1.21 | −1.46 | −1.78 | Zfp26 |
| 1427129_a_at | Lgx only | 1545 | 1252 | 1132 | 930 | −1.23 | −1.36 | −1.66 | Hnrpr |
| 1427132_at | Lgx only | 348 | 463 | 524 | 564 | 1.33 | 1.51 | 1.62 | Sbf2 |
| 1427139_at | Lgx only | 795 | 940 | 925 | 1054 | 1.18 | 1.16 | 1.33 | Adamts10 |
| 1427144_at | Res & Lgx | 1131 | 1047 | 680 | 637 | −1.08 | −1.66 | −1.78 | Hnrpll |
| 1427146_at | Res & Lgx | 673 | 839 | 1204 | 1613 | 1.25 | 1.79 | 2.40 | AI790298 |
| 1427165_at | Res & Lgx | 1308 | 1233 | 814 | 792 | −1.06 | −1.61 | −1.65 | Il13ra1 |
| 1427166_a_at | Lgx only | 1401 | 1747 | 2035 | 2399 | 1.25 | 1.45 | 1.71 | Spg7 |
| 1427170_at | All | 74 | 25 | 24 | 13 | −2.94 | −3.10 | −5.66 | Psma8 |
| 1427197_at | Lgx only | 496 | 530 | 403 | 303 | 1.07 | −1.23 | −1.64 | Atr |
| 1427201_at | Lgx only | 551 | 457 | 565 | 769 | −1.20 | 1.03 | 1.40 | Mustn1 |
| 1427228_at | Lgx only | 2099 | 2379 | 3387 | 3964 | 1.13 | 1.61 | 1.89 | Palld |
| 1427239_at | CR & Lgx | 552 | 808 | 637 | 680 | 1.46 | 1.15 | 1.23 | Ift122 |
| 1427240_at | Lgx only | 815 | 1013 | 1033 | 1520 | 1.24 | 1.27 | 1.86 | Dock6 |
| 1427241_at | Lgx only | 610 | 547 | 387 | 384 | −1.12 | −1.58 | −1.59 | Papolg |
| 1427260_a_at | Res & Lgx | 2978 | 2624 | 2355 | 1823 | −1.13 | −1.26 | −1.63 | Tpm3 |
| 1427266_at | All | 1181 | 848 | 784 | 732 | −1.39 | −1.51 | −1.61 | Pbrm1 |
| 1427296_at | Res & Lgx | 4758 | 5054 | 3684 | 3677 | 1.06 | −1.29 | −1.29 | BC010304 |
| 1427312_at | Lgx only | 12654 | 14384 | 15939 | 18697 | 1.14 | 1.26 | 1.48 | Cmya5 |
| 1427314_at | Res & Lgx | 2995 | 2439 | 2188 | 1689 | −1.23 | −1.37 | −1.77 | Tmed7 |
| 1427319_at | CR & Lgx | 694 | 468 | 551 | 427 | −1.48 | −1.26 | −1.63 | A230046K03Rik |
| 1427342_at | Res & Lgx | 802 | 683 | 597 | 499 | −1.18 | −1.35 | −1.61 | Fastkd1 |
| 1427395_a_at | Lgx only | 74 | 37 | 30 | 33 | −2.00 | −2.51 | −2.27 | Aldh1a3 |
| 1427418_a_at | Lgx only | 1864 | 1555 | 1250 | 796 | −1.20 | −1.49 | −2.34 | Hif1a |
| 1427432_a_at | Lgx only | 2372 | 2225 | 1569 | 1292 | −1.07 | −1.51 | −1.84 | Sfrs10 |
| 1427447_a_at | Res & Lgx | 1080 | 1415 | 1561 | 1854 | 1.31 | 1.44 | 1.72 | Triobp |
| 1427490_at | Lgx only | 356 | 348 | 274 | 236 | −1.02 | −1.30 | −1.51 | Abcb7 |
| 1427529_at | Res only | 151 | 90 | 67 | 82 | −1.68 | −2.25 | −1.84 | Fzd9 |
| 1427555_at | Lgx only | 35 | 64 | 76 | 96 | 1.85 | 2.18 | 2.76 | Mll2 |
| 1427557_at | Lgx only | 180 | 201 | 256 | 281 | 1.11 | 1.42 | 1.56 | Alg12 |
| 1427604_a_at | Lgx only | 871 | 1023 | 1176 | 1356 | 1.17 | 1.35 | 1.56 | Atp9a |
| 1427661_a_at | Res & Lgx | 382 | 474 | 594 | 597 | 1.24 | 1.56 | 1.56 | Tssc4 |
| 1427689_a_at | Lgx only | 1261 | 1266 | 1376 | 1652 | 1.00 | 1.09 | 1.31 | Tnip1 |
| 1427720_a_at | Lgx only | 2747 | 2851 | 3040 | 3415 | 1.04 | 1.11 | 1.24 | Rrp1 |
| 1427728_at | CR only | 34 | 97 | 56 | 57 | 2.88 | 1.65 | 1.68 | Chrng |
| 1427735_a_at | CR & Res | 8359 | 12700 | 12493 | 12108 | 1.52 | 1.49 | 1.45 | Acta1 |
| 1427873_at | Lgx only | 17 | 54 | 56 | 110 | 3.19 | 3.31 | 6.50 | Defcr15 |
| 1427874_at | Lgx only | 1399 | 1574 | 1815 | 1899 | 1.13 | 1.30 | 1.36 | Zfp313 |
| 1427876_at | Lgx only | 1630 | 1676 | 1431 | 1226 | 1.03 | −1.14 | −1.33 | Zc3h15 |
| 1427886_at | Lgx only | 969 | 755 | 581 | 374 | −1.28 | −1.67 | −2.59 | Pom121 |
| 1427888_a_at | CR & Lgx | 5357 | 6838 | 6596 | 7987 | 1.28 | 1.23 | 1.49 | Spna2 |
| 1427894_at | Lgx only | 472 | 396 | 538 | 682 | −1.19 | 1.14 | 1.44 | Vasn |
| 1427898_at | Lgx only | 822 | 714 | 616 | 515 | −1.15 | −1.33 | −1.60 | Rnf6 |
| 1427901_at | Lgx only | 3381 | 3126 | 2782 | 2128 | −1.08 | −1.22 | −1.59 | Mrps18c |
| 1427903_at | Res & Lgx | 5849 | 5774 | 4732 | 4224 | −1.01 | −1.24 | −1.38 | Phpt1 |
| 1427913_at | Lgx only | 2402 | 2230 | 1819 | 1452 | −1.08 | −1.32 | −1.65 | Rwdd1 |
| 1427918_a_at | Lgx only | 7544 | 7921 | 8074 | 9445 | 1.05 | 1.07 | 1.25 | Rhoq |
| 1427929_a_at | Lgx only | 610 | 746 | 507 | 763 | 1.22 | −1.20 | 1.25 | Pdxk |
| 1427943_at | Lgx only | 6234 | 5441 | 5157 | 3929 | −1.15 | −1.21 | −1.59 | Acyp2 |
| 1427947_at | CR only | 493 | 623 | 501 | 547 | 1.26 | 1.02 | 1.11 | BC028440 |
| 1427955_a_at | Lgx only | 6984 | 7396 | 5948 | 4819 | 1.06 | −1.17 | −1.45 | Deb1 |
| 1427971_at | Lgx only | 957 | 802 | 781 | 608 | −1.19 | −1.22 | −1.57 | Cdc73 |
| 1427983_at | Lgx only | 514 | 395 | 447 | 362 | −1.30 | −1.15 | −1.42 | Suhw3 |
| 1427990_at | Lgx only | 355 | 290 | 266 | 214 | −1.22 | −1.34 | −1.66 | Usp45 |
| 1427996_at | Lgx only | 850 | 752 | 733 | 605 | −1.13 | −1.16 | −1.41 | BC028528 |
| 1427997_at | Res & Lgx | 3311 | 2612 | 2104 | 1880 | −1.27 | −1.57 | −1.76 | 1110007M04Rik |
| 1428029_a_at | Res & Lgx | 4750 | 5138 | 6149 | 6341 | 1.08 | 1.29 | 1.33 | H2afv |
| 1428061_at | Res & Lgx | 945 | 762 | 578 | 474 | −1.24 | −1.64 | −1.99 | Hat1 |
| 1428064_at | Lgx only | 545 | 548 | 561 | 729 | 1.01 | 1.03 | 1.34 | Centd2 |
| 1428071_at | Lgx only | 1332 | 1392 | 1595 | 1813 | 1.04 | 1.20 | 1.36 | 1110038D17Rik |
| 1428083_at | CR & Lgx | 11028 | 17747 | 12934 | 18091 | 1.61 | 1.17 | 1.64 | 2310043N10Rik |
| 1428084_at | Lgx only | 1028 | 928 | 751 | 646 | −1.11 | −1.37 | −1.59 | Krr1 |
| 1428100_at | Lgx only | 710 | 679 | 484 | 450 | −1.05 | −1.47 | −1.58 | Sfrs1 |
| 1428103_at | CR & Lgx | 1072 | 830 | 813 | 700 | −1.29 | −1.32 | −1.53 | Adam10 |
| 1428124_at | Lgx only | 422 | 302 | 322 | 263 | −1.40 | −1.31 | −1.60 | Gtf2e1 |
| 1428128_at | Res & Lgx | 1502 | 1659 | 2529 | 2521 | 1.10 | 1.68 | 1.68 | 4921506J03Rik |
| 1428134_at | Lgx only | 16660 | 18474 | 18163 | 20707 | 1.11 | 1.09 | 1.24 | Coq9 |
| 1428143_a_at | Res & Lgx | 6354 | 8097 | 11246 | 12003 | 1.27 | 1.77 | 1.89 | Pnpla2 |
| 1428158_at | Res & Lgx | 1535 | 1812 | 2043 | 2286 | 1.18 | 1.33 | 1.49 | Akt1s1 |
| 1428160_at | Lgx only | 2716 | 2030 | 1757 | 1025 | −1.34 | −1.55 | −2.65 | Ndufab1 |
| 1428173_at | Lgx only | 437 | 537 | 595 | 762 | 1.23 | 1.36 | 1.74 | Eml2 |
| 1428179_at | Lgx only | 43162 | 47906 | 48747 | 57849 | 1.11 | 1.13 | 1.34 | Ndufv2 |
| 1428182_at | Lgx only | 840 | 966 | 980 | 1096 | 1.15 | 1.17 | 1.30 | Prpsap1 |
| 1428198_at | Lgx only | 360 | 322 | 266 | 217 | −1.12 | −1.36 | −1.66 | Adal |
| 1428201_at | CR only | 2131 | 2525 | 2452 | 2729 | 1.19 | 1.15 | 1.28 | 2310036O22Rik |
| 1428206_at | Res only | 104 | 76 | 36 | 108 | −1.38 | −2.90 | 1.04 | Ccdc69 |
| 1428213_at | Res & Lgx | 2496 | 2367 | 1629 | 1372 | −1.05 | −1.53 | −1.82 | Nsmce4a |
| 1428230_at | CR only | 963 | 819 | 985 | 833 | −1.18 | 1.02 | −1.16 | Prkcn |
| 1428235_at | Lgx only | 19980 | 23234 | 20415 | 24655 | 1.16 | 1.02 | 1.23 | Sdhd |
| 1428236_at | Lgx only | 556 | 408 | 377 | 306 | −1.36 | −1.48 | −1.81 | Acbd5 |
| 1428244_at | Lgx only | 821 | 1001 | 1063 | 1344 | 1.22 | 1.29 | 1.64 | Larp1 |
| 1428250_at | Lgx only | 150 | 122 | 155 | 259 | −1.23 | 1.03 | 1.73 | Gper |
| 1428251_at | Lgx only | 464 | 397 | 316 | 330 | −1.17 | −1.47 | −1.40 | Smchd1 |
| 1428253_at | Res & Lgx | 1966 | 1803 | 1418 | 1374 | −1.09 | −1.39 | −1.43 | Chmp2b |
| 1428256_at | Res & Lgx | 2199 | 1822 | 1588 | 1576 | −1.21 | −1.39 | −1.40 | 2310047H23Rik |
| 1428260_at | Res & Lgx | 200 | 180 | 123 | 107 | −1.11 | −1.62 | −1.88 | Spg3a |
| 1428277_at | Lgx only | 2436 | 2410 | 1959 | 1529 | −1.01 | −1.24 | −1.59 | Otud6b |
| 1428282_at | Lgx only | 1073 | 922 | 1060 | 757 | −1.16 | −1.01 | −1.42 | Tbce |
| 1428299_at | Res & Lgx | 1060 | 1010 | 682 | 653 | −1.05 | −1.55 | −1.62 | Dyrk1a |
| 1428307_at | Lgx only | 1160 | 1032 | 931 | 745 | −1.12 | −1.24 | −1.56 | Zdhhc13 |
| 1428316_a_at | Res & Lgx | 6077 | 4954 | 4136 | 3051 | −1.23 | −1.47 | −1.99 | Fundc2 |
| 1428332_at | CR & Lgx | 2444 | 3279 | 2758 | 3376 | 1.34 | 1.13 | 1.38 | Pik3ip1 |
| 1428335_a_at | Lgx only | 810 | 785 | 691 | 580 | −1.03 | −1.17 | −1.40 | Scfd1 |
| 1428357_at | Res only | 190 | 206 | 336 | 219 | 1.08 | 1.77 | 1.15 | 2610019F03Rik |
| 1428365_a_at | Lgx only | 2043 | 2396 | 2896 | 3413 | 1.17 | 1.42 | 1.67 | Lonp1 |
| 1428366_at | All | 798 | 646 | 577 | 485 | −1.23 | −1.38 | −1.65 | 1600027N09Rik |
| 1428370_at | CR only | 128 | 228 | 179 | 125 | 1.78 | 1.40 | −1.03 | 1500011B03Rik |
| 1428380_at | Res & Lgx | 2352 | 2151 | 1639 | 1394 | −1.09 | −1.44 | −1.69 | 0610007C21Rik |
| 1428382_at | Lgx only | 964 | 1129 | 1473 | 1531 | 1.17 | 1.53 | 1.59 | Smarcc2 |
| 1428391_at | Lgx only | 267 | 295 | 348 | 432 | 1.10 | 1.30 | 1.62 | Rab3il1 |
| 1428395_at | Lgx only | 487 | 508 | 647 | 727 | 1.04 | 1.33 | 1.49 | Smurf1 |
| 1428405_at | Lgx only | 9641 | 10670 | 11324 | 12596 | 1.11 | 1.17 | 1.31 | Hcfc1r1 |
| 1428412_at | Lgx only | 2036 | 2091 | 2325 | 2629 | 1.03 | 1.14 | 1.29 | Tm9sf3 |
| 1428421_a_at | Lgx only | 2913 | 2410 | 2394 | 2019 | −1.21 | −1.22 | −1.44 | Glod4 |
| 1428423_at | Lgx only | 1714 | 1643 | 1657 | 1494 | −1.04 | −1.03 | −1.15 | Pcgf3 |
| 1428427_at | CR only | 92 | 36 | 39 | 101 | −2.53 | −2.37 | 1.10 | Fbxl2 |
| 1428431_at | Res & Lgx | 1390 | 1453 | 1755 | 1963 | 1.04 | 1.26 | 1.41 | 2310047A01Rik |
| 1428436_at | Res & Lgx | 1165 | 1047 | 815 | 839 | −1.11 | −1.43 | −1.39 | Lsm14a |
| 1428440_at | Lgx only | 8819 | 10649 | 10766 | 12943 | 1.21 | 1.22 | 1.47 | Slc25a12 |
| 1428441_at | Lgx only | 2992 | 2477 | 1975 | 1655 | −1.21 | −1.51 | −1.81 | Cisd2 |
| 1428443_a_at | Lgx only | 732 | 902 | 816 | 1006 | 1.23 | 1.11 | 1.37 | Rap1gap |
| 1428465_at | Lgx only | 6696 | 7010 | 5690 | 4717 | 1.05 | −1.18 | −1.42 | Tmem147 |
| 1428468_at | All | 950 | 768 | 805 | 688 | −1.24 | −1.18 | −1.38 | 3110043O21Rik |
| 1428476_a_at | Lgx only | 441 | 575 | 672 | 824 | 1.30 | 1.52 | 1.87 | Elac2 |
| 1428494_a_at | Lgx only | 3176 | 3065 | 2853 | 2679 | −1.04 | −1.11 | −1.19 | Polr2i |
| 1428495_at | Res & Lgx | 2251 | 1796 | 1512 | 1278 | −1.25 | −1.49 | −1.76 | 2410003K15Rik |
| 1428503_a_at | Lgx only | 2042 | 1913 | 1596 | 1490 | −1.07 | −1.28 | −1.37 | Nkiras1 |
| 1428505_at | Lgx only | 4503 | 4374 | 3908 | 2889 | −1.03 | −1.15 | −1.56 | Ccdc90b |
| 1428507_at | Lgx only | 1620 | 1610 | 1433 | 1331 | −1.01 | −1.13 | −1.22 | Hdhd2 |
| 1428508_at | Lgx only | 415 | 458 | 547 | 572 | 1.10 | 1.32 | 1.38 | Tbc1d2b |
| 1428510_at | Lgx only | 331 | 473 | 485 | 525 | 1.43 | 1.46 | 1.58 | Lphn1 |
| 1428515_at | Lgx only | 845 | 790 | 736 | 569 | −1.07 | −1.15 | −1.48 | 2410012H22Rik |
| 1428519_at | Lgx only | 3006 | 3200 | 2041 | 1928 | 1.06 | −1.47 | −1.56 | 2610528E23Rik |
| 1428540_at | Lgx only | 162 | 219 | 253 | 370 | 1.35 | 1.56 | 2.28 | 3321401G04Rik |
| 1428544_at | Res & Lgx | 1458 | 1331 | 1205 | 1043 | −1.10 | −1.21 | −1.40 | 0610007L01Rik |
| 1428549_at | Lgx only | 404 | 587 | 708 | 973 | 1.45 | 1.75 | 2.41 | Ccdc3 |
| 1428551_at | All | 518 | 361 | 300 | 223 | −1.44 | −1.72 | −2.32 | Trmt11 |
| 1428552_at | Res & Lgx | 2497 | 2020 | 1544 | 1257 | −1.24 | −1.62 | −1.99 | 2610001J05Rik |
| 1428554_a_at | Lgx only | 2768 | 2661 | 2626 | 2233 | −1.04 | −1.05 | −1.24 | 1810035L17Rik |
| 1428564_at | Lgx only | 417 | 459 | 555 | 738 | 1.10 | 1.33 | 1.77 | Zfp579 |
| 1428580_at | Lgx only | 1441 | 1418 | 1326 | 1067 | −1.02 | −1.09 | −1.35 | Blvra |
| 1428585_at | Lgx only | 720 | 831 | 840 | 1309 | 1.15 | 1.17 | 1.82 | Actn1 |
| 1428587_at | Res & Lgx | 2171 | 2060 | 1437 | 1374 | −1.05 | −1.51 | −1.58 | Tmem41b |
| 1428589_at | Lgx only | 7082 | 6392 | 5436 | 4990 | −1.11 | −1.30 | −1.42 | Mrpl41 |
| 1428593_at | Res only | 436 | 336 | 287 | 244 | −1.30 | −1.52 | −1.79 | 1700029F09Rik |
| 1428594_at | Lgx only | 1864 | 1998 | 1599 | 1255 | 1.07 | −1.17 | −1.48 | Garnl1 |
| 1428598_at | All | 663 | 946 | 941 | 976 | 1.43 | 1.42 | 1.47 | Tbc1d7 |
| 1428613_at | Res only | 548 | 583 | 786 | 754 | 1.06 | 1.43 | 1.38 | Ldhd |
| 1428615_at | CR only | 1077 | 892 | 1088 | 1153 | −1.21 | 1.01 | 1.07 | P2ry5 |
| 1428617_at | Res & Lgx | 1207 | 1018 | 969 | 859 | −1.19 | −1.25 | −1.41 | Hcfc2 |
| 1428626_at | Lgx only | 750 | 726 | 685 | 550 | −1.03 | −1.09 | −1.36 | Lysmd2 |
| 1428651_at | CR & Lgx | 5284 | 4538 | 5141 | 4573 | −1.16 | −1.03 | −1.16 | Klhl24 |
| 1428652_at | Res & Lgx | 341 | 312 | 224 | 199 | −1.09 | −1.53 | −1.72 | 0610010F05Rik |
| 1428668_at | Lgx only | 921 | 942 | 1096 | 1235 | 1.02 | 1.19 | 1.34 | Acbd3 |
| 1428682_at | CR only | 181 | 79 | 159 | 142 | −2.29 | −1.14 | −1.27 | Zc3h6 |
| 1428691_at | Lgx only | 1373 | 1505 | 1521 | 1856 | 1.10 | 1.11 | 1.35 | Chd2 |
| 1428697_at | Lgx only | 1082 | 1076 | 1176 | 1398 | −1.01 | 1.09 | 1.29 | Dpp8 |
| 1428707_at | Lgx only | 2825 | 3531 | 3687 | 4608 | 1.25 | 1.31 | 1.63 | Ptms |
| 1428715_at | Res & Lgx | 561 | 444 | 367 | 278 | −1.26 | −1.53 | −2.02 | 2810423A18Rik |
| 1428722_at | Lgx only | 59642 | 62216 | 62442 | 77221 | 1.04 | 1.05 | 1.29 | Ckmt2 |
| 1428723_at | Lgx only | 404 | 425 | 515 | 657 | 1.05 | 1.27 | 1.62 | 2310047M10Rik |
| 1428728_at | CR only | 232 | 387 | 266 | 290 | 1.67 | 1.15 | 1.25 | Ddx51 |
| 1428731_at | Lgx only | 946 | 1052 | 960 | 1147 | 1.11 | 1.01 | 1.21 | Usp54 |
| 1428748_at | Lgx only | 426 | 369 | 313 | 221 | −1.16 | −1.36 | −1.93 | 5830428H23Rik |
| 1428749_at | Res & Lgx | 631 | 573 | 372 | 387 | −1.10 | −1.69 | −1.63 | Dmxl2 |
| 1428758_at | CR & Res | 2386 | 1678 | 1803 | 1967 | −1.42 | −1.32 | −1.21 | Tmem86a |
| 1428767_at | Lgx only | 347 | 345 | 449 | 482 | −1.00 | 1.30 | 1.39 | Gsdmdc1 |
| 1428769_at | Res only | 568 | 531 | 411 | 491 | −1.07 | −1.38 | −1.16 | Tatdn3 |
| 1428782_a_at | Res & Lgx | 40904 | 44865 | 48798 | 54806 | 1.10 | 1.19 | 1.34 | Uqcrc1 |
| 1428786_at | Res only | 261 | 274 | 406 | 363 | 1.05 | 1.55 | 1.39 | Nckap1l |
| 1428789_at | CR only | 425 | 281 | 383 | 383 | −1.51 | −1.11 | −1.11 | Ralgps2 |
| 1428791_at | Lgx only | 2024 | 2245 | 2339 | 2646 | 1.11 | 1.16 | 1.31 | Ube2h |
| 1428807_at | Lgx only | 24 | 19 | 27 | 66 | −1.30 | 1.10 | 2.72 | Pabpc3 |
| 1428810_at | Lgx only | 1298 | 1336 | 1428 | 1643 | 1.03 | 1.10 | 1.27 | 2700097O09Rik |
| 1428812_at | Res only | 600 | 608 | 802 | 795 | 1.01 | 1.34 | 1.33 | 1700040L02Rik |
| 1428829_at | CR & Lgx | 827 | 624 | 677 | 551 | −1.32 | −1.22 | −1.50 | 6820401H01Rik |
| 1428831_at | Lgx only | 626 | 553 | 446 | 354 | −1.13 | −1.40 | −1.77 | 6230429P13Rik |
| 1428835_at | Lgx only | 1354 | 1406 | 2031 | 2409 | 1.04 | 1.50 | 1.78 | Myh14 |
| 1428845_at | Res & Lgx | 2313 | 2038 | 1791 | 1813 | −1.14 | −1.29 | −1.28 | Bclaf1 |
| 1428848_a_at | Lgx only | 2317 | 2709 | 2961 | 3010 | 1.17 | 1.28 | 1.30 | Macf1 |
| 1428884_at | Lgx only | 1347 | 1481 | 1302 | 1707 | 1.10 | −1.03 | 1.27 | Tmem57 |
| 1428890_at | Lgx only | 1103 | 993 | 869 | 687 | −1.11 | −1.27 | −1.61 | Fem1c |
| 1428897_at | Res & Lgx | 1192 | 1158 | 601 | 686 | −1.03 | −1.98 | −1.74 | 2610029I01Rik |
| 1428899_at | Res & Lgx | 6189 | 6125 | 4734 | 4774 | −1.01 | −1.31 | −1.30 | Tmem182 |
| 1428914_at | Res & Lgx | 1933 | 2302 | 2457 | 2903 | 1.19 | 1.27 | 1.50 | 2310014D11Rik |
| 1428919_at | Res & Lgx | 753 | 645 | 488 | 360 | −1.17 | −1.54 | −2.09 | Fgfr1op |
| 1428926_at | Lgx only | 1791 | 2110 | 2525 | 2734 | 1.18 | 1.41 | 1.53 | 1110003O08Rik |
| 1428945_at | Res only | 511 | 424 | 286 | 365 | −1.21 | −1.79 | −1.40 | Ube1l2 |
| 1428949_at | Lgx only | 1079 | 913 | 908 | 601 | −1.18 | −1.19 | −1.80 | Xpot |
| 1428982_at | Res & Lgx | 465 | 367 | 261 | 211 | −1.27 | −1.78 | −2.20 | Atad2b |
| 1428998_at | Lgx only | 1201 | 1404 | 1356 | 1719 | 1.17 | 1.13 | 1.43 | Phf3 |
| 1429001_at | Res only | 370 | 340 | 478 | 410 | −1.09 | 1.29 | 1.11 | Pir |
| 1429024_at | Lgx only | 3812 | 4605 | 4484 | 5982 | 1.21 | 1.18 | 1.57 | Rbm20 |
| 1429028_at | Lgx only | 460 | 366 | 348 | 309 | −1.26 | −1.32 | −1.49 | Dock11 |
| 1429034_at | Lgx only | 277 | 217 | 197 | 144 | −1.28 | −1.41 | −1.93 | Eme2 |
| 1429042_at | Res & Lgx | 583 | 493 | 381 | 355 | −1.18 | −1.53 | −1.64 | 2010200O16Rik |
| 1429057_at | Res & Lgx | 412 | 390 | 249 | 239 | −1.06 | −1.65 | −1.73 | Narg1l |
| 1429070_at | Lgx only | 166 | 214 | 221 | 297 | 1.29 | 1.33 | 1.79 | 4933440H19Rik |
| 1429085_at | Lgx only | 3041 | 3379 | 3563 | 3775 | 1.11 | 1.17 | 1.24 | Vezf1 |
| 1429103_at | Res & Lgx | 4218 | 4035 | 2877 | 2623 | −1.05 | −1.47 | −1.61 | Tomm22 |
| 1429107_at | Lgx only | 2462 | 2746 | 3278 | 3325 | 1.12 | 1.33 | 1.35 | Zfp650 |
| 1429119_at | Lgx only | 962 | 917 | 756 | 744 | −1.05 | −1.27 | −1.29 | Iah1 |
| 1429121_at | Lgx only | 375 | 263 | 322 | 170 | −1.43 | −1.16 | −2.20 | 4921517N04Rik |
| 1429137_at | Lgx only | 193 | 143 | 138 | 92 | −1.36 | −1.40 | −2.09 | 2810422O20Rik |
| 1429144_at | Res only | 14307 | 13184 | 10505 | 13791 | −1.09 | −1.36 | −1.04 | Prei4 |
| 1429146_at | Lgx only | 1179 | 1070 | 980 | 860 | −1.10 | −1.20 | −1.37 | 6620401M08Rik |
| 1429155_at | Res only | 996 | 805 | 760 | 789 | −1.24 | −1.31 | −1.26 | 4933411K20Rik |
| 1429160_at | Res & Lgx | 684 | 523 | 484 | 470 | −1.31 | −1.41 | −1.45 | Mtif3 |
| 1429183_at | Lgx only | 4351 | 3822 | 4249 | 3307 | −1.14 | −1.02 | −1.32 | Pkp2 |
| 1429186_a_at | Lgx only | 1223 | 1135 | 973 | 934 | −1.08 | −1.26 | −1.31 | Cdadc1 |
| 1429188_at | Lgx only | 1122 | 1148 | 907 | 855 | 1.02 | −1.24 | −1.31 | Cox11 |
| 1429194_at | Lgx only | 546 | 487 | 456 | 394 | −1.12 | −1.20 | −1.39 | Tigd2 |
| 1429196_at | Lgx only | 1381 | 1472 | 2063 | 2670 | 1.07 | 1.49 | 1.93 | Rabgap1l |
| 1429206_at | Lgx only | 3457 | 4642 | 4046 | 4930 | 1.34 | 1.17 | 1.43 | Rhobtb1 |
| 1429209_at | CR only | 179 | 101 | 118 | 196 | −1.78 | −1.52 | 1.09 | Col23a1 |
| 1429223_a_at | Res & Lgx | 2792 | 2374 | 2100 | 1610 | −1.18 | −1.33 | −1.73 | Hfe2 |
| 1429243_at | Res & Lgx | 597 | 537 | 380 | 369 | −1.11 | −1.57 | −1.62 | 1110054O05Rik |
| 1429253_at | Lgx only | 562 | 445 | 513 | 371 | −1.26 | −1.10 | −1.52 | Zmym4 |
| 1429264_at | Lgx only | 496 | 426 | 359 | 315 | −1.16 | −1.38 | −1.58 | C030044B11Rik |
| 1429278_at | Res only | 1094 | 1016 | 863 | 869 | −1.08 | −1.27 | −1.26 | Nubpl |
| 1429281_at | Res & Lgx | 323 | 264 | 201 | 185 | −1.22 | −1.60 | −1.75 | 2610008E11Rik |
| 1429300_at | Lgx only | 742 | 943 | 806 | 1172 | 1.27 | 1.09 | 1.58 | Ankrd9 |
| 1429321_at | Lgx only | 284 | 215 | 514 | 719 | −1.32 | 1.81 | 2.53 | Rnf149 |
| 1429328_at | Lgx only | 5245 | 4514 | 4643 | 3961 | −1.16 | −1.13 | −1.32 | Nsfl1c |
| 1429335_at | Lgx only | 320 | 284 | 287 | 200 | −1.13 | −1.12 | −1.60 | Snapc1 |
| 1429352_at | CR only | 291 | 116 | 140 | 150 | −2.52 | −2.08 | −1.95 | Mocos |
| 1429362_a_at | Res & Lgx | 1201 | 1528 | 1807 | 2347 | 1.27 | 1.50 | 1.95 | Sf3b2 |
| 1429364_at | Lgx only | 108 | 72 | 103 | 47 | −1.51 | −1.05 | −2.30 | 4930579G24Rik |
| 1429367_at | Lgx only | 693 | 717 | 798 | 936 | 1.03 | 1.15 | 1.35 | Wipi2 |
| 1429375_at | Lgx only | 1484 | 1372 | 1276 | 913 | −1.08 | −1.16 | −1.63 | Anapc10 |
| 1429395_at | Lgx only | 138 | 142 | 96 | 54 | 1.02 | −1.45 | −2.55 | Gstcd |
| 1429400_at | Lgx only | 189 | 176 | 206 | 314 | −1.07 | 1.09 | 1.66 | Clcn5 |
| 1429407_at | Lgx only | 188 | 211 | 333 | 337 | 1.12 | 1.77 | 1.79 | Pex11c |
| 1429413_at | Res & Lgx | 292 | 246 | 187 | 156 | −1.18 | −1.56 | −1.88 | Cpm |
| 1429425_at | Res & Lgx | 3510 | 3354 | 2840 | 2532 | −1.05 | −1.24 | −1.39 | Rnf139 |
| 1429454_at | Res & Lgx | 1848 | 1764 | 1340 | 1334 | −1.05 | −1.38 | −1.38 | Gapvd1 |
| 1429460_at | CR only | 15 | 60 | 60 | 28 | 4.09 | 4.13 | 1.89 | Gpr115 |
| 1429486_at | CR only | 1047 | 1648 | 912 | 1004 | 1.57 | −1.15 | −1.04 | Pfkfb2 |
| 1429487_at | Lgx only | 1134 | 1266 | 1668 | 1601 | 1.12 | 1.47 | 1.41 | Ppp1r12a |
| 1429505_at | CR & Lgx | 2791 | 1949 | 2450 | 1714 | −1.43 | −1.14 | −1.63 | 2310076G13Rik |
| 1429514_at | Lgx only | 4969 | 4087 | 4128 | 2951 | −1.22 | −1.20 | −1.68 | Ppap2b |
| 1429521_at | Lgx only | 323 | 250 | 235 | 153 | −1.29 | −1.37 | −2.12 | Alkbh8 |
| 1429532_at | Res only | 865 | 998 | 1089 | 938 | 1.15 | 1.26 | 1.08 | Morc2a |
| 1429534_a_at | Lgx only | 10910 | 12210 | 12327 | 12968 | 1.12 | 1.13 | 1.19 | Immt |
| 1429553_at | Lgx only | 151 | 181 | 260 | 574 | 1.20 | 1.73 | 3.81 | Cilp2 |
| 1429556_at | Lgx only | 2326 | 2174 | 1662 | 1363 | −1.07 | −1.40 | −1.71 | 2610024B07Rik |
| 1429570_at | Res only | 126 | 63 | 36 | 102 | −2.00 | −3.45 | −1.24 | Mlkl |
| 1429621_at | Lgx only | 839 | 1173 | 1240 | 1343 | 1.40 | 1.48 | 1.60 | Cand2 |
| 1429648_at | Lgx only | 637 | 552 | 475 | 461 | −1.15 | −1.34 | −1.38 | Slc35a3 |
| 1429681_a_at | Lgx only | 21171 | 22951 | 25817 | 28204 | 1.08 | 1.22 | 1.33 | Gpsn2 |
| 1429698_at | Res & Lgx | 163 | 155 | 82 | 89 | −1.05 | −1.99 | −1.83 | Mterf |
| 1429710_at | Res & Lgx | 839 | 763 | 558 | 459 | −1.10 | −1.51 | −1.83 | Styx |
| 1429723_at | Res & Lgx | 2893 | 2604 | 1673 | 1495 | −1.11 | −1.73 | −1.94 | 6330409N04Rik |
| 1429764_at | CR only | 2988 | 2220 | 3256 | 2552 | −1.35 | 1.09 | −1.17 | 1500005K14Rik |
| 1429771_at | Lgx only | 927 | 823 | 776 | 635 | −1.13 | −1.19 | −1.46 | 3110073H01Rik |
| 1429783_at | Res & Lgx | 13193 | 13213 | 10275 | 9645 | 1.00 | −1.28 | −1.37 | Pdlim5 |
| 1429819_at | Lgx only | 393 | 378 | 337 | 275 | −1.04 | −1.17 | −1.43 | Nmnat1 |
| 1429836_at | CR only | 142 | 251 | 216 | 269 | 1.78 | 1.53 | 1.90 | Ugcgl2 |
| 1429860_at | CR only | 43 | 117 | 154 | 113 | 2.69 | 3.56 | 2.61 | LOC677447 |
| 1429888_a_at | Lgx only | 4996 | 5288 | 5443 | 6090 | 1.06 | 1.09 | 1.22 | Hspb2 |
| 1429915_at | Lgx only | 56 | 63 | 79 | 116 | 1.12 | 1.42 | 2.08 | 4930426L09Rik |
| 1429918_at | CR only | 433 | 297 | 409 | 416 | −1.46 | −1.06 | −1.04 | Arhgap20 |
| 1429961_at | CR & Lgx | 308 | 198 | 313 | 200 | −1.55 | 1.02 | −1.54 | 1700021C14Rik |
| 1429990_at | Lgx only | 114 | 138 | 174 | 186 | 1.21 | 1.52 | 1.63 | Hyal4 |
| 1430000_at | CR only | 71 | 24 | 62 | 56 | −2.92 | −1.16 | −1.27 | B230117O15Rik |
| 1430045_at | CR & Res | 416 | 259 | 269 | 292 | −1.61 | −1.55 | −1.42 | Tsnax |
| 1430078_a_at | Lgx only | 284 | 264 | 259 | 154 | −1.07 | −1.09 | −1.85 | Ogg1 |
| 1430089_at | Lgx only | 355 | 291 | 227 | 142 | −1.22 | −1.57 | −2.50 | 5830469G19Rik |
| 1430095_at | Lgx only | 90 | 95 | 135 | 240 | 1.06 | 1.50 | 2.66 | D930020B18Rik |
| 1430123_a_at | Lgx only | 13843 | 13323 | 16691 | 18357 | −1.04 | 1.21 | 1.33 | Akr1a4 |
| 1430137_at | Res only | 148 | 166 | 220 | 222 | 1.12 | 1.49 | 1.51 | LOC100043489 |
| 1430170_at | Res only | 162 | 131 | 69 | 89 | −1.24 | −2.35 | −1.82 | Bbs10 |
| 1430224_at | Res only | 101 | 97 | 238 | 112 | −1.03 | 2.36 | 1.12 | Wfdc3 |
| 1430253_at | Lgx only | 114 | 159 | 173 | 252 | 1.40 | 1.52 | 2.22 | 2900006B11Rik |
| 1430292_a_at | Res & Lgx | 3466 | 3219 | 2398 | 2125 | −1.08 | −1.45 | −1.63 | 1810030N24Rik |
| 1430309_at | Res & Lgx | 1747 | 1890 | 2535 | 2577 | 1.08 | 1.45 | 1.47 | Nipbl |
| 1430378_at | Res & Lgx | 205 | 160 | 103 | 72 | −1.28 | −2.00 | −2.85 | 2900011G08Rik |
| 1430388_a_at | Lgx only | 1818 | 2657 | 2347 | 2718 | 1.46 | 1.29 | 1.49 | Sulf2 |
| 1430474_a_at | Res & Lgx | 8337 | 8097 | 5357 | 5222 | −1.03 | −1.56 | −1.60 | Mtch2 |
| 1430518_at | Lgx only | 85 | 28 | 55 | 17 | −3.02 | −1.56 | −5.03 | 5430402E10Rik |
| 1430519_a_at | Lgx only | 939 | 787 | 784 | 667 | −1.19 | −1.20 | −1.41 | Cnot7 |
| 1430527_a_at | Res & Lgx | 2831 | 2873 | 3201 | 3440 | 1.01 | 1.13 | 1.22 | Rnf167 |
| 1430544_at | Lgx only | 259 | 358 | 390 | 468 | 1.39 | 1.51 | 1.81 | 5830404H04Rik |
| 1430656_a_at | Res & Lgx | 3948 | 3401 | 2556 | 2175 | −1.16 | −1.54 | −1.82 | Asnsd1 |
| 1430676_at | CR & Lgx | 26 | 92 | 89 | 122 | 3.52 | 3.39 | 4.65 | Col19a1 |
| 1430685_at | Res only | 17 | 41 | 35 | 64 | 2.38 | 2.07 | 3.78 | 6330503C03Rik |
| 1430736_at | Lgx only | 176 | 135 | 173 | 48 | −1.31 | −1.02 | −3.70 | 9030411M15Rik |
| 1430768_at | Lgx only | 206 | 134 | 121 | 101 | −1.54 | −1.70 | −2.03 | 9530018H14Rik |
| 1430770_at | Res & Lgx | 56 | 28 | 13 | 12 | −1.97 | −4.28 | −4.68 | 3110080E11Rik |
| 1430781_at | Lgx only | 164 | 131 | 118 | 78 | −1.26 | −1.39 | −2.11 | Ak7 |
| 1430799_at | Res & Lgx | 68 | 35 | 14 | 18 | −1.91 | −4.91 | −3.76 | 5830432E09Rik |
| 1430818_at | CR only | 37 | 121 | 118 | 66 | 3.27 | 3.20 | 1.78 | Tmc1 |
| 1430835_at | CR only | 112 | 29 | 98 | 107 | −3.85 | −1.14 | −1.05 | Ccdc125 |
| 1430883_at | CR only | 103 | 36 | 71 | 54 | −2.83 | −1.44 | −1.89 | 4933402C05Rik |
| 1430910_at | CR only | 173 | 44 | 108 | 126 | −3.91 | −1.60 | −1.37 | 4930544L04Rik |
| 1430999_a_at | Res & Lgx | 840 | 643 | 415 | 329 | −1.31 | −2.02 | −2.55 | Scoc |
| 1431043_at | Lgx only | 596 | 596 | 738 | 893 | −1.00 | 1.24 | 1.50 | Kbtbd5 |
| 1431255_at | CR only | 604 | 385 | 610 | 572 | −1.57 | 1.01 | −1.06 | Calr3 |
| 1431287_at | CR only | 71 | 140 | 68 | 62 | 1.97 | −1.05 | −1.15 | Pcm1 |
| 1431293_a_at | Res & Lgx | 2705 | 1856 | 1662 | 1146 | −1.46 | −1.63 | −2.36 | Cldnd1 |
| 1431302_a_at | CR & Lgx | 4139 | 2946 | 3637 | 2685 | −1.41 | −1.14 | −1.54 | Nudt7 |
| 1431322_at | Lgx only | 704 | 836 | 854 | 982 | 1.19 | 1.21 | 1.40 | Igsf3 |
| 1431415_a_at | Lgx only | 681 | 643 | 595 | 493 | −1.06 | −1.14 | −1.38 | Tbpl1 |
| 1431428_a_at | Lgx only | 1082 | 1202 | 1304 | 1403 | 1.11 | 1.21 | 1.30 | Nosip |
| 1431429_a_at | Lgx only | 858 | 839 | 1095 | 1129 | −1.02 | 1.28 | 1.32 | Arl4a |
| 1431473_at | Lgx only | 18 | 7 | 25 | 81 | −2.53 | 1.36 | 4.43 | 5330423I11Rik |
| 1431498_at | Res only | 94 | 120 | 187 | 171 | 1.28 | 1.99 | 1.82 | 9530097N15Rik |
| 1431551_at | Lgx only | 62 | 105 | 104 | 164 | 1.69 | 1.68 | 2.64 | 2610028D06Rik |
| 1431561_a_at | Res only | 485 | 676 | 690 | 684 | 1.39 | 1.42 | 1.41 | Dhx34 |
| 1431587_at | Res only | 70 | 55 | 24 | 53 | −1.28 | −2.90 | −1.32 | Ccdc7 |
| 1431610_at | Res only | 49 | 101 | 141 | 107 | 2.05 | 2.87 | 2.18 | 5330439A09Rik |
| 1431618_a_at | CR & Res | 325 | 230 | 221 | 179 | −1.41 | −1.47 | −1.82 | D14Ertd581e |
| 1431619_a_at | Lgx only | 3465 | 3627 | 4405 | 4627 | 1.05 | 1.27 | 1.34 | Dtnbp1 |
| 1431679_at | Res only | 49 | 89 | 165 | 104 | 1.80 | 3.35 | 2.11 | 2510042H12Rik |
| 1431746_a_at | Lgx only | 3679 | 3526 | 2924 | 2509 | −1.04 | −1.26 | −1.47 | Ube1c |
| 1431785_at | Lgx only | 175 | 194 | 145 | 90 | 1.11 | −1.20 | −1.94 | Rnaset2a |
| 1431796_at | Lgx only | 31 | 106 | 75 | 91 | 3.40 | 2.40 | 2.93 | 2810430I11Rik |
| 1431804_a_at | Res & Lgx | 690 | 548 | 337 | 279 | −1.26 | −2.04 | −2.47 | Sp3 |
| 1431822_a_at | Res & Lgx | 3622 | 3320 | 2534 | 2374 | −1.09 | −1.43 | −1.53 | Azi2 |
| 1431827_a_at | Lgx only | 896 | 797 | 717 | 590 | −1.12 | −1.25 | −1.52 | Tlk2 |
| 1431853_at | CR only | 92 | 40 | 75 | 92 | −2.33 | −1.22 | 1.00 | 4933413C19Rik |
| 1431893_a_at | Res & Lgx | 638 | 758 | 379 | 436 | 1.19 | −1.68 | −1.46 | Pdss1 |
| 1431900_a_at | CR only | 32 | 90 | 31 | 85 | 2.79 | −1.06 | 2.64 | Foxa3 |
| 1431934_at | Lgx only | 72 | 109 | 81 | 157 | 1.51 | 1.13 | 2.18 | 4930505O20Rik |
| 1431986_at | Res only | 197 | 193 | 296 | 133 | −1.02 | 1.50 | −1.48 | 4933421A08Rik |
| 1431998_at | CR only | 69 | 14 | 61 | 59 | −4.95 | −1.14 | −1.16 | 4930432L08Rik |
| 1432000_a_at | Lgx only | 342 | 378 | 448 | 482 | 1.11 | 1.31 | 1.41 | Dedd |
| 1432016_a_at | CR only | 31956 | 37162 | 31250 | 32250 | 1.16 | −1.02 | 1.01 | Idh3a |
| 1432057_a_at | Lgx only | 662 | 743 | 770 | 911 | 1.12 | 1.16 | 1.38 | Prdm5 |
| 1432073_at | Res & Lgx | 3205 | 3584 | 2249 | 2107 | 1.12 | −1.43 | −1.52 | 1700113I22Rik |
| 1432122_at | Lgx only | 128 | 89 | 121 | 44 | −1.44 | −1.05 | −2.91 | Lrrc44 |
| 1432158_a_at | Res & Lgx | 2091 | 1924 | 1569 | 1489 | −1.09 | −1.33 | −1.40 | Trappc2 |
| 1432207_a_at | Lgx only | 454 | 448 | 459 | 350 | −1.01 | 1.01 | −1.30 | Toe1 |
| 1432248_at | Lgx only | 31 | 74 | 52 | 105 | 2.36 | 1.66 | 3.36 | 5430402P08Rik |
| 1432271_a_at | Res & Lgx | 1856 | 1636 | 1172 | 967 | −1.13 | −1.58 | −1.92 | Dcun1d5 |
| 1432348_at | Lgx only | 59 | 61 | 97 | 183 | 1.03 | 1.63 | 3.09 | 4930524O07Rik |
| 1432369_at | CR & Res | 60 | 139 | 152 | 155 | 2.30 | 2.52 | 2.57 | 3010027C24Rik |
| 1432420_a_at | Lgx only | 1929 | 1605 | 1516 | 1137 | −1.20 | −1.27 | −1.70 | 2310002L09Rik |
| 1432444_a_at | Lgx only | 1027 | 964 | 956 | 784 | −1.07 | −1.08 | −1.31 | Eapp |
| 1432457_at | Res only | 99 | 147 | 183 | 188 | 1.48 | 1.85 | 1.90 | 4930448F12Rik |
| 1432466_a_at | Lgx only | 9714 | 10651 | 13047 | 18186 | 1.10 | 1.34 | 1.87 | Apoe |
| 1432533_a_at | Lgx only | 372 | 420 | 476 | 655 | 1.13 | 1.28 | 1.76 | Slc35a2 |
| 1432543_a_at | Res only | 510 | 488 | 700 | 806 | −1.04 | 1.37 | 1.58 | Klf13 |
| 1432560_at | Res only | 52 | 58 | 122 | 70 | 1.13 | 2.37 | 1.35 | 1700127D06Rik |
| 1432590_at | CR only | 156 | 51 | 107 | 119 | −3.09 | −1.46 | −1.31 | 4930573O21Rik |
| 1432625_at | Res only | 32 | 59 | 123 | 62 | 1.81 | 3.78 | 1.90 | 5830487K18Rik |
| 1432626_at | Lgx only | 132 | 227 | 247 | 311 | 1.72 | 1.87 | 2.36 | 5730507A11Rik |
| 1432648_at | Res only | 67 | 77 | 17 | 67 | 1.16 | −4.01 | 1.01 | 4930466F19Rik |
| 1432662_at | Res only | 37 | 77 | 112 | 109 | 2.11 | 3.05 | 2.97 | 0610042E11Rik |
| 1432735_at | Lgx only | 256 | 258 | 314 | 474 | 1.01 | 1.23 | 1.86 | 1700017H01Rik |
| 1432930_at | Res only | 200 | 222 | 294 | 287 | 1.11 | 1.47 | 1.43 | 4930453O09Rik |
| 1433048_at | CR only | 185 | 107 | 139 | 196 | −1.74 | −1.33 | 1.06 | 4933428L01Rik |
| 1433132_at | CR only | 86 | 27 | 80 | 127 | −3.21 | −1.07 | 1.47 | Edaradd |
| 1433148_at | CR only | 226 | 484 | 386 | 319 | 2.14 | 1.71 | 1.41 | 4930513N20Rik |
| 1433164_at | Res only | 54 | 99 | 180 | 154 | 1.82 | 3.31 | 2.83 | 4930570E01Rik |
| 1433207_at | Lgx only | 57 | 67 | 66 | 136 | 1.17 | 1.14 | 2.37 | 5033430J17Rik |
| 1433241_at | Lgx only | 142 | 102 | 87 | 51 | −1.39 | −1.63 | −2.79 | 9430013L17Rik |
| 1433253_at | Lgx only | 40 | 88 | 74 | 128 | 2.21 | 1.86 | 3.24 | 5033423K11Rik |
| 1433314_at | Lgx only | 77 | 51 | 77 | 21 | −1.52 | 1.00 | −3.65 | 4930486A15Rik |
| 1433442_at | Lgx only | 2641 | 2244 | 1952 | 1694 | −1.18 | −1.35 | −1.56 | Klhl9 |
| 1433464_at | Lgx only | 1806 | 2481 | 2178 | 2563 | 1.37 | 1.21 | 1.42 | Ipo13 |
| 1433503_at | Res & Lgx | 5903 | 5778 | 4609 | 4467 | −1.02 | −1.28 | −1.32 | Zadh1 |
| 1433504_at | Lgx only | 18431 | 20243 | 22647 | 25548 | 1.10 | 1.23 | 1.39 | Pygb |
| 1433514_at | Lgx only | 1169 | 1037 | 957 | 909 | −1.13 | −1.22 | −1.29 | Etnk1 |
| 1433518_at | Lgx only | 529 | 567 | 612 | 674 | 1.07 | 1.16 | 1.27 | Lcmt2 |
| 1433519_at | Lgx only | 2411 | 2000 | 2022 | 1774 | −1.21 | −1.19 | −1.36 | Nucks1 |
| 1433520_at | Lgx only | 1068 | 1427 | 1464 | 1816 | 1.34 | 1.37 | 1.70 | Scap |
| 1433521_at | Res & Lgx | 1059 | 954 | 812 | 613 | −1.11 | −1.31 | −1.73 | Ankrd13c |
| 1433522_at | Lgx only | 465 | 508 | 536 | 616 | 1.09 | 1.15 | 1.32 | Pskh1 |
| 1433528_at | Lgx only | 1899 | 1634 | 1420 | 1242 | −1.16 | −1.34 | −1.53 | Gtf2a2 |
| 1433537_at | Lgx only | 1183 | 1089 | 1042 | 737 | −1.09 | −1.14 | −1.60 | 4833408C14Rik |
| 1433539_at | Res & Lgx | 2660 | 2213 | 1775 | 1485 | −1.20 | −1.50 | −1.79 | Commd3 |
| 1433544_at | Lgx only | 1808 | 1547 | 1540 | 1070 | −1.17 | −1.17 | −1.69 | Als2cr2 |
| 1433555_at | Lgx only | 416 | 379 | 412 | 237 | −1.10 | −1.01 | −1.75 | Eaf1 |
| 1433556_at | Res & Lgx | 138 | 182 | 263 | 232 | 1.32 | 1.90 | 1.68 | Centa1 |
| 1433561_at | Lgx only | 660 | 543 | 419 | 307 | −1.22 | −1.58 | −2.15 | Centb2 |
| 1433575_at | Lgx only | 3214 | 2986 | 2497 | 1537 | −1.08 | −1.29 | −2.09 | Sox4 |
| 1433585_at | Lgx only | 1024 | 898 | 841 | 644 | −1.14 | −1.22 | −1.59 | Tnpo1 |
| 1433597_at | Res & Lgx | 3769 | 3787 | 2160 | 1597 | 1.00 | −1.75 | −2.36 | 9430010O03Rik |
| 1433599_at | Lgx only | 331 | 323 | 351 | 222 | −1.03 | 1.06 | −1.49 | Baz1a |
| 1433645_at | Lgx only | 1238 | 1288 | 985 | 887 | 1.04 | −1.26 | −1.40 | Slc44a1 |
| 1433648_at | Lgx only | 3984 | 3473 | 3149 | 2552 | −1.15 | −1.26 | −1.56 | Spag9 |
| 1433656_a_at | Lgx only | 1182 | 1107 | 1036 | 879 | −1.07 | −1.14 | −1.34 | Gnl3 |
| 1433664_at | Res & Lgx | 664 | 522 | 428 | 377 | −1.27 | −1.55 | −1.76 | Ube2q2 |
| 1433676_at | Res & Lgx | 12728 | 12400 | 8809 | 8342 | −1.03 | −1.44 | −1.53 | Wnk1 |
| 1433682_at | Lgx only | 618 | 849 | 914 | 1124 | 1.37 | 1.48 | 1.82 | Arhgef17 |
| 1433686_at | Lgx only | 489 | 533 | 546 | 724 | 1.09 | 1.12 | 1.48 | Cabin1 |
| 1433691_at | Res & Lgx | 10244 | 10216 | 6379 | 5292 | −1.00 | −1.61 | −1.94 | Ppp1r3c |
| 1433698_a_at | Res & Lgx | 2618 | 2633 | 2921 | 3059 | 1.01 | 1.12 | 1.17 | Txnl4 |
| 1433700_at | Res & Lgx | 1009 | 844 | 673 | 749 | −1.19 | −1.50 | −1.35 | 4933433P14Rik |
| 1433705_at | Lgx only | 300 | 269 | 253 | 170 | −1.11 | −1.19 | −1.77 | Zfp213 |
| 1433712_at | Lgx only | 362 | 437 | 443 | 538 | 1.20 | 1.22 | 1.48 | AW555464 |
| 1433717_at | All | 1355 | 1876 | 1743 | 2058 | 1.38 | 1.29 | 1.52 | D19Wsu162e |
| 1433718_a_at | Lgx only | 862 | 744 | 773 | 554 | −1.16 | −1.12 | −1.56 | LOC100047028 |
| 1433722_at | Lgx only | 1946 | 2601 | 2972 | 3714 | 1.34 | 1.53 | 1.91 | Akap13 |
| 1433725_at | Res & Lgx | 840 | 1078 | 1085 | 1123 | 1.28 | 1.29 | 1.34 | Acvr1b |
| 1433727_at | Lgx only | 79 | 101 | 90 | 160 | 1.28 | 1.15 | 2.03 | BC038479 |
| 1433733_a_at | Lgx only | 179 | 226 | 163 | 115 | 1.26 | −1.10 | −1.55 | Cry1 |
| 1433738_at | Res & Lgx | 1127 | 966 | 802 | 666 | −1.17 | −1.40 | −1.69 | Papd5 |
| 1433741_at | Lgx only | 877 | 693 | 635 | 628 | −1.26 | −1.38 | −1.40 | Cd38 |
| 1433746_at | Lgx only | 484 | 409 | 361 | 275 | −1.18 | −1.34 | −1.76 | Wdr3 |
| 1433751_at | Lgx only | 580 | 428 | 435 | 248 | −1.36 | −1.33 | −2.34 | Slc39a10 |
| 1433760_a_at | Lgx only | 732 | 943 | 926 | 1027 | 1.29 | 1.26 | 1.40 | Rhbdd3 |
| 1433765_at | Lgx only | 521 | 587 | 784 | 834 | 1.13 | 1.50 | 1.60 | Ube2o |
| 1433770_at | Lgx only | 4705 | 4826 | 3514 | 3354 | 1.03 | −1.34 | −1.40 | Dpysl2 |
| 1433772_at | Res & Lgx | 820 | 636 | 421 | 437 | −1.29 | −1.95 | −1.87 | Stch |
| 1433790_at | CR only | 350 | 155 | 281 | 350 | −2.26 | −1.25 | −1.00 | Troap |
| 1433794_at | Lgx only | 1506 | 1268 | 1106 | 881 | −1.19 | −1.36 | −1.71 | Setx |
| 1433799_at | Lgx only | 877 | 735 | 582 | 537 | −1.19 | −1.51 | −1.63 | Rdh13 |
| 1433808_at | Lgx only | 912 | 1110 | 1207 | 1392 | 1.22 | 1.32 | 1.53 | D330001F17Rik |
| 1433811_at | Res & Lgx | 1795 | 2006 | 2548 | 2534 | 1.12 | 1.42 | 1.41 | Mllt6 |
| 1433847_at | Lgx only | 747 | 626 | 504 | 348 | −1.19 | −1.48 | −2.15 | D330017J20Rik |
| 1433868_at | Lgx only | 702 | 663 | 569 | 482 | −1.06 | −1.23 | −1.46 | Btbd3 |
| 1433875_at | Lgx only | 1471 | 1304 | 1208 | 1141 | −1.13 | −1.22 | −1.29 | 4732418C07Rik |
| 1433883_at | Lgx only | 3597 | 3657 | 2717 | 2316 | 1.02 | −1.32 | −1.55 | Tpm4 |
| 1433891_at | Res & Lgx | 1002 | 916 | 685 | 639 | −1.09 | −1.46 | −1.57 | Lgr4 |
| 1433897_at | Res & Lgx | 1769 | 1483 | 1048 | 773 | −1.19 | −1.69 | −2.29 | Al597468 |
| 1433898_at | Lgx only | 236 | 213 | 201 | 140 | −1.11 | −1.18 | −1.69 | AV025504 |
| 1433910_at | Lgx only | 1523 | 1421 | 1465 | 1231 | −1.07 | −1.04 | −1.24 | Zcchc6 |
| 1433914_at | Res & Lgx | 589 | 442 | 369 | 340 | −1.33 | −1.60 | −1.73 | Al747699 |
| 1433918_at | Res & Lgx | 1665 | 1645 | 1006 | 852 | −1.01 | −1.65 | −1.95 | Atg4d |
| 1433922_at | Res & Lgx | 1341 | 1190 | 903 | 781 | −1.13 | −1.48 | −1.72 | Rab35 |
| 1433926_at | Res & Lgx | 1929 | 1830 | 1548 | 1580 | −1.05 | −1.25 | −1.22 | Dync1li2 |
| 1433931_at | Lgx only | 1207 | 1431 | 1706 | 2152 | 1.18 | 1.41 | 1.78 | C030046I01Rik |
| 1433952_at | Res & Lgx | 1764 | 2403 | 2540 | 2663 | 1.36 | 1.44 | 1.51 | Tufm |
| 1433953_at | Lgx only | 1145 | 1007 | 993 | 868 | −1.14 | −1.15 | −1.32 | Zfp277 |
| 1433979_at | Lgx only | 1885 | 2263 | 1938 | 2261 | 1.20 | 1.03 | 1.20 | Rbms2 |
| 1433986_at | Lgx only | 2432 | 2354 | 2145 | 2176 | −1.03 | −1.13 | −1.12 | BC024659 |
| 1434001_at | Res & Lgx | 591 | 503 | 394 | 398 | −1.17 | −1.50 | −1.48 | Ttc9c |
| 1434008_at | Lgx only | 1391 | 940 | 1006 | 885 | −1.48 | −1.38 | −1.57 | Scn4b |
| 1434009_at | Res & Lgx | 624 | 786 | 1028 | 1225 | 1.26 | 1.65 | 1.96 | Grlf1 |
| 1434018_at | Lgx only | 996 | 1035 | 1127 | 1368 | 1.04 | 1.13 | 1.37 | BC043098 |
| 1434059_at | Lgx only | 519 | 522 | 671 | 756 | 1.00 | 1.29 | 1.46 | B230312A22Rik |
| 1434064_at | Res & Lgx | 474 | 589 | 715 | 770 | 1.24 | 1.51 | 1.62 | Tmem142c |
| 1434066_at | Lgx only | 807 | 953 | 1127 | 1361 | 1.18 | 1.40 | 1.69 | Gtf3c1 |
| 1434067_at | Res only | 21 | 38 | 74 | 62 | 1.83 | 3.54 | 2.96 | Al662270 |
| 1434072_at | Lgx only | 893 | 774 | 728 | 646 | −1.15 | −1.23 | −1.38 | Smcr7 |
| 1434075_at | Res & Lgx | 1023 | 923 | 673 | 623 | −1.11 | −1.52 | −1.64 | BC030336 |
| 1434082_at | Lgx only | 1059 | 957 | 940 | 850 | −1.11 | −1.13 | −1.25 | Pctk2 |
| 1434084_at | Lgx only | 536 | 417 | 380 | 265 | −1.28 | −1.41 | −2.02 | 5730601F06Rik |
| 1434088_at | Lgx only | 814 | 957 | 921 | 1043 | 1.18 | 1.13 | 1.28 | Zkscan17 |
| 1434096_at | Lgx only | 760 | 778 | 873 | 1035 | 1.02 | 1.15 | 1.36 | Slc4a4 |
| 1434105_at | Lgx only | 1682 | 1795 | 2023 | 2368 | 1.07 | 1.20 | 1.41 | Epm2aip1 |
| 1434115_at | Lgx only | 3429 | 4304 | 3972 | 4713 | 1.26 | 1.16 | 1.37 | Cdh13 |
| 1434131_at | Lgx only | 1087 | 1126 | 1252 | 1367 | 1.04 | 1.15 | 1.26 | Rufy1 |
| 1434135_at | Lgx only | 634 | 615 | 511 | 451 | −1.03 | −1.24 | −1.40 | B3galnt2 |
| 1434138_at | Lgx only | 910 | 747 | 789 | 685 | −1.22 | −1.15 | −1.33 | Prune |
| 1434140_at | CR only | 1207 | 1781 | 1342 | 1311 | 1.48 | 1.11 | 1.09 | Mcf2l |
| 1434153_at | CR & Lgx | 845 | 1513 | 953 | 1127 | 1.79 | 1.13 | 1.33 | Shb |
| 1434174_at | Res only | 619 | 536 | 463 | 444 | −1.16 | −1.34 | −1.40 | Lysmd3 |
| 1434200_at | Res & Lgx | 604 | 514 | 322 | 304 | −1.17 | −1.88 | −1.99 | BC010981 |
| 1434205_at | Res & Lgx | 3650 | 3118 | 2521 | 1621 | −1.17 | −1.45 | −2.25 | Ppp2r5c |
| 1434234_at | Lgx only | 23 | 72 | 52 | 115 | 3.07 | 2.22 | 4.92 | Zfp341 |
| 1434238_at | Lgx only | 417 | 408 | 282 | 247 | −1.02 | −1.48 | −1.69 | Taf2 |
| 1434248_at | Res only | 842 | 810 | 1061 | 848 | −1.04 | 1.26 | 1.01 | Prkch |
| 1434268_at | Lgx only | 761 | 830 | 965 | 1097 | 1.09 | 1.27 | 1.44 | Adar |
| 1434271_at | Res & Lgx | 520 | 568 | 711 | 747 | 1.09 | 1.37 | 1.44 | Gba2 |
| 1434273_at | Lgx only | 11070 | 12667 | 12227 | 13559 | 1.14 | 1.10 | 1.22 | BC034069 |
| 1434277_a_at | CR only | 774 | 1200 | 842 | 881 | 1.55 | 1.09 | 1.14 | Ypel2 |
| 1434278_at | Lgx only | 1459 | 881 | 1015 | 473 | −1.66 | −1.44 | −3.09 | Mtm1 |
| 1434281_at | Lgx only | 1018 | 999 | 812 | 698 | −1.02 | −1.25 | −1.46 | 1500034J01Rik |
| 1434283_at | Res & Lgx | 893 | 1206 | 1278 | 1477 | 1.35 | 1.43 | 1.65 | LOC100044968 |
| 1434284_at | Res & Lgx | 829 | 710 | 613 | 588 | −1.17 | −1.35 | −1.41 | Bdp1 |
| 1434296_at | Lgx only | 291 | 265 | 237 | 224 | −1.10 | −1.23 | −1.30 | BC049349 |
| 1434303_at | Lgx only | 1077 | 1189 | 2323 | 2458 | 1.10 | 2.16 | 2.28 | Raph1 |
| 1434320_at | Lgx only | 512 | 413 | 369 | 377 | −1.24 | −1.39 | −1.36 | Gtf3c4 |
| 1434328_at | Lgx only | 2487 | 2492 | 2004 | 1703 | 1.00 | −1.24 | −1.46 | Rpl15 |
| 1434339_at | Res & Lgx | 966 | 835 | 676 | 499 | −1.16 | −1.43 | −1.94 | Fnbp1l |
| 1434344_at | Lgx only | 1331 | 1208 | 1085 | 944 | −1.10 | −1.23 | −1.41 | Gpkow |
| 1434354_at | Lgx only | 3473 | 3028 | 2675 | 2382 | −1.15 | −1.30 | −1.46 | Maob |
| 1434356_a_at | Lgx only | 6321 | 5829 | 5156 | 4746 | −1.08 | −1.23 | −1.33 | Psma5 |
| 1434372_at | Lgx only | 2170 | 1834 | 1771 | 1504 | −1.18 | −1.23 | −1.44 | AW112010 |
| 1434378_a_at | Lgx only | 952 | 996 | 1039 | 1163 | 1.05 | 1.09 | 1.22 | Mxd4 |
| 1434387_at | Lgx only | 2313 | 2828 | 3081 | 3362 | 1.22 | 1.33 | 1.45 | Itfg3 |
| 1434392_at | Res & Lgx | 1484 | 1245 | 1133 | 925 | −1.19 | −1.31 | −1.61 | Usp34 |
| 1434402_at | Lgx only | 683 | 631 | 619 | 432 | −1.08 | −1.10 | −1.58 | Samd8 |
| 1434405_at | Res & Lgx | 1119 | 991 | 685 | 678 | −1.13 | −1.63 | −1.65 | Fnip1 |
| 1434422_at | CR & Lgx | 232 | 140 | 152 | 120 | −1.66 | −1.53 | −1.94 | AI428479 |
| 1434441_at | Lgx only | 1752 | 1522 | 1369 | 1299 | −1.15 | −1.28 | −1.35 | 1110018J18Rik |
| 1434442_at | CR only | 391 | 549 | 429 | 449 | 1.40 | 1.10 | 1.15 | Stbd1 |
| 1434461_at | Res & Lgx | 1215 | 1185 | 926 | 822 | −1.03 | −1.31 | −1.48 | Zfp715 |
| 1434487_at | Lgx only | 1586 | 1697 | 2179 | 2499 | 1.07 | 1.37 | 1.58 | Mef2d |
| 1434493_at | Lgx only | 158 | 164 | 101 | 106 | 1.03 | −1.57 | −1.50 | 1810022K09Rik |
| 1434511_at | Lgx only | 1331 | 1195 | 1108 | 920 | −1.11 | −1.20 | −1.45 | Phkb |
| 1434516_at | Lgx only | 1502 | 1422 | 1254 | 1162 | −1.06 | −1.20 | −1.29 | Pstk |
| 1434518_at | Res & Lgx | 918 | 972 | 1104 | 1186 | 1.06 | 1.20 | 1.29 | Phka2 |
| 1434558_at | Lgx only | 280 | 256 | 227 | 155 | −1.09 | −1.23 | −1.81 | Wdr47 |
| 1434565_at | Res & Lgx | 1072 | 1078 | 773 | 729 | 1.01 | −1.39 | −1.47 | Cgrrf1 |
| 1434586_a_at | Lgx only | 2001 | 1938 | 1529 | 1209 | −1.03 | −1.31 | −1.66 | Ptdss2 |
| 1434597_at | Lgx only | 2702 | 3318 | 3044 | 3746 | 1.23 | 1.13 | 1.39 | Larp5 |
| 1434604_at | Lgx only | 1414 | 1229 | 1298 | 1039 | −1.15 | −1.09 | −1.36 | Eif5b |
| 1434610_at | Res & Lgx | 2684 | 3974 | 4970 | 6496 | 1.48 | 1.85 | 2.42 | Plec1 |
| 1434613_at | Lgx only | 2786 | 2883 | 1811 | 1538 | 1.03 | −1.54 | −1.81 | 1810013L24Rik |
| 1434625_at | Lgx only | 882 | 647 | 703 | 609 | −1.36 | −1.25 | −1.45 | 4930432O21Rik |
| 1434642_at | Res & Lgx | 3093 | 2964 | 4527 | 4450 | −1.04 | 1.46 | 1.44 | Hsd17b11 |
| 1434647_at | Lgx only | 754 | 584 | 603 | 458 | −1.29 | −1.25 | −1.65 | Egflam |
| 1434648_a_at | Lgx only | 900 | 688 | 589 | 361 | −1.31 | −1.53 | −2.50 | Ccm2 |
| 1434655_at | Lgx only | 206 | 244 | 274 | 363 | 1.18 | 1.33 | 1.76 | Foxk1 |
| 1434665_at | Lgx only | 1767 | 1845 | 1861 | 2289 | 1.04 | 1.05 | 1.30 | Aga |
| 1434671_at | Lgx only | 1246 | 959 | 1311 | 840 | −1.30 | 1.05 | −1.48 | B230337E12Rik |
| 1434672_at | Lgx only | 1613 | 1191 | 1046 | 779 | −1.35 | −1.54 | −2.07 | Gpr22 |
| 1434714_at | Lgx only | 227 | 243 | 341 | 399 | 1.07 | 1.50 | 1.76 | Ero1lb |
| 1434736_at | Lgx only | 894 | 1036 | 1010 | 1326 | 1.16 | 1.13 | 1.48 | Hlf |
| 1434765_at | Lgx only | 788 | 738 | 602 | 509 | −1.07 | −1.31 | −1.55 | Ep300 |
| 1434775_at | Lgx only | 573 | 537 | 690 | 773 | −1.07 | 1.20 | 1.35 | Pard3 |
| 1434791_at | Lgx only | 746 | 909 | 934 | 1128 | 1.22 | 1.25 | 1.51 | Atp6v0a2 |
| 1434792_at | Lgx only | 639 | 423 | 604 | 401 | −1.51 | −1.06 | −1.60 | 2010320M18Rik |
| 1434805_at | Lgx only | 439 | 529 | 521 | 670 | 1.20 | 1.19 | 1.53 | Mllt1 |
| 1434822_at | Lgx only | 1213 | 1095 | 1009 | 970 | −1.11 | −1.20 | −1.25 | Pphln1 |
| 1434824_at | Lgx only | 1740 | 2163 | 2205 | 2627 | 1.24 | 1.27 | 1.51 | Baz1b |
| 1434826_at | Lgx only | 548 | 525 | 447 | 344 | −1.04 | −1.23 | −1.59 | Rfesd |
| 1434835_at | Lgx only | 1066 | 952 | 926 | 824 | −1.12 | −1.15 | −1.29 | Wapal |
| 1434838_at | Lgx only | 4161 | 4913 | 5731 | 5398 | 1.18 | 1.38 | 1.30 | Kcng2 |
| 1434864_at | Res & Lgx | 690 | 688 | 467 | 358 | −1.00 | −1.48 | −1.93 | Nipa1 |
| 1434891_at | Lgx only | 949 | 1098 | 1092 | 1555 | 1.16 | 1.15 | 1.64 | Ptgfrn |
| 1434896_at | Res & Lgx | 968 | 863 | 618 | 562 | −1.12 | −1.57 | −1.72 | Zfp422-rs1 |
| 1434900_at | Lgx only | 265 | 301 | 367 | 406 | 1.13 | 1.38 | 1.53 | Mkl1 |
| 1434904_at | Res & Lgx | 462 | 569 | 661 | 898 | 1.23 | 1.43 | 1.94 | Hivep2 |
| 1434909_at | Res & Lgx | 2412 | 2355 | 1922 | 1743 | −1.02 | −1.26 | −1.38 | Rragd |
| 1434916_at | Lgx only | 470 | 406 | 406 | 314 | −1.16 | −1.16 | −1.50 | Vkorc1l1 |
| 1434927_at | Lgx only | 38292 | 44321 | 50306 | 62045 | 1.16 | 1.31 | 1.62 | Hspb7 |
| 1434930_at | Lgx only | 665 | 821 | 984 | 1031 | 1.23 | 1.48 | 1.55 | Tpcn1 |
| 1434934_at | Res & Lgx | 4033 | 3850 | 2890 | 2753 | −1.05 | −1.40 | −1.46 | Atpaf1 |
| 1434936_at | Lgx only | 374 | 413 | 491 | 592 | 1.10 | 1.31 | 1.58 | Hirip3 |
| 1434942_at | Res & Lgx | 901 | 696 | 666 | 528 | −1.30 | −1.35 | −1.71 | Esf1 |
| 1434944_at | Lgx only | 6233 | 7918 | 9727 | 9182 | 1.27 | 1.56 | 1.47 | Dmpk |
| 1434967_at | Res & Lgx | 535 | 436 | 358 | 324 | −1.23 | −1.49 | −1.65 | Zswim6 |
| 1434978_at | Lgx only | 1957 | 2176 | 1745 | 1353 | 1.11 | −1.12 | −1.45 | 4933403F05Rik |
| 1434981_at | Res only | 109 | 143 | 215 | 123 | 1.31 | 1.97 | 1.12 | E130303B06Rik |
| 1434997_at | Lgx only | 592 | 529 | 618 | 756 | −1.12 | 1.04 | 1.28 | Cdc2l6 |
| 1435016_at | Lgx only | 3394 | 3426 | 2781 | 2210 | 1.01 | −1.22 | −1.54 | Trak2 |
| 1435017_at | Lgx only | 3573 | 4157 | 4007 | 4259 | 1.16 | 1.12 | 1.19 | Mel13 |
| 1435018_at | Lgx only | 872 | 1011 | 949 | 1112 | 1.16 | 1.09 | 1.28 | 5930434B04Rik |
| 1435032_at | Lgx only | 747 | 869 | 992 | 1031 | 1.16 | 1.33 | 1.38 | Golgb1 |
| 1435091_at | Lgx only | 591 | 460 | 474 | 418 | −1.29 | −1.25 | −1.41 | Zfp568 |
| 1435117_a_at | Lgx only | 460 | 552 | 597 | 615 | 1.20 | 1.30 | 1.34 | Rbj |
| 1435135_at | Res & Lgx | 6817 | 7003 | 9784 | 10952 | 1.03 | 1.44 | 1.61 | Aadacl1 |
| 1435153_at | Lgx only | 764 | 784 | 688 | 591 | 1.03 | −1.11 | −1.29 | Btbd6 |
| 1435169_at | CR only | 1062 | 1377 | 1382 | 1273 | 1.30 | 1.30 | 1.20 | A930001N09Rik |
| 1435180_at | Lgx only | 460 | 729 | 770 | 1246 | 1.58 | 1.67 | 2.71 | Podn |
| 1435183_at | All | 452 | 596 | 827 | 928 | 1.32 | 1.83 | 2.05 | Tbkbp1 |
| 1435224_at | Lgx only | 1147 | 1340 | 1495 | 1677 | 1.17 | 1.30 | 1.46 | Crebbp |
| 1435242_at | Res & Lgx | 596 | 524 | 452 | 456 | −1.14 | −1.32 | −1.31 | Pds5b |
| 1435248_a_at | Lgx only | 793 | 736 | 625 | 445 | −1.08 | −1.27 | −1.78 | Btaf1 |
| 1435250_at | Res & Lgx | 509 | 498 | 329 | 263 | −1.02 | −1.55 | −1.93 | Ints8 |
| 1435261_at | Res & Lgx | 2639 | 2800 | 3398 | 4623 | 1.06 | 1.29 | 1.75 | 4732416N19Rik |
| 1435295_at | Res only | 394 | 484 | 605 | 566 | 1.23 | 1.54 | 1.44 | Dopey1 |
| 1435340_at | Lgx only | 409 | 634 | 611 | 728 | 1.55 | 1.49 | 1.78 | Jmjd2a |
| 1435349_at | Lgx only | 646 | 526 | 503 | 317 | −1.23 | −1.28 | −2.04 | Nrp2 |
| 1435351_at | CR only | 601 | 471 | 619 | 512 | −1.28 | 1.03 | −1.18 | 2310026E23Rik |
| 1435360_at | Lgx only | 1234 | 1322 | 1550 | 1828 | 1.07 | 1.26 | 1.48 | Zfp651 |
| 1435377_at | Res & Lgx | 561 | 417 | 320 | 302 | −1.34 | −1.75 | −1.86 | 2410002O22Rik |
| 1435378_at | Lgx only | 977 | 1013 | 1221 | 1341 | 1.04 | 1.25 | 1.37 | 2210020M01Rik |
| 1435386_at | Lgx only | 966 | 1325 | 1564 | 2231 | 1.37 | 1.62 | 2.31 | Vwf |
| 1435437_at | Lgx only | 1823 | 1595 | 1637 | 1411 | −1.14 | −1.11 | −1.29 | Setd7 |
| 1435441_at | Res only | 366 | 339 | 487 | 474 | −1.08 | 1.33 | 1.29 | Ablim2 |
| 1435442_at | Res & Lgx | 1660 | 1466 | 1220 | 1214 | −1.13 | −1.36 | −1.37 | Wdsof1 |
| 1435443_at | Lgx only | 715 | 866 | 940 | 998 | 1.21 | 1.31 | 1.40 | Eya3 |
| 1435461_at | Res & Lgx | 2568 | 2130 | 1753 | 1415 | −1.21 | −1.46 | −1.81 | Magi3 |
| 1435490_at | CR only | 110 | 246 | 176 | 195 | 2.23 | 1.60 | 1.77 | Hk3 |
| 1435505_at | Res only | 1051 | 1219 | 1346 | 1482 | 1.16 | 1.28 | 1.41 | Dmwd |
| 1435524_at | Lgx only | 1085 | 1246 | 1347 | 1415 | 1.15 | 1.24 | 1.30 | 2010109N14Rik |
| 1435526_at | Lgx only | 566 | 469 | 430 | 359 | −1.21 | −1.32 | −1.58 | Tor1aip2 |
| 1435527_at | Lgx only | 1442 | 1796 | 2081 | 2372 | 1.24 | 1.44 | 1.64 | Nfic |
| 1435529_at | Res & Lgx | 949 | 677 | 595 | 504 | −1.40 | −1.60 | −1.88 | OTTMUSG00000016644 |
| 1435543_at | Lgx only | 986 | 868 | 772 | 684 | −1.14 | −1.28 | −1.44 | LOC100048863 |
| 1435547_at | Lgx only | 1442 | 1468 | 1270 | 1189 | 1.02 | −1.14 | −1.21 | ENSMUSG00000075401 |
| 1435548_at | Lgx only | 726 | 703 | 596 | 466 | −1.03 | −1.22 | −1.56 | Mrs2l |
| 1435549_at | Lgx only | 434 | 440 | 504 | 549 | 1.01 | 1.16 | 1.26 | Trpm4 |
| 1435554_at | Res & Lgx | 1663 | 1372 | 1080 | 922 | −1.21 | −1.54 | −1.80 | Tmcc3 |
| 1435556_at | Lgx only | 530 | 463 | 373 | 373 | −1.15 | −1.42 | −1.42 | Zfp597 |
| 1435589_at | Res & Lgx | 510 | 650 | 831 | 1114 | 1.27 | 1.63 | 2.18 | Ccdc85b |
| 1435641_at | Res & Lgx | 437 | 400 | 308 | 300 | −1.09 | −1.42 | −1.45 | 9530018I07Rik |
| 1435655_at | Lgx only | 248 | 223 | 189 | 131 | −1.11 | −1.31 | −1.89 | Snora65 |
| 1435674_at | Lgx only | 361 | 478 | 368 | 510 | 1.33 | 1.02 | 1.41 | Rhobtb2 |
| 1435679_at | Lgx only | 1778 | 1866 | 1603 | 1228 | 1.05 | −1.11 | −1.45 | Optn |
| 1435693_at | CR only | 348 | 448 | 320 | 452 | 1.29 | −1.09 | 1.30 | Mall |
| 1435695_a_at | Lgx only | 876 | 756 | 620 | 449 | −1.16 | −1.41 | −1.95 | A030007L17Rik |
| 1435743_at | Res & Lgx | 987 | 808 | 671 | 634 | −1.22 | −1.47 | −1.56 | Klhl23 |
| 1435754_at | Res & Lgx | 1015 | 818 | 741 | 714 | −1.24 | −1.37 | −1.42 | Zyg11b |
| 1435768_at | Lgx only | 919 | 909 | 725 | 632 | −1.01 | −1.27 | −1.46 | Arid4b |
| 1435774_at | Lgx only | 487 | 394 | 360 | 292 | −1.24 | −1.35 | −1.67 | AV024533 |
| 1435777_at | Lgx only | 1189 | 995 | 853 | 715 | −1.19 | −1.39 | −1.66 | E030018N11Rik |
| 1435782_at | Lgx only | 166 | 244 | 291 | 295 | 1.47 | 1.75 | 1.78 | LOC668206 |
| 1435808_at | Res & Lgx | 579 | 704 | 924 | 1015 | 1.22 | 1.60 | 1.75 | A230051G13Rik |
| 1435813_at | Lgx only | 2373 | 3193 | 3181 | 3356 | 1.35 | 1.34 | 1.41 | Mypn |
| 1435864_a_at | Res & Lgx | 2251 | 2148 | 1526 | 1411 | −1.05 | −1.48 | −1.59 | 1810063B05Rik |
| 1435874_at | Lgx only | 1005 | 1045 | 939 | 727 | 1.04 | −1.07 | −1.38 | Prkab2 |
| 1435900_at | Res & Lgx | 775 | 768 | 643 | 518 | −1.01 | −1.20 | −1.49 | Zbtb43 |
| 1435912_at | Lgx only | 670 | 587 | 579 | 475 | −1.14 | −1.16 | −1.41 | Ubxd7 |
| 1435947_at | Lgx only | 647 | 572 | 495 | 456 | −1.13 | −1.31 | −1.42 | 2810455D13Rik |
| 1436014_a_at | CR only | 449 | 349 | 494 | 518 | −1.28 | 1.10 | 1.16 | Rusc1 |
| 1436026_at | Lgx only | 476 | 418 | 620 | 736 | −1.14 | 1.30 | 1.54 | Zfp703 |
| 1436033_at | Lgx only | 879 | 984 | 1031 | 1238 | 1.12 | 1.17 | 1.41 | BC031353 |
| 1436041_at | CR & Lgx | 1831 | 1425 | 1788 | 1414 | −1.28 | −1.02 | −1.29 | LOC100046086 |
| 1436045_at | Lgx only | 381 | 321 | 282 | 216 | −1.19 | −1.35 | −1.76 | Tsga10 |
| 1436059_at | Res & Lgx | 254 | 366 | 397 | 446 | 1.44 | 1.56 | 1.75 | Rfx1 |
| 1436075_at | Lgx only | 280 | 256 | 422 | 622 | −1.09 | 1.51 | 2.22 | Sfrp5 |
| 1436081_a_at | Lgx only | 1053 | 1205 | 1255 | 1434 | 1.14 | 1.19 | 1.36 | Zfp414 |
| 1436112_at | Res only | 158 | 196 | 325 | 246 | 1.24 | 2.06 | 1.55 | AI118078 |
| 1436113_a_at | Res only | 2773 | 2845 | 2148 | 2176 | 1.03 | −1.29 | −1.27 | St13 |
| 1436121_a_at | Lgx only | 1870 | 1812 | 1483 | 1122 | −1.03 | −1.26 | −1.67 | Nsmce1 |
| 1436122_at | Res only | 242 | 299 | 396 | 310 | 1.24 | 1.64 | 1.28 | Zfp667 |
| 1436157_at | Lgx only | 1826 | 1496 | 1478 | 1265 | −1.22 | −1.24 | −1.44 | Ccar1 |
| 1436188_a_at | CR only | 7033 | 11508 | 7677 | 9091 | 1.64 | 1.09 | 1.29 | Ndrg4 |
| 1436208_at | CR & Lgx | 551 | 716 | 663 | 794 | 1.30 | 1.20 | 1.44 | Asb1 |
| 1436214_at | Lgx only | 567 | 465 | 395 | 293 | −1.22 | −1.44 | −1.93 | 1110028C15Rik |
| 1436215_at | Lgx only | 642 | 508 | 549 | 433 | −1.26 | −1.17 | −1.48 | Ipmk |
| 1436233_at | Lgx only | 1039 | 871 | 997 | 736 | −1.19 | −1.04 | −1.41 | Btnl9 |
| 1436240_at | Lgx only | 382 | 383 | 308 | 256 | 1.00 | −1.24 | −1.49 | Sost |
| 1436243_at | Lgx only | 2945 | 3472 | 2166 | 1917 | 1.18 | −1.36 | −1.54 | Frmd5 |
| 1436275_at | Lgx only | 5589 | 6926 | 4236 | 4096 | 1.24 | −1.32 | −1.36 | Kcnip2 |
| 1436299_at | Res & Lgx | 1490 | 1306 | 1113 | 877 | −1.14 | −1.34 | −1.70 | Gls |
| 1436310_at | Lgx only | 199 | 301 | 245 | 313 | 1.51 | 1.23 | 1.57 | Gemin5 |
| 1436332_at | Lgx only | 32378 | 38509 | 38078 | 46348 | 1.19 | 1.18 | 1.43 | Hspb6 |
| 1436339_at | Lgx only | 6840 | 6976 | 10138 | 9417 | 1.02 | 1.48 | 1.38 | 1810058I24Rik |
| 1436342_a_at | Lgx only | 1693 | 1473 | 1337 | 1170 | −1.15 | −1.27 | −1.45 | D19Ertd721e |
| 1436367_at | Lgx only | 3325 | 3749 | 4038 | 4164 | 1.13 | 1.21 | 1.25 | C130094E24 |
| 1436377_at | Res only | 595 | 716 | 830 | 810 | 1.20 | 1.40 | 1.36 | Gpr137 |
| 1436408_at | Lgx only | 88 | 45 | 76 | 29 | −1.95 | −1.16 | −3.02 | Rprml |
| 1436425_at | Lgx only | 182 | 121 | 125 | 126 | −1.50 | −1.45 | −1.45 | Ankrd38 |
| 1436446_at | Res & Lgx | 675 | 568 | 441 | 416 | −1.19 | −1.53 | −1.62 | 2310007O11Rik |
| 1436505_at | CR only | 722 | 511 | 816 | 739 | −1.41 | 1.13 | 1.02 | Ppig |
| 1436511_at | Res & Lgx | 675 | 634 | 510 | 460 | −1.06 | −1.32 | −1.47 | BC031781 |
| 1436521_at | Lgx only | 995 | 1371 | 1104 | 1496 | 1.38 | 1.11 | 1.50 | Slc36a2 |
| 1436537_at | CR only | 624 | 518 | 554 | 573 | −1.20 | −1.13 | −1.09 | Zfp629 |
| 1436538_at | Res & Lgx | 425 | 320 | 201 | 116 | −1.33 | −2.12 | −3.68 | Ankrd37 |
| 1436546_at | Res & Lgx | 1262 | 1105 | 994 | 881 | −1.14 | −1.27 | −1.43 | Lix1l |
| 1436547_at | CR only | 380 | 285 | 282 | 352 | −1.33 | −1.34 | −1.08 | Dgke |
| 1436562_at | Lgx only | 579 | 545 | 498 | 363 | −1.06 | −1.16 | −1.59 | Ddx58 |
| 1436584_at | Res & Lgx | 825 | 721 | 541 | 545 | −1.15 | −1.53 | −1.51 | Spry2 |
| 1436594_at | Lgx only | 180 | 124 | 152 | 103 | −1.45 | −1.18 | −1.74 | Zfp719 |
| 1436609_a_at | Lgx only | 4537 | 4960 | 5297 | 5655 | 1.09 | 1.17 | 1.25 | Lrpap1 |
| 1436650_at | CR & Lgx | 729 | 1049 | 944 | 1072 | 1.44 | 1.30 | 1.47 | Filip1 |
| 1436665_a_at | Lgx only | 3944 | 5547 | 5624 | 7673 | 1.41 | 1.43 | 1.95 | Ltbp4 |
| 1436739_at | Lgx only | 2627 | 2725 | 2561 | 2047 | 1.04 | −1.03 | −1.28 | Agtr1a |
| 1436747_at | Lgx only | 2419 | 2508 | 2836 | 3129 | 1.04 | 1.17 | 1.29 | 1110014K08Rik |
| 1436797_a_at | Lgx only | 1519 | 1814 | 1790 | 2289 | 1.19 | 1.18 | 1.51 | Surf4 |
| 1436809_a_at | All | 1520 | 2518 | 2464 | 2994 | 1.66 | 1.62 | 1.97 | Spin1 |
| 1436817_at | Lgx only | 1182 | 1030 | 929 | 752 | −1.15 | −1.27 | −1.57 | Exoc5 |
| 1436842_at | Res & Lgx | 3911 | 3696 | 2482 | 2369 | −1.06 | −1.58 | −1.65 | B230380D07Rik |
| 1436844_at | Lgx only | 536 | 431 | 451 | 371 | −1.24 | −1.19 | −1.44 | AW046287 |
| 1436865_at | CR only | 308 | 173 | 311 | 209 | −1.78 | 1.01 | −1.47 | Slc26a11 |
| 1436867_at | CR only | 23230 | 28014 | 23888 | 25409 | 1.21 | 1.03 | 1.09 | Srl |
| 1436883_at | Lgx only | 558 | 425 | 421 | 399 | −1.31 | −1.32 | −1.40 | Mbtps2 |
| 1436918_at | Lgx only | 857 | 823 | 801 | 562 | −1.04 | −1.07 | −1.53 | LOC100044376 |
| 1436947_a_at | Lgx only | 4903 | 4267 | 3399 | 2703 | −1.15 | −1.44 | −1.81 | Txnl1 |
| 1436984_at | Lgx only | 752 | 734 | 574 | 497 | −1.02 | −1.31 | −1.51 | Abi2 |
| 1436985_at | Res & Lgx | 1800 | 1660 | 1487 | 1422 | −1.08 | −1.21 | −1.27 | Zfp644 |
| 1436999_at | Res only | 619 | 512 | 464 | 480 | −1.21 | −1.33 | −1.29 | 5033414K04Rik |
| 1437026_at | Lgx only | 472 | 457 | 287 | 194 | −1.03 | −1.65 | −2.44 | BC057893 |
| 1437069_at | Lgx only | 1632 | 1471 | 1282 | 972 | −1.11 | −1.27 | −1.68 | Osbpl8 |
| 1437077_at | Res & Lgx | 2086 | 1987 | 1493 | 1649 | −1.05 | −1.40 | −1.26 | Dcun1d2 |
| 1437092_at | Res & Lgx | 535 | 440 | 282 | 286 | −1.22 | −1.90 | −1.87 | LOC100048376 |
| 1437111_at | Lgx only | 474 | 441 | 430 | 320 | −1.08 | −1.10 | −1.48 | Zc3h12c |
| 1437136_at | Res only | 60 | 130 | 240 | 141 | 2.17 | 4.00 | 2.35 | 5830436I19Rik |
| 1437143_a_at | All | 4498 | 3397 | 2547 | 1894 | −1.32 | −1.77 | −2.38 | Txndc1 |
| 1437148_at | CR & Lgx | 7652 | 9004 | 8835 | 9409 | 1.18 | 1.15 | 1.23 | Arpc2 |
| 1437149_at | Lgx only | 2370 | 2826 | 3182 | 3347 | 1.19 | 1.34 | 1.41 | Slc6a6 |
| 1437151_at | Lgx only | 1757 | 1972 | 2085 | 2237 | 1.12 | 1.19 | 1.27 | Usp22 |
| 1437216_at | Lgx only | 363 | 289 | 295 | 210 | −1.25 | −1.23 | −1.73 | Ccdc88a |
| 1437236_a_at | Lgx only | 449 | 348 | 371 | 302 | −1.29 | −1.21 | −1.49 | Zfp110 |
| 1437241_at | CR only | 760 | 1025 | 755 | 926 | 1.35 | −1.01 | 1.22 | Klf11 |
| 1437283_at | Lgx only | 376 | 462 | 480 | 662 | 1.23 | 1.28 | 1.76 | Tnpo2 |
| 1437287_at | Res & Lgx | 2217 | 1647 | 1611 | 1014 | −1.35 | −1.38 | −2.19 | 1110020G09Rik |
| 1437290_at | Lgx only | 1027 | 946 | 711 | 722 | −1.09 | −1.44 | −1.42 | Impad1 |
| 1437354_at | Res & Lgx | 1278 | 1069 | 925 | 811 | −1.20 | −1.38 | −1.58 | C230091D08Rik |
| 1437382_at | Lgx only | 1211 | 1154 | 1069 | 922 | −1.05 | −1.13 | −1.31 | Acvr2a |
| 1437394_at | Lgx only | 343 | 383 | 446 | 567 | 1.12 | 1.30 | 1.65 | Centg2 |
| 1437397_at | Res & Lgx | 277 | 210 | 181 | 160 | −1.32 | −1.53 | −1.73 | Prlr |
| 1437398_a_at | All | 781 | 1052 | 1289 | 1566 | 1.35 | 1.65 | 2.01 | Aldh9a1 |
| 1437403_at | Lgx only | 41 | 70 | 69 | 98 | 1.72 | 1.69 | 2.41 | Samd5 |
| 1437405_a_at | Lgx only | 5956 | 7647 | 8743 | 11879 | 1.28 | 1.47 | 1.99 | Igfbp4 |
| 1437426_at | Lgx only | 275 | 200 | 273 | 162 | −1.37 | −1.01 | −1.70 | Wac |
| 1437432_a_at | Res only | 220 | 153 | 98 | 165 | −1.44 | −2.25 | −1.33 | Trim12 |
| 1437442_at | Res & Lgx | 1223 | 963 | 774 | 621 | −1.27 | −1.58 | −1.97 | Pcdh7 |
| 1437449_at | Lgx only | 727 | 595 | 602 | 560 | −1.22 | −1.21 | −1.30 | Rsad1 |
| 1437482_at | Res & Lgx | 7128 | 6639 | 4396 | 3270 | −1.07 | −1.62 | −2.18 | Srd5a2l2 |
| 1437484_at | Res & Lgx | 321 | 372 | 527 | 541 | 1.16 | 1.64 | 1.68 | Zbtb5 |
| 1437513_a_at | Lgx only | 5779 | 5896 | 6781 | 7125 | 1.02 | 1.17 | 1.23 | Serinc1 |
| 1437533_at | Res & Lgx | 2000 | 1894 | 1502 | 1237 | −1.06 | −1.33 | −1.62 | Birc4 |
| 1437537_at | Res & Lgx | 249 | 339 | 340 | 398 | 1.36 | 1.36 | 1.60 | Casp9 |
| 1437704_at | Res & Lgx | 194 | 157 | 376 | 378 | −1.23 | 1.93 | 1.94 | 2900024O10Rik |
| 1437729_at | Lgx only | 1474 | 1094 | 793 | 412 | −1.35 | −1.86 | −3.58 | Rpl27a |
| 1437740_at | Lgx only | 763 | 1122 | 1005 | 1343 | 1.47 | 1.32 | 1.76 | Plekhm2 |
| 1437741_at | Lgx only | 2327 | 2390 | 1778 | 1560 | 1.03 | −1.31 | −1.49 | Rab21 |
| 1437785_at | Lgx only | 474 | 397 | 463 | 337 | −1.19 | −1.03 | −1.41 | Adamts9 |
| 1437869_at | Res & Lgx | 14570 | 13664 | 11826 | 11731 | −1.07 | −1.23 | −1.24 | 3222402P14Rik |
| 1437875_at | Lgx only | 2753 | 2607 | 2485 | 2181 | −1.06 | −1.11 | −1.26 | Bicd2 |
| 1437900_at | Lgx only | 566 | 564 | 505 | 437 | −1.00 | −1.12 | −1.30 | 4930523C07Rik |
| 1437917_at | Lgx only | 370 | 334 | 293 | 223 | −1.11 | −1.26 | −1.65 | D530037H12Rik |
| 1438024_at | Res & Lgx | 3610 | 3226 | 2484 | 2237 | −1.12 | −1.45 | −1.61 | Ccdc90a |
| 1438026_at | Lgx only | 623 | 498 | 498 | 393 | −1.25 | −1.25 | −1.58 | Zfp560 |
| 1438045_at | Lgx only | 737 | 678 | 702 | 495 | −1.09 | −1.05 | −1.49 | Eea1 |
| 1438047_at | Res & Lgx | 1053 | 1176 | 1296 | 1256 | 1.12 | 1.23 | 1.19 | Zfp384 |
| 1438062_at | CR only | 588 | 372 | 440 | 434 | −1.58 | −1.34 | −1.36 | 4832420A03Rik |
| 1438077_at | Lgx only | 18 | 38 | 17 | 62 | 2.11 | −1.09 | 3.42 | Nlrp4a |
| 1438097_at | Lgx only | 611 | 574 | 490 | 391 | −1.06 | −1.25 | −1.56 | Rab20 |
| 1438169_a_at | Lgx only | 747 | 494 | 424 | 347 | −1.51 | −1.76 | −2.15 | Frmd4b |
| 1438195_at | Lgx only | 2915 | 3104 | 2321 | 2279 | 1.06 | −1.26 | −1.28 | Gpd1l |
| 1438208_at | Res only | 286 | 277 | 424 | 361 | −1.03 | 1.48 | 1.26 | Taok2 |
| 1438213_at | Lgx only | 15 | 30 | 39 | 79 | 2.00 | 2.61 | 5.30 | A830018L16Rik |
| 1438229_at | Res only | 843 | 887 | 609 | 701 | 1.05 | −1.38 | −1.20 | Pggt1b |
| 1438238_at | Lgx only | 231 | 217 | 198 | 106 | −1.07 | −1.17 | −2.18 | 2010315B03Rik |
| 1438241_at | Lgx only | 525 | 593 | 734 | 837 | 1.13 | 1.40 | 1.59 | Rgma |
| 1438258_at | CR & Res | 3530 | 2388 | 4783 | 3703 | −1.48 | 1.35 | 1.05 | Vldlr |
| 1438340_at | Lgx only | 219 | 117 | 157 | 125 | −1.86 | −1.40 | −1.75 | A930006D11Rik |
| 1438349_at | Lgx only | 195 | 145 | 121 | 75 | −1.34 | −1.61 | −2.60 | BC043476 |
| 1438400_at | Lgx only | 1226 | 1403 | 1426 | 1614 | 1.14 | 1.16 | 1.32 | 4632411B12Rik |
| 1438416_at | Lgx only | 1855 | 2525 | 2386 | 2992 | 1.36 | 1.29 | 1.61 | Med16 |
| 1438422_at | Res & Lgx | 1011 | 996 | 736 | 664 | −1.01 | −1.37 | −1.52 | Lrrc20 |
| 1438444_at | Lgx only | 9 | 29 | 35 | 26 | 3.33 | 3.99 | 3.02 | Spink10 |
| 1438510_a_at | Lgx only | 910 | 841 | 730 | 535 | −1.08 | −1.25 | −1.70 | Hars |
| 1438512_at | Res only | 68 | 151 | 177 | 104 | 2.21 | 2.59 | 1.52 | BC048679 |
| 1438532_at | Lgx only | 292 | 307 | 310 | 176 | 1.05 | 1.06 | −1.66 | Hmcn1 |
| 1438610_a_at | Lgx only | 887 | 695 | 489 | 435 | −1.28 | −1.82 | −2.04 | Cryz |
| 1438658_a_at | All | 1738 | 1228 | 1017 | 715 | −1.42 | −1.71 | −2.43 | Edg3 |
| 1438673_at | Lgx only | 759 | 586 | 535 | 411 | −1.29 | −1.42 | −1.85 | Slc4a7 |
| 1438676_at | CR & Lgx | 499 | 296 | 432 | 285 | −1.68 | −1.16 | −1.75 | Mpa2l |
| 1438678_at | Lgx only | 3261 | 2863 | 2608 | 2178 | −1.14 | −1.25 | −1.50 | 1500011K16Rik |
| 1438691_at | Res only | 664 | 655 | 437 | 488 | −1.01 | −1.52 | −1.36 | Zzef1 |
| 1438693_at | Res & Lgx | 311 | 353 | 461 | 515 | 1.14 | 1.48 | 1.66 | Tmem110 |
| 1438895_at | Res only | 571 | 519 | 762 | 647 | −1.10 | 1.33 | 1.13 | A430102J17Rik |
| 1438908_at | Lgx only | 246 | 204 | 303 | 415 | −1.21 | 1.23 | 1.69 | Map3k12 |
| 1438909_at | Lgx only | 51 | 62 | 76 | 173 | 1.23 | 1.49 | 3.41 | Sccpdh |
| 1438910_a_at | Lgx only | 3956 | 3657 | 4466 | 5068 | −1.08 | 1.13 | 1.28 | Stom |
| 1439008_at | Lgx only | 251 | 272 | 389 | 478 | 1.09 | 1.55 | 1.91 | Zfp319 |
| 1439010_at | Lgx only | 1107 | 987 | 790 | 675 | −1.12 | −1.40 | −1.64 | Larp4 |
| 1439024_at | CR only | 297 | 211 | 222 | 198 | −1.41 | −1.34 | −1.50 | Bag4 |
| 1439029_at | Lgx only | 629 | 843 | 709 | 1038 | 1.34 | 1.13 | 1.65 | Gpt2 |
| 1439055_at | Lgx only | 13 | 23 | 23 | 25 | 1.76 | 1.80 | 1.89 | OTTMUSG- |
| | | | | | | | | | 00000001305 |
| 1439078_at | Lgx only | 188 | 135 | 137 | 90 | −1.39 | −1.37 | −2.08 | Klhl4 |
| 1439143_at | CR & Lgx | 411 | 604 | 540 | 582 | 1.47 | 1.32 | 1.42 | A930018M24Rik |
| 1439153_at | Lgx only | 917 | 1123 | 739 | 645 | 1.22 | −1.24 | −1.42 | Ibrdc2 |
| 1439189_at | CR only | 592 | 915 | 521 | 472 | 1.54 | −1.14 | −1.25 | D630023B12Rik |
| 1439244_a_at | Lgx only | 2004 | 1777 | 2537 | 3175 | −1.13 | 1.27 | 1.58 | Tnrc6a |
| 1439266_a_at | Res & Lgx | 524 | 404 | 272 | 229 | −1.30 | −1.93 | −2.29 | Polr3k |
| 1439364_a_at | Lgx only | 1881 | 2097 | 2565 | 2782 | 1.11 | 1.36 | 1.48 | Mmp2 |
| 1439397_at | All | 322 | 190 | 197 | 172 | −1.69 | −1.63 | −1.88 | Fmn1 |
| 1439460_a_at | All | 2411 | 1816 | 1398 | 1125 | −1.33 | −1.73 | −2.14 | Zfp289 |
| 1439483_at | Res only | 47 | 63 | 129 | 96 | 1.33 | 2.71 | 2.02 | AI506816 |
| 1439488_at | Lgx only | 427 | 660 | 693 | 889 | 1.55 | 1.62 | 2.08 | Dot1l |
| 1439509_at | Res only | 161 | 198 | 257 | 226 | 1.22 | 1.59 | 1.40 | 2900008C10Rik |
| 1439529_at | Res & Lgx | 293 | 347 | 451 | 554 | 1.18 | 1.54 | 1.89 | A430110N23Rik |
| 1439675_at | Res only | 1299 | 1589 | 964 | 977 | 1.22 | −1.35 | −1.33 | 4933429D07Rik |
| 1439793_at | Lgx only | 539 | 391 | 382 | 304 | −1.38 | −1.41 | −1.77 | Gja3 |
| 1439797_at | Lgx only | 324 | 388 | 353 | 514 | 1.20 | 1.09 | 1.59 | Ppard |
| 1439815_at | Lgx only | 1354 | 1602 | 1962 | 2028 | 1.18 | 1.45 | 1.50 | Heatr5b |
| 1439821_at | Res only | 147 | 79 | 51 | 75 | −1.86 | −2.91 | −1.96 | Lrp2bp |
| 1440018_at | Lgx only | 32 | 47 | 70 | 136 | 1.48 | 2.21 | 4.30 | A330043J11Rik |
| 1440096_at | Lgx only | 360 | 318 | 281 | 177 | −1.13 | −1.28 | −2.03 | Ecm2 |
| 1440143_at | Lgx only | 141 | 181 | 180 | 383 | 1.28 | 1.28 | 2.71 | Pigz |
| 1440232_at | Res only | 47 | 67 | 145 | 121 | 1.41 | 3.07 | 2.55 | EG622645 |
| 1440234_at | Lgx only | 1187 | 1250 | 1054 | 915 | 1.05 | −1.13 | −1.30 | 1810012P15Rik |
| 1440279_at | Lgx only | 326 | 267 | 247 | 189 | −1.22 | −1.32 | −1.73 | Txndc10 |
| 1440335_at | Lgx only | 2118 | 2238 | 2120 | 1677 | 1.06 | 1.00 | −1.26 | LOC100046468 |
| 1440478_at | Res only | 176 | 92 | 68 | 123 | −1.90 | −2.57 | −1.43 | LOC100047601 |
| 1440480_at | Lgx only | 60 | 174 | 203 | 293 | 2.89 | 3.38 | 4.87 | AB182283 |
| 1440520_a_at | CR only | 147 | 330 | 264 | 161 | 2.24 | 1.79 | 1.09 | 1700051A21Rik |
| 1440537_at | Res only | 313 | 264 | 495 | 395 | −1.19 | 1.58 | 1.26 | Kcnv2 |
| 1440542_at | Res only | 82 | 53 | 18 | 48 | −1.54 | −4.45 | −1.72 | 7420416P09Rik |
| 1440561_at | Lgx only | 65 | 85 | 111 | 158 | 1.30 | 1.70 | 2.43 | C87259 |
| 1440739_at | CR only | 524 | 357 | 538 | 443 | −1.47 | 1.03 | −1.18 | Vegfc |
| 1440781_at | CR only | 133 | 48 | 101 | 60 | −2.75 | −1.32 | −2.21 | B830007D08Rik |
| 1440831_at | Res only | 1257 | 1218 | 923 | 944 | −1.03 | −1.36 | −1.33 | Bach1 |
| 1440838_at | Lgx only | 431 | 523 | 579 | 624 | 1.21 | 1.34 | 1.45 | AI852064 |
| 1440841_at | Lgx only | 745 | 669 | 622 | 529 | −1.11 | −1.20 | −1.41 | BB217526 |
| 1440849_at | CR only | 660 | 558 | 616 | 573 | −1.18 | −1.07 | −1.15 | 6330417G04Rik |
| 1440874_at | CR only | 311 | 151 | 297 | 337 | −2.06 | −1.05 | 1.08 | Slco5a1 |
| 1440908_at | Lgx only | 346 | 365 | 317 | 547 | 1.06 | −1.09 | 1.58 | D030063E12 |
| 1440934_at | Lgx only | 102 | 101 | 173 | 224 | −1.00 | 1.70 | 2.20 | AW742931 |
| 1440969_at | All | 199 | 103 | 102 | 67 | −1.93 | −1.94 | −2.95 | BC030308 |
| 1441001_at | Lgx only | 52 | 82 | 140 | 150 | 1.59 | 2.70 | 2.91 | AI225934 |
| 1441075_at | Lgx only | 317 | 274 | 260 | 171 | −1.16 | −1.22 | −1.86 | Nostrin |
| 1441081_a_at | Lgx only | 202 | 199 | 256 | 320 | −1.02 | 1.27 | 1.58 | 1110038B12Rik |
| 1441139_at | CR only | 2413 | 2791 | 2076 | 2479 | 1.16 | −1.16 | 1.03 | ENSMUSG- |
| | | | | | | | | | 00000071543 |
| 1441174_a_at | Res & Lgx | 123 | 85 | 72 | 87 | −1.44 | −1.71 | −1.41 | Lmln |
| 1441229_at | Res & Lgx | 174 | 114 | 73 | 70 | −1.52 | −2.36 | −2.48 | D230019N24Rik |
| 1441266_at | Res & Lgx | 812 | 636 | 542 | 457 | −1.28 | −1.50 | −1.78 | Strn3 |
| 1441320_a_at | Res & Lgx | 602 | 634 | 812 | 873 | 1.05 | 1.35 | 1.45 | AI413194 |
| 1441438_at | CR only | 269 | 183 | 277 | 225 | −1.47 | 1.03 | −1.20 | Gpc6 |
| 1441536_at | Lgx only | 18 | 44 | 32 | 86 | 2.39 | 1.74 | 4.68 | Hmgcs1 |
| 1441590_at | Lgx only | 1067 | 1051 | 1373 | 1614 | −1.01 | 1.29 | 1.51 | Kcnj5 |
| 1441970_at | Lgx only | 419 | 391 | 340 | 302 | −1.07 | −1.23 | −1.39 | E430010N07Rik |
| 1441987_at | Res & Lgx | 585 | 488 | 465 | 395 | −1.20 | −1.26 | −1.48 | Mbd5 |
| 1442002_at | Lgx only | 348 | 254 | 303 | 186 | −1.37 | −1.15 | −1.87 | 7030402D04Rik |
| 1442027_at | Lgx only | 1531 | 1126 | 1055 | 790 | −1.36 | −1.45 | −1.94 | Nbeal1 |
| 1442050_at | Res & Lgx | 328 | 298 | 521 | 618 | −1.10 | 1.59 | 1.88 | Zfp608 |
| 1442064_at | Res & Lgx | 84 | 144 | 242 | 284 | 1.71 | 2.88 | 3.37 | AW556556 |
| 1442090_at | Res only | 33 | 77 | 108 | 83 | 2.30 | 3.24 | 2.47 | 8030463A06Rik |
| 1442174_at | Lgx only | 360 | 314 | 499 | 581 | −1.15 | 1.39 | 1.61 | Tspan18 |
| 1442186_at | Res & Lgx | 215 | 323 | 448 | 443 | 1.50 | 2.09 | 2.06 | LOC100045002 |
| 1442362_at | Res & Lgx | 165 | 113 | 51 | 64 | −1.45 | −3.22 | −2.59 | Gm104 |
| 1442434_at | Res only | 466 | 588 | 765 | 673 | 1.26 | 1.64 | 1.44 | D8Ertd82e |
| 1442659_at | Lgx only | 332 | 320 | 312 | 473 | −1.04 | −1.07 | 1.42 | Pcdh9 |
| 1442695_at | Lgx only | 49 | 67 | 48 | 156 | 1.35 | −1.03 | 3.15 | C030007I01Rik |
| 1442732_at | CR only | 380 | 683 | 596 | 574 | 1.80 | 1.57 | 1.51 | Hadhb |
| 1442757_at | CR & Res | 432 | 288 | 287 | 372 | −1.50 | −1.50 | −1.16 | Lrch1 |
| 1442867_at | Res only | 135 | 148 | 262 | 224 | 1.10 | 1.94 | 1.65 | Megf11 |
| 1442926_at | Lgx only | 10 | 49 | 42 | 75 | 5.07 | 4.40 | 7.80 | 1700011B04Rik |
| 1443103_at | CR only | 69 | 23 | 45 | 45 | −2.98 | −1.53 | −1.53 | D830046C22Rik |
| 1443416_at | Lgx only | 197 | 253 | 192 | 328 | 1.28 | −1.03 | 1.66 | C79741 |
| 1443483_at | Lgx only | 59 | 59 | 64 | 17 | 1.01 | 1.08 | −3.39 | Xlr5a |
| 1443632_at | CR only | 10172 | 12920 | 10060 | 11150 | 1.27 | −1.01 | 1.10 | Obscn |
| 1443682_at | Res only | 259 | 192 | 424 | 371 | −1.35 | 1.64 | 1.43 | AI662476 |
| 1443854_at | Lgx only | 405 | 288 | 380 | 192 | −1.41 | −1.07 | −2.11 | Hand2 |
| 1443856_at | Lgx only | 726 | 687 | 651 | 538 | −1.06 | −1.11 | −1.35 | Rabep1 |
| 1443896_at | Lgx only | 748 | 888 | 896 | 1033 | 1.19 | 1.20 | 1.38 | Tbc1d5 |
| 1443932_at | Res & Lgx | 2283 | 1935 | 1827 | 1552 | −1.18 | −1.25 | −1.47 | Klhdc1 |
| 1443935_at | Res only | 401 | 429 | 494 | 464 | 1.07 | 1.23 | 1.16 | BC032203 |
| 1444041_at | Lgx only | 146 | 127 | 94 | 62 | −1.15 | −1.55 | −2.35 | AU041133 |
| 1444112_at | Lgx only | 1457 | 1326 | 1343 | 1080 | −1.10 | −1.09 | −1.35 | ENSMUSG- |
| | | | | | | | | | 00000074466 |
| 1444217_at | Lgx only | 212 | 168 | 299 | 382 | −1.26 | 1.41 | 1.80 | Mrpl38 |
| 1444254_at | CR only | 84 | 28 | 74 | 105 | −2.95 | −1.12 | 1.26 | Tns4 |
| 1444294_at | Lgx only | 127 | 204 | 249 | 265 | 1.61 | 1.97 | 2.10 | 4930551A22Rik |
| 1444370_at | CR only | 46 | 16 | 60 | 24 | −2.80 | 1.31 | −1.91 | C77058 |
| 1444456_at | Lgx only | 245 | 268 | 240 | 176 | 1.09 | −1.02 | −1.39 | 9030425P06Rik |
| 1444494_at | All | 2200 | 1830 | 1616 | 1601 | −1.20 | −1.36 | −1.37 | Kbtbd10 |
| 1444537_at | Lgx only | 219 | 296 | 347 | 346 | 1.35 | 1.59 | 1.58 | AI429363 |
| 1444684_at | Lgx only | 50 | 23 | 43 | 16 | −2.18 | −1.15 | −3.18 | 8030475D13Rik |
| 1444766_at | Lgx only | 107 | 102 | 101 | 31 | −1.04 | −1.06 | −3.48 | Atxn7l1 |
| 1445191_at | CR only | 187 | 125 | 201 | 197 | −1.50 | 1.08 | 1.05 | Exdl1 |
| 1445459_at | Lgx only | 50 | 100 | 93 | 186 | 2.00 | 1.87 | 3.72 | Sstr5 |
| 1446062_at | Res only | 58 | 100 | 121 | 102 | 1.74 | 2.10 | 1.77 | B830028B13Rik |
| 1446214_at | CR only | 18 | 68 | 77 | 45 | 3.67 | 4.16 | 2.45 | D430018E03Rik |
| 1446678_at | Lgx only | 37 | 60 | 62 | 121 | 1.62 | 1.68 | 3.26 | D14Ertd16e |
| 1446781_at | CR only | 95 | 21 | 55 | 68 | −4.54 | −1.73 | −1.39 | D8Ertd54e |
| 1446812_at | Res & Lgx | 890 | 716 | 584 | 552 | −1.24 | −1.52 | −1.61 | LOC100040515 |
| 1446842_at | CR only | 68 | 13 | 26 | 27 | −5.38 | −2.60 | −2.47 | D4Ertd571e |
| 1447035_at | Lgx only | 41 | 91 | 109 | 139 | 2.21 | 2.63 | 3.36 | A230091C14Rik |
| 1447095_at | Lgx only | 109 | 130 | 166 | 195 | 1.19 | 1.52 | 1.79 | C86942 |
| 1447923_at | Lgx only | 781 | 678 | 544 | 448 | −1.15 | −1.44 | −1.74 | 1810026B05Rik |
| 1447967_at | Res only | 256 | 260 | 440 | 360 | 1.01 | 1.72 | 1.41 | Tmem69 |
| 1447981_at | CR only | 68 | 13 | 66 | 56 | −5.40 | −1.03 | −1.21 | C78441 |
| 1448091_at | CR & Lgx | 18 | 85 | 41 | 75 | 4.74 | 2.28 | 4.20 | D15Ertd50e |
| 1448100_at | Res & Lgx | 4461 | 4342 | 3733 | 3467 | −1.03 | −1.20 | −1.29 | 4833439L19Rik |
| 1448116_at | Res & Lgx | 3337 | 3991 | 4522 | 5277 | 1.20 | 1.35 | 1.58 | Ube1x |
| 1448122_at | CR & Lgx | 8342 | 9257 | 8220 | 9546 | 1.11 | −1.01 | 1.14 | Tcp1 |
| 1448143_at | Lgx only | 6719 | 7951 | 7830 | 8811 | 1.18 | 1.17 | 1.31 | Aldh2 |
| 1448145_at | Lgx only | 304 | 323 | 454 | 535 | 1.06 | 1.50 | 1.76 | Wwp2 |
| 1448148_at | Res & Lgx | 1666 | 2147 | 2587 | 3224 | 1.29 | 1.55 | 1.94 | Grn |
| 1448153_at | Lgx only | 60488 | 68353 | 63338 | 84246 | 1.13 | 1.05 | 1.39 | Cox5a |
| 1448154_at | Lgx only | 19344 | 22511 | 19920 | 25912 | 1.16 | 1.03 | 1.34 | Ndrg2 |
| 1448163_at | Lgx only | 798 | 1050 | 981 | 1240 | 1.32 | 1.23 | 1.55 | Gnpda1 |
| 1448167_at | Lgx only | 684 | 808 | 806 | 1130 | 1.18 | 1.18 | 1.65 | Ifngr1 |
| 1448174_at | Lgx only | 1875 | 1928 | 2181 | 2232 | 1.03 | 1.16 | 1.19 | Cul1 |
| 1448181_at | CR only | 1549 | 2211 | 1745 | 1742 | 1.43 | 1.13 | 1.12 | Klf15 |
| 1448185_at | Lgx only | 5495 | 7055 | 5636 | 8302 | 1.28 | 1.03 | 1.51 | Herpud1 |
| 1448188_at | Lgx only | 5526 | 6650 | 8466 | 12288 | 1.20 | 1.53 | 2.22 | Ucp2 |
| 1448189_a_at | Lgx only | 2277 | 2853 | 3180 | 3351 | 1.25 | 1.40 | 1.47 | Flii |
| 1448196_at | Res & Lgx | 3368 | 2651 | 2008 | 1537 | −1.27 | −1.68 | −2.19 | Mat2b |
| 1448198_a_at | Lgx only | 38229 | 43229 | 40539 | 48367 | 1.13 | 1.06 | 1.27 | Ndufb8 |
| 1448199_at | Res only | 679 | 631 | 507 | 543 | −1.08 | −1.34 | −1.25 | Ankrd10 |
| 1448206_at | Lgx only | 6419 | 7041 | 7577 | 10844 | 1.10 | 1.18 | 1.69 | Psma2 |
| 1448208_at | Lgx only | 632 | 474 | 499 | 348 | −1.34 | −1.27 | −1.82 | Smad1 |
| 1448209_a_at | Lgx only | 246 | 316 | 363 | 502 | 1.29 | 1.48 | 2.04 | Slc22a17 |
| 1448212_at | Lgx only | 1004 | 973 | 746 | 707 | −1.03 | −1.35 | −1.42 | Tnfsf5ip1 |
| 1448221_at | CR only | 4042 | 4996 | 4086 | 4247 | 1.24 | 1.01 | 1.05 | Bat1a |
| 1448224_at | Lgx only | 1460 | 1359 | 1117 | 995 | −1.07 | −1.31 | −1.47 | Tfam |
| 1448225_at | Lgx only | 498 | 691 | 692 | 796 | 1.39 | 1.39 | 1.60 | Gpaa1 |
| 1448240_at | Lgx only | 1199 | 1319 | 1410 | 1668 | 1.10 | 1.18 | 1.39 | Mbtps1 |
| 1448242_at | Lgx only | 1162 | 1336 | 1617 | 1835 | 1.15 | 1.39 | 1.58 | Sec61a1 |
| 1448244_at | Lgx only | 1573 | 1505 | 1192 | 813 | −1.05 | −1.32 | −1.93 | Lypla1 |
| 1448252_a_at | Lgx only | 7588 | 7012 | 6014 | 5451 | −1.08 | −1.26 | −1.39 | Eef1b2 |
| 1448258_a_at | Lgx only | 6413 | 6297 | 5305 | 4246 | −1.02 | −1.21 | −1.51 | Spcs1 |
| 1448269_a_at | Res & Lgx | 613 | 517 | 374 | 350 | −1.19 | −1.64 | −1.75 | Klhl13 |
| 1448276_at | Lgx only | 1808 | 2156 | 1862 | 2730 | 1.19 | 1.03 | 1.51 | Tspan4 |
| 1448284_a_at | Lgx only | 37586 | 40364 | 44938 | 56730 | 1.07 | 1.20 | 1.51 | Ndufc1 |
| 1448287_at | Lgx only | 4436 | 4040 | 3639 | 2830 | −1.10 | −1.22 | −1.57 | Rpo1-3 |
| 1448304_a_at | Res & Lgx | 3053 | 2867 | 2183 | 1770 | −1.06 | −1.40 | −1.72 | Rab6 |
| 1448313_at | Lgx only | 2609 | 2807 | 3061 | 3302 | 1.08 | 1.17 | 1.27 | Tpp1 |
| 1448325_at | Res & Lgx | 757 | 893 | 1112 | 1080 | 1.18 | 1.47 | 1.43 | Myd116 |
| 1448327_at | Lgx only | 30728 | 36837 | 37776 | 43889 | 1.20 | 1.23 | 1.43 | Actn2 |
| 1448330_at | Res & Lgx | 7365 | 8521 | 9720 | 11395 | 1.16 | 1.32 | 1.55 | Gstm1 |
| 1448336_at | Lgx only | 1711 | 1622 | 1611 | 1433 | −1.05 | −1.06 | −1.19 | Drg1 |
| 1448339_at | Res & Lgx | 4104 | 3995 | 2913 | 2634 | −1.03 | −1.41 | −1.56 | Tmem30a |
| 1448341_a_at | Lgx only | 321 | 317 | 503 | 653 | −1.01 | 1.57 | 2.04 | Stxbp2 |
| 1448345_at | Lgx only | 1081 | 1303 | 1204 | 1435 | 1.21 | 1.11 | 1.33 | Tomm34 |
| 1448346_at | CR only | 2920 | 2207 | 2287 | 2450 | −1.32 | −1.28 | −1.19 | Cfl1 |
| 1448351_at | Lgx only | 1035 | 1248 | 1300 | 1665 | 1.21 | 1.26 | 1.61 | Coro1b |
| 1448356_at | Lgx only | 2083 | 1903 | 1705 | 1572 | −1.09 | −1.22 | −1.32 | Ube2d2 |
| 1448362_at | Lgx only | 1772 | 2097 | 2189 | 2519 | 1.18 | 1.24 | 1.42 | Dnajc7 |
| 1448363_at | Res & Lgx | 2486 | 2740 | 3232 | 3785 | 1.10 | 1.30 | 1.52 | Yap1 |
| 1448365_at | Res & Lgx | 1790 | 1723 | 1184 | 1227 | −1.04 | −1.51 | −1.46 | Exosc7 |
| 1448379_at | Res only | 647 | 563 | 413 | 518 | −1.15 | −1.57 | −1.25 | Pot1a |
| 1448380_at | Res & Lgx | 1020 | 1208 | 1439 | 1659 | 1.19 | 1.41 | 1.63 | Lgals3bp |
| 1448388_a_at | Lgx only | 2505 | 2405 | 1802 | 1158 | −1.04 | −1.39 | −2.16 | 1110002B05Rik |
| 1448402_at | Lgx only | 1415 | 1869 | 2287 | 2869 | 1.32 | 1.62 | 2.03 | Tln1 |
| 1448412_a_at | Lgx only | 3472 | 4076 | 4244 | 4721 | 1.17 | 1.22 | 1.36 | Tsc22d4 |
| 1448415_a_at | Lgx only | 387 | 438 | 593 | 653 | 1.13 | 1.53 | 1.69 | Sema3b |
| 1448416_at | Lgx only | 16601 | 20394 | 19345 | 31156 | 1.23 | 1.17 | 1.88 | Mgp |
| 1448417_at | Lgx only | 1000 | 1181 | 1301 | 1299 | 1.18 | 1.30 | 1.30 | Ninj1 |
| 1448429_at | Res & Lgx | 3339 | 3198 | 2421 | 1974 | −1.04 | −1.38 | −1.69 | Gyg |
| 1448430_a_at | CR & Lgx | 8516 | 10720 | 9639 | 10638 | 1.26 | 1.13 | 1.25 | Naca |
| 1448432_at | Lgx only | 369 | 514 | 544 | 661 | 1.40 | 1.48 | 1.79 | Plcd1 |
| 1448438_at | Lgx only | 2204 | 1814 | 1971 | 1577 | −1.22 | −1.12 | −1.40 | Derl2 |
| 1448463_at | Lgx only | 4645 | 4072 | 3480 | 3091 | −1.14 | −1.33 | −1.50 | 4933434E20Rik |
| 1448467_a_at | Lgx only | 1218 | 1447 | 1612 | 1817 | 1.19 | 1.32 | 1.49 | Ehbp1l1 |
| 1448476_at | CR & Lgx | 5612 | 6609 | 6127 | 7217 | 1.18 | 1.09 | 1.29 | Nap1l4 |
| 1448480_at | Res & Lgx | 1000 | 728 | 556 | 360 | −1.37 | −1.80 | −2.78 | Nip7 |
| 1448484_at | Res & Lgx | 5744 | 5110 | 3530 | 3419 | −1.12 | −1.63 | −1.68 | Amd1 |
| 1448488_at | CR & Lgx | 831 | 1036 | 919 | 1013 | 1.25 | 1.11 | 1.22 | Mrps5 |
| 1448492_a_at | Res & Lgx | 4733 | 4441 | 3842 | 3020 | −1.07 | −1.23 | −1.57 | Psmd12 |
| 1448493_at | CR & Lgx | 9202 | 6905 | 8442 | 6228 | −1.33 | −1.09 | −1.48 | Paip2 |
| 1448495_at | Lgx only | 723 | 911 | 890 | 1046 | 1.26 | 1.23 | 1.45 | Tsta3 |
| 1448498_at | Lgx only | 793 | 1061 | 951 | 1228 | 1.34 | 1.20 | 1.55 | Rps6ka4 |
| 1448505_at | Res & Lgx | 1738 | 1400 | 1033 | 860 | −1.24 | −1.68 | −2.02 | C1d |
| 1448508_at | Lgx only | 237 | 284 | 322 | 442 | 1.20 | 1.36 | 1.86 | Traf3ip2 |
| 1448517_at | Lgx only | 2047 | 2144 | 1562 | 1376 | 1.05 | −1.31 | −1.49 | Timm22 |
| 1448527_at | Lgx only | 1955 | 1753 | 1345 | 1338 | −1.12 | −1.45 | −1.46 | Pdcd10 |
| 1448533_at | Res & Lgx | 3076 | 2967 | 2613 | 2263 | −1.04 | −1.18 | −1.36 | Tbcb |
| 1448535_at | Lgx only | 306 | 250 | 237 | 154 | −1.22 | −1.29 | −1.98 | Elp4 |
| 1448536_at | Lgx only | 4579 | 4812 | 3261 | 2727 | 1.05 | −1.40 | −1.68 | Lsm3 |
| 1448537_at | Res & Lgx | 2417 | 2026 | 1762 | 1591 | −1.19 | −1.37 | −1.52 | Ttc1 |
| 1448543_at | Res & Lgx | 1715 | 1494 | 908 | 822 | −1.15 | −1.89 | −2.09 | Slmo2 |
| 1448548_at | Lgx only | 1328 | 1385 | 1288 | 1631 | 1.04 | −1.03 | 1.23 | Tulp4 |
| 1448549_a_at | Res only | 1179 | 1068 | 850 | 1106 | −1.10 | −1.39 | −1.07 | Dpagt1 |
| 1448559_at | Lgx only | 5131 | 5547 | 6227 | 7143 | 1.08 | 1.21 | 1.39 | Flot1 |
| 1448564_at | Res only | 511 | 609 | 713 | 573 | 1.19 | 1.40 | 1.12 | Cib1 |
| 1448565_at | Lgx only | 2831 | 2386 | 2740 | 1942 | −1.19 | −1.03 | −1.46 | Ppp1r11 |
| 1448567_at | Lgx only | 590 | 668 | 763 | 806 | 1.13 | 1.29 | 1.37 | Tmem115 |
| 1448568_a_at | CR only | 688 | 524 | 629 | 533 | −1.31 | −1.09 | −1.29 | Slc20a1 |
| 1448570_at | Lgx only | 1666 | 1576 | 1258 | 948 | −1.06 | −1.32 | −1.76 | Gmfb |
| 1448579_at | Lgx only | 2689 | 2976 | 3392 | 3692 | 1.11 | 1.26 | 1.37 | Glg1 |
| 1448585_at | Res only | 751 | 809 | 578 | 655 | 1.08 | −1.30 | −1.15 | Gtf2h4 |
| 1448591_at | CR only | 1010 | 720 | 1099 | 736 | −1.40 | 1.09 | −1.37 | Ctss |
| 1448613_at | Lgx only | 1206 | 1308 | 1426 | 1969 | 1.08 | 1.18 | 1.63 | Ecm1 |
| 1448615_at | Res & Lgx | 769 | 858 | 1028 | 1113 | 1.12 | 1.34 | 1.45 | Ccs |
| 1448621_a_at | Res & Lgx | 7092 | 7555 | 5213 | 5291 | 1.07 | −1.36 | −1.34 | Smpd1 |
| 1448623_at | Lgx only | 1699 | 1287 | 1178 | 807 | −1.32 | −1.44 | −2.11 | Tmem123 |
| 1448625_at | Res & Lgx | 1612 | 1957 | 2268 | 2558 | 1.21 | 1.41 | 1.59 | Golga2 |
| 1448637_at | Lgx only | 1033 | 1248 | 1507 | 1924 | 1.21 | 1.46 | 1.86 | Med25 |
| 1448638_at | Res only | 68 | 79 | 140 | 150 | 1.17 | 2.06 | 2.21 | Mtbp |
| 1448644_at | Lgx only | 1286 | 1471 | 1597 | 1684 | 1.14 | 1.24 | 1.31 | Pef1 |
| 1448645_at | Lgx only | 2239 | 2328 | 1993 | 1844 | 1.04 | −1.12 | −1.21 | Msl31 |
| 1448649_at | Lgx only | 540 | 500 | 484 | 392 | −1.08 | −1.12 | −1.38 | Enpep |
| 1448684_at | Res & Lgx | 4396 | 3940 | 3485 | 3517 | −1.12 | −1.26 | −1.25 | Ppp1r2 |
| 1448696_at | Res only | 95 | 115 | 47 | 40 | 1.20 | −2.03 | −2.37 | Heph |
| 1448700_at | Lgx only | 4037 | 3143 | 2765 | 2331 | −1.28 | −1.46 | −1.73 | G0s2 |
| 1448717_at | CR only | 889 | 671 | 980 | 830 | −1.32 | 1.10 | −1.07 | Gcdh |
| 1448720_at | Lgx only | 342 | 284 | 279 | 229 | −1.21 | −1.23 | −1.49 | Lrrc40 |
| 1448724_at | Res only | 4162 | 4532 | 2682 | 2820 | 1.09 | −1.55 | −1.48 | Cish |
| 1448727_at | Lgx only | 269 | 370 | 398 | 484 | 1.37 | 1.48 | 1.80 | Tle6 |
| 1448729_a_at | CR & Lgx | 1559 | 1304 | 1446 | 1143 | −1.20 | −1.08 | −1.36 | 39329 |
| 1448737_at | Lgx only | 4695 | 4240 | 4264 | 3613 | −1.11 | −1.10 | −1.30 | Tspan7 |
| 1448760_at | Lgx only | 643 | 554 | 539 | 457 | −1.16 | −1.19 | −1.41 | Zfp68 |
| 1448762_at | Res & Lgx | 1357 | 1116 | 910 | 676 | −1.22 | −1.49 | −2.01 | Rad17 |
| 1448769_at | Lgx only | 2415 | 2426 | 2058 | 1875 | 1.00 | −1.17 | −1.29 | Slc35b1 |
| 1448770_a_at | Lgx only | 6679 | 6835 | 5272 | 3909 | 1.02 | −1.27 | −1.71 | Atpif1 |
| 1448771_a_at | Lgx only | 49028 | 51355 | 54395 | 64994 | 1.05 | 1.11 | 1.33 | Fth1 |
| 1448788_at | Res & Lgx | 1474 | 1445 | 1194 | 1065 | −1.02 | −1.24 | −1.38 | Cd200 |
| 1448792_a_at | Res only | 137 | 80 | 49 | 1448 | −1.71 | −2.79 | 10.55 | Cyp2f2 |
| 1448797_at | Res & Lgx | 945 | 842 | 674 | 584 | −1.12 | −1.40 | −1.62 | Elk3 |
| 1448809_at | Res & Lgx | 2605 | 2313 | 1681 | 1641 | −1.13 | −1.55 | −1.59 | Cse1l |
| 1448810_at | Res & Lgx | 397 | 445 | 638 | 701 | 1.12 | 1.61 | 1.77 | Gne |
| 1448826_at | Lgx only | 95590 | 115497 | 118388 | 173815 | 1.21 | 1.24 | 1.82 | Myh6 |
| 1448830_at | Lgx only | 5138 | 4291 | 4152 | 3187 | −1.20 | −1.24 | −1.61 | Dusp1 |
| 1448835_at | Res & Lgx | 5346 | 5044 | 3924 | 3656 | −1.06 | −1.36 | −1.46 | E2f6 |
| 1448838_at | Lgx only | 386 | 350 | 281 | 214 | −1.10 | −1.37 | −1.81 | Topors |
| 1448840_at | Lgx only | 461 | 474 | 456 | 753 | 1.03 | −1.01 | 1.63 | Tmub1 |
| 1448844_at | Lgx only | 1373 | 1278 | 1179 | 983 | −1.07 | −1.17 | −1.40 | Cyb5b |
| 1448853_at | Lgx only | 5831 | 5559 | 4659 | 4095 | −1.05 | −1.25 | −1.42 | Synj2bp |
| 1448856_a_at | Res & Lgx | 2552 | 2477 | 2140 | 2204 | −1.03 | −1.19 | −1.16 | Msra |
| 1448860_at | Res only | 17 | 35 | 50 | 64 | 2.14 | 3.02 | 3.84 | Rem2 |
| 1448864_at | Lgx only | 3508 | 4978 | 4574 | 4515 | 1.42 | 1.30 | 1.29 | Snrk |
| 1448867_at | Lgx only | 2465 | 2370 | 1926 | 1977 | −1.04 | −1.28 | −1.25 | Tmem9b |
| 1448883_at | Lgx only | 986 | 1264 | 880 | 1339 | 1.28 | −1.12 | 1.36 | Lgmn |
| 1448884_at | Lgx only | 742 | 688 | 554 | 408 | −1.08 | −1.34 | −1.82 | Gtf2e2 |
| 1448885_at | CR only | 299 | 214 | 340 | 415 | −1.40 | 1.14 | 1.39 | Rap2b |
| 1448893_at | Lgx only | 851 | 1373 | 1459 | 1947 | 1.61 | 1.71 | 2.29 | Ncor2 |
| 1448894_at | Lgx only | 832 | 852 | 961 | 1170 | 1.02 | 1.15 | 1.41 | Akr1b8 |
| 1448900_at | Lgx only | 4895 | 4713 | 4330 | 3890 | −1.04 | −1.13 | −1.26 | D16H22S680E |
| 1448903_at | Lgx only | 7279 | 5973 | 5176 | 3901 | −1.22 | −1.41 | −1.87 | 39340 |
| 1448917_at | Lgx only | 1574 | 1229 | 1326 | 1221 | −1.28 | −1.19 | −1.29 | Med30 |
| 1448918_at | Lgx only | 1286 | 1478 | 1757 | 1991 | 1.15 | 1.37 | 1.55 | Slco3a1 |
| 1448943_at | Lgx only | 4428 | 4185 | 3289 | 2623 | −1.06 | −1.35 | −1.69 | Nrp1 |
| 1448947_at | Lgx only | 2361 | 2309 | 1900 | 1544 | −1.02 | −1.24 | −1.53 | 2810004N23Rik |
| 1448948_at | Lgx only | 1247 | 1146 | 1163 | 1058 | −1.09 | −1.07 | −1.18 | Rag1ap1 |
| 1448956_at | Lgx only | 3964 | 4452 | 6177 | 9601 | 1.12 | 1.56 | 2.42 | Stard10 |
| 1448960_at | Lgx only | 1075 | 1032 | 1164 | 1411 | −1.04 | 1.08 | 1.31 | Cxxc5 |
| 1448970_at | Lgx only | 3836 | 3551 | 3364 | 2960 | −1.08 | −1.14 | −1.30 | Slc25a46 |
| 1448971_at | Lgx only | 389 | 353 | 307 | 236 | −1.10 | −1.26 | −1.65 | 2410022L05Rik |
| 1448987_at | Lgx only | 43424 | 51619 | 52391 | 80140 | 1.19 | 1.21 | 1.85 | Acadl |
| 1448993_at | Res & Lgx | 2740 | 2404 | 1834 | 1517 | −1.14 | −1.49 | −1.81 | Atg3 |
| 1449014_at | Res only | 881 | 843 | 642 | 705 | −1.05 | −1.37 | −1.25 | Lactb |
| 1449025_at | Lgx only | 548 | 576 | 657 | 774 | 1.05 | 1.20 | 1.41 | Ifit3 |
| 1449026_at | Res & Lgx | 503 | 552 | 779 | 792 | 1.10 | 1.55 | 1.57 | Ifnar1 |
| 1449042_at | Lgx only | 936 | 870 | 1132 | 1241 | −1.08 | 1.21 | 1.33 | Ctcf |
| 1449044_at | Lgx only | 1437 | 1195 | 953 | 801 | −1.20 | −1.51 | −1.79 | Eef1e1 |
| 1449045_at | Lgx only | 952 | 1152 | 1135 | 1427 | 1.21 | 1.19 | 1.50 | Afg3l1 |
| 1449046_a_at | Lgx only | 1537 | 1426 | 1431 | 1340 | −1.08 | −1.07 | −1.15 | Josd2 |
| 1449058_at | Lgx only | 143 | 148 | 92 | 232 | 1.04 | −1.55 | 1.63 | Gli1 |
| 1449062_at | Lgx only | 347 | 452 | 475 | 608 | 1.30 | 1.37 | 1.75 | Khk |
| 1449063_at | Res only | 1262 | 1084 | 930 | 1007 | −1.16 | −1.36 | −1.25 | Sec22b |
| 1449071_at | Lgx only | 25977 | 29211 | 40937 | 73543 | 1.12 | 1.58 | 2.83 | Myl7 |
| 1449072_a_at | Lgx only | 1737 | 1759 | 1601 | 1044 | 1.01 | −1.09 | −1.66 | N6amt2 |
| 1449078_at | Res & Lgx | 1395 | 1028 | 1025 | 751 | −1.36 | −1.36 | −1.86 | St3gal6 |
| 1449080_at | All | 447 | 289 | 283 | 232 | −1.55 | −1.58 | −1.93 | Hdac2 |
| 1449088_at | Lgx only | 1611 | 1389 | 2284 | 2373 | −1.16 | 1.42 | 1.47 | Fbp2 |
| 1449096_at | Lgx only | 1028 | 1107 | 863 | 714 | 1.08 | −1.19 | −1.44 | Ccdc127 |
| 1449106_at | Lgx only | 16622 | 17727 | 24198 | 34324 | 1.07 | 1.46 | 2.07 | Gpx3 |
| 1449108_at | Lgx only | 7575 | 7320 | 6088 | 4914 | −1.03 | −1.24 | −1.54 | Fdx1 |
| 1449113_at | Res only | 848 | 830 | 647 | 687 | −1.02 | −1.31 | −1.23 | Gpbp1l1 |
| 1449123_at | Lgx only | 84 | 43 | 111 | 29 | −1.96 | 1.32 | −2.93 | Itih3 |
| 1449124_at | Res & Lgx | 310 | 370 | 480 | 455 | 1.19 | 1.55 | 1.47 | Rgl1 |
| 1449125_at | Lgx only | 202 | 143 | 142 | 96 | −1.42 | −1.43 | −2.11 | Tnfaip8l1 |
| 1449138_at | Lgx only | 3517 | 3750 | 4329 | 4360 | 1.07 | 1.23 | 1.24 | Sf3b1 |
| 1449140_at | Lgx only | 273 | 266 | 217 | 136 | −1.03 | −1.26 | −2.00 | Nudcd2 |
| 1449145_a_at | Lgx only | 11453 | 13083 | 13559 | 16570 | 1.14 | 1.18 | 1.45 | Cav1 |
| 1449151_at | Lgx only | 508 | 569 | 665 | 675 | 1.12 | 1.31 | 1.33 | Pctk3 |
| 1449187_at | Lgx only | 529 | 457 | 392 | 333 | −1.16 | −1.35 | −1.59 | Pdgfa |
| 1449217_at | Lgx only | 230 | 198 | 132 | 111 | −1.16 | −1.74 | −2.07 | Casp8ap2 |
| 1449256_a_at | Lgx only | 2956 | 2919 | 3638 | 3430 | −1.01 | 1.23 | 1.16 | Rab11a |
| 1449269_at | Lgx only | 168 | 147 | 139 | 61 | −1.15 | −1.21 | −2.75 | F5 |
| 1449281_at | Lgx only | 1873 | 2466 | 2400 | 2905 | 1.32 | 1.28 | 1.55 | Nrtn |
| 1449298_a_at | Lgx only | 237 | 268 | 246 | 322 | 1.13 | 1.04 | 1.36 | Pde1a |
| 1449300_at | Lgx only | 553 | 544 | 493 | 380 | −1.02 | −1.12 | −1.46 | Cttnbp2nl |
| 1449304_at | Lgx only | 463 | 404 | 649 | 649 | −1.15 | 1.40 | 1.40 | 2310061J03Rik |
| 1449333_at | Lgx only | 549 | 662 | 682 | 852 | 1.21 | 1.24 | 1.55 | Sf3a1 |
| 1449335_at | Lgx only | 5926 | 8089 | 8111 | 11301 | 1.37 | 1.37 | 1.91 | Timp3 |
| 1449338_at | Lgx only | 3319 | 3175 | 3019 | 2290 | −1.05 | −1.10 | −1.45 | D10Ertd641e |
| 1449345_at | Res only | 139 | 99 | 60 | 69 | −1.40 | −2.32 | −2.02 | Ccdc34 |
| 1449348_at | Lgx only | 2011 | 1876 | 1690 | 1475 | −1.07 | −1.19 | −1.36 | Mpp6 |
| 1449354_at | Res & Lgx | 959 | 1115 | 1378 | 1597 | 1.16 | 1.44 | 1.67 | Zrsr1 |
| 1449355_a_at | Lgx only | 676 | 967 | 976 | 975 | 1.43 | 1.44 | 1.44 | Eps15l1 |
| 1449356_at | Lgx only | 1478 | 999 | 1176 | 608 | −1.48 | −1.26 | −2.43 | Asb5 |
| 1449357_at | Res & Lgx | 286 | 192 | 172 | 175 | −1.49 | −1.67 | −1.64 | 2310030G06Rik |
| 1449363_at | Res only | 306 | 165 | 208 | 182 | −1.86 | −1.47 | −1.68 | Atf3 |
| 1449368_at | Lgx only | 25201 | 21347 | 33485 | 36603 | −1.18 | 1.33 | 1.45 | Dcn |
| 1449372_at | Res only | 1022 | 867 | 1303 | 1225 | −1.18 | 1.27 | 1.20 | Dnajc3a |
| 1449388_at | Lgx only | 707 | 620 | 583 | 439 | −1.14 | −1.21 | −1.61 | Thbs4 |
| 1449396_at | Lgx only | 250 | 389 | 302 | 405 | 1.56 | 1.21 | 1.62 | Aoc3 |
| 1449398_at | Lgx only | 8560 | 9487 | 11462 | 12020 | 1.11 | 1.34 | 1.40 | Rpl3l |
| 1449400_at | Lgx only | 22 | 43 | 45 | 102 | 1.93 | 2.04 | 4.58 | Csl |
| 1449408_at | Lgx only | 3014 | 3550 | 3724 | 3878 | 1.18 | 1.24 | 1.29 | Jam2 |
| 1449491_at | Lgx only | 1509 | 1650 | 1845 | 1973 | 1.09 | 1.22 | 1.31 | Card10 |
| 1449505_at | Res & Lgx | 2070 | 2142 | 1758 | 1736 | 1.03 | −1.18 | −1.19 | Kpna1 |
| 1449511_a_at | Lgx only | 661 | 691 | 781 | 854 | 1.05 | 1.18 | 1.29 | Ssbp4 |
| 1449514_at | CR only | 2473 | 1904 | 2807 | 2301 | −1.30 | 1.14 | −1.07 | Grk5 |
| 1449547_at | Res & Lgx | 2990 | 2491 | 1986 | 1510 | −1.20 | −1.51 | −1.98 | Asb14 |
| 1449551_at | Lgx only | 1623 | 1886 | 1869 | 1988 | 1.16 | 1.15 | 1.22 | Myo1c |
| 1449553_at | CR & Lgx | 868 | 640 | 1034 | 1201 | −1.36 | 1.19 | 1.38 | 2610200G18Rik |
| 1449566_at | Lgx only | 2519 | 2746 | 3461 | 3832 | 1.09 | 1.37 | 1.52 | Nkx2-5 |
| 1449575_a_at | Res & Lgx | 13795 | 15659 | 17129 | 18543 | 1.14 | 1.24 | 1.34 | Gstp1 |
| 1449588_at | Lgx only | 588 | 491 | 438 | 385 | −1.20 | −1.34 | −1.53 | Abca4 |
| 1449592_at | CR only | 1101 | 791 | 1255 | 1092 | −1.39 | 1.14 | −1.01 | Tcf15 |
| 1449813_at | Res only | 276 | 352 | 86 | 217 | 1.27 | −3.22 | −1.27 | Zfp30 |
| 1449818_at | CR only | 1563 | 2002 | 1765 | 1942 | 1.28 | 1.13 | 1.24 | Abcb4 |
| 1449842_at | Res & Lgx | 718 | 590 | 533 | 420 | −1.22 | −1.35 | −1.71 | 1810059G22Rik |
| 1449845_a_at | Lgx only | 745 | 860 | 974 | 1211 | 1.16 | 1.31 | 1.63 | Ephb4 |
| 1449849_a_at | Lgx only | 371 | 465 | 477 | 508 | 1.26 | 1.29 | 1.37 | Fbxl6 |
| 1449851_at | Lgx only | 442 | 679 | 726 | 810 | 1.54 | 1.64 | 1.83 | Per1 |
| 1449852_a_at | CR only | 6223 | 8480 | 5751 | 5735 | 1.36 | −1.08 | −1.09 | Ehd4 |
| 1449860_at | Lgx only | 730 | 574 | 585 | 447 | −1.27 | −1.25 | −1.63 | Higd1b |
| 1449872_at | Lgx only | 1222 | 1194 | 1026 | 765 | −1.02 | −1.19 | −1.60 | Hspb3 |
| 1449935_a_at | CR only | 3719 | 4445 | 4158 | 4213 | 1.20 | 1.12 | 1.13 | Dnaja3 |
| 1449942_a_at | Lgx only | 2010 | 2379 | 2324 | 2472 | 1.18 | 1.16 | 1.23 | Ilk |
| 1449944_a_at | Res only | 767 | 785 | 647 | 741 | 1.02 | −1.19 | −1.03 | Sec61a2 |
| 1449964_a_at | Lgx only | 2099 | 2072 | 3263 | 3012 | −1.01 | 1.55 | 1.43 | Mlycd |
| 1449969_at | Lgx only | 1482 | 1463 | 1356 | 1042 | −1.01 | −1.09 | −1.42 | Tmod4 |
| 1450016_at | Lgx only | 26432 | 25470 | 23049 | 19945 | −1.04 | −1.15 | −1.33 | Ccng1 |
| 1450023_at | Lgx only | 732 | 879 | 873 | 1230 | 1.20 | 1.19 | 1.68 | Gtpbp1 |
| 1450031_at | Lgx only | 1227 | 1176 | 1239 | 982 | −1.04 | 1.01 | −1.25 | Aff4 |
| 1450054_at | Lgx only | 2952 | 3635 | 3914 | 4173 | 1.23 | 1.33 | 1.41 | Add1 |
| 1450067_a_at | Res & Lgx | 2875 | 2957 | 2406 | 2199 | 1.03 | −1.19 | −1.31 | 1810034K20Rik |
| 1450086_at | Lgx only | 172 | 236 | 254 | 316 | 1.37 | 1.48 | 1.84 | Gmeb1 |
| 1450095_a_at | Lgx only | 387 | 339 | 395 | 207 | −1.14 | 1.02 | −1.87 | Acyp1 |
| 1450122_at | Lgx only | 539 | 488 | 473 | 337 | −1.11 | −1.14 | −1.60 | Ptprg |
| 1450123_at | Lgx only | 11629 | 13643 | 15609 | 19737 | 1.17 | 1.34 | 1.70 | Ryr2 |
| 1450138_a_at | Lgx only | 3319 | 3219 | 2766 | 2714 | −1.03 | −1.20 | −1.22 | Serpinb6a |
| 1450180_a_at | Lgx only | 38 | 71 | 80 | 181 | 1.85 | 2.09 | 4.70 | Rara |
| 1450195_at | Lgx only | 35 | 102 | 106 | 144 | 2.91 | 3.04 | 4.11 | Ndst4 |
| 1450199_a_at | Lgx only | 795 | 979 | 986 | 1080 | 1.23 | 1.24 | 1.36 | Stab1 |
| 1450203_at | Lgx only | 5141 | 5970 | 4705 | 4331 | 1.16 | −1.09 | −1.19 | Smyd1 |
| 1450308_a_at | Res & Lgx | 373 | 389 | 657 | 764 | 1.04 | 1.76 | 2.05 | Xrn1 |
| 1450355_a_at | Res only | 432 | 329 | 314 | 351 | −1.31 | −1.38 | −1.23 | Capg |
| 1450361_at | CR only | 129 | 52 | 132 | 65 | −2.47 | 1.02 | −1.99 | Prop1 |
| 1450376_at | Lgx only | 2003 | 2127 | 2276 | 2577 | 1.06 | 1.14 | 1.29 | Mxi1 |
| 1450377_at | Lgx only | 165 | 219 | 246 | 321 | 1.33 | 1.49 | 1.94 | LOC640441 |
| 1450395_at | Lgx only | 1580 | 1719 | 1822 | 2403 | 1.09 | 1.15 | 1.52 | Slc22a5 |
| 1450405_at | Res & Lgx | 725 | 643 | 354 | 320 | −1.13 | −2.05 | −2.27 | Mrpl19 |
| 1450409_a_at | Lgx only | 1748 | 1899 | 2253 | 2570 | 1.09 | 1.29 | 1.47 | 4930570C03Rik |
| 1450415_at | CR only | 56 | 17 | 56 | 44 | −3.33 | −1.01 | −1.29 | Pde6a |
| 1450424_a_at | CR only | 188 | 296 | 252 | 253 | 1.58 | 1.34 | 1.34 | Il18bp |
| 1450431_a_at | Res & Lgx | 11524 | 12897 | 16998 | 18939 | 1.12 | 1.48 | 1.64 | Nedd4 |
| 1450435_at | Lgx only | 277 | 284 | 353 | 397 | 1.03 | 1.27 | 1.44 | L1cam |
| 1450449_a_at | Lgx only | 3983 | 4570 | 4434 | 5830 | 1.15 | 1.11 | 1.46 | 2900002H16Rik |
| 1450490_at | Lgx only | 417 | 575 | 570 | 646 | 1.38 | 1.37 | 1.55 | Kcna7 |
| 1450519_a_at | Lgx only | 4324 | 5666 | 5682 | 6518 | 1.31 | 1.31 | 1.51 | Prkaca |
| 1450531_at | CR only | 102 | 24 | 54 | 78 | −4.30 | −1.90 | −1.30 | H2-Bl |
| 1450584_at | Res only | 25 | 69 | 107 | 61 | 2.76 | 4.32 | 2.45 | Hoxd11 |
| 1450623_at | Lgx only | 3269 | 3884 | 4094 | 4247 | 1.19 | 1.25 | 1.30 | Gnb2 |
| 1450627_at | Res & Lgx | 10383 | 11098 | 8410 | 8645 | 1.07 | −1.23 | −1.20 | Ank |
| 1450649_at | Lgx only | 1489 | 966 | 883 | 760 | −1.54 | −1.69 | −1.96 | Gng10 |
| 1450650_at | Lgx only | 987 | 1096 | 1732 | 1739 | 1.11 | 1.76 | 1.76 | Myo10 |
| 1450662_at | Lgx only | 584 | 632 | 750 | 830 | 1.08 | 1.28 | 1.42 | Tesk1 |
| 1450664_at | CR & Lgx | 1309 | 969 | 977 | 814 | −1.35 | −1.34 | −1.61 | Gabpa |
| 1450670_at | Lgx only | 884 | 1083 | 1043 | 1276 | 1.22 | 1.18 | 1.44 | Dbh |
| 1450672_a_at | Lgx only | 892 | 966 | 977 | 1154 | 1.08 | 1.10 | 1.29 | Trex1 |
| 1450678_at | CR only | 384 | 292 | 365 | 338 | −1.32 | −1.05 | −1.14 | Itgb2 |
| 1450690_at | Lgx only | 1537 | 1178 | 953 | 774 | −1.30 | −1.61 | −1.98 | Ranbp2 |
| 1450691_at | Res only | 621 | 793 | 919 | 894 | 1.28 | 1.48 | 1.44 | Caskin2 |
| 1450700_at | Res & Lgx | 1859 | 1642 | 1543 | 1404 | −1.13 | −1.20 | −1.32 | Cdc42ep3 |
| 1450706_a_at | Lgx only | 2959 | 2743 | 2744 | 2114 | −1.08 | −1.08 | −1.40 | Arl3 |
| 1450714_at | Res & Lgx | 2451 | 2021 | 1702 | 1453 | −1.21 | −1.44 | −1.69 | Azin1 |
| 1450729_at | Lgx only | 637 | 561 | 625 | 508 | −1.14 | −1.02 | −1.25 | Hs2st1 |
| 1450735_at | Res & Lgx | 2114 | 2019 | 1724 | 1463 | −1.05 | −1.23 | −1.45 | Pno1 |
| 1450738_at | Lgx only | 938 | 886 | 1089 | 1210 | −1.06 | 1.16 | 1.29 | Kif21a |
| 1450740_a_at | Res & Lgx | 4235 | 4039 | 3546 | 3025 | −1.05 | −1.19 | −1.40 | Mapre1 |
| 1450744_at | Lgx only | 488 | 355 | 297 | 213 | −1.38 | −1.65 | −2.29 | Ell2 |
| 1450759_at | Res only | 852 | 857 | 1146 | 1021 | 1.01 | 1.35 | 1.20 | Bmp6 |
| 1450791_at | Lgx only | 15042 | 15107 | 14450 | 8913 | 1.00 | −1.04 | −1.69 | Nppb |
| 1450798_at | Lgx only | 819 | 1305 | 1266 | 1651 | 1.59 | 1.54 | 2.01 | Tnxb |
| 1450801_at | Res & Lgx | 37 | 93 | 146 | 227 | 2.53 | 3.98 | 6.20 | Adam21 |
| 1450816_at | Res only | 384 | 341 | 301 | 362 | −1.13 | −1.27 | −1.06 | Polg2 |
| 1450839_at | Res only | 1123 | 1002 | 829 | 1015 | −1.12 | −1.35 | −1.11 | D0H4S114 |
| 1450840_a_at | Lgx only | 50799 | 56471 | 65223 | 75285 | 1.11 | 1.28 | 1.48 | Rpl39 |
| 1450842_a_at | CR & Lgx | 2062 | 1704 | 1758 | 1347 | −1.21 | −1.17 | −1.53 | Cenpa |
| 1450857_a_at | Lgx only | 2960 | 3430 | 3914 | 4745 | 1.16 | 1.32 | 1.60 | Col1a2 |
| 1450866_a_at | Lgx only | 3015 | 3253 | 3652 | 3857 | 1.08 | 1.21 | 1.28 | Mrpl17 |
| 1450878_at | Lgx only | 2049 | 1928 | 1635 | 1368 | −1.06 | −1.25 | −1.50 | Sri |
| 1450879_at | Lgx only | 610 | 710 | 716 | 993 | 1.16 | 1.17 | 1.63 | Atp9b |
| 1450883_a_at | CR only | 31708 | 23985 | 32717 | 30704 | −1.32 | 1.03 | −1.03 | Cd36 |
| 1450890_a_at | Lgx only | 948 | 726 | 729 | 585 | −1.31 | −1.30 | −1.62 | Abi1 |
| 1450891_at | Lgx only | 5718 | 5089 | 4197 | 3750 | −1.12 | −1.36 | −1.53 | Srp19 |
| 1450894_a_at | Res & Lgx | 2906 | 3585 | 3746 | 3815 | 1.23 | 1.29 | 1.31 | Ap2m1 |
| 1450897_at | Lgx only | 1688 | 1363 | 1427 | 1165 | −1.24 | −1.18 | −1.45 | Arhgap5 |
| 1450903_at | Lgx only | 1020 | 958 | 834 | 627 | −1.06 | −1.22 | −1.63 | Rad23b |
| 1450927_at | Lgx only | 514 | 591 | 685 | 712 | 1.15 | 1.33 | 1.39 | Lztr1 |
| 1450934_at | Res & Lgx | 22353 | 18933 | 16228 | 15963 | −1.18 | −1.38 | −1.40 | Eif4a2 |
| 1450948_a_at | Lgx only | 827 | 736 | 541 | 458 | −1.12 | −1.53 | −1.81 | Mrpl1 |
| 1450953_at | Res & Lgx | 2794 | 2661 | 2247 | 2084 | −1.05 | −1.24 | −1.34 | Ciao1 |
| 1450957_a_at | Lgx only | 14361 | 13299 | 20795 | 20648 | −1.08 | 1.45 | 1.44 | Sqstm1 |
| 1450958_at | Lgx only | 5954 | 4641 | 4404 | 4138 | −1.28 | −1.35 | −1.44 | Tm4sf1 |
| 1450965_at | Res & Lgx | 1995 | 2296 | 2538 | 3135 | 1.15 | 1.27 | 1.57 | Tex261 |
| 1450966_at | Lgx only | 927 | 836 | 863 | 679 | −1.11 | −1.07 | −1.37 | Crot |
| 1450968_at | Lgx only | 39927 | 41899 | 42555 | 55205 | 1.05 | 1.07 | 1.38 | Uqcrfs1 |
| 1450970_at | Lgx only | 25949 | 32719 | 31690 | 36332 | 1.26 | 1.22 | 1.40 | Got1 |
| 1450971_at | Res & Lgx | 551 | 827 | 965 | 1069 | 1.50 | 1.75 | 1.94 | Gadd45b |
| 1450974_at | Res & Lgx | 776 | 1077 | 1180 | 1212 | 1.39 | 1.52 | 1.56 | Timp4 |
| 1450994_at | Lgx only | 2773 | 2136 | 2256 | 1821 | −1.30 | −1.23 | −1.52 | Rock1 |
| 1451002_at | Lgx only | 54266 | 62734 | 61354 | 75692 | 1.16 | 1.13 | 1.39 | Aco2 |
| 1451006_at | Lgx only | 1143 | 1483 | 1634 | 1746 | 1.30 | 1.43 | 1.53 | Xdh |
| 1451010_at | Lgx only | 656 | 786 | 728 | 858 | 1.20 | 1.11 | 1.31 | Nol11 |
| 1451017_at | Lgx only | 1586 | 1728 | 1871 | 2173 | 1.09 | 1.18 | 1.37 | Ergic3 |
| 1451019_at | Lgx only | 1338 | 1281 | 1678 | 2014 | −1.04 | 1.25 | 1.51 | Ctsf |
| 1451022_at | Lgx only | 545 | 506 | 713 | 852 | −1.08 | 1.31 | 1.56 | Lrp6 |
| 1451025_at | Lgx only | 2443 | 2161 | 1929 | 1347 | −1.13 | −1.27 | −1.81 | Arl1 |
| 1451050_at | Lgx only | 3147 | 2948 | 2847 | 2290 | −1.07 | −1.11 | −1.37 | Nt5c3 |
| 1451051_a_at | Lgx only | 837 | 1040 | 1137 | 1455 | 1.24 | 1.36 | 1.74 | Scyl1 |
| 1451067_at | Lgx only | 3259 | 4112 | 4448 | 4416 | 1.26 | 1.37 | 1.36 | Sgta |
| 1451070_at | Lgx only | 2031 | 2545 | 2472 | 3265 | 1.25 | 1.22 | 1.61 | Gdi1 |
| 1451074_at | Res & Lgx | 4210 | 3824 | 3180 | 2816 | −1.10 | −1.32 | −1.49 | Rnf13 |
| 1451096_at | Lgx only | 29178 | 32946 | 32150 | 36689 | 1.13 | 1.10 | 1.26 | Ndufs2 |
| 1451099_at | Lgx only | 535 | 685 | 718 | 838 | 1.28 | 1.34 | 1.57 | Mbc2 |
| 1451104_a_at | CR & Lgx | 1056 | 1430 | 1492 | 1790 | 1.35 | 1.41 | 1.69 | Snrp70 |
| 1451118_a_at | Lgx only | 790 | 918 | 855 | 1042 | 1.16 | 1.08 | 1.32 | 2410018C17Rik |
| 1451119_a_at | Lgx only | 2418 | 2685 | 3222 | 3877 | 1.11 | 1.33 | 1.60 | Fbln1 |
| 1451121_a_at | Res & Lgx | 1112 | 1325 | 1636 | 1783 | 1.19 | 1.47 | 1.60 | Gltscr2 |
| 1451126_at | Lgx only | 4627 | 4444 | 5283 | 5589 | −1.04 | 1.14 | 1.21 | Maf1 |
| 1451134_a_at | Res & Lgx | 1207 | 953 | 612 | 601 | −1.27 | −1.97 | −2.01 | Tm2d2 |
| 1451144_at | All | 1442 | 1150 | 986 | 886 | −1.25 | −1.46 | −1.63 | Bxdc2 |
| 1451159_at | Res & Lgx | 4282 | 5561 | 6247 | 8339 | 1.30 | 1.46 | 1.95 | Arhgef12 |
| 1451168_a_at | Res & Lgx | 4732 | 5809 | 6829 | 7722 | 1.23 | 1.44 | 1.63 | Arhgdia |
| 1451177_at | Res & Lgx | 6681 | 6017 | 4465 | 4080 | −1.11 | −1.50 | −1.64 | Dnajb4 |
| 1451187_at | Lgx only | 4203 | 4110 | 3516 | 3225 | −1.02 | −1.20 | −1.30 | O610037P05Rik |
| 1451204_at | Res only | 2057 | 1633 | 1312 | 1410 | −1.26 | −1.57 | −1.46 | Scara5 |
| 1451217_a_at | Lgx only | 6459 | 5713 | 5401 | 4388 | −1.13 | −1.20 | −1.47 | Immp1l |
| 1451219_at | Lgx only | 915 | 777 | 738 | 662 | −1.18 | −1.24 | −1.38 | Ormdl1 |
| 1451223_a_at | Lgx only | 2697 | 2588 | 2232 | 1739 | −1.04 | −1.21 | −1.55 | Btf3l4 |
| 1451225_at | Lgx only | 6229 | 6428 | 6944 | 7210 | 1.03 | 1.11 | 1.16 | Ptpn11 |
| 1451226_at | Lgx only | 1149 | 1523 | 1685 | 2053 | 1.32 | 1.47 | 1.79 | Pex6 |
| 1451232_at | Lgx only | 6475 | 6003 | 4860 | 4346 | −1.08 | −1.33 | −1.49 | Cd151 |
| 1451244_a_at | Lgx only | 1155 | 1068 | 902 | 915 | −1.08 | −1.28 | −1.26 | Zfp422 |
| 1451245_at | Lgx only | 1838 | 1659 | 1561 | 1417 | −1.11 | −1.18 | −1.30 | Lrrc3b |
| 1451248_at | Res & Lgx | 1477 | 1443 | 1184 | 1086 | −1.02 | −1.25 | −1.36 | Prmt7 |
| 1451254_at | Res & Lgx | 1081 | 1005 | 719 | 681 | −1.08 | −1.50 | −1.59 | Ikbkap |
| 1451269_at | Lgx only | 1025 | 970 | 886 | 831 | −1.06 | −1.16 | −1.23 | Pdzd11 |
| 1451272_a_at | Res & Lgx | 1518 | 1300 | 972 | 856 | −1.17 | −1.56 | −1.77 | Ube2f |
| 1451274_at | Lgx only | 18853 | 24930 | 23212 | 26074 | 1.32 | 1.23 | 1.38 | Ogdh |
| 1451281_at | Res only | 182 | 134 | 126 | 176 | −1.36 | −1.44 | −1.03 | Zscan12 |
| 1451284_at | Lgx only | 824 | 1042 | 937 | 1011 | 1.26 | 1.14 | 1.23 | Yipf3 |
| 1451285_at | Lgx only | 1415 | 1654 | 1921 | 2284 | 1.17 | 1.36 | 1.61 | Fus |
| 1451290_at | Lgx only | 19210 | 20375 | 21862 | 26463 | 1.06 | 1.14 | 1.38 | Map1lc3a |
| 1451291_at | CR only | 985 | 1221 | 1225 | 1080 | 1.24 | 1.24 | 1.10 | Obfc2b |
| 1451293_at | CR only | 334 | 573 | 472 | 317 | 1.71 | 1.41 | −1.05 | Rrp9 |
| 1451295_a_at | Res & Lgx | 645 | 582 | 1919 | 1477 | −1.11 | 2.98 | 2.29 | Chd4 |
| 1451297_at | Res only | 17 | 25 | 80 | 44 | 1.45 | 4.66 | 2.55 | Gulo |
| 1451298_at | Lgx only | 536 | 562 | 684 | 976 | 1.05 | 1.28 | 1.82 | Plekhh3 |
| 1451312_at | Lgx only | 31762 | 35863 | 37350 | 45671 | 1.13 | 1.18 | 1.44 | Ndufs7 |
| 1451316_a_at | Res & Lgx | 5859 | 5369 | 3411 | 3242 | −1.09 | −1.72 | −1.81 | Picalm |
| 1451343_at | Lgx only | 832 | 752 | 752 | 595 | −1.11 | −1.11 | −1.40 | Vps36 |
| 1451344_at | Lgx only | 284 | 314 | 447 | 534 | 1.11 | 1.57 | 1.88 | Tmem119 |
| 1451349_at | Lgx only | 129 | 111 | 75 | 50 | −1.16 | −1.73 | −2.56 | BC020077 |
| 1451364_at | Res & Lgx | 1068 | 1040 | 814 | 549 | −1.03 | −1.31 | −1.94 | Polr3gl |
| 1451369_at | Lgx only | 1208 | 1284 | 1405 | 1759 | 1.06 | 1.16 | 1.46 | Commd5 |
| 1451381_at | Lgx only | 3059 | 2545 | 2352 | 2042 | −1.20 | −1.30 | −1.50 | 1810020D17Rik |
| 1451382_at | Res & Lgx | 247 | 447 | 470 | 562 | 1.81 | 1.90 | 2.28 | Chac1 |
| 1451388_a_at | Lgx only | 1122 | 1092 | 1224 | 1351 | −1.03 | 1.09 | 1.20 | Atp11b |
| 1451405_at | Lgx only | 3973 | 4289 | 5228 | 5041 | 1.08 | 1.32 | 1.27 | Pcca |
| 1451415_at | CR only | 1529 | 2052 | 1641 | 1570 | 1.34 | 1.07 | 1.03 | 1810011O10Rik |
| 1451420_at | Res & Lgx | 2878 | 2478 | 2242 | 2325 | −1.16 | −1.28 | −1.24 | Ccdc47 |
| 1451427_a_at | Lgx only | 1907 | 2012 | 2224 | 2426 | 1.05 | 1.17 | 1.27 | Egfl7 |
| 1451448_a_at | Lgx only | 1765 | 1548 | 1206 | 984 | −1.14 | −1.46 | −1.79 | 1110005A03Rik |
| 1451453_at | Lgx only | 246 | 248 | 265 | 336 | 1.01 | 1.08 | 1.36 | Dapk2 |
| 1451455_at | Res only | 273 | 220 | 478 | 374 | −1.24 | 1.75 | 1.37 | Thnsl2 |
| 1451462_a_at | Lgx only | 1261 | 1287 | 1200 | 958 | 1.02 | −1.05 | −1.32 | Ifnar2 |
| 1451465_at | Lgx only | 1407 | 1531 | 1643 | 1908 | 1.09 | 1.17 | 1.36 | Ubl7 |
| 1451471_at | Lgx only | 421 | 391 | 360 | 290 | −1.08 | −1.17 | −1.45 | Ears2 |
| 1451488_at | Lgx only | 7929 | 9254 | 9001 | 10760 | 1.17 | 1.14 | 1.36 | 1110028A07Rik |
| 1451502_at | CR only | 160 | 305 | 224 | 282 | 1.90 | 1.40 | 1.76 | Pla2g10 |
| 1451508_at | Lgx only | 1347 | 1314 | 1107 | 988 | −1.02 | −1.22 | −1.36 | Larp2 |
| 1451523_a_at | Lgx only | 1126 | 879 | 1016 | 827 | −1.28 | −1.11 | −1.36 | Mif4gd |
| 1451538_at | Lgx only | 135 | 123 | 153 | 242 | −1.10 | 1.13 | 1.78 | Sox9 |
| 1451553_at | Lgx only | 713 | 650 | 619 | 510 | −1.10 | −1.15 | −1.40 | Art5 |
| 1451561_at | Res & Lgx | 450 | 622 | 809 | 978 | 1.38 | 1.80 | 2.18 | Prr12 |
| 1451583_a_at | Res & Lgx | 69 | 119 | 158 | 261 | 1.72 | 2.29 | 3.78 | BC025076 |
| 1451604_a_at | Lgx only | 658 | 716 | 707 | 849 | 1.09 | 1.07 | 1.29 | Acvrl1 |
| 1451622_at | Lgx only | 2006 | 2187 | 2476 | 2643 | 1.09 | 1.23 | 1.32 | Lmbrd1 |
| 1451663_a_at | Lgx only | 743 | 755 | 847 | 912 | 1.02 | 1.14 | 1.23 | Trim3 |
| 1451665_a_at | Res & Lgx | 5435 | 4923 | 4504 | 4166 | −1.10 | −1.21 | −1.30 | Ap4s1 |
| 1451674_at | Lgx only | 114 | 131 | 180 | 195 | 1.15 | 1.57 | 1.70 | Slc12a5 |
| 1451678_at | Res & Lgx | 2482 | 2409 | 1553 | 1507 | −1.03 | −1.60 | −1.65 | Narf |
| 1451700_a_at | Res & Lgx | 2076 | 2271 | 2807 | 2740 | 1.09 | 1.35 | 1.32 | 1110007L15Rik |
| 1451728_at | Lgx only | 792 | 823 | 931 | 1122 | 1.04 | 1.17 | 1.42 | Wdr13 |
| 1451741_a_at | Lgx only | 732 | 614 | 585 | 518 | −1.19 | −1.25 | −1.41 | Cdk7 |
| 1451742_a_at | Lgx only | 14534 | 14261 | 12397 | 11426 | −1.02 | −1.17 | −1.27 | Ugp2 |
| 1451782_a_at | Lgx only | 2095 | 2186 | 2027 | 2552 | 1.04 | −1.03 | 1.22 | Slc29a1 |
| 1451789_a_at | CR only | 651 | 1037 | 851 | 972 | 1.59 | 1.31 | 1.49 | Ryk |
| 1451803_a_at | Lgx only | 6764 | 8556 | 8339 | 9104 | 1.26 | 1.23 | 1.35 | Vegfb |
| 1451820_at | Lgx only | 131 | 214 | 241 | 536 | 1.64 | 1.84 | 4.10 | Diras1 |
| 1451839_a_at | CR only | 890 | 1444 | 1029 | 1071 | 1.62 | 1.16 | 1.20 | Pde7a |
| 1451854_a_at | Lgx only | 383 | 572 | 575 | 694 | 1.49 | 1.50 | 1.81 | Shroom3 |
| 1451857_a_at | Lgx only | 194 | 239 | 319 | 373 | 1.23 | 1.64 | 1.92 | Notum |
| 1451883_at | Res & Lgx | 401 | 497 | 932 | 825 | 1.24 | 2.32 | 2.06 | ENSMUSG- |
| | | | | | | | | | 00000074670 |
| 1451902_at | Lgx only | 572 | 427 | 446 | 344 | −1.34 | −1.28 | −1.66 | Zfp758 |
| 1451911_a_at | Lgx only | 784 | 928 | 1055 | 1218 | 1.18 | 1.35 | 1.55 | Ace |
| 1451974_at | Lgx only | 3494 | 3897 | 3875 | 4394 | 1.12 | 1.11 | 1.26 | Osbpl2 |
| 1451984_at | Res & Lgx | 1996 | 1833 | 1480 | 1574 | −1.09 | −1.35 | −1.27 | Hnrpul1 |
| 1452012_a_at | Lgx only | 688 | 561 | 589 | 433 | −1.23 | −1.17 | −1.59 | Exosc1 |
| 1452024_a_at | Lgx only | 972 | 977 | 1305 | 1295 | 1.00 | 1.34 | 1.33 | Ldb1 |
| 1452043_at | Lgx only | 638 | 593 | 728 | 510 | −1.07 | 1.14 | −1.25 | 2310011J03Rik |
| 1452047_at | Res & Lgx | 2343 | 2094 | 1524 | 1383 | −1.12 | −1.54 | −1.69 | Cacybp |
| 1452057_at | Lgx only | 1040 | 1024 | 1176 | 1469 | −1.02 | 1.13 | 1.41 | Actr1b |
| 1452058_a_at | Res & Lgx | 8691 | 8360 | 5764 | 5728 | −1.04 | −1.51 | −1.52 | Rnf11 |
| 1452072_at | Res & Lgx | 1185 | 937 | 765 | 822 | −1.27 | −1.55 | −1.44 | Myct1 |
| 1452080_a_at | Lgx only | 1213 | 1057 | 971 | 775 | −1.15 | −1.25 | −1.57 | Dcun1d1 |
| 1452088_at | Res only | 153 | 231 | 81 | 127 | 1.51 | −1.89 | −1.21 | Zbed3 |
| 1452091_a_at | CR & Lgx | 852 | 1127 | 962 | 1169 | 1.32 | 1.13 | 1.37 | Rbm28 |
| 1452110_at | Lgx only | 459 | 361 | 351 | 327 | −1.27 | −1.31 | −1.41 | Mtrr |
| 1452130_at | Res & Lgx | 1764 | 1802 | 966 | 869 | 1.02 | −1.83 | −2.03 | Txndc14 |
| 1452140_at | Res & Lgx | 867 | 934 | 1137 | 1294 | 1.08 | 1.31 | 1.49 | Tbc1d20 |
| 1452141_a_at | Lgx only | 25419 | 26171 | 28265 | 32953 | 1.03 | 1.11 | 1.30 | Sepp1 |
| 1452143_at | All | 21772 | 24259 | 24942 | 27902 | 1.11 | 1.15 | 1.28 | Spnb2 |
| 1452145_at | Lgx only | 524 | 698 | 748 | 1076 | 1.33 | 1.43 | 2.06 | H6pd |
| 1452152_at | Lgx only | 2710 | 2333 | 2165 | 1785 | −1.16 | −1.25 | −1.52 | Clint1 |
| 1452155_a_at | Res & Lgx | 3205 | 3897 | 4271 | 4549 | 1.22 | 1.33 | 1.42 | Ddx17 |
| 1452156_a_at | Lgx only | 4703 | 5715 | 5941 | 7648 | 1.22 | 1.26 | 1.63 | Nisch |
| 1452159_at | Res & Lgx | 1379 | 1311 | 1056 | 977 | −1.05 | −1.31 | −1.41 | 2310001A20Rik |
| 1452173_at | Lgx only | 26826 | 30831 | 34012 | 38315 | 1.15 | 1.27 | 1.43 | Hadha |
| 1452174_at | Res & Lgx | 500 | 634 | 880 | 1000 | 1.27 | 1.76 | 2.00 | Srebf2 |
| 1452202_at | Lgx only | 1135 | 1145 | 1336 | 1475 | 1.01 | 1.18 | 1.30 | Pde2a |
| 1452203_at | All | 2414 | 1686 | 1491 | 1035 | −1.43 | −1.62 | −2.33 | Obfc2a |
| 1452208_at | Lgx only | 485 | 620 | 638 | 696 | 1.28 | 1.31 | 1.43 | Prdm4 |
| 1452213_at | Res & Lgx | 1320 | 1264 | 1036 | 845 | −1.04 | −1.27 | −1.56 | Tex2 |
| 1452214_at | Lgx only | 1513 | 1185 | 1126 | 850 | −1.28 | −1.34 | −1.78 | Skil |
| 1452221_a_at | Lgx only | 1063 | 1125 | 1281 | 1393 | 1.06 | 1.20 | 1.31 | Cxxc1 |
| 1452222_at | Lgx only | 1656 | 1643 | 1732 | 2090 | −1.01 | 1.05 | 1.26 | Utrn |
| 1452225_at | Lgx only | 1335 | 1061 | 1047 | 1000 | −1.26 | −1.28 | −1.33 | 2010106G01Rik |
| 1452250_a_at | Lgx only | 2260 | 2785 | 2667 | 3592 | 1.23 | 1.18 | 1.59 | Col6a2 |
| 1452262_at | Lgx only | 708 | 642 | 550 | 557 | −1.10 | −1.29 | −1.27 | Grpel2 |
| 1452286_at | Res & Lgx | 6581 | 4659 | 2979 | 2444 | −1.41 | −2.21 | −2.69 | Slain2 |
| 1452291_at | Lgx only | 564 | 461 | 566 | 360 | −1.22 | 1.00 | −1.57 | Centd1 |
| 1452292_at | Lgx only | 1094 | 1334 | 1435 | 1847 | 1.22 | 1.31 | 1.69 | Ap2b1 |
| 1452296_at | Lgx only | 237 | 266 | 412 | 579 | 1.12 | 1.74 | 2.45 | Slit3 |
| 1452308_a_at | Lgx only | 4905 | 5839 | 6466 | 6497 | 1.19 | 1.32 | 1.32 | Atp1a2 |
| 1452309_at | Lgx only | 721 | 839 | 763 | 1132 | 1.16 | 1.06 | 1.57 | Cgnl1 |
| 1452318_a_at | Res & Lgx | 536 | 385 | 278 | 289 | −1.39 | −1.93 | −1.86 | Hspa1b |
| 1452319_at | CR only | 283 | 194 | 251 | 220 | −1.46 | −1.13 | −1.28 | Zfp82 |
| 1452327_at | Lgx only | 904 | 1190 | 1305 | 1397 | 1.32 | 1.44 | 1.55 | Iqsec1 |
| 1452329_at | Res & Lgx | 265 | 311 | 451 | 486 | 1.17 | 1.71 | 1.84 | Plekhn1 |
| 1452330_a_at | Lgx only | 2664 | 2991 | 3519 | 4501 | 1.12 | 1.32 | 1.69 | Mxra8 |
| 1452333_at | Res & Lgx | 475 | 512 | 815 | 822 | 1.08 | 1.71 | 1.73 | Smarca2 |
| 1452335_at | Res & Lgx | 485 | 467 | 382 | 319 | −1.04 | −1.27 | −1.52 | Mfsd8 |
| 1452339_at | Lgx only | 320 | 425 | 415 | 535 | 1.33 | 1.30 | 1.67 | Adamts7 |
| 1452374_at | Lgx only | 756 | 605 | 602 | 553 | −1.25 | −1.26 | −1.37 | Zfp322a |
| 1452375_at | Lgx only | 1059 | 1280 | 1336 | 1441 | 1.21 | 1.26 | 1.36 | Aldh4a1 |
| 1452395_at | Res & Lgx | 550 | 421 | 386 | 295 | −1.31 | −1.42 | −1.87 | Med19 |
| 1452398_at | Lgx only | 1052 | 998 | 837 | 640 | −1.05 | −1.26 | −1.64 | Plce1 |
| 1452401_at | Lgx only | 1940 | 1839 | 1632 | 1573 | −1.05 | −1.19 | −1.23 | Wtap |
| 1452411_at | Lgx only | 261 | 364 | 308 | 474 | 1.40 | 1.18 | 1.82 | Lrrc1 |
| 1452432_at | Lgx only | 2342 | 2445 | 2339 | 1802 | 1.04 | −1.00 | −1.30 | Tfpi |
| 1452446_a_at | Lgx only | 1260 | 1260 | 1436 | 1569 | 1.00 | 1.14 | 1.25 | Tmub2 |
| 1452462_a_at | Lgx only | 342 | 469 | 538 | 591 | 1.37 | 1.58 | 1.73 | Banp |
| 1452469_a_at | Res & Lgx | 1833 | 2687 | 2690 | 3184 | 1.47 | 1.47 | 1.74 | Smtn |
| 1452472_at | Lgx only | 128 | 142 | 189 | 242 | 1.11 | 1.47 | 1.89 | Rtp3 |
| 1452499_a_at | Res only | 705 | 659 | 530 | 573 | −1.07 | −1.33 | −1.23 | Kif2a |
| 1452502_at | Lgx only | 190 | 121 | 131 | 88 | −1.56 | −1.45 | −2.15 | ENSMUSG- |
| | | | | | | | | | 00000050599 |
| 1452509_at | CR only | 114 | 33 | 80 | 89 | −3.44 | −1.44 | −1.29 | Usp9y |
| 1452587_at | Lgx only | 3962 | 3990 | 3352 | 3268 | 1.01 | −1.18 | −1.21 | Actr2 |
| 1452596_at | Lgx only | 4654 | 3854 | 3014 | 2105 | −1.21 | −1.54 | −2.21 | Polr2k |
| 1452601_a_at | Lgx only | 978 | 969 | 1221 | 1242 | −1.01 | 1.25 | 1.27 | Acbd6 |
| 1452607_at | Lgx only | 978 | 917 | 707 | 587 | −1.07 | −1.38 | −1.67 | 2610030H06Rik |
| 1452608_at | Res & Lgx | 727 | 527 | 412 | 452 | −1.38 | −1.76 | −1.61 | Mycbp |
| 1452625_at | Lgx only | 772 | 766 | 950 | 1078 | −1.01 | 1.23 | 1.40 | Kctd2 |
| 1452653_at | Lgx only | 1121 | 1286 | 1501 | 1804 | 1.15 | 1.34 | 1.61 | Slc25a22 |
| 1452657_at | Lgx only | 1898 | 1692 | 1530 | 1326 | −1.12 | −1.24 | −1.43 | Ap1s2 |
| 1452661_at | Res & Lgx | 4325 | 6173 | 1466 | 1666 | 1.43 | −2.95 | −2.60 | Tfrc |
| 1452689_at | Res only | 1202 | 1315 | 1457 | 1353 | 1.09 | 1.21 | 1.13 | Zfp512 |
| 1452694_at | Lgx only | 1776 | 1941 | 2134 | 2382 | 1.09 | 1.20 | 1.34 | Ihpk1 |
| 1452698_at | Lgx only | 1101 | 1252 | 1139 | 1500 | 1.14 | 1.04 | 1.36 | Tsfm |
| 1452709_at | Res & Lgx | 590 | 680 | 819 | 833 | 1.15 | 1.39 | 1.41 | Poldip3 |
| 1452714_at | Lgx only | 872 | 1040 | 976 | 1284 | 1.19 | 1.12 | 1.47 | Tanc1 |
| 1452737_at | Lgx only | 1487 | 1388 | 1103 | 899 | −1.07 | −1.35 | −1.65 | 2810008M24Rik |
| 1452745_at | Lgx only | 788 | 1066 | 1236 | 1408 | 1.35 | 1.57 | 1.79 | 1810044A24Rik |
| 1452749_at | Lgx only | 1136 | 1094 | 893 | 738 | −1.04 | −1.27 | −1.54 | Papd1 |
| 1452753_at | Res only | 837 | 908 | 529 | 625 | 1.08 | −1.58 | −1.34 | Foxk2 |
| 1452761_a_at | Lgx only | 485 | 513 | 599 | 1042 | 1.06 | 1.24 | 2.15 | 8430436O14Rik |
| 1452767_at | Lgx only | 2638 | 2966 | 3499 | 3821 | 1.12 | 1.33 | 1.45 | Rrbp1 |
| 1452776_a_at | Res only | 663 | 841 | 1073 | 970 | 1.27 | 1.62 | 1.46 | Nub1 |
| 1452813_a_at | Res & Lgx | 2436 | 2172 | 1960 | 1741 | −1.12 | −1.24 | −1.40 | Tmem188 |
| 1452843_at | Res & Lgx | 3451 | 4038 | 4593 | 5346 | 1.17 | 1.33 | 1.55 | Il6st |
| 1452844_at | Res only | 751 | 823 | 1059 | 1074 | 1.10 | 1.41 | 1.43 | Pou6f1 |
| 1452856_at | Res only | 739 | 644 | 536 | 657 | −1.15 | −1.38 | −1.13 | Crebzf |
| 1452866_at | CR & Res | 2752 | 2260 | 2177 | 2385 | −1.22 | −1.26 | −1.15 | Nars |
| 1452867_at | Lgx only | 2212 | 1946 | 1780 | 1556 | −1.14 | −1.24 | −1.42 | Col4a3bp |
| 1452871_at | Lgx only | 416 | 352 | 302 | 259 | −1.18 | −1.38 | −1.61 | Neil1 |
| 1452874_at | Lgx only | 2271 | 2311 | 2091 | 1836 | 1.02 | −1.09 | −1.24 | 2510003E04Rik |
| 1452877_at | Lgx only | 2296 | 2197 | 1472 | 1361 | −1.05 | −1.56 | −1.69 | 2700029M09Rik |
| 1452885_at | Lgx only | 1428 | 1377 | 2182 | 2172 | −1.04 | 1.53 | 1.52 | Sfrs2ip |
| 1452897_at | CR & Lgx | 696 | 543 | 615 | 531 | −1.28 | −1.13 | −1.31 | Cdc2l5 |
| 1452901_at | Lgx only | 1170 | 1145 | 777 | 693 | −1.02 | −1.50 | −1.69 | Creb1 |
| 1452913_at | Lgx only | 2974 | 3092 | 3677 | 4408 | 1.04 | 1.24 | 1.48 | Pcp4l1 |
| 1452918_at | Res & Lgx | 829 | 733 | 480 | 371 | −1.13 | −1.73 | −2.23 | D19Ertd737e |
| 1452920_a_at | Lgx only | 1190 | 1193 | 1012 | 760 | 1.00 | −1.18 | −1.57 | Ppil2 |
| 1452952_at | Res & Lgx | 922 | 773 | 689 | 632 | −1.19 | −1.34 | −1.46 | 9030418K01Rik |
| 1452953_at | Res & Lgx | 992 | 827 | 679 | 580 | −1.20 | −1.46 | −1.71 | Fam18b |
| 1452960_at | Lgx only | 245 | 207 | 208 | 163 | −1.18 | −1.18 | −1.51 | Scyl3 |
| 1452965_at | Res only | 29 | 40 | 111 | 84 | 1.36 | 3.79 | 2.86 | Ankrd13d |
| 1452972_at | Lgx only | 1013 | 814 | 912 | 729 | −1.25 | −1.11 | −1.39 | Ttc32 |
| 1452977_at | Lgx only | 531 | 595 | 622 | 751 | 1.12 | 1.17 | 1.42 | Zhx3 |
| 1452985_at | Lgx only | 878 | 899 | 1344 | 1287 | 1.02 | 1.53 | 1.47 | Uaca |
| 1452999_at | Lgx only | 842 | 889 | 680 | 565 | 1.06 | −1.24 | −1.49 | Smndc1 |
| 1453007_at | CR only | 283 | 386 | 348 | 337 | 1.36 | 1.23 | 1.19 | 3110082I17Rik |
| 1453013_at | Res & Lgx | 382 | 472 | 620 | 612 | 1.23 | 1.62 | 1.60 | Zfp740 |
| 1453014_a_at | Lgx only | 1817 | 2542 | 2796 | 3042 | 1.40 | 1.54 | 1.67 | Sec31a |
| 1453023_at | Lgx only | 617 | 600 | 801 | 808 | −1.03 | 1.30 | 1.31 | Ankhd1 |
| 1453028_at | Lgx only | 215 | 210 | 184 | 94 | −1.03 | −1.17 | −2.29 | 4631424J17Rik |
| 1453039_at | Lgx only | 590 | 726 | 780 | 855 | 1.23 | 1.32 | 1.45 | Zfp335 |
| 1453058_at | Lgx only | 174 | 168 | 136 | 55 | −1.04 | −1.28 | −3.18 | Wdr5b |
| 1453059_at | Lgx only | 8551 | 8336 | 7741 | 7324 | −1.03 | −1.10 | −1.17 | 2310046A06Rik |
| 1453062_at | CR only | 223 | 400 | 295 | 433 | 1.80 | 1.33 | 1.95 | A930026I22Rik |
| 1453097_a_at | Lgx only | 1577 | 1678 | 1749 | 2148 | 1.06 | 1.11 | 1.36 | Ubtf |
| 1453119_at | Res & Lgx | 559 | 481 | 365 | 290 | −1.16 | −1.53 | −1.92 | Otud1 |
| 1453129_a_at | Lgx only | 557 | 617 | 773 | 837 | 1.11 | 1.39 | 1.50 | Rgs12 |
| 1453137_at | Lgx only | 1023 | 1022 | 916 | 767 | −1.00 | −1.12 | −1.33 | Fbxo30 |
| 1453149_at | Lgx only | 290 | 191 | 219 | 111 | −1.52 | −1.33 | −2.61 | Slc25a32 |
| 1453154_at | CR only | 79 | 24 | 84 | 44 | −3.27 | 1.06 | −1.78 | 1700029M20Rik |
| 1453155_at | Lgx only | 2675 | 2692 | 2995 | 3076 | 1.01 | 1.12 | 1.15 | Tmem50a |
| 1453160_at | Lgx only | 738 | 596 | 734 | 458 | −1.24 | −1.01 | −1.61 | Med13 |
| 1453180_at | Res only | 782 | 784 | 635 | 639 | 1.00 | −1.23 | −1.23 | 6530404N21Rik |
| 1453187_at | All | 605 | 410 | 365 | 248 | −1.48 | −1.66 | −2.44 | Ociad2 |
| 1453191_at | Lgx only | 374 | 503 | 391 | 738 | 1.34 | 1.04 | 1.97 | Col27a1 |
| 1453196_a_at | Res & Lgx | 254 | 340 | 389 | 411 | 1.34 | 1.53 | 1.62 | Oasl2 |
| 1453206_at | Res & Lgx | 890 | 1145 | 1255 | 1633 | 1.29 | 1.41 | 1.84 | Acad9 |
| 1453212_at | Lgx only | 319 | 237 | 226 | 141 | −1.34 | −1.41 | −2.27 | Zfp383 |
| 1453224_at | Res & Lgx | 809 | 940 | 1461 | 1631 | 1.16 | 1.81 | 2.02 | Zfand5 |
| 1453257_at | Lgx only | 853 | 753 | 660 | 637 | −1.13 | −1.29 | −1.34 | Agpat5 |
| 1453271_at | Res & Lgx | 1296 | 1098 | 939 | 900 | −1.18 | −1.38 | −1.44 | Phf14 |
| 1453296_at | CR only | 113 | 32 | 90 | 134 | −3.51 | −1.25 | 1.19 | Tmem103 |
| 1453312_at | Lgx only | 790 | 818 | 632 | 503 | 1.04 | −1.25 | −1.57 | Iqwd1 |
| 1453377_at | Res & Lgx | 606 | 603 | 406 | 399 | −1.00 | −1.49 | −1.52 | Sh2d4a |
| 1453391_at | CR only | 28 | 75 | 84 | 70 | 2.64 | 2.97 | 2.47 | Speer7-ps1 |
| 1453399_at | Res & Lgx | 607 | 726 | 1159 | 1232 | 1.20 | 1.91 | 2.03 | Ccnt2 |
| 1453412_a_at | CR only | 2089 | 2711 | 2591 | 2801 | 1.30 | 1.24 | 1.34 | Sec14l1 |
| 1453486_a_at | Lgx only | 1455 | 1240 | 1142 | 1034 | −1.17 | −1.27 | −1.41 | Scube2 |
| 1453494_at | Res only | 98 | 79 | 44 | 66 | −1.23 | −2.21 | −1.49 | 4921513H07Rik |
| 1453502_at | Lgx only | 77 | 79 | 94 | 27 | 1.02 | 1.22 | −2.82 | 2210408I21Rik |
| 1453552_at | CR & Lgx | 1567 | 1069 | 1454 | 980 | −1.47 | −1.08 | −1.60 | 2310014F07Rik |
| 1453572_a_at | Lgx only | 2411 | 2727 | 2793 | 3149 | 1.13 | 1.16 | 1.31 | Plp2 |
| 1453592_at | Lgx only | 3846 | 3055 | 3329 | 2431 | −1.26 | −1.16 | −1.58 | Lrrc39 |
| 1453673_at | Lgx only | 76 | 65 | 94 | 172 | −1.18 | 1.24 | 2.26 | LOC100046982 |
| 1453728_a_at | Lgx only | 3261 | 3568 | 2905 | 2627 | 1.09 | −1.12 | −1.24 | Mrps17 |
| 1453729_a_at | Lgx only | 41216 | 43763 | 46983 | 55549 | 1.06 | 1.14 | 1.35 | Rpl37 |
| 1453731_a_at | Lgx only | 3325 | 2887 | 2681 | 2049 | −1.15 | −1.24 | −1.62 | Tmem77 |
| 1453739_at | Res & Lgx | 450 | 425 | 316 | 266 | −1.06 | −1.42 | −1.69 | Tmem126b |
| 1453740_a_at | Lgx only | 2063 | 2311 | 2547 | 2926 | 1.12 | 1.24 | 1.42 | Ccnl2 |
| 1453761_at | Res only | 74 | 144 | 179 | 199 | 1.95 | 2.43 | 2.71 | Phf6 |
| 1453795_at | Lgx only | 1896 | 2038 | 2175 | 2434 | 1.08 | 1.15 | 1.28 | Fahd2a |
| 1453804_a_at | Lgx only | 804 | 758 | 637 | 604 | −1.06 | −1.26 | −1.33 | Orc4l |
| 1453821_at | Res only | 642 | 596 | 1039 | 923 | −1.08 | 1.62 | 1.44 | N6amt1 |
| 1453850_at | Lgx only | 43 | 35 | 37 | 131 | −1.24 | −1.19 | 3.01 | 1500002I01Rik |
| 1453851_a_at | Lgx only | 352 | 496 | 457 | 640 | 1.41 | 1.30 | 1.82 | Gadd45g |
| 1453865_a_at | Res & Lgx | 869 | 969 | 1199 | 1124 | 1.12 | 1.38 | 1.29 | Otud5 |
| 1453866_a_at | Lgx only | 43 | 74 | 75 | 126 | 1.71 | 1.73 | 2.91 | Xk |
| 1453898_at | CR only | 501 | 793 | 335 | 384 | 1.58 | −1.49 | −1.30 | Itgb1bp3 |
| 1453913_a_at | Lgx only | 849 | 962 | 1213 | 1299 | 1.13 | 1.43 | 1.53 | Tap2 |
| 1453976_at | CR only | 143 | 76 | 125 | 134 | −1.89 | −1.15 | −1.07 | 4432414F05Rik |
| 1453983_a_at | Lgx only | 927 | 804 | 894 | 635 | −1.15 | −1.04 | −1.46 | Mett10d |
| 1454017_at | Res & Lgx | 25 | 38 | 74 | 84 | 1.54 | 3.00 | 3.41 | 4921509O07Rik |
| 1454020_at | Lgx only | 51 | 52 | 75 | 163 | 1.04 | 1.49 | 3.21 | 4930554H23Rik |
| 1454021_a_at | CR only | 1143 | 1359 | 1194 | 1279 | 1.19 | 1.04 | 1.12 | Exosc10 |
| 1454023_a_at | Lgx only | 385 | 503 | 469 | 613 | 1.31 | 1.22 | 1.59 | D1Bwg1363e |
| 1454034_a_at | Lgx only | 569 | 709 | 792 | 894 | 1.25 | 1.39 | 1.57 | Usp21 |
| 1454047_a_at | CR only | 276 | 183 | 203 | 229 | −1.51 | −1.36 | −1.20 | 2410017P07Rik |
| 1454074_a_at | Res & Lgx | 1611 | 1425 | 1123 | 900 | −1.13 | −1.43 | −1.79 | Rsrc2 |
| 1454092_a_at | Res only | 593 | 635 | 442 | 463 | 1.07 | −1.34 | −1.28 | Gtf2h3 |
| 1454109_a_at | Res & Lgx | 591 | 481 | 406 | 366 | −1.23 | −1.46 | −1.61 | Jmjd6 |
| 1454116_a_at | Lgx only | 1801 | 1594 | 1238 | 1086 | −1.13 | −1.46 | −1.66 | Mterfd1 |
| 1454189_at | Lgx only | 73 | 110 | 141 | 142 | 1.51 | 1.93 | 1.94 | 4930557J02Rik |
| 1454206_a_at | Lgx only | 392 | 464 | 580 | 728 | 1.18 | 1.48 | 1.86 | Adam15 |
| 1454214_a_at | Lgx only | 746 | 743 | 637 | 541 | −1.00 | −1.17 | −1.38 | 2410019A14Rik |
| 1454236_a_at | Res only | 269 | 286 | 159 | 248 | 1.06 | −1.69 | −1.08 | Ppp4r1l |
| 1454395_at | Lgx only | 439 | 558 | 623 | 761 | 1.27 | 1.42 | 1.73 | 4632404M16Rik |
| 1454455_at | Lgx only | 72 | 65 | 114 | 167 | −1.10 | 1.59 | 2.33 | 6530413G14Rik |
| 1454466_at | CR only | 80 | 13 | 67 | 52 | −6.28 | −1.18 | −1.54 | 4933407I18Rik |
| 1454606_at | Lgx only | 1497 | 1775 | 1754 | 1897 | 1.19 | 1.17 | 1.27 | 4933426M11Rik |
| 1454611_a_at | Res only | 4696 | 4347 | 3422 | 3420 | −1.08 | −1.37 | −1.37 | Calm1 |
| 1454612_at | Lgx only | 610 | 597 | 535 | 437 | −1.02 | −1.14 | −1.40 | Mex3c |
| 1454613_at | Lgx only | 978 | 1329 | 1415 | 1720 | 1.36 | 1.45 | 1.76 | Dpysl3 |
| 1454619_at | Lgx only | 1392 | 1555 | 1880 | 2331 | 1.12 | 1.35 | 1.67 | Tmem112b |
| 1454626_at | Lgx only | 3655 | 3568 | 2821 | 2614 | −1.02 | −1.30 | −1.40 | Cltc |
| 1454631_at | Lgx only | 639 | 592 | 552 | 433 | −1.08 | −1.16 | −1.48 | Gtf2a1 |
| 1454638_a_at | Lgx only | 1004 | 949 | 703 | 592 | −1.06 | −1.43 | −1.70 | Pah |
| 1454644_at | Lgx only | 470 | 566 | 617 | 705 | 1.20 | 1.31 | 1.50 | 6330569M22Rik |
| 1454645_at | Res & Lgx | 3953 | 5231 | 5398 | 6226 | 1.32 | 1.37 | 1.57 | Mgrn1 |
| 1454646_at | Res only | 5976 | 6134 | 4613 | 4866 | 1.03 | −1.30 | −1.23 | Tcp11l2 |
| 1454647_at | Lgx only | 3249 | 2669 | 2518 | 2112 | −1.22 | −1.29 | −1.54 | Acad11 |
| 1454654_at | Lgx only | 1244 | 1418 | 1330 | 1645 | 1.14 | 1.07 | 1.32 | Dirc2 |
| 1454658_at | Res only | 790 | 826 | 1079 | 882 | 1.05 | 1.37 | 1.12 | Ilvbl |
| 1454666_at | Lgx only | 2164 | 1836 | 1500 | 1233 | −1.18 | −1.44 | −1.76 | LOC100046855 |
| 1454670_at | Lgx only | 1041 | 1359 | 1101 | 1577 | 1.31 | 1.06 | 1.51 | Rere |
| 1454679_at | Lgx only | 374 | 407 | 335 | 481 | 1.09 | −1.12 | 1.29 | D8Ertd457e |
| 1454682_at | Lgx only | 1062 | 918 | 870 | 788 | −1.16 | −1.22 | −1.35 | A430005L14Rik |
| 1454693_at | Lgx only | 512 | 617 | 534 | 706 | 1.20 | 1.04 | 1.38 | Hdac4 |
| 1454697_at | Lgx only | 1244 | 1274 | 1567 | 1632 | 1.02 | 1.26 | 1.31 | Tloc1 |
| 1454704_at | Lgx only | 8488 | 8673 | 9500 | 10160 | 1.02 | 1.12 | 1.20 | Scarb2 |
| 1454706_at | Res & Lgx | 841 | 933 | 1147 | 1655 | 1.11 | 1.36 | 1.97 | Uvrag |
| 1454709_at | CR only | 1558 | 1256 | 1428 | 1353 | −1.24 | −1.09 | −1.15 | Tmem64 |
| 1454718_at | CR only | 405 | 601 | 518 | 565 | 1.48 | 1.28 | 1.40 | Nagpa |
| 1454723_at | Lgx only | 553 | 511 | 424 | 378 | −1.08 | −1.31 | −1.46 | 1110033M05Rik |
| 1454727_at | Lgx only | 1394 | 1215 | 977 | 798 | −1.15 | −1.43 | −1.75 | Afap1l1 |
| 1454730_at | All | 769 | 590 | 524 | 464 | −1.30 | −1.47 | −1.66 | Tapt1 |
| 1454733_at | Res only | 911 | 985 | 1221 | 1082 | 1.08 | 1.34 | 1.19 | Nod1 |
| 1454739_at | Res & Lgx | 644 | 552 | 440 | 462 | −1.17 | −1.46 | −1.39 | Cdc27 |
| 1454745_at | Lgx only | 3289 | 3105 | 2800 | 2804 | −1.06 | −1.17 | −1.17 | Arhgap29 |
| 1454749_at | Lgx only | 684 | 403 | 1251 | 1482 | −1.70 | 1.83 | 2.17 | Pcnt |
| 1454753_at | Lgx only | 1587 | 2106 | 2105 | 2294 | 1.33 | 1.33 | 1.45 | Rnpepl1 |
| 1454759_at | Lgx only | 438 | 629 | 684 | 820 | 1.43 | 1.56 | 1.87 | Git1 |
| 1454780_at | Res & Lgx | 369 | 411 | 559 | 501 | 1.12 | 1.52 | 1.36 | Galntl4 |
| 1454797_at | Res & Lgx | 1016 | 1208 | 1395 | 1607 | 1.19 | 1.37 | 1.58 | Tmem55b |
| 1454801_at | Lgx only | 432 | 318 | 409 | 291 | −1.36 | −1.06 | −1.48 | Ankrd28 |
| 1454813_at | Lgx only | 14224 | 13070 | 12391 | 10412 | −1.09 | −1.15 | −1.37 | Ccdc72 |
| 1454820_at | Res & Lgx | 41 | 115 | 186 | 231 | 2.83 | 4.59 | 5.68 | BC037034 |
| 1454834_at | Lgx only | 2775 | 2711 | 2558 | 2415 | −1.02 | −1.08 | −1.15 | Nfib |
| 1454848_at | Lgx only | 465 | 542 | 638 | 645 | 1.16 | 1.37 | 1.39 | Ppp1r12c |
| 1454861_at | Lgx only | 629 | 688 | 770 | 967 | 1.09 | 1.22 | 1.54 | Txlna |
| 1454868_at | Lgx only | 1589 | 2148 | 2178 | 2607 | 1.35 | 1.37 | 1.64 | D4Ertd429e |
| 1454887_at | Lgx only | 747 | 899 | 852 | 1049 | 1.20 | 1.14 | 1.41 | Pak2 |
| 1454888_at | Res & Lgx | 1357 | 1167 | 902 | 903 | −1.16 | −1.50 | −1.50 | Pfdn4 |
| 1454892_at | Lgx only | 2663 | 2785 | 3279 | 3697 | 1.05 | 1.23 | 1.39 | Pitpnb |
| 1454894_at | Lgx only | 1514 | 1552 | 1292 | 1165 | 1.03 | −1.17 | −1.30 | LOC100045522 |
| 1454895_at | Res & Lgx | 580 | 737 | 920 | 969 | 1.27 | 1.59 | 1.67 | Cybasc3 |
| 1454906_at | Res & Lgx | 1336 | 1248 | 925 | 804 | −1.07 | −1.44 | −1.66 | Rarb |
| 1454914_at | Lgx only | 1116 | 1031 | 928 | 845 | −1.08 | −1.20 | −1.32 | 2610101N10Rik |
| 1454915_at | Res & Lgx | 351 | 379 | 517 | 591 | 1.08 | 1.47 | 1.68 | Rab3gap2 |
| 1454921_at | Lgx only | 2860 | 2668 | 2533 | 1763 | −1.07 | −1.13 | −1.62 | Gm561 |
| 1454922_at | Res & Lgx | 927 | 893 | 748 | 601 | −1.04 | −1.24 | −1.54 | Wdr92 |
| 1454928_at | Lgx only | 431 | 638 | 710 | 848 | 1.48 | 1.65 | 1.97 | Safb |
| 1454930_at | CR only | 902 | 684 | 840 | 692 | −1.32 | −1.07 | −1.30 | Tbcel |
| 1454934_at | CR only | 783 | 941 | 754 | 809 | 1.20 | −1.04 | 1.03 | Ppm1f |
| 1454937_at | Lgx only | 411 | 351 | 295 | 228 | −1.17 | −1.39 | −1.80 | B630005N14Rik |
| 1454938_at | Lgx only | 1417 | 1321 | 1222 | 1011 | −1.07 | −1.16 | −1.40 | Snx13 |
| 1454955_at | Lgx only | 6741 | 6181 | 6145 | 5319 | −1.09 | −1.10 | −1.27 | Ipo7 |
| 1454960_at | Lgx only | 737 | 915 | 886 | 1114 | 1.24 | 1.20 | 1.51 | Smad3 |
| 1454964_at | CR only | 4312 | 5102 | 4189 | 4991 | 1.18 | −1.03 | 1.16 | BC021395 |
| 1454977_at | Lgx only | 235 | 290 | 342 | 395 | 1.23 | 1.46 | 1.68 | AU020772 |
| 1454979_at | Lgx only | 940 | 990 | 1118 | 1206 | 1.05 | 1.19 | 1.28 | Diap1 |
| 1454980_at | Lgx only | 2726 | 3305 | 3169 | 3859 | 1.21 | 1.16 | 1.42 | 4930402E16Rik |
| 1454985_at | Lgx only | 374 | 389 | 391 | 531 | 1.04 | 1.05 | 1.42 | D030051N19Rik |
| 1454991_at | Res & Lgx | 1439 | 1242 | 1003 | 894 | −1.16 | −1.43 | −1.61 | Slc7a1 |
| 1454997_at | CR only | 2397 | 3094 | 2233 | 2451 | 1.29 | −1.07 | 1.02 | Msrb3 |
| 1455014_at | Res & Lgx | 1453 | 1054 | 833 | 759 | −1.38 | −1.75 | −1.91 | AV009015 |
| 1455018_at | Lgx only | 1301 | 1669 | 1774 | 2383 | 1.28 | 1.36 | 1.83 | Lmtk2 |
| 1455025_at | Res only | 1073 | 1005 | 788 | 849 | −1.07 | −1.36 | −1.26 | Paqr9 |
| 1455051_at | Lgx only | 500 | 578 | 720 | 851 | 1.16 | 1.44 | 1.70 | Rnf31 |
| 1455061_a_at | Lgx only | 19692 | 20976 | 24991 | 27704 | 1.07 | 1.27 | 1.41 | Acaa2 |
| 1455081_at | Lgx only | 393 | 471 | 526 | 526 | 1.20 | 1.34 | 1.34 | Txnl4b |
| 1455082_at | Lgx only | 455 | 375 | 356 | 312 | −1.21 | −1.28 | −1.46 | Cblb |
| 1455090_at | Lgx only | 2902 | 3308 | 2931 | 3642 | 1.14 | 1.01 | 1.26 | Angptl2 |
| 1455105_at | Lgx only | 1072 | 1049 | 833 | 740 | −1.02 | −1.29 | −1.45 | Ptpn12 |
| 1455118_at | Res & Lgx | 1938 | 1850 | 1436 | 1478 | −1.05 | −1.35 | −1.31 | D9Ertd402e |
| 1455131_at | Lgx only | 2218 | 2140 | 2041 | 1890 | −1.04 | −1.09 | −1.17 | Opa3 |
| 1455142_at | Lgx only | 409 | 403 | 306 | 230 | −1.02 | −1.34 | −1.78 | Socs4 |
| 1455143_at | Res & Lgx | 326 | 441 | 617 | 772 | 1.35 | 1.89 | 2.36 | Nlgn2 |
| 1455152_at | Res & Lgx | 2267 | 2135 | 1838 | 1619 | −1.06 | −1.23 | −1.40 | Al462493 |
| 1455153_at | Lgx only | 505 | 453 | 471 | 342 | −1.11 | −1.07 | −1.47 | Zfp236 |
| 1455155_at | Lgx only | 1223 | 1091 | 976 | 862 | −1.12 | −1.25 | −1.42 | Lsm14b |
| 1455157_a_at | Res & Lgx | 788 | 737 | 1180 | 1481 | −1.07 | 1.50 | 1.88 | BC039210 |
| 1455159_at | Lgx only | 527 | 455 | 447 | 310 | −1.16 | −1.18 | −1.70 | Appl1 |
| 1455164_at | Lgx only | 1752 | 2160 | 2211 | 2588 | 1.23 | 1.26 | 1.48 | Cdgap |
| 1455166_at | Res & Lgx | 1049 | 897 | 812 | 763 | −1.17 | −1.29 | −1.37 | Arl5b |
| 1455188_at | Res only | 445 | 474 | 263 | 378 | 1.06 | −1.69 | −1.18 | Ephb1 |
| 1455197_at | Res only | 89 | 155 | 200 | 137 | 1.74 | 2.24 | 1.54 | Rnd1 |
| 1455204_at | Res & Lgx | 1784 | 1576 | 1400 | 1239 | −1.13 | −1.27 | −1.44 | Pitpnc1 |
| 1455205_a_at | Lgx only | 1304 | 1651 | 1627 | 1812 | 1.27 | 1.25 | 1.39 | Usp19 |
| 1455207_at | Lgx only | 687 | 606 | 578 | 507 | −1.13 | −1.19 | −1.36 | 2410017P09Rik |
| 1455210_at | Lgx only | 573 | 682 | 716 | 811 | 1.19 | 1.25 | 1.42 | Zhx2 |
| 1455226_at | Res & Lgx | 980 | 1142 | 1530 | 1869 | 1.17 | 1.56 | 1.91 | Spnb1 |
| 1455244_at | Res & Lgx | 3049 | 2993 | 2023 | 1746 | −1.02 | −1.51 | −1.75 | Daam1 |
| 1455267_at | Lgx only | 1101 | 1050 | 793 | 685 | −1.05 | −1.39 | −1.61 | Esrrg |
| 1455268_at | Res & Lgx | 575 | 477 | 313 | 321 | −1.20 | −1.84 | −1.79 | Dph3 |
| 1455285_at | Res & Lgx | 760 | 771 | 569 | 445 | 1.01 | −1.34 | −1.71 | Slc31a1 |
| 1455288_at | Res & Lgx | 1136 | 1274 | 1630 | 1957 | 1.12 | 1.43 | 1.72 | 1110036O03Rik |
| 1455293_at | Lgx only | 249 | 208 | 259 | 124 | −1.20 | 1.04 | −2.02 | Leo1 |
| 1455296_at | Lgx only | 784 | 1049 | 929 | 1119 | 1.34 | 1.19 | 1.43 | LOC100047385 |
| 1455300_at | Res & Lgx | 629 | 510 | 389 | 317 | −1.23 | −1.62 | −1.98 | E130014J05Rik |
| 1455307_at | Lgx only | 487 | 544 | 697 | 794 | 1.12 | 1.43 | 1.63 | BC037112 |
| 1455309_at | Res & Lgx | 761 | 829 | 1263 | 1387 | 1.09 | 1.66 | 1.82 | Tmem16f |
| 1455312_at | Lgx only | 785 | 972 | 944 | 1067 | 1.24 | 1.20 | 1.36 | Phc3 |
| 1455320_at | CR & Lgx | 3658 | 2918 | 2870 | 2793 | −1.25 | −1.27 | −1.31 | Al480535 |
| 1455340_at | Res & Lgx | 576 | 384 | 329 | 275 | −1.50 | −1.75 | −2.09 | D030011O10Rik |
| 1455349_at | Lgx only | 697 | 526 | 549 | 463 | −1.32 | −1.27 | −1.50 | LOC100048397 |
| 1455353_at | Lgx only | 968 | 809 | 836 | 782 | −1.20 | −1.16 | −1.24 | Tmcc1 |
| 1455356_at | Lgx only | 320 | 299 | 499 | 662 | −1.07 | 1.56 | 2.07 | Camsap1 |
| 1455387_at | Res & Lgx | 1358 | 1115 | 995 | 758 | −1.22 | −1.36 | −1.79 | Nufip2 |
| 1455390_at | Lgx only | 1575 | 1427 | 1439 | 1118 | −1.10 | −1.09 | −1.41 | Alkbh6 |
| 1455434_a_at | Res & Lgx | 6402 | 5782 | 5247 | 4407 | −1.11 | −1.22 | −1.45 | Ktn1 |
| 1455442_at | Res & Lgx | 74 | 104 | 176 | 197 | 1.41 | 2.37 | 2.67 | Slc6a19 |
| 1455450_at | Lgx only | 1232 | 1200 | 1327 | 1592 | −1.03 | 1.08 | 1.29 | Ptpn3 |
| 1455456_a_at | Lgx only | 1242 | 1516 | 1332 | 1587 | 1.22 | 1.07 | 1.28 | Timm50 |
| 1455462_at | Res & Lgx | 218 | 251 | 334 | 356 | 1.15 | 1.53 | 1.63 | Adcy2 |
| 1455479_a_at | Res & Lgx | 12338 | 12135 | 7678 | 6396 | −1.02 | −1.61 | −1.93 | Ube2d3 |
| 1455482_at | All | 516 | 724 | 797 | 1018 | 1.40 | 1.54 | 1.97 | Ap2a2 |
| 1455491_at | Res & Lgx | 1099 | 1208 | 811 | 846 | 1.10 | −1.36 | −1.30 | Hnrph3 |
| 1455506_at | Lgx only | 5998 | 5890 | 8637 | 9404 | −1.02 | 1.44 | 1.57 | Slc25a34 |
| 1455508_at | Lgx only | 354 | 367 | 312 | 217 | 1.04 | −1.14 | −1.63 | A530082C11Rik |
| 1455538_at | Lgx only | 942 | 726 | 609 | 472 | −1.30 | −1.55 | −2.00 | 6330403M23Rik |
| 1455585_at | Lgx only | 601 | 526 | 486 | 404 | −1.14 | −1.24 | −1.49 | Rnf168 |
| 1455587_at | Res only | 11 | 16 | 20 | 21 | 1.39 | 1.75 | 1.82 | BC030183 |
| 1455588_at | Res & Lgx | 1065 | 816 | 578 | 556 | −1.30 | −1.84 | −1.91 | Lyrm4 |
| 1455655_a_at | Res & Lgx | 1506 | 1115 | 925 | 831 | −1.35 | −1.63 | −1.81 | Tardbp |
| 1455688_at | Lgx only | 951 | 817 | 745 | 702 | −1.16 | −1.28 | −1.35 | Ddr2 |
| 1455689_at | Lgx only | 270 | 358 | 391 | 488 | 1.33 | 1.45 | 1.81 | Fzd10 |
| 1455700_at | Res & Lgx | 1328 | 1183 | 811 | 664 | −1.12 | −1.64 | −2.00 | Mterfd3 |
| 1455702_at | Lgx only | 584 | 734 | 962 | 803 | 1.26 | 1.65 | 1.37 | Wdr22 |
| 1455733_at | Res & Lgx | 135 | 216 | 228 | 227 | 1.60 | 1.69 | 1.68 | Taok3 |
| 1455734_at | CR & Lgx | 1289 | 881 | 1118 | 921 | −1.46 | −1.15 | −1.40 | Crbn |
| 1455741_a_at | Res & Lgx | 1206 | 1346 | 1718 | 2034 | 1.12 | 1.42 | 1.69 | Ece1 |
| 1455750_at | Res & Lgx | 1072 | 1041 | 733 | 717 | −1.03 | −1.46 | −1.50 | A230067G21Rik |
| 1455757_at | Res & Lgx | 522 | 526 | 371 | 305 | 1.01 | −1.41 | −1.71 | D3Ertd254e |
| 1455794_at | Lgx only | 566 | 625 | 809 | 1034 | 1.10 | 1.43 | 1.83 | Smtnl2 |
| 1455832_a_at | Res only | 420 | 352 | 325 | 287 | −1.19 | −1.29 | −1.46 | Umps |
| 1455854_a_at | Res & Lgx | 470 | 513 | 771 | 829 | 1.09 | 1.64 | 1.76 | Ssh1 |
| 1455870_at | Lgx only | 2176 | 2652 | 2908 | 3503 | 1.22 | 1.34 | 1.61 | Akap2 |
| 1455873_a_at | Lgx only | 271 | 383 | 402 | 483 | 1.41 | 1.48 | 1.78 | Vps18 |
| 1455884_at | Lgx only | 508 | 747 | 821 | 851 | 1.47 | 1.62 | 1.67 | Dpp9 |
| 1455914_at | Lgx only | 168 | 138 | 101 | 93 | −1.22 | −1.66 | −1.81 | Al987944 |
| 1455915_at | CR only | 191 | 132 | 150 | 138 | −1.44 | −1.27 | −1.38 | Galnt4 |
| 1455922_at | Lgx only | 1213 | 1256 | 1440 | 1560 | 1.04 | 1.19 | 1.29 | Rab3gap1 |
| 1455936_a_at | CR & Lgx | 1540 | 2056 | 1827 | 2200 | 1.33 | 1.19 | 1.43 | Rbpms |
| 1455944_at | Lgx only | 153 | 127 | 219 | 289 | −1.21 | 1.43 | 1.88 | Zfp516 |
| 1455945_at | Lgx only | 192 | 166 | 136 | 117 | −1.16 | −1.41 | −1.65 | Zfp817 |
| 1456046_at | Lgx only | 3587 | 3017 | 2786 | 2266 | −1.19 | −1.29 | −1.58 | Cd93 |
| 1456058_at | Lgx only | 807 | 745 | 690 | 598 | −1.08 | −1.17 | −1.35 | Rbm27 |
| 1456059_at | Lgx only | 4664 | 4475 | 3594 | 3214 | −1.04 | −1.30 | −1.45 | Psmd11 |
| 1456061_at | Res & Lgx | 740 | 574 | 371 | 258 | −1.29 | −1.99 | −2.87 | Gimap8 |
| 1456065_at | CR only | 128 | 49 | 171 | 119 | −2.60 | 1.33 | −1.07 | Ubash3a |
| 1456092_at | Lgx only | 86 | 52 | 58 | 26 | −1.67 | −1.49 | −3.35 | Kctd7 |
| 1456099_at | Res only | 128 | 174 | 254 | 207 | 1.35 | 1.98 | 1.61 | D930017J03Rik |
| 1456161_at | Lgx only | 403 | 283 | 297 | 279 | −1.42 | −1.36 | −1.44 | O610040B10Rik |
| 1456169_at | Res only | 128 | 171 | 178 | 144 | 1.34 | 1.39 | 1.13 | EG226654 |
| 1456210_at | Lgx only | 150 | 178 | 101 | 68 | 1.18 | −1.48 | −2.21 | 5430407P10Rik |
| 1456241_a_at | Lgx only | 3667 | 3705 | 4635 | 4984 | 1.01 | 1.26 | 1.36 | 1810073N04Rik |
| 1456257_at | Res only | 495 | 565 | 327 | 409 | 1.14 | −1.51 | −1.21 | C130065N10Rik |
| 1456315_a_at | Res & Lgx | 5639 | 5549 | 3253 | 2716 | −1.02 | −1.73 | −2.08 | Ptpla |
| 1456398_at | Lgx only | 1951 | 1675 | 1544 | 1204 | −1.16 | −1.26 | −1.62 | Tug1 |
| 1456487_at | Lgx only | 270 | 438 | 425 | 592 | 1.62 | 1.57 | 2.19 | Adcy1 |
| 1456599_at | CR only | 106 | 165 | 118 | 109 | 1.55 | 1.11 | 1.03 | Nxt2 |
| 1456604_a_at | Lgx only | 2365 | 2051 | 1676 | 1367 | −1.15 | −1.41 | −1.73 | Pcmt1 |
| 1456611_at | Lgx only | 462 | 581 | 505 | 608 | 1.26 | 1.09 | 1.31 | D430015B01Rik |
| 1456625_at | Lgx only | 192 | 140 | 145 | 126 | −1.37 | −1.33 | −1.52 | Aasdhppt |
| 1456643_at | Lgx only | 386 | 308 | 278 | 233 | −1.25 | −1.39 | −1.65 | 9230114K14Rik |
| 1456659_at | Lgx only | 319 | 363 | 307 | 153 | 1.14 | −1.04 | −2.08 | LOC552902 |
| 1456727_a_at | Res & Lgx | 2775 | 2975 | 4693 | 5107 | 1.07 | 1.69 | 1.84 | Csnk1d |
| 1456768_a_at | Lgx only | 1333 | 1602 | 1733 | 2588 | 1.20 | 1.30 | 1.94 | Mmm2 |
| 1456774_at | Lgx only | 324 | 594 | 806 | 961 | 1.83 | 2.49 | 2.97 | Ppp1r13l |
| 1456777_at | CR & Lgx | 38 | 14 | 75 | 13 | −2.79 | 1.95 | −3.02 | Mgam |
| 1456827_at | Lgx only | 196 | 148 | 119 | 61 | −1.33 | −1.64 | −3.22 | AA987161 |
| 1456836_at | CR only | 151 | 39 | 112 | 115 | −3.85 | −1.34 | −1.31 | Itk |
| 1456871_a_at | Res only | 1810 | 1658 | 2405 | 2210 | −1.09 | 1.33 | 1.22 | Phf20l1 |
| 1456888_at | Lgx only | 252 | 244 | 313 | 371 | −1.03 | 1.24 | 1.47 | Pfkfb4 |
| 1456896_at | Res only | 473 | 464 | 642 | 654 | −1.02 | 1.36 | 1.38 | 6720462K09Rik |
| 1456914_at | Lgx only | 239 | 197 | 190 | 142 | −1.21 | −1.26 | −1.69 | Slc16a4 |
| 1457058_at | Lgx only | 245 | 319 | 349 | 556 | 1.31 | 1.43 | 2.27 | Adamts2 |
| 1457111_at | Res only | 267 | 251 | 189 | 227 | −1.07 | −1.41 | −1.18 | AA415038 |
| 1457276_at | Res only | 149 | 225 | 240 | 200 | 1.51 | 1.61 | 1.35 | Snf1lk2 |
| 1457285_at | Lgx only | 1194 | 1178 | 764 | 664 | −1.01 | −1.56 | −1.80 | Zfp187 |
| 1457334_at | All | 286 | 516 | 618 | 643 | 1.80 | 2.16 | 2.25 | C130057M05Rik |
| 1457401_at | Lgx only | 63 | 89 | 114 | 167 | 1.41 | 1.81 | 2.64 | Dnahc9 |
| 1457448_at | CR only | 154 | 79 | 90 | 117 | −1.94 | −1.71 | −1.32 | Pnpla1 |
| 1457501_at | Res only | 20 | 78 | 111 | 63 | 3.99 | 5.68 | 3.23 | RP23-233B9.8 |
| 1457508_at | Res & Lgx | 400 | 281 | 286 | 239 | −1.42 | −1.40 | −1.67 | C430003N24Rik |
| 1457557_at | Lgx only | 81 | 31 | 72 | 23 | −2.59 | −1.13 | −3.55 | A330076H08Rik |
| 1457626_at | Res only | 58 | 84 | 135 | 152 | 1.44 | 2.31 | 2.59 | D3Wsu106e |
| 1457671_at | Lgx only | 767 | 700 | 555 | 483 | −1.10 | −1.38 | −1.59 | 9330120H11Rik |
| 1457681_at | Lgx only | 1108 | 992 | 1385 | 1651 | −1.12 | 1.25 | 1.49 | 2610301F02Rik |
| 1457707_at | Res & Lgx | 55 | 71 | 153 | 177 | 1.29 | 2.78 | 3.21 | Mctp2 |
| 1457745_at | Res only | 361 | 391 | 519 | 505 | 1.08 | 1.44 | 1.40 | Gpr4 |
| 1457747_at | Res only | 8 | 14 | 30 | 35 | 1.77 | 3.81 | 4.34 | 9330109K16Rik |
| 1457801_at | CR only | 268 | 388 | 343 | 329 | 1.45 | 1.28 | 1.23 | 9930024M15Rik |
| 1458058_at | Lgx only | 149 | 104 | 122 | 59 | −1.44 | −1.21 | −2.53 | 7030407E18Rik |
| 1458190_at | Res only | 120 | 146 | 291 | 240 | 1.21 | 2.41 | 2.00 | Arhgap4 |
| 1458311_at | Res only | 249 | 304 | 433 | 381 | 1.22 | 1.74 | 1.53 | Usp36 |
| 1458353_at | Res only | 162 | 269 | 340 | 244 | 1.66 | 2.10 | 1.51 | Nwd1 |
| 1458438_at | Lgx only | 240 | 181 | 180 | 118 | −1.33 | −1.34 | −2.03 | Ccdc122 |
| 1458455_at | Lgx only | 566 | 438 | 512 | 358 | −1.29 | −1.11 | −1.58 | Abra |
| 1458461_at | Res only | 12 | 21 | 40 | 20 | 1.66 | 3.22 | 1.57 | E330021D16Rik |
| 1458478_at | Res only | 65 | 48 | 12 | 53 | −1.36 | −5.63 | −1.23 | 9230110F11Rik |
| 1458482_at | Lgx only | 1340 | 1453 | 1156 | 1017 | 1.08 | −1.16 | −1.32 | Tnni3k |
| 1458624_at | Res & Lgx | 1590 | 1831 | 2206 | 2675 | 1.15 | 1.39 | 1.68 | Rbm24 |
| 1458780_at | Res only | 20 | 68 | 74 | 55 | 3.33 | 3.61 | 2.70 | D8Ertd620e |
| 1458863_at | Res only | 152 | 101 | 37 | 85 | −1.50 | −4.15 | −1.79 | 6330415G19Rik |
| 1459108_a_at | Res & Lgx | 219 | 259 | 328 | 445 | 1.18 | 1.50 | 2.03 | Yeats2 |
| 1459220_at | Lgx only | 138 | 217 | 222 | 276 | 1.57 | 1.61 | 2.00 | C78651 |
| 1459363_at | Lgx only | 312 | 405 | 407 | 506 | 1.30 | 1.31 | 1.62 | Atxn2 |
| 1459578_at | Res only | 54 | 61 | 155 | 88 | 1.12 | 2.85 | 1.62 | AA407175 |
| 1460033_at | CR only | 74 | 180 | 125 | 105 | 2.42 | 1.69 | 1.42 | C030002C11Rik |
| 1460053_at | CR only | 242 | 144 | 218 | 221 | −1.68 | −1.11 | −1.10 | Smyd4 |
| 1460113_at | CR only | 104 | 187 | 144 | 134 | 1.80 | 1.38 | 1.29 | B930093H17Rik |
| 1460165_at | Res & Lgx | 9064 | 9043 | 7129 | 6522 | −1.00 | −1.27 | −1.39 | Ppp1ca |
| 1460167_at | Lgx only | 988 | 1120 | 1063 | 1253 | 1.13 | 1.08 | 1.27 | Aldh7a1 |
| 1460169_a_at | Lgx only | 2453 | 3136 | 3555 | 4351 | 1.28 | 1.45 | 1.77 | Pctk1 |
| 1460177_at | Res & Lgx | 1301 | 1575 | 1640 | 1671 | 1.21 | 1.26 | 1.28 | Cndp2 |
| 1460184_at | Lgx only | 36182 | 38955 | 42606 | 49147 | 1.08 | 1.18 | 1.36 | Hadh |
| 1460189_at | Res & Lgx | 4600 | 3988 | 3323 | 2755 | −1.15 | −1.38 | −1.67 | Wdr23 |
| 1460194_at | Lgx only | 12145 | 13386 | 14000 | 14613 | 1.10 | 1.15 | 1.20 | Phyh |
| 1460196_at | Lgx only | 1079 | 1068 | 1158 | 1385 | −1.01 | 1.07 | 1.28 | Cbr1 |
| 1460210_at | Lgx only | 683 | 848 | 775 | 1026 | 1.24 | 1.13 | 1.50 | Pkd1 |
| 1460214_at | CR only | 27 | 97 | 92 | 108 | 3.67 | 3.46 | 4.07 | Pcp4 |
| 1460216_at | Lgx only | 5275 | 5712 | 6310 | 7377 | 1.08 | 1.20 | 1.40 | Acads |
| 1460230_at | Lgx only | 106 | 114 | 120 | 192 | 1.07 | 1.13 | 1.81 | Syn2 |
| 1460239_at | Lgx only | 3477 | 3308 | 3484 | 3037 | −1.05 | 1.00 | −1.15 | Tspan13 |
| 1460251_at | Lgx only | 464 | 467 | 407 | 262 | 1.01 | −1.14 | −1.77 | Fas |
| 1460254_at | Res & Lgx | 1563 | 1408 | 1168 | 924 | −1.11 | −1.34 | −1.69 | 1810049H13Rik |
| 1460271_at | CR only | 157 | 66 | 118 | 125 | −2.38 | −1.33 | −1.26 | Trem3 |
| 1460276_a_at | Lgx only | 822 | 886 | 967 | 1097 | 1.08 | 1.18 | 1.33 | Gpr175 |
| 1460321_at | CR only | 129 | 52 | 117 | 105 | −2.48 | −1.10 | −1.23 | Cntn4 |
| 1460326_at | Res & Lgx | 1395 | 1325 | 1867 | 1690 | −1.05 | 1.34 | 1.21 | Pik3ca |
| 1460328_at | Res & Lgx | 619 | 628 | 880 | 862 | 1.01 | 1.42 | 1.39 | Brd3 |
| 1460329_at | Res only | 713 | 637 | 458 | 504 | −1.12 | −1.56 | −1.42 | LOC675709 |
| 1460330_at | Lgx only | 1601 | 1521 | 1413 | 1219 | −1.05 | −1.13 | −1.31 | Anxa3 |
| 1460331_at | Res & Lgx | 5861 | 5156 | 4527 | 4030 | −1.14 | −1.29 | −1.45 | Tm9sf2 |
| 1460336_at | Lgx only | 2689 | 2686 | 4293 | 5218 | −1.00 | 1.60 | 1.94 | Ppargc1a |
| 1460337_at | CR only | 4383 | 5948 | 3663 | 4114 | 1.36 | −1.20 | −1.07 | Sh3kbp1 |
| 1460344_at | Lgx only | 1002 | 1085 | 1313 | 1398 | 1.08 | 1.31 | 1.40 | 2310033F14Rik |
| 1460396_at | Lgx only | 483 | 621 | 790 | 817 | 1.29 | 1.63 | 1.69 | Ddx54 |
| 1460409_at | Lgx only | 1345 | 1292 | 1713 | 2001 | −1.04 | 1.27 | 1.49 | Cpt1a |
| 1460412_at | CR only | 58 | 20 | 45 | 57 | −2.84 | −1.30 | −1.03 | 1600015H20Rik |
| 1460420_a_at | Lgx only | 634 | 820 | 934 | 1142 | 1.29 | 1.47 | 1.80 | Egfr |
| 1460428_at | Lgx only | 919 | 958 | 1120 | 1123 | 1.04 | 1.22 | 1.22 | Ankrd13a |
| 1460432_a_at | Lgx only | 12140 | 9937 | 10609 | 8730 | −1.22 | −1.14 | −1.39 | Eif3e |
| 1460433_at | Lgx only | 662 | 763 | 813 | 1034 | 1.15 | 1.23 | 1.56 | Entpd6 |
| 1460435_at | Lgx only | 573 | 713 | 699 | 935 | 1.25 | 1.22 | 1.63 | 1500002O20Rik |
| 1460444_at | Lgx only | 329 | 323 | 411 | 491 | −1.02 | 1.25 | 1.49 | Arrb1 |
| 1460500_at | Lgx only | 313 | 176 | 158 | 98 | −1.78 | −1.98 | −3.20 | 5033421C21Rik |
| 1460510_a_at | Res & Lgx | 3505 | 3184 | 2702 | 2908 | −1.10 | −1.30 | −1.21 | Coq10b |
| 1460539_at | Res only | 68 | 139 | 141 | 153 | 2.04 | 2.07 | 2.25 | 4933404K13Rik |
| 1460547_a_at | Res & Lgx | 4912 | 3450 | 2670 | 1733 | −1.42 | −1.84 | −2.84 | Hnrpk |
| 1460552_at | Lgx only | 2039 | 2109 | 2286 | 2886 | 1.03 | 1.12 | 1.42 | Ascc3l1 |
| 1460557_at | Lgx only | 1577 | 1866 | 1401 | 1297 | 1.18 | −1.13 | −1.22 | Supv3l1 |
| 1460559_at | Lgx only | 2459 | 2747 | 3020 | 4193 | 1.12 | 1.23 | 1.71 | Ankrd25 |
| 1460570_at | Lgx only | 90 | 123 | 168 | 176 | 1.36 | 1.87 | 1.95 | Pgbd5 |
| 1460573_at | Res & Lgx | 838 | 611 | 460 | 341 | −1.37 | −1.82 | −2.46 | Al848100 |
| 1460576_at | Res & Lgx | 1390 | 1299 | 1107 | 878 | −1.07 | −1.26 | −1.58 | LOC100047539 |
| 1460580_at | Lgx only | 950 | 1086 | 1029 | 1226 | 1.14 | 1.08 | 1.29 | Pcnx |
| 1460586_at | Lgx only | 802 | 890 | 990 | 1316 | 1.11 | 1.23 | 1.64 | Megf8 |
| 1460603_at | Res only | 703 | 632 | 490 | 590 | −1.11 | −1.43 | −1.19 | Samd9l |
| 1460607_at | CR only | 43 | 102 | 138 | 99 | 2.38 | 3.21 | 2.30 | Igsf11 |
| 1460610_at | Lgx only | 191 | 208 | 233 | 328 | 1.09 | 1.22 | 1.72 | Agbl5 |
| 1460614_at | Lgx only | 331 | 289 | 291 | 199 | −1.14 | −1.14 | −1.66 | LOC100045020 |
| 1460624_at | Res & Lgx | 363 | 266 | 217 | 198 | −1.36 | −1.67 | −1.83 | 6330564D18Rik |
| 1460643_at | Lgx only | 270 | 360 | 304 | 400 | 1.34 | 1.13 | 1.48 | Ell |
| 1460644_at | Lgx only | 2028 | 2060 | 2523 | 2936 | 1.02 | 1.24 | 1.45 | Bckdk |
| 1460645_at | Res & Lgx | 1384 | 1178 | 880 | 926 | −1.17 | −1.57 | −1.49 | Chordc1 |
| 1460648_at | Lgx only | 1100 | 1245 | 1478 | 1574 | 1.13 | 1.34 | 1.43 | Nr2f6 |
| 1460674_at | Lgx only | 477 | 623 | 657 | 970 | 1.31 | 1.38 | 2.04 | Paqr7 |
| 1460675_at | Lgx only | 412 | 429 | 642 | 729 | 1.04 | 1.56 | 1.77 | Igsf8 |
| 1460695_a_at | Lgx only | 2491 | 2664 | 2132 | 1926 | 1.07 | −1.17 | −1.29 | 2010111I01Rik |
| 1460704_at | Lgx only | 664 | 756 | 815 | 930 | 1.14 | 1.23 | 1.40 | Rfng |
| 1460716_a_at | Lgx only | 2287 | 2216 | 2090 | 1679 | −1.03 | −1.09 | −1.36 | Cbfb |
| 1460720_at | Res & Lgx | 1749 | 2092 | 2554 | 2716 | 1.20 | 1.46 | 1.55 | Trpc4ap |
| 1460732_a_at | Lgx only | 495 | 527 | 571 | 770 | 1.06 | 1.15 | 1.56 | Ppl |
| AFFX-b-ActinMur/ | Res & Lgx | 37967 | 41791 | 48255 | 54572 | 1.10 | 1.27 | 1.44 | Actb |
| M12481_3_at | | | | | | | | | |
| AFFX-GapdhMur/ | Lgx only | 90974 | 104691 | 98912 | 122441 | 1.15 | 1.09 | 1.35 | Gapdh |
| M32599_M_at | | | | | | | | | |

Several genes of particular interest showed expression patterns indicating that compounds of the present invention (Longevinex®) up-regulated survival/longevity genes or down-regulate genes whose expression enhances cellular damage to a greater extent than resveratrol:
(A) The sirtuin family of genes, and in particular Sirtuin 1, are thought to be critical mediators of extended lifespans (Boily, G. et al. (2008) “SirT1 Regulates Energy Metabolism And Response To Caloric Restriction In Mice,” PLoS ONE 3(3):e1759; Huang, J. et al. (2008) “SIRT1 Overexpression Antagonizes Cellular Senescence with Activated ERK/S6k1 Signaling in Human Diploid Fibroblasts,” PLoS ONE 3(3):e1710). Whereas mice receiving resveratrol showed only a 1.22 fold decrease in expression and mice subjected to a calorie restricted diet showed only a 1.12 fold reduction in Sirtuin 1 expression, expression of Sirtuin 1 was found to be decreased 1.71 fold in mice receiving Longevinex®.
(B) Pgc-1α (peroxisome proliferative activated receptor, gamma, coactivator 1 alpha; ppargc1a) is a transcriptional co-factor that controls energy metabolism and mitochondrial biogenesis; its expression is increased in skeletal muscle tissue upon long-term calorie restriction (Conley, K. E. et al. (2007) “Mitochondrial Dysfunction and Age,” Curr. Opin. Clin. Nutr. Metab. Care. 10(6):688-692; Wu, Z. et al. (2007) “Targeting PGC-1 Alpha To Control Energy Homeostasis,” Expert Opin. Ther. Targets 11(10):1329-1338). Whereas mice receiving resveratrol showed only a 1.6 fold increase in expression and mice subjected to a calorie restricted diet showed no increase in Pgc-1α expression, mice receiving Longevinex® showed a 1.94 fold increase in Pgc-1α expression.
(C) Uncoupling protein-3 is believed to be a target of Pgc-1α and to play a role in fatty acid metabolism; its expression is increased in cardiac tissue upon long-term calorie restriction (Bézaire, V. et al. (Epub 2007 Jan. 3) “Uncoupling Protein-3: Clues In An Ongoing Mitochondrial Mystery,” FASEB J. 21(2):312-324; Chan, C. B. et al. (2006) “Uncoupling Proteins: Role In Insulin Resistance And Insulin Insufficiency,” Curr. Diabetes Rev. 2(3):271-283). Whereas mice receiving resveratrol showed only a 2.02 fold increase in expression and mice subjected to a calorie restricted diet showed only a 1.8 fold increase in uncoupling protein-3 expression, mice receiving Longevinex® showed a 2.79 fold increase in uncoupling protein-3 expression.
(D) Pyruvate dehydrogenase kinase 4 coordinates fuel selection during fasting to promote fatty acid metabolism (Sugden, M. C. et al. (2006) “Mechanisms Underlying Regulation Of The Expression And Activities Of The Mammalian Pyruvate Dehydrogenase Kinases,” Arch. Physiol. Biochem. 112(3):139-149; Pilegaard, H. et al. (2004) “Transcriptional Regulation Of Pyruvate Dehydrogenase Kinase 4 In Skeletal Muscle During And After Exercise,” Proc. Nutr. Soc. 63(2):221-226; Sugden, M. C. (2003) “PDK4: A factor in fatness?,” Obes. Res. 11(2):167-169). It is a target of Pgc-1α and is induced in multiple tissues by long-term calorie restriction. Whereas mice receiving resveratrol showed only a 2.78 fold increase in expression and mice subjected to a calorie restricted diet showed only a 1.48 fold increase in pyruvate dehydrogenase kinase 4 expression, mice receiving Longevinex® showed a 3.25 fold increase in pyruvate dehydrogenase kinase 4 expression.

Analysis of the genes up-regulated or down-regulated by a compound of the present invention (Longevinex®) revealed that oxidative phosphorylation genes, which are involved in mitochondrial ATP production, were markedly up-regulated (Table 4).

TABLE 4

| FC CR | FC RES | FCLGX | Gene |
|---|---|---|---|
| 1.11 | 1.14 | 1.32 | Ndufa5 |
| −1.00 | −1.20 | −1.42 | Ndufaf1 |
| −1.04 | −1.13 | −1.22 | Ndufb3 |
| 1.13 | 1.06 | 1.27 | Ndufb8 |
| 1.12 | 1.18 | 1.28 | Ndufb7 |
| −1.34 | −1.55 | −2.65 | Ndufab1 |
| 1.07 | 1.20 | 1.51 | Ndufc1 |
| −1.07 | −1.30 | −1.39 | Ndufc2 |
| 1.08 | 1.11 | 1.37 | Ndufs1 |
| 1.13 | 1.10 | 1.26 | Ndufs2 |
| 1.09 | 1.12 | 1.23 | Ndufs3 |
| 1.04 | 1.19 | 1.33 | Ndufs5 |
| 1.13 | 1.18 | 1.44 | Ndufs7 |
| −1.02 | 1.03 | −1.23 | Ndufs8 |
| 1.14 | 1.18 | 1.21 | Ndufv1 |
| 1.11 | 1.13 | 1.34 | Ndufv2 |
| 1.17 | 1.13 | 1.43 | Sdha |
| 1.16 | 1.02 | 1.23 | Sdhd |
| 1.46 | 1.29 | 1.49 | Sulf2 |
| 1.01 | −1.25 | −1.33 | Uqcc |
| 1.10 | 1.19 | 1.34 | Uqcrc1 |
| 1.05 | 1.07 | 1.38 | Uqcrfs1 |
| 1.20 | 1.50 | 1.94 | Cox4i2 |
| 1.13 | 1.05 | 1.39 | Cox5a |
| 1.23 | 1.13 | 1.61 | Cox8a |

### Example 4

### Biochemical Pathways Affected by the Compositions of the Present Invention

Recent research has suggested that complex traits are emergent properties of molecular networks that are modulated by complex genetic loci and environmental factors. Chen, Y. et al. (Epub 2008 Mar. 16) “Variations In DNA Elucidate Molecular Networks That Cause Disease,” Nature 452(7186):429-435).

Indeed, research within the last decade has revealed that most chronic illnesses such as cancer, cardiovascular and pulmonary diseases, neurological diseases, diabetes, and autoimmune diseases exhibit dysregulation of multiple cell signaling pathways (Harikumar, K. B. et al. (Epub Feb. 15, 2008) “Resveratrol: A Multitargeted Agent For Age-Associated Chronic Diseases,” Cell Cycle. 2008:7(8)). The compounds of the present invention were therefore evaluated for their effect on the expression of biochemical pathways and were found to affect the expression of genes involved in 220 biological processes (P<0.05) by (Table 5).

TABLE 5

| | | | | Number | | | |
|---|---|---|---|---|---|---|---|
| | | | Changed | of Genes | | | |
| GO ID | Biological Processes | Treatment | by LT-CR | in Series | CR | RES | LGX |
| GO: 0051128 | Regulation Of Cellular Component | CR only | 0.0277 | 51 | 5 | — | — |
| | Organization And Biogenesis | | | | | | |
| GO: 0001558 | Regulation Of Cell Growth | CR only | — | 74 | 5 | — | — |
| GO: 0006820 | Anion Transport | CR only | — | 155 | 6 | — | — |
| GO: 0008361 | Regulation Of Cell Size | CR only | — | 102 | 6 | — | — |
| GO: 0016049 | Cell Growth | CR only | — | 90 | 5 | — | — |
| GO: 0030217 | T Cell Differentiation | CR only | — | 55 | 3 | — | — |
| GO: 0030595 | Leukocyte Chemotaxis | CR only | — | 18 | 2 | — | — |
| GO: 0045580 | Regulation Of T Cell Differentiation | CR only | — | 15 | 2 | — | — |
| GO: 0045792 | Negative Regulation Of Cell Size | CR only | — | 16 | 2 | — | — |
| GO: 0048705 | Skeletal Morphogenesis | CR only | — | 20 | 2 | — | — |
| GO: 0051246 | Regulation Of Protein Metabolic Process | CR only | — | 204 | 8 | — | — |
| GO: 0033554 | Cellular Response To Stress | RES only | 0.0074 | 14 | — | 3 | — |
| GO: 0006888 | ER To Golgi Vesicle-Mediated Transport | RES only | 0.0284 | 16 | — | 3 | — |
| GO: 0000723 | Telomere Maintenance | RES only | — | 17 | — | 3 | — |
| GO: 0001958 | Endochondral Ossification | RES only | — | 8 | — | 2 | — |
| GO: 0006281 | DNA Repair | RES only | — | 178 | — | 13 | — |
| GO: 0006353 | Transcription Termination | RES only | — | 6 | — | 2 | — |
| GO: 0006446 | Regulation Of Translational Initiation | RES only | — | 20 | — | 4 | — |
| GO: 0006596 | Polyamine Biosynthetic Process | RES only | — | 5 | — | 2 | — |
| GO: 0006625 | Protein Targeting To Peroxisome | RES only | — | 5 | — | 2 | — |
| GO: 0006825 | Copper Ion Transport | RES only | — | 9 | — | 3 | — |
| GO: 0006919 | Caspase Activation | RES only | — | 16 | — | 3 | — |
| GO: 0006974 | Response To DNA Damage Stimulus | RES only | — | 217 | — | 15 | — |
| GO: 0006983 | ER Overload Response | RES only | — | 5 | — | 2 | — |
| GO: 0007017 | Microtubule-Based Process | RES only | — | 155 | — | 12 | — |
| GO: 0007091 | Mitotic Metaphase/Anaphase Transition | RES only | — | 8 | — | 2 | — |
| GO: 0007143 | Female Meiosis | RES only | — | 8 | — | 2 | — |
| GO: 0008299 | Isoprenoid Biosynthetic Process | RES only | — | 19 | — | 3 | — |
| GO: 0045351 | Interferon Type I Biosynthetic Process | RES only | — | 6 | — | 2 | — |
| GO: 0045577 | Regulation Of B Cell Differentiation | RES only | — | 8 | — | 2 | — |
| GO: 0046330 | Positive Regulation Of JNK Cascade | RES only | — | 8 | — | 2 | — |
| GO: 0048193 | Golgi Vesicle Transport | RES only | — | 37 | — | 6 | — |
| GO: 0050673 | Epithelial Cell Proliferation | RES only | — | 30 | — | 4 | — |
| GO: 0006119 | Oxidative Phosphorylation | LGX only | 0.0001 | 39 | — | — | 10 |
| GO: 0042773 | ATP Synthesis Coupled Electron | LGX only | 0.0019 | 11 | — | — | 5 |
| | Transport | | | | | | |
| GO: 0030036 | Actin Cytoskeleton Organization And | LGX only | 0.0024 | 146 | — | — | 34 |
| | Biogenesis | | | | | | |
| GO: 0006629 | Lipid Metabolic Process | LGX only | 0.0146 | 535 | — | — | 89 |
| GO: 0044255 | Cellular Lipid Metabolic Process | LGX only | 0.0147 | 459 | — | — | 80 |
| GO: 0001701 | In Utero Embryonic Development | LGX only | 0.0195 | 101 | — | — | 19 |
| GO: 0040008 | Regulation Of Growth | LGX only | 0.0242 | 135 | — | — | 23 |
| GO: 0000375 | RNA Splicing, Via Transesterification | LGX only | 0.0251 | 39 | — | — | 10 |
| | Reactions | | | | | | |
| GO: 0000398 | Nuclear Mrna Splicing, Via Spliceosome | LGX only | 0.0251 | 39 | — | — | 10 |
| GO: 0006366 | Transcription From RNA Polymerase II | LGX only | 0.0264 | 392 | — | — | 72 |
| | Promoter | | | | | | |
| GO: 0006357 | Regulation Of Transcription From RNA | LGX only | 0.0276 | 351 | — | — | 59 |
| | Polymerase II Promoter | | | | | | |
| GO: 0016044 | Membrane Organization And Biogenesis | LGX only | 0.0292 | 209 | — | — | 34 |
| GO: 0006066 | Alcohol Metabolic Process | LGX only | 0.0299 | 222 | — | — | 41 |
| GO: 0065002 | Intracellular Protein Transport Across A | LGX only | 0.0372 | 59 | — | — | 16 |
| | Membrane | | | | | | |
| GO: 0006099 | Tricarboxylic Acid Cycle | LGX only | 0.0396 | 23 | — | — | 7 |
| GO: 0009060 | Aerobic Respiration | LGX only | 0.0396 | 24 | — | — | 7 |
| GO: 0044265 | Cellular Macromolecule Catabolic | LGX only | 0.0417 | 201 | — | — | 41 |
| | Process | | | | | | |
| GO: 0006006 | Glucose Metabolic Process | LGX only | 0.0441 | 83 | — | — | 16 |
| GO: 0045333 | Cellular Respiration | LGX only | 0.0484 | 28 | — | — | 8 |
| GO: 0000038 | Very-Long-Chain Fatty Acid Metabolic | LGX only | — | 5 | — | — | 3 |
| | Process | | | | | | |
| GO: 0000059 | Protein Import Into Nucleus, Docking | LGX only | — | 15 | — | — | 7 |
| GO: 0000186 | Activation Of MAPKK Activity | LGX only | — | 10 | — | — | 5 |
| GO: 0001525 | Angiogenesis | LGX only | — | 124 | — | — | 26 |
| GO: 0001568 | Blood Vessel Development | LGX only | — | 188 | — | — | 42 |
| GO: 0001570 | Vasculogenesis | LGX only | — | 27 | — | — | 8 |
| GO: 0001839 | Neural Plate Morphogenesis | LGX only | — | 40 | — | — | 9 |
| GO: 0001841 | Neural Tube Formation | LGX only | — | 39 | — | — | 9 |
| GO: 0001843 | Neural Tube Closure | LGX only | — | 29 | — | — | 7 |
| GO: 0001935 | Endothelial Cell Proliferation | LGX only | — | 8 | — | — | 4 |
| GO: 0002026 | Cardiac Inotropy | LGX only | — | 10 | — | — | 4 |
| GO: 0003007 | Heart Morphogenesis | LGX only | — | 32 | — | — | 8 |
| GO: 0005978 | Glycogen Biosynthetic Process | LGX only | — | 11 | — | — | 5 |
| GO: 0006007 | Glucose Catabolic Process | LGX only | — | 44 | — | — | 11 |
| GO: 0006098 | Pentose-Phosphate Shunt | LGX only | — | 7 | — | — | 3 |
| GO: 0006118 | Electron Transport | LGX only | — | 303 | — | — | 65 |
| GO: 0006120 | Mitochondrial Electron Transport, | LGX only | — | 6 | — | — | 5 |
| | NADH To Ubiquinone | | | | | | |
| GO: 0006171 | Camp Biosynthetic Process | LGX only | — | 14 | — | — | 5 |
| GO: 0006259 | DNA Metabolic Process | LGX only | — | 524 | — | — | 77 |
| GO: 0006323 | DNA Packaging | LGX only | — | 209 | — | — | 37 |
| GO: 0006325 | Establishment And/Or Maintenance Of | LGX only | — | 203 | — | — | 34 |
| | Chromatin Architecture | | | | | | |
| GO: 0006333 | Chromatin Assembly Or Disassembly | LGX only | — | 80 | — | — | 15 |
| GO: 0006352 | Transcription Initiation | LGX only | — | 27 | — | — | 7 |
| GO: 0006354 | RNA Elongation | LGX only | — | 5 | — | — | 3 |
| GO: 0006367 | Transcription Initiation From RNA | LGX only | — | 11 | — | — | 5 |
| | Polymerase II Promoter | | | | | | |
| GO: 0006396 | RNA Processing | LGX only | — | 319 | — | — | 63 |
| GO: 0006397 | Mrna Processing | LGX only | — | 213 | — | — | 43 |
| GO: 0006414 | Translational Elongation | LGX only | — | 19 | — | — | 6 |
| GO: 0006461 | Protein Complex Assembly | LGX only | — | 122 | — | — | 28 |
| GO: 0006468 | Protein Amino Acid Phosphorylation | LGX only | — | 545 | — | — | 83 |
| GO: 0006470 | Protein Amino Acid Dephosphorylation | LGX only | — | 99 | — | — | 18 |
| GO: 0006473 | Protein Amino Acid Acetylation | LGX only | — | 12 | — | — | 4 |
| GO: 0006508 | Proteolysis | LGX only | — | 545 | — | — | 85 |
| GO: 0006520 | Amino Acid Metabolic Process | LGX only | — | 198 | — | — | 34 |
| GO: 0006606 | Protein Import Into Nucleus | LGX only | — | 53 | — | — | 13 |
| GO: 0006612 | Protein Targeting To Membrane | LGX only | — | 15 | — | — | 5 |
| GO: 0006631 | Fatty Acid Metabolic Process | LGX only | — | 142 | — | — | 35 |
| GO: 0006635 | Fatty Acid Beta-Oxidation | LGX only | — | 13 | — | — | 6 |
| GO: 0006638 | Neutral Lipid Metabolic Process | LGX only | — | 21 | — | — | 6 |
| GO: 0006641 | Triacylglycerol Metabolic Process | LGX only | — | 17 | — | — | 6 |
| GO: 0006662 | Glycerol Ether Metabolic Process | LGX only | — | 23 | — | — | 6 |
| GO: 0006766 | Vitamin Metabolic Process | LGX only | — | 57 | — | — | 14 |
| GO: 0006807 | Nitrogen Compound Metabolic Process | LGX only | — | 315 | — | — | 49 |
| GO: 0006869 | Lipid Transport | LGX only | — | 67 | — | — | 16 |
| GO: 0006913 | Nucleocytoplasmic Transport | LGX only | — | 89 | — | — | 23 |
| GO: 0006914 | Autophagy | LGX only | — | 21 | — | — | 8 |
| GO: 0007031 | Peroxisome Organization And | LGX only | — | 22 | — | — | 7 |
| | Biogenesis | | | | | | |
| GO: 0007182 | Common-Partner SMAD Protein | LGX only | — | 8 | — | — | 4 |
| | Phosphorylation | | | | | | |
| GO: 0007190 | Adenylate Cyclase Activation | LGX only | — | 12 | — | — | 4 |
| GO: 0007242 | Intracellular Signaling Cascade | LGX only | — | 915 | — | — | 133 |
| GO: 0007369 | Gastrulation | LGX only | — | 55 | — | — | 13 |
| GO: 0007498 | Mesoderm Development | LGX only | — | 44 | — | — | 13 |
| GO: 0007507 | Heart Development | LGX only | — | 158 | — | — | 39 |
| GO: 0007512 | Adult Heart Development | LGX only | — | 9 | — | — | 4 |
| GO: 0007517 | Muscle Development | LGX only | — | 105 | — | — | 19 |
| GO: 0008016 | Regulation Of Heart Contraction | LGX only | — | 27 | — | — | 9 |
| GO: 0008286 | Insulin Receptor Signaling Pathway | LGX only | — | 24 | — | — | 7 |
| GO: 0009308 | Amine Metabolic Process | LGX only | — | 294 | — | — | 46 |
| GO: 0009653 | Anatomical Structure Morphogenesis | LGX only | — | 993 | — | — | 143 |
| GO: 0009790 | Embryonic Development | LGX only | — | 387 | — | — | 61 |
| GO: 0009792 | Embryonic Development Ending In Birth | LGX only | — | 190 | — | — | 32 |
| | Or Egg Hatching | | | | | | |
| GO: 0010003 | Gastrulation (Sensu Mammalia) | LGX only | — | 17 | — | — | 6 |
| GO: 0015804 | Neutral Amino Acid Transport | LGX only | — | 6 | — | — | 3 |
| GO: 0015908 | Fatty Acid Transport | LGX only | — | 6 | — | — | 3 |
| GO: 0016071 | Mrna Metabolic Process | LGX only | — | 240 | — | — | 45 |
| GO: 0016192 | Vesicle-Mediated Transport | LGX only | — | 365 | — | — | 61 |
| GO: 0016310 | Phosphorylation | LGX only | — | 601 | — | — | 95 |
| GO: 0016311 | Dephosphorylation | LGX only | — | 111 | — | — | 20 |
| GO: 0016481 | Negative Regulation Of Transcription | LGX only | — | 223 | — | — | 40 |
| GO: 0016485 | Protein Processing | LGX only | — | 58 | — | — | 14 |
| GO: 0016540 | Protein Autoprocessing | LGX only | — | 30 | — | — | 9 |
| GO: 0016567 | Protein Ubiquitination | LGX only | — | 36 | — | — | 9 |
| GO: 0016568 | Chromatin Modification | LGX only | — | 152 | — | — | 27 |
| GO: 0016574 | Histone Ubiquitination | LGX only | — | 5 | — | — | 3 |
| GO: 0019395 | Fatty Acid Oxidation | LGX only | — | 20 | — | — | 8 |
| GO: 0019752 | Carboxylic Acid Metabolic Process | LGX only | — | 403 | — | — | 78 |
| GO: 0030163 | Protein Catabolic Process | LGX only | — | 162 | — | — | 31 |
| GO: 0030239 | Myofibril Assembly | LGX only | — | 12 | — | — | 5 |
| GO: 0030323 | Respiratory Tube Development | LGX only | — | 58 | — | — | 12 |
| GO: 0030324 | Lung Development | LGX only | — | 57 | — | — | 12 |
| GO: 0030855 | Epithelial Cell Differentiation | LGX only | — | 34 | — | — | 8 |
| GO: 0030856 | Regulation Of Epithelial Cell | LGX only | — | 7 | — | — | 3 |
| | Differentiation | | | | | | |
| GO: 0030865 | Cortical Cytoskeleton Organization And | LGX only | — | 10 | — | — | 5 |
| | Biogenesis | | | | | | |
| GO: 0031032 | Actomyosin Structure Organization And | LGX only | — | 16 | — | — | 5 |
| | Biogenesis | | | | | | |
| GO: 0032147 | Activation Of Protein Kinase Activity | LGX only | — | 28 | — | — | 8 |
| GO: 0035051 | Cardiac Cell Differentiation | LGX only | — | 13 | — | — | 6 |
| GO: 0035239 | Tube Morphogenesis | LGX only | — | 128 | — | — | 25 |
| GO: 0035295 | Tube Development | LGX only | — | 174 | — | — | 36 |
| GO: 0042254 | Ribosome Biogenesis And Assembly | LGX only | — | 97 | — | — | 18 |
| GO: 0042692 | Muscle Cell Differentiation | LGX only | — | 58 | — | — | 14 |
| GO: 0043009 | Chordate Embryonic Development | LGX only | — | 187 | — | — | 32 |
| GO: 0043087 | Regulation Of Gtpase Activity | LGX only | — | 59 | — | — | 12 |
| GO: 0043623 | Cellular Protein Complex Assembly | LGX only | — | 39 | — | — | 11 |
| GO: 0043631 | RNA Polyadenylation | LGX only | — | 11 | — | — | 4 |
| GO: 0044257 | Cellular Protein Catabolic Process | LGX only | — | 116 | — | — | 27 |
| GO: 0045214 | Sarcomere Organization | LGX only | — | 9 | — | — | 4 |
| GO: 0045761 | Regulation Of Adenylate Cyclase | LGX only | — | 16 | — | — | 5 |
| | Activity | | | | | | |
| GO: 0045893 | Positive Regulation Of Transcription, | LGX only | — | 225 | — | — | 44 |
| | DNA-Dependent | | | | | | |
| GO: 0045944 | Positive Regulation Of Transcription | LGX only | — | 186 | — | — | 34 |
| | From RNA Polymerase II Promoter | | | | | | |
| GO: 0046058 | Camp Metabolic Process | LGX only | — | 17 | — | — | 5 |
| GO: 0046777 | Protein Amino Acid | LGX only | — | 29 | — | — | 9 |
| | Autophosphorylation | | | | | | |
| GO: 0048276 | Gastrulation (Sensu Vertebrata) | LGX only | — | 24 | — | — | 7 |
| GO: 0048514 | Blood Vessel Morphogenesis | LGX only | — | 160 | — | — | 36 |
| GO: 0048646 | Anatomical Structure Formation | LGX only | — | 171 | — | — | 34 |
| GO: 0050658 | RNA Transport | LGX only | — | 48 | — | — | 11 |
| GO: 0051028 | Mrna Transport | LGX only | — | 45 | — | — | 11 |
| GO: 0051146 | Striated Muscle Cell Differentiation | LGX only | — | 26 | — | — | 11 |
| GO: 0051170 | Nuclear Import | LGX only | — | 54 | — | — | 13 |
| GO: 0055001 | Muscle Cell Development | LGX only | — | 13 | — | — | 5 |
| GO: 0055002 | Striated Muscle Cell Development | LGX only | — | 12 | — | — | 5 |
| GO: 0055007 | Cardiac Muscle Cell Differentiation | LGX only | — | 9 | — | — | 6 |
| GO: 0055012 | Ventricular Cardiac Muscle Cell | LGX only | — | 6 | — | — | 4 |
| | Differentiation | | | | | | |
| GO: 0051016 | Barbed-End Actin Filament Capping | CR & RES | 0.0487 | 17 | 2 | 3 | — |
| GO: 0030029 | Actin Filament-Based Process | CR & LGX | 0.0049 | 157 | 6 | — | 37 |
| GO: 0006084 | Acetyl-Coa Metabolic Process | CR & LGX | 0.0068 | 33 | 3 | — | 11 |
| GO: 0045941 | Positive Regulation Of Transcription | CR & LGX | 0.0119 | 267 | 8 | — | 48 |
| GO: 0006915 | Apoptosis | CR & LGX | 0.0172 | 537 | 14 | — | 84 |
| GO: 0012501 | Programmed Cell Death | CR & LGX | 0.0198 | 544 | 14 | — | 84 |
| GO: 0046356 | Acetyl-Coa Catabolic Process | CR & LGX | 0.0396 | 24 | 2 | — | 8 |
| GO: 0008219 | Cell Death | CR & LGX | 0.0410 | 564 | 14 | — | 84 |
| GO: 0006364 | Rrna Processing | CR & LGX | — | 50 | 3 | — | 11 |
| GO: 0006519 | Amino Acid And Derivative Metabolic | CR & LGX | — | 253 | 8 | — | 41 |
| | Process | | | | | | |
| GO: 0006796 | Phosphate Metabolic Process | CR & LGX | — | 714 | 17 | — | 114 |
| GO: 0006839 | Mitochondrial Transport | CR & LGX | — | 20 | 2 | — | 9 |
| GO: 0007005 | Mitochondrion Organization And | CR & LGX | — | 58 | 4 | — | 19 |
| | Biogenesis | | | | | | |
| GO: 0007167 | Enzyme Linked Receptor Protein | CR & LGX | — | 252 | 9 | — | 46 |
| | Signaling Pathway | | | | | | |
| GO: 0007179 | Transforming Growth Factor Beta | CR & LGX | — | 42 | 3 | — | 11 |
| | Receptor Signaling Pathway | | | | | | |
| GO: 0009056 | Catabolic Process | CR & LGX | — | 474 | 13 | — | 79 |
| GO: 0016265 | Death | CR & LGX | — | 564 | 14 | — | 84 |
| GO: 0030833 | Regulation Of Actin Filament | CR & LGX | — | 10 | 2 | — | 4 |
| | Polymerization | | | | | | |
| GO: 0008104 | Protein Localization | RES & LGX | 0.0007 | 663 | — | 38 | 127 |
| GO: 0015031 | Protein Transport | RES & LGX | 0.0011 | 581 | — | 38 | 121 |
| GO: 0045184 | Establishment Of Protein Localization | RES & LGX | 0.0028 | 610 | — | 38 | 123 |
| GO: 0006886 | Intracellular Protein Transport | RES & LGX | 0.0098 | 357 | — | 26 | 75 |
| GO: 0006605 | Protein Targeting | RES & LGX | 0.0099 | 161 | — | 12 | 34 |
| GO: 0044249 | Cellular Biosynthetic Process | RES & LGX | 0.0107 | 710 | — | 45 | 117 |
| GO: 0009058 | Biosynthetic Process | RES & LGX | 0.0120 | 979 | — | 60 | 155 |
| GO: 0006412 | Translation | RES & LGX | 0.0364 | 338 | — | 25 | 62 |
| GO: 0044262 | Cellular Carbohydrate Metabolic Process | RES & LGX | 0.0471 | 221 | — | 18 | 49 |
| GO: 0005975 | Carbohydrate Metabolic Process | RES & LGX | — | 316 | — | 21 | 60 |
| GO: 0005976 | Polysaccharide Metabolic Process | RES & LGX | — | 42 | — | 9 | 15 |
| GO: 0005977 | Glycogen Metabolic Process | RES & LGX | — | 31 | — | 7 | 13 |
| GO: 0006091 | Generation Of Precursor Metabolites | RES & LGX | — | 390 | — | 24 | 88 |
| | And Energy | | | | | | |
| GO: 0006112 | Energy Reserve Metabolic Process | RES & LGX | — | 35 | — | 7 | 13 |
| GO: 0006413 | Translational Initiation | RES & LGX | — | 40 | — | 6 | 9 |
| GO: 0006511 | Ubiquitin-Dependent Protein Catabolic | RES & LGX | — | 109 | — | 9 | 27 |
| | Process | | | | | | |
| GO: 0006512 | Ubiquitin Cycle | RES & LGX | — | 356 | — | 27 | 79 |
| GO: 0007178 | Transmembrane Receptor Protein | RES & LGX | — | 75 | — | 7 | 19 |
| | Serine/Threonine Kinase Signaling | | | | | | |
| | Pathway | | | | | | |
| GO: 0007264 | Small Gtpase Mediated Signal | RES & LGX | — | 320 | — | 22 | 62 |
| | Transduction | | | | | | |
| GO: 0008380 | RNA Splicing | RES & LGX | — | 162 | — | 12 | 30 |
| GO: 0009059 | Macromolecule Biosynthetic Process | RES & LGX | — | 525 | — | 35 | 90 |
| GO: 0019941 | Modification-Dependent Protein | RES & LGX | — | 111 | — | 9 | 27 |
| | Catabolic Process | | | | | | |
| GO: 0043085 | Positive Regulation Of Enzyme Activity | RES & LGX | — | 40 | — | 5 | 11 |
| GO: 0043280 | Positive Regulation Of Caspase Activity | RES & LGX | — | 17 | — | 3 | 5 |
| GO: 0043281 | Regulation Of Caspase Activity | RES & LGX | — | 27 | — | 5 | 8 |
| GO: 0045454 | Cell Redox Homeostasis | RES & LGX | — | 40 | — | 6 | 9 |
| GO: 0050790 | Regulation Of Catalytic Activity | RES & LGX | — | 241 | — | 21 | 53 |
| GO: 0008064 | Regulation Of Actin Polymerization | All | 0.0139 | 30 | 5 | 4 | 9 |
| | And/Or Depolymerization | | | | | | |
| GO: 0030832 | Regulation Of Actin Filament Length | All | 0.0139 | 31 | 5 | 4 | 9 |
| GO: 0046907 | Intracellular Transport | All | 0.0287 | 523 | 16 | 43 | 113 |
| GO: 0008154 | Actin Polymerization And/Or | All | 0.0414 | 39 | 5 | 6 | 12 |
| | Depolymerization | | | | | | |
| GO: 0051649 | Establishment Of Cellular Localization | All | 0.0467 | 653 | 17 | 45 | 125 |
| GO: 0006457 | Protein Folding | All | — | 124 | 6 | 13 | 34 |
| GO: 0006996 | Organelle Organization And Biogenesis | All | — | 897 | 27 | 53 | 158 |
| GO: 0007010 | Cytoskeleton Organization And | All | — | 403 | 12 | 26 | 70 |
| | Biogenesis | | | | | | |
| GO: 0007018 | Microtubule-Based Movement | All | — | 72 | 4 | 9 | 14 |
| GO: 0030041 | Actin Filament Polymerization | All | — | 17 | 2 | 3 | 7 |
| GO: 0051258 | Protein Polymerization | All | — | 32 | 6 | 6 | 8 |

Calorie restriction affected genes associated with 5% of these processes, administration of resveratrol affected genes associated with 10% of these processes. Compounds of the present invention (e.g., Longevinex®) were found to affect 85% of these processes. Administration of resveratrol to calorie restricted mice failed to affect any genes in any of these processes. Administration of Longevinex® to calorie restricted mice was found to affect genes associated with 8% of these processes. Administration of both resveratrol and Longevinex® was found to affect genes associated with 12% of these processes. Table 6 shows the modulation of the genes of the oxidative phosphorylation pathway (GO:0006119) caused by calorie restriction (CR), resveratrol alone (Res), or the compositions of the present invention (LGX).

TABLE 6
Modulation Of The Genes Of The Oxidative
Phosphorylation Pathway (GO: 0006119)

| | Fold Change | | |
|---|---|---|---|
| Gene | CR | Res | LGX |
| Atp6v0d1 | 1.03 | −1.21 | −1.19 |
| Atp6v1b2 | 1.27 | −1.16 | −1.23 |
| Ndufv1 | 1.14 | 1.18 | 1.21 |
| Ndufc2 | −1.07 | −1.30 | −1.39 |
| Atp6v1d | 1.13 | −1.09 | −1.05 |
| Ndufs3 | 1.09 | 1.12 | 1.23 |
| Atp6v1f | −1.14 | −1.21 | −1.44 |
| Ndufs7 | 1.13 | 1.18 | 1.44 |
| Uqcrh | 1.05 | −1.40 | −1.34 |
| Atp5j | 1.07 | −1.00 | 1.24 |
| Cyc1 | 1.14 | 1.08 | 1.29 |
| Atp5b | 1.10 | 1.01 | 1.27 |
| Atp5k | 1.05 | 1.02 | 1.24 |
| Atp5a1 | 1.09 | 1.02 | 1.29 |
| Atp5h | 1.07 | 1.04 | 1.37 |
| 1110020P15Rik | 1.06 | 1.09 | 1.26 |
| Ndufs1 | 1.08 | 1.11 | 1.37 |
| Atp5f1 | 1.09 | 1.08 | 1.19 |
| Uqcr | 1.03 | 1.10 | 1.30 |
| Atp6v1a | −1.04 | −1.18 | −1.35 |
| Ndufb9 | 1.07 | 1.07 | 1.25 |
| Atp6v1h | −1.17 | −1.19 | −1.11 |
| Uqcrb | −1.09 | −1.00 | −1.22 |
| Msh2 | 1.05 | −1.03 | −1.15 |
| Atp6v1c1 | −1.10 | −1.07 | −1.06 |
| Atp6v1c2 | 2.17 | 1.42 | −1.00 |
| Atp6v0d2 | 1.04 | 1.65 | 1.21 |
| Atp7a | −1.13 | −1.18 | −1.29 |
| Atp5c1 | −1.16 | −1.28 | −1.16 |
| Atp5g1 | 1.26 | −1.58 | −1.17 |
| Atp6v1e1 | 1.05 | −1.42 | −1.42 |
| Atp6v1e2 | 1.11 | 1.28 | 1.30 |
| Ndufa7 | −1.07 | −1.14 | −1.07 |
| Atp5g3 | 1.06 | −1.11 | −1.01 |

Table 7 shows the modulation of the genes of the glucose metabolism pathway (GO:0006006) caused by calorie restriction (CR), resveratrol alone (Res), or the compositions of the present invention (LGX).

TABLE 7
Modulation Of The Genes Of The Glucose
Metabolism Pathway (GO: 0006006)

| | Fold Change | | |
|---|---|---|---|
| Gene | CR | Res | LGX |
| Pgam2 | 1.27 | 1.38 | 1.81 |
| Pkm2 | 1.31 | 1.34 | 1.70 |
| Pgls | 1.28 | 1.24 | 1.42 |
| Eno3 | 1.17 | 1.26 | 1.37 |
| Atf3 | −1.86 | −1.47 | −1.68 |
| Pdk4 | 1.48 | 2.78 | 3.25 |
| H6pd | 1.33 | 1.43 | 2.06 |
| Pcx | 1.40 | 1.33 | 1.60 |
| Fbp2 | −1.16 | 1.42 | 1.47 |
| Pfkp | 1.32 | 1.13 | 1.24 |
| Pik3ca | −1.05 | 1.34 | 1.21 |
| Hibadh | 1.09 | 1.17 | 1.15 |
| Sds | −1.65 | −1.16 | 2.01 |
| Hk3 | 2.23 | 1.60 | 1.77 |
| Hkdc1 | 2.64 | 2.10 | 1.68 |
| Ldhb | 1.21 | 1.35 | 1.60 |
| Mdh1 | 1.07 | 1.08 | 1.41 |
| Ogdh | 1.32 | 1.23 | 1.38 |
| Gapdh | 1.15 | 1.09 | 1.35 |
| Pdha1 | 1.12 | 1.09 | 1.30 |
| Tpi1 | 1.15 | 1.06 | 1.24 |
| Mdh2 | 1.10 | 1.01 | 1.15 |
| Pgm211 | 1.19 | 1.31 | 1.11 |
| Pdk1 | −1.01 | 1.01 | −1.18 |
| Hk1 | 1.12 | −1.24 | −1.34 |
| Prkaa1 | −1.08 | 1.17 | −1.38 |
| Mapk14 | −1.10 | −1.13 | −1.51 |
| Pck1 | 1.68 | 2.36 | 4.03 |
| Aldoart1 | 2.17 | 2.37 | 3.08 |
| G6pd2 | 1.93 | 1.37 | 2.90 |
| Ldhal6b | 1.15 | 1.45 | 2.73 |
| Ldhc | 1.94 | 2.25 | 2.44 |
| Gpd1 | 1.83 | 1.51 | 2.41 |
| Adipoq | 1.74 | 1.24 | 2.14 |
| Eno2 | −1.19 | 1.32 | 1.86 |
| Akt1 | 1.11 | 1.66 | 1.73 |
| Bpgm | −1.28 | 1.35 | 1.57 |
| Pdha2 | 1.78 | −1.03 | 1.57 |
| Npy1r | 1.11 | 1.37 | 1.53 |
| Gapdhs | 1.35 | 1.62 | 1.50 |
| G6pdx | 1.30 | 1.00 | 1.38 |
| Adpgk | −1.02 | 1.24 | 1.37 |
| Ins1 | 1.19 | 1.49 | 1.37 |
| Aldoc | −1.32 | 1.14 | 1.32 |
| Pdk2 | 1.11 | 1.11 | 1.24 |
| Aldob | −1.06 | 1.24 | 1.21 |
| Pdk3 | −2.02 | −1.04 | 1.21 |
| Pgm1 | 1.05 | 1.14 | 1.21 |
| Fbp1 | −1.50 | −1.25 | 1.20 |
| S1c2a8 | 1.11 | 1.30 | 1.19 |
| Gpd2 | 1.48 | −1.41 | 1.17 |
| Gck | 1.16 | 1.26 | 1.16 |
| Pgd | 1.20 | −1.05 | 1.16 |
| Dcxr | 1.05 | 1.08 | 1.14 |
| Rpia | 1.04 | 1.06 | 1.12 |
| Tnf | −1.16 | −1.73 | 1.11 |
| Atf4 | −1.06 | 1.07 | 1.10 |
| Nr3c1 | 1.14 | −1.14 | 1.10 |
| Taldo1 | 1.12 | 1.04 | 1.08 |
| Pgam1 | 1.15 | 1.01 | 1.07 |
| Aldoart2 | −1.05 | 1.16 | 1.05 |
| Ldha | 1.10 | −1.03 | 1.04 |
| Lrrc16 | 1.00 | −1.10 | 1.03 |
| Pgm2 | 1.11 | 1.09 | 1.03 |
| Pfkm | 1.13 | 1.07 | 1.02 |
| Pgk2 | −1.79 | 1.29 | −1.01 |
| 6430537H07Rik | −1.26 | −1.73 | −1.04 |
| Pck2 | 1.06 | −1.09 | −1.05 |
| Bad | 1.05 | 1.10 | −1.07 |
| Dlat | 1.05 | −1.09 | −1.07 |
| Dhtkd1 | −1.45 | −1.32 | −1.08 |
| Pfk1 | 1.19 | −1.15 | −1.11 |
| Car5a | −2.59 | 1.19 | −1.14 |
| Pdx1 | −3.03 | −1.37 | −1.14 |
| Cacnala | −1.31 | 1.03 | −1.21 |
| Acn9 | −1.04 | −1.08 | −1.22 |
| Fabp5 | 1.14 | −1.13 | −1.26 |
| Ppara | 1.14 | −1.06 | −1.28 |
| Uevld | −1.01 | −1.12 | −1.28 |
| Pgm3 | 1.13 | −1.09 | −1.29 |
| Ganc | −1.26 | 1.08 | −1.30 |
| Lep | −1.57 | −1.96 | −1.43 |
| Pklr | 1.12 | −1.08 | −1.47 |
| G6pc | 1.42 | −1.62 | −1.71 |
| Onecut1 | −1.32 | −2.13 | −2.05 |

Table 8 shows the modulation of the genes of the tricarboxylic acid metabolism pathway (GO:0006099) caused by calorie restriction (CR), resveratrol alone (Res), or the compositions of the present invention (LGX).

TABLE 8
Modulation Of The Genes Of The Tricarboxylic
Acid Metabolism Pathway (GO: 0006099)

| | Fold Change | | |
|---|---|---|---|
| Gene | CR | Res | LGX |
| Idh3b | 1.22 | 1.30 | 2.06 |
| Cs | 1.22 | 1.07 | 1.46 |
| Sdhd | 1.16 | 1.02 | 1.23 |
| Sdha | 1.17 | 1.13 | 1.43 |
| Sdhb | 1.08 | 1.13 | 1.42 |
| Mdh1 | 1.07 | 1.08 | 1.41 |
| Aco2 | 1.16 | 1.13 | 1.39 |
| Idh2 | 1.12 | 1.21 | 1.36 |
| 2610507B11Rik | 1.10 | 1.06 | 1.25 |
| Dlst | 1.21 | 1.08 | 1.24 |
| Fh1 | −1.01 | 1.05 | 1.18 |
| Mdh2 | 1.10 | 1.01 | 1.15 |
| Idh3a | 1.16 | −1.02 | 1.01 |
| Sdhc | −1.01 | −1.06 | −1.20 |
| Suclg2 | −1.03 | −1.00 | −1.25 |
| Polr3h | −1.19 | −1.20 | −1.34 |
| Sucla2 | 1.01 | −1.01 | 1.05 |
| Suclg1 | 1.08 | −1.07 | 1.00 |
| Mdh1b | −1.23 | −1.20 | −1.00 |
| Atp5g3 | 1.06 | −1.11 | −1.01 |
| Idh3g | 1.02 | −1.06 | −1.02 |
| Aco1 | 1.00 | −1.01 | −1.05 |

Table 9 shows the modulation of the genes of the fatty acid metabolism pathway (GO:0006631) caused by calorie restriction (CR), resveratrol alone (Res), or the compositions of the present invention (LGX).

TABLE 9
Modulation Of The Genes Of The Fatty
Acid Metabolism Pathway (GO: 0006631)

| | Fold Change | | |
|---|---|---|---|
| Gene | CR | Res | LGX |
| Ucp3 | 1.80 | 2.02 | 2.79 |
| Pex5 | 1.43 | 1.70 | 2.17 |
| Syk | 1.73 | 1.76 | 2.09 |
| Tnxb | 1.59 | 1.54 | 2.01 |
| Acox3 | 1.48 | 1.70 | 1.84 |
| Scap | 1.34 | 1.37 | 1.70 |
| Hadhb | 1.80 | 1.57 | 1.51 |
| Pex7 | −1.16 | −1.36 | −1.44 |
| Slc27a1 | 1.06 | 2.03 | 2.42 |
| Prkar2b | 1.95 | 2.01 | 2.24 |
| Acadl | 1.19 | 1.21 | 1.85 |
| Crat | 1.16 | 1.45 | 1.71 |
| Tnfrsf1a | −1.01 | 1.53 | 1.47 |
| Cav1 | 1.14 | 1.18 | 1.45 |
| Cpt1b | 1.07 | 1.27 | 1.45 |
| Hsd17b4 | 1.15 | 1.25 | 1.44 |
| Acaa2 | 1.07 | 1.27 | 1.41 |
| Gpam | −1.09 | 1.34 | 1.39 |
| Hadh | 1.08 | 1.18 | 1.36 |
| Fads3 | 1.13 | 1.38 | 1.35 |
| Lypla2 | −1.00 | −1.31 | −1.13 |
| Acsl6 | −1.15 | −1.23 | −1.26 |
| Elovl5 | −1.12 | −1.68 | −1.91 |
| Aasdh | −1.06 | −1.69 | −2.00 |
| Qk | −1.06 | −1.52 | −2.08 |
| Acox2 | 1.17 | 1.01 | 2.52 |
| Acot5 | 2.89 | 6.34 | 2.39 |
| Ptgis | 1.16 | 1.17 | 1.66 |
| Ppard | 1.20 | 1.09 | 1.59 |
| Acsf3 | 1.28 | 1.17 | 1.57 |
| Cpt1a | −1.04 | 1.27 | 1.49 |
| Hadha | 1.15 | 1.27 | 1.43 |
| Mlycd | −1.01 | 1.55 | 1.43 |
| Acads | 1.08 | 1.20 | 1.40 |
| Acadvl | 1.06 | 1.14 | 1.38 |
| Acsl1 | 1.05 | 1.21 | 1.38 |
| Apoa2 | 1.24 | 1.29 | 1.34 |
| Agpat6 | 1.15 | 1.15 | 1.32 |
| Echdc2 | 1.06 | 1.19 | 1.32 |
| Ech1 | 1.00 | 1.06 | 1.30 |
| Dci | 1.06 | 1.13 | 1.24 |
| Tpi1 | 1.15 | 1.06 | 1.24 |
| Mecr | −1.04 | 1.09 | 1.21 |
| Phyh | 1.10 | 1.15 | 1.20 |
| Ltc4s | 1.46 | −1.05 | 1.19 |
| Elovl1 | −1.03 | 1.15 | 1.18 |
| Scp2 | −1.02 | 1.06 | 1.16 |
| Acot7 | 1.20 | 1.03 | 1.06 |
| Ptgcs2 | 1.20 | 1.04 | −1.03 |
| Echs1 | 1.07 | 1.01 | −1.09 |
| Mcat | 1.04 | 1.01 | −1.15 |
| Acsl3 | −1.91 | −2.10 | −1.17 |
| 2010111I01Rik | 1.07 | −1.17 | −1.29 |
| Acsl4 | −1.34 | −1.20 | −1.35 |
| Crot | −1.11 | −1.07 | −1.37 |
| Prkaa1 | −1.08 | 1.17 | −1.38 |
| Prkab2 | 1.04 | −1.07 | −1.38 |
| Mapk14 | −1.10 | −1.13 | −1.51 |
| Lypla1 | −1.05 | −1.32 | −1.93 |
| Ndufab1 | −1.34 | −1.55 | −2.65 |
| Scd1 | 2.18 | 1.67 | 3.27 |
| Fasn | 2.54 | 1.54 | 3.10 |
| Agt | 1.42 | 1.57 | 2.50 |
| Baat | 2.33 | 2.78 | 2.42 |
| Elovl6 | 2.36 | 1.27 | 2.38 |
| Alox8 | 1.27 | 1.12 | 2.31 |
| Lcn5 | 1.58 | 1.74 | 2.30 |
| Acot4 | 1.58 | 1.65 | 2.24 |
| Acsm3 | −1.03 | 1.12 | 2.21 |
| Adipoq | 1.74 | 1.24 | 2.14 |
| Mlstd1 | −1.18 | 1.29 | 1.78 |
| Slc27a3 | 1.31 | 1.84 | 1.65 |
| Acsbg1 | 1.64 | 2.24 | 1.62 |
| Acot12 | −1.09 | −1.16 | 1.59 |
| Acot2 | −1.35 | 2.13 | 1.58 |
| Abat | −1.16 | 1.03 | 1.56 |
| Lta4h | 1.38 | 1.68 | 1.49 |
| Elovl3 | −1.12 | 1.11 | 1.47 |
| Acsm1 | 1.27 | 1.25 | 1.43 |
| Alox15 | 1.12 | 1.10 | 1.43 |
| Lypla3 | 1.31 | 1.42 | 1.41 |
| Slc27a2 | −1.52 | −1.96 | 1.41 |
| Elovl7 | 1.41 | 1.20 | 1.39 |
| Fads2 | 1.10 | 1.15 | 1.39 |
| Ptgs1 | 1.18 | 1.26 | 1.37 |
| Brca1 | −1.08 | 1.29 | 1.36 |
| Ptgs2 | 1.01 | 1.25 | 1.35 |
| Scd2 | 1.19 | −1.03 | 1.33 |
| Aacs | 1.35 | −1.12 | 1.32 |
| Alox5 | 1.18 | 1.22 | 1.32 |
| Fads1 | 1.19 | 1.27 | 1.28 |
| Ces3 | −1.16 | 1.06 | 1.23 |
| Pdpn | −1.14 | −1.04 | 1.22 |
| Aloxe3 | −1.14 | 1.00 | 1.21 |
| Ggtla1 | −1.03 | 1.15 | 1.20 |
| Slc27a4 | 1.23 | 1.25 | 1.20 |
| Acsl5 | 1.19 | 1.04 | 1.19 |
| Acot11 | 1.22 | −1.08 | 1.18 |
| Alox12e | −2.02 | −2.10 | 1.18 |
| Aldh5a1 | 1.25 | 1.15 | 1.17 |
| Adipor2 | −1.16 | 1.11 | 1.15 |
| Hao3 | −1.71 | −4.04 | 1.13 |
| Olah | 1.33 | −1.02 | 1.11 |
| Myo5a | 1.21 | 1.09 | 1.09 |
| Tyrp1 | −1.43 | −1.18 | 1.09 |
| Acot8 | 1.05 | −1.05 | 1.08 |
| Acsm5 | −1.38 | 1.14 | 1.08 |
| Hnf1a | 1.20 | 1.81 | 1.06 |
| Fccr1a | 1.55 | 1.73 | 1.05 |
| Fa2h | −1.27 | 1.18 | 1.04 |
| Prkag2 | −1.05 | −1.16 | 1.03 |
| Alox12 | 1.04 | 1.08 | 1.02 |
| Prkab1 | −1.12 | 1.04 | 1.02 |
| Ehhadh | −1.02 | 1.12 | 1.00 |
| Pecr | −1.01 | −1.03 | 1.00 |
| Degs1 | 1.02 | −1.16 | −1.00 |
| Scd3 | −1.37 | 1.36 | −1.01 |
| Acadm | −1.02 | −1.10 | −1.02 |
| Pccb | 1.01 | −1.01 | −1.02 |
| Prkag1 | 1.09 | −1.14 | −1.02 |
| Acox1 | −1.06 | 1.06 | −1.03 |
| Cpt2 | −1.14 | 1.01 | −1.03 |
| Tbxas1 | 1.00 | −1.06 | −1.03 |
| Pex13 | −1.05 | −1.03 | −1.04 |
| C1qtnf2 | −1.23 | −1.02 | −1.05 |
| Slc27a5 | −1.57 | −1.03 | −1.05 |
| Ptgcs3 | −1.08 | −1.03 | −1.07 |
| Acoxl | −1.85 | −1.39 | −1.08 |
| Alox5ap | 1.07 | −1.08 | −1.09 |
| Ankrd23 | 1.06 | 1.23 | −1.09 |
| Adipor1 | 1.08 | 1.03 | −1.10 |
| Cyb5 | −1.10 | −1.21 | −1.10 |
| Ptgds | −1.29 | 1.05 | −1.10 |
| Ptgds2 | −1.77 | −1.02 | −1.12 |
| Acsm2 | −1.33 | 1.06 | −1.19 |
| Cpt1c | −1.69 | 1.29 | −1.22 |
| Ncf1 | −1.16 | 1.07 | −1.25 |
| Ppara | 1.14 | −1.06 | −1.28 |
| Elovl4 | 1.00 | 1.45 | −1.29 |
| Mlstd2 | −1.20 | −1.19 | −1.29 |
| Oxsm | −1.02 | 1.04 | −1.29 |
| Plp1 | −1.03 | 1.06 | −1.29 |
| Cryl1 | −1.44 | −1.14 | −1.31 |
| Prkaa2 | −1.17 | −1.22 | −1.31 |
| Elovl2 | −1.91 | −1.31 | −1.34 |
| Rnpep | 1.13 | −1.07 | −1.36 |
| Acsbg2 | −1.55 | −1.14 | −1.37 |
| Ptgcs | −1.05 | −1.30 | −1.38 |
| Prkag3 | −1.24 | 1.34 | −1.45 |
| Hpgd | 1.17 | −1.14 | −2.59 |

A study of the expression of 20,341 genes in cardiac tissue revealed that 2,829 genes exhibited statistically significant differences in expression (P<0.01). Of these, 7% (approximately 189 genes) exhibited altered expression in animals subjected only to calorie reduced diets; 8% (approximately 226 genes) exhibited altered expression in animals receiving only resveratrol; no additional genes exhibited altered expression in animals that received resveratrol and which were subjected to calorie reduced diets. In contrast, 61% of the 20,341 genes (approximately 1,729 genes) exhibited altered expression in animals receiving only compounds of the present invention (e.g., Longevinex®); an additional 2% of the genes (approximately 56 genes) exhibited altered expression in animals that had received compounds of the present invention (e.g., Longevinex®) and which had been subjected to calorie reduced diets; an additional 21% of the genes (approximately 594 genes) exhibited altered expression in animals that had received compounds of the present invention (e.g., Longevinex®) and resveratrol; an additional 1% of the genes (approximately 28 genes) exhibited altered expression in animals that had received compounds of the present invention (e.g., Longevinex®), resveratrol and which had been subjected to calorie reduced diets.

The above data demonstrates that compounds of the present invention (e.g., Longevinex®) were effective in modulating gene expression in hear tissue to an extent surpassing even that of calorie restriction. Similar effects have been observed in non-heart tissue. A study of the expression of 20,341 genes in brain tissue revealed that 3,572 genes exhibited statistically significant differences in expression (P<0.01). Of these, 124 genes exhibited altered expression in animals subjected only to calorie reduced diets; 424 genes exhibited altered expression in animals receiving only resveratrol; 10 genes exhibited altered expression in animals that received resveratrol and which were subjected to calorie reduced diets. In contrast, 2,560 genes exhibited altered expression in animals receiving only compounds of the present invention (e.g., Longevinex®); 19 additional genes exhibited altered expression in animals that had received compounds of the present invention (e.g., Longevinex®) and which had been subjected to calorie reduced diets; 430 additional genes exhibited altered expression in animals that had received compounds of the present invention (e.g., Longevinex®) and resveratrol; 5 additional genes exhibited altered expression in animals that had received compounds of the present invention (e.g., Longevinex®), resveratrol and which had been subjected to calorie reduced diets.

### Example 4

### Model Mechanism of Action of the Compositions of the Present Invention

The compounds of the present invention were thus found to greatly exceed the modulation of gene expression observed upon calorie restriction and to alter the expression of genes in key patyhways of lipid metabolism, glucose metabolism, oxidative phosphorylation, the Kreb's cycle, ATP synthesis and fatty acid β oxidation. In summary, the compounds of the present invention were found to have a greater specific activity than resveratrol alone, both in terms of the number of genes and the number of different biochemical pathways affected. The results are significant since caloric restriction (CR) is considered the unequivocal method of prolonging life in all forms of life. Generally, reduction of 50% of caloric intake doubles the lifespan of any organism. The above-described experiments demonstrate that the compositions of the present invention exert a more powerful influence over genome expression than resveratrol or CR, and marks the first time any technology has been shown to exceed the effects of CR. Furthermore, the compositions of the present invention were found to influence genome expression at an earlier stage of life than CR (which requires a life-long adherence to a CR diet to differentiate genes).

Without intending to be bound by any mechanism of action, the above results suggest that the compounds of the present invention act by enhancing the activity of the forkhead Foxo1 (daf-16, dFoxO) transcription factor (FIG. 5). Studies in model organisms has shown that Foxo1 mediates lifespan expression by enhancing gene expression. Insulin/IGF-1 signaling phosphorylates Foxo1, thereby causing it to be excluded from the nucleus and downregulating its actions. The compounds of the present invention decrease insulin and IGF-1 signaling thereby decreasing Foxo1 phosphorylation. Consistent with this model are the observations that the insulin receptor signaling pathway (e.g., GO:008286; genes lde, lgfbp4, and lgfbp6) is affected by the compounds of the present invention. Expression of Foxo1 is increased by 1.75 fold. The compounds of the present invention mediate decreased glycolysis and increased gluconeogenesis (e.g., GO:0006006), enhanced Pgc-1α expression (thereby leading to stimulation of Pdk4 expression (e.g., a 1.94 fold increase in Ppargc1α and a 3.25 fold increase in Pdk4), increased expression of lipid metabolism genes (e.g., a 2.79 fold increase in Ucp3, 1.49 fold increase in Cpt1a, and a 1.45 fold increase in Cpt1b). Lipid and fatty acid metabolism genes GO:0006629 and GO:0006635 are uniquely affected by the compounds of the present invention. The compounds of the present invention thus exert a more pronounced favorable effect on key processes affected by calorie restriction and resveratrol (e.g., chromatin remodeling, transcription from RNA polymerase II promoter, and the ubiquitin cycle. Genes GO:0006333 and GO:0006367 are uniquely affected by the compounds of the present invention; Gene GO:0006512 is affected by resveratrol and Longevinex®. Thus, in sum, a proposed mechanism of action is that the compositions of the present invention deliver resveratrol to cells, where it passes through cell walls, enters the cytoplasm, and facilitates the translocation of Foxo1 gene into the cell nucleus, which produces the longevity effects.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-20. (canceled)

21. A method of treating macular degeneration, comprising administering to said individual with macular degeneration, a therapeutically effective amount of a resveratrol-containing composition comprising:
trans-resveratrol; and
a metal chelating agent;
wherein said therapeutically effective amount is sufficient to ameliorate a symptom associated with macular degeneration.

22. The method of claim 21, wherein the metal chelating agent is phytic acid.

23. The method of claim 21, wherein the composition further comprises quercetin.

24. The method of claim 21, wherein the composition further comprises one or more of fisetin, myricetin, or kaempferol.

25. The method of claim 21, wherein the composition further comprises Vitamin D.

26. The method of claim 21, wherein the composition further comprises hyaluronic acid.

27. The method of claim 21, wherein the composition further comprises hyaluronic acid and vitamin D.

28. The method of claim 21, wherein said trans-resveratrol is encapsulated to thereby substantially preserve the activity of said composition from loss of activity due to exposure of said trans-resveratrol to light or oxygen.

29. The method of claim 28, wherein encapsulation of resveratrol is a microencapsulation.

30. The method of claim 21, wherein the composition further comprises one or more of vitamin E, lutein and alpha lipoic acid.

31. The method of claim 21, wherein the composition ameliorates a symptom of macular degeneration by one or more of:
(a) limiting oxidation in retinal tissues;
(b) inhibiting accumulation of lipofuscin deposits;
(c) inhibiting formation of drusen; and
(d) limiting calcification to retinal tissues.

32. A method of treating macular degeneration, comprising administering to said individual with macular degeneration, a capsule comprising:
trans-resveratrol present in an amount of 100 mg per capsule;
phytic acid present in an amount of 75 mg per capsule; and
quercetin dihydrate present in an amount of 25 mg per capsule.

33. The method of claim 32, wherein the composition further comprises rice bran oil in an amount of 380 mg per capsule.

34. The method of claim 32, wherein the composition further comprises an emulsifier in an amount of 55 mg per capsule.

35. The method of claim 32, wherein the composition is a capsule that is administered orally.

36. A composition for the treatment of macular degeneration, comprising administering to said individual with macular degeneration, said composition comprising:
trans-resveratrol; and
a metal chelating agent;
wherein said composition is administered in a therapeutically effective amount sufficient to ameliorate a symptom associated with macular degeneration, and wherein said composition is microencapsulated.

37. The composition of claim 36, wherein the metal chelating agent is phytic acid.

38. The composition of claim 36, wherein the composition further comprises quercetin.

39. The composition of claim 36, wherein the composition further comprises hyaluronic acid.

40. The composition of claim 36, wherein the composition further comprises Vitamin D.
